(19) United States
(12) Patent Application Publication
(10) Pub. No.: US 2004/0176431 A1
(43) Pub. Date: Sep. 9, 2004

(54) Antiproliferative 2-(sulfo-phenyl)-aminothiazole derivatives and pharmaceutical compositions, and methods for their use

(75) Inventors: Wesley K.M. Chong, Encinitas, CA (US); Shao Song Chu, Encinitas, CA (US); Rohit K. Duvadie, San Diego, CA (US); James Na, San Diego, CA (US); Lana Schaffer, San Diego, CA (US); Yi Yang, San Diego, CA (US); Lin Li, San Diego, CA (US)

Correspondence Address: AGOURON PHARMACEUTICALS, INC., 10350 NORTH TORREY PINES ROAD, LA JOLLA, CA, 92037, US

(73) Assignee: Pfizer, Inc.

(21) Appl. No.: 10/776,450

(22) Filed: Feb. 11, 2004

Related U.S. Application Data
(60) Provisional application No. 60/447,329, filed on Feb. 12, 2003.

(51) Int. Cl. A61K 31/427; A61K 31/426; C07D 417/02

(52) U.S. Cl. 514/370; 548/181; 548/194

(57) ABSTRACT

Aminothiazole compounds substituted with sulfur-containing groups are represented by the Formula (I), and their pharmaceutically acceptable salts, prodrugs, active metabolites, and pharmaceutically acceptable salts of said metabolites are described.

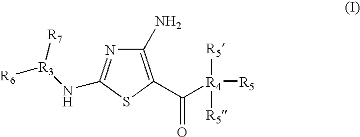

These agents modulate and/or inhibit the cell proliferation and activity of protein kinases and are useful as pharmaceuticals for treating malignancies and other disorders.

### CROSS-REFERENCES TO RELATED APPLICATIONS

This application claims the benefit of U.S. Provisional Application Serial No. 60/447,329, filed Feb. 12, 2003, which is hereby incorporated by reference in its entirety.

### FIELD OF THE INVENTION

This invention is directed to compounds with {2-(sulfo-phenyl)-aminothiazole nuclei that mediate and/or inhibit proliferation, and to pharmaceutical compositions containing such compounds. The invention is also directed to the therapeutic or prophylactic use of such compounds and compositions, and to methods of treating cancer, viral, microbial, and/or parasitic colonization/infection, as well as other disease states associated with unwanted proliferation, by administering effective amounts of such compounds.

### BACKGROUND OF THE INVENTION

Cell proliferation occurs in response to various stimuli and may stem from de-regulation of the cell division cycle (or cell cycle), the process by which cells multiply and divide. Hyperproliferative disease states, including cancer, are characterized by cells rampantly winding through the cell cycle with uncontrolled vigor due to, for example, damage to the genes that directly or indirectly regulate progression through the cycle. Thus, agents that modulate the cell cycle, and thus hyperproliferation, could be used to treat various disease states associated with uncontrolled or unwanted cell proliferation.

Mechanisms of cell proliferation are under active investigation at cellular and molecular levels. At the cellular level, de-regulation of signaling pathways, loss of cell cycle controls, unbridled angiogenesis or stimulation of inflammatory pathways are under scrutiny, while at the molecular level, these processes are modulated by various proteins, among which protein kinases are prominent suspects. Overall abatement of proliferation may also result from programmed cell death, or apoptosis, which is also regulated via multiple pathways, some involving proteolytic enzyme proteins.

Among the candidate regulatory proteins, protein kinases are a family of enzymes that catalyze phosphorylation of the hydroxyl group of specific tyrosine, serine or threonine residues in proteins. Typically, such phosphorylation dramatically perturbs the function of the protein, and thus protein kinases are pivotal in the regulation of a wide variety of cellular processes.

For example, without wishing to be bound to a particular theory, it is believed that as inhibitors of protein kinases, such as, for example, cyclin dependent kinases (“CDK”), the inventive agents can modulate the level of cellular RNA and DNA synthesis and therefore are expected to be useful in the treatment of viral infections such as HIV, human papilloma virus, herpesvirus, Epstein-Barr virus, adenovirus, Sindbis virus, poxvirus and the like. (See Schang, et al, J. Virol. 74, 2107-2120 (2000)). Additionally, CDK5 has been implicated in the phosphorylation of tau protein, suggesting potential methods of treating or preventing Alzheimer's disease (Hosoi, et al, J. Biochem. (Tokyo), 117, 741-749 (1995)). CDKs are serine-threonine protein kinases that play critical roles in regulating the transitions between different phases of the cell-cycle, such as the progression from a quiescent stage in G₁ (the gap between mitosis and the onset of DNA replication for a new round of cell division) to S (the period of active DNA synthesis), or the progression from G₂ to M phase, in which active mitosis and cell-division occurs. CDK complexes are formed through association of a regulatory cyclin subunit (e.g., cyclin A, B1, B2, D1, D2, D3, and E) and a catalytic kinase subunit (e.g., CDK1, CDK2, CDK4, CDK5, and CDK6). As the name implies, the CDKs display an absolute dependence on the cyclin subunit in order to phosphorylate their target substrates, and different kinase/cyclin pairs function to regulate progression through specific phases of the cell-cycle.

A large number of small molecule ATP-site antagonists have thus far been identified as CDK inhibitors. (See, Webster, Exp. Opin. Invest. Drugs, 7, 865-887 (1998); Stover, et al, Curr. Opin. Drug Disc. Dev., 2, 274-285 (1999); Gray, et al, Curr. Med. Chem., 6, 859-875 (1999); Sielecki, et al, J. Med. Chem.; 43,1-18 (2000); Crews, et al, Curr. Opin. Chem. Biol., 4, 47-53 (2000); Buolamwini, Curr. Pharm. Des. 6, 379-392 (2000); Rosania, Exp. Opin. Ther. Patents, 10, 215-230 (2000), Toogood, Med. Res. Rev., 21, 487-498 (2001), and Kimball, et al, Ann. Rep. Med. Chem., 36, 139-148 (2001).

There is still a need, however, for more potent inhibitors of protein kinases. Moreover, as is understood by those skilled in the art, it is desirable for kinase inhibitors to possess both high affinity for the target kinase as well as high selectivity versus other protein kinases.

Among others, the following patent publications disclose thiazole compounds: WIPO International Publication Nos. WO 99/21845 and WO00/75120 disclose 2,4-diaminothiazoles used as CDK or kinase inhibitors respectively. Very recently, Roche disclosed diaminothiazoles in WIPO International Publication No. WO 02/57261. After an early report of 2,4-diaminothiazoles in Gewald, et al, J. Prakt. Chem., 35, 97-104 (1967), subsequent modified preparations—prior to the patents above—were seen in Rajasekharan, et al, Synthesis, 353-355 (1986), Jenardanan, et al, Syn. Comm., 27, 3457-3462 (1997), and Binu, et al, Org. Prep. Proced. Intl., 30, 93-96 (1998). Yet another extension of the methodology recently appeared in Devi, et al, Syn. Comm., 32, 1523-1528 (2002), which alluded to the preparation of a combinatorial library of 2,4-diaminothiazoles. This was realized from another recent modification from Masquelin, et al, Tetrahedron 57, 153-156 (2001), which was adapted to solid support in Baer, et al, J. Comb. Chem., 3, 16-19 (2001). WIPO International Publication No. WO 99/62890 discloses isothiazoles used as anticancer agents; WO 98/04536 describes thiazoles used as protein kinase C inhibitors; EP 816362A (1998) discloses thiazoles used principally for dopamine D4 receptor antagonists. Aminothiazoles were reported in U.S. Pat. No. 6,262,096, WIPO International Publication Nos. WO 01/44241, WO 01/44242, and aminobenzothiazoles in WO 99/24035. WIPO International Publication No. WO 00/17175 describes other aminothiazoles used as p38 mitogen-activated protein (MAP) kinase inhibitors, and WO 00/26202, WO 00/26203, and U.S. Pat. No. 6,114,365 describe aminothiazoles and ureidothiazoles used as anti-tumor agents. WIPO International Publication Nos. WO 99/21845 and WO 03/04467 describe aminothiazole benzamide derivatives with anti-proliferative activity. The present invention however is based on the discovery that aminothiazole compounds having a sulfur-containing group are more potent than the corresponding aminothiazole compounds without the sulfur-containing group. Thus, the inventive compounds show generally more potent cell growth inhibition than the compounds described in WIPO International Publication Nos. WO 99/21845 and WO 03/04467.

### SUMMARY OF INVENTION

The present invention relates to compounds of Formula (I), which prevent cellular proliferation. The compounds are also useful for mediating the activity of protein kinases. More particularly, the compounds are useful as anti-angiogenesis agents and as agents for modulating and/or inhibiting the activity of various enzymes, for example protein kinases, thus providing treatments for cancer or other diseases associated with uncontrolled (or abnormal) cellular proliferation.

In one embodiment, the invention relates to compounds of the Formula (I):

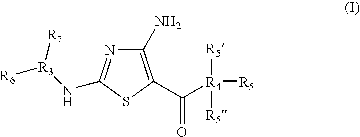

wherein:

R₃ is a monocycle selected from the group consisting of C₃-C₁₀ cycloalkyl, 3-10 membered heterocycloalkyl, aryl and 3-10 membered heteroaryl;

R₄ is a moiety selected from the group consisting of C₂-C₁₄ alkyl, C₃-C₁₀ cycloalkyl, 3-10 membered heterocycloalkyl, aryl and 3-10 membered heteroaryl, wherein R₄ is unsubstituted or substituted with 1 to 4 R₁₀ groups;

R₅ is a moiety selected from the group consisting of hydroxyl, halo, C₁-C₁₄ alkyl, C₁-C₁₄ alkoxyl, acyl, amide and nitro;

R₅′ and R₅′ are independently selected from hydrogen, hydroxyl, halo, C₁₋₁₄ alkyl, C₁-C₁₄ alkoxyl, acyl, amide, amino, acetamido and nitro;

R₆ is a group selected from the following formulae:

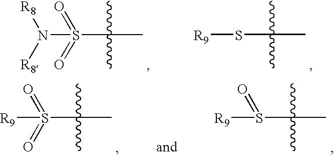

wherein:

R₈ is hydrogen, C₁-C₃ alkyl, C₃-C₁₀ cycloalkyl, or C₁-C₁₄ alkoxyl;

R_{8′} is an C₃-C₁₄ alkyl, 2 to 9 membered heteroalkyl, acyl, C₁-C₃ alkyl-nitrile, C₁-C₃ alkyl-carboxamide, C₁-C₄ alkyl-heterocycloalkyl, C₁-C₄ alkyl-aryl, C₁-C₄ alkyl-heteroaryl, C₃-C₁₀ cycloalkyl, 3-10 membered heterocycloalkyl, aryl or 3-10 membered heteroaryl, or together with R₈ cyclizes to form an unsubstituted or substituted C₃-C₁₀ cycloalkyl, 3-10 membered heterocycloalkyl, aryl or 3-10 membered heteroaryl, with the proviso that R is not

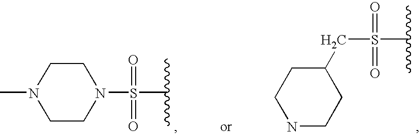

and wherein R_{8′} is unsubstituted or substituted with 1 to 4 R₁₀ groups;

R₉ is hydrogen, or a moiety selected from the group consisting of an C₁-C₉ alkyl, C₂-C₉ alkenyl, 2-9 membered heteroalkenyl, C₁-C₉ alkylamide, C₁-C₉ alkyl-carboxamide, 2-9 membered heteroalkyl, C₁-C₄ alkyl-cycloalkyl, C₁-C₄ alkyl-heterocycloalkyl, C₁-C₄ alkyl-aryl, C₁-C₄ alkyl-heteroaryl, C₃-C₁₀ cycloalkyl, 3-10 membered heterocycloalkyl, aryl and 3-10 membered heteroaryl, with the proviso that R₆ is not

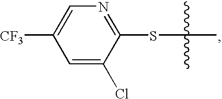

and wherein R₉ is unsubstituted or substituted with 1 to 4 R₁₀ groups;

R₇ is a moiety selected from the group consisting of hydrogen, hydroxyl, halo, C₁-C₁₄ alkyl, C₁-C₁₄ alkoxyl, acyl, amide and nitro;

wherein each R₁₀ is independently selected from halo, cyano, nitro, trifluoromethoxy, trifluoromethyl, azido, hydroxyl, C₁-C₆ alkoxyl, C₁-C₁₀ alkyl, C₂-C₆ alkenyl, C₂-C₆ alkynyl, —C(O)Rₐ, —C(O)OR_{b}, —OC(O)R_{b}, —NR_{b}C(O)R_{c}, —C(O)NR_{b}R_{c}, —NR_{b}R_{c}, —NR_{b}OR_{c}, —S(O)ⱼ(C₁-C₆ alkyl) wherein j is an integer from 0 to 2, —(CR_{d}Rₑ)ₜ(C₃-C₁₀ cycloalkyl), —(CR_{d}Rₑ)ₜ(aryl), —(CR_{d}Rₑ)ₜ(4-10 membered heterocycloalkyl), —(CR_{d}Rₑ)ₜ(4-10 membered heteroaryl), —(CR_{d}Rₑ)_{q}C(O)(CR_{d}Rₑ)ₜ(C₃-C₁₀ cycloalkyl), —(CR_{d}Rₑ)_{q}C(O)(CR_{d}Rₑ)ₜ(aryl), —(CR_{d}Rₑ)_{q}C(O)(CR_{d}Rₑ)ₜ(4-10 membered heterocycloalkyl),

—(CR_{d}Rₑ)_{q}C(O)(CR_{d}Rₑ)ₜ(4-10 membered heteroaryl), —(CR_{d}Rₑ)ₜO(CR_{d}Rₑ)_{q}(C₃-C₁₀ cycloalkyl), —(CR_{d}Rₑ)ₜO(CR_{d}Rₑ)_{q}(aryl), —(CR_{d}Rₑ)ₜO(CR_{d}Rₑ)_{q}(4-10 membered heterocycloalkyl), —(CR_{d}Rₑ)ₜO(CR_{d}Rₑ)_{q}(4-10 membered heteroaryl), —(CR_{d}Rₑ)_{q}SO₂(CR_{d}Rₑ)ₜ(C₃-C₁₀ cycloalkyl), —(CR_{d}Rₑ)_{q}SO₂(CR_{d}Rₑ)ₜ(aryl), and —(CR_{d}Rₑ)_{q}SO₂(CR_{d}Rₑ)ₜ(4-10 membered heterocycloalkyl), —(CR_{d}Rₑ)_{q}SO₂(CR_{d}Rₑ)ₜ(4-10 membered

heteroaryl), wherein Rₐ is selected from the group consisting of halo, hydroxyl, —NR_{d}Rₑ C₁-C₆ alkyl, trifluoromethyl, C₁-C₆ alkoxyl, and trifluoromethoxy, R_{b} and R_{c} are independently selected from H, C₁-C₆ alkyl, —(CR_{d}Rₑ)ₜ(C₃-C₁₀ cycloalkyl), —(CR_{d}Rₑ)ₜ(aryl), —(CR_{d}Rₑ)ₜ(4-10 membered heterocycloalkyl), and

—(CR_{d}Rₑ)ₜ(4-10 membered heteroaryl), wherein q and t are each independently an integer from 0 to 5, R_{d} and Rₑ are independently H or C₁-C₆ alkyl, wherein 1 or 2 ring carbon atoms of the heterocyclic and heteroaryl moieties of the foregoing R₁₀ groups are unsubstituted or substituted with an oxo (═O) moiety, and the alkyl, alkenyl, alkynyl, aryl and heterocyclic and heteroaryl moieties of the foregoing R₁₀ groups are unsubstituted or substituted with 1 to 3 substituents independently selected from halo, cyano, nitro, trifluoromethyl, trifluoromethoxy, azido, —OR_{b}, —C(O)R_{b}, —C(O)OR_{b}, —NR_{b}C(O)R_{c}, —C(O)NR_{b}R_{c}, —NR_{b}R_{c}, —NR_{b}OR_{c}, C₁-C₆ alkyl, C₂-C₆ alkenyl, C₂-C₆ alkynyl, —(CR_{d}Rₑ)ₜ(C₃-C₁₀ cycloalkyl), —(CR_{d}Rₑ)ₜ(aryl), —(CR_{d}Rₑ)ₜ(4-10 membered heterocycloalkyl), and

—(CR_{d}Rₑ)ₜ(4-10 membered heteroaryl);

and wherein any of the above-mentioned substituents comprising a CH₃ (methyl), CH₂ (methylene), or CH (methane) group which is not attached to a halogeno, SO or SO₂ group or to a N, O, or S is unsubstituted or substituted with a substituent from the group selected from hydroxyl, halo, C₁-C₄ alkyl, C₁-C₄ alkoxyl and —NR_{d}Rₑ wherein R_{d} and Rₑ are as defined above;

or a pharmaceutically acceptable salt of a compound of the Formula (I), or a multimer, prodrug or pharmaceutically active metabolite of a compound of the Formula (I) or pharmaceutically acceptable salt thereof.

The invention is also directed to a pharmaceutical composition comprising an effective amount of an agent to inhibit cellular proliferation and a pharmaceutically acceptable carrier, said agent being selected from the group consisting of compounds, multimers, pharmaceutically acceptable salts, prodrugs, and active metabolites of the

compounds of Formula (I). Advantageous methods of making the compounds of the Formula (I) are also described.

The invention also relates to a method of inhibiting a CDK selected from CDK2, CDK4, CDK6 or CDK complex, comprising administering an effective amount of a compound of Formula (I), or a multimer, pharmaceutically acceptable salt, prodrug, or active metabolite thereof.

The invention also relates to a method of treating cellular proliferative diseases, comprising administering an effective amount of a compound of formual (I), or a multimer, pharmaceutically acceptable salt, prodrug, or active metabolite thereof.

The invention also relates to a method of treating proliferative diseases such as cancer, autoimmune diseases, viral diseases, fungal diseases, neurodegenerative disorders and cardiovascular disease.

In a preferred embodiment, the invention relates to compounds having Formula (II):

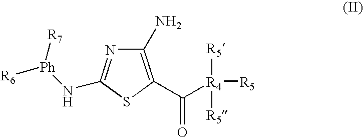

wherein:

R₄, R₅, R₅′, R₅″, R₆ and R₇ are as defined above, and Ph is phenyl.

In a preferred embodiment, the invention relates to compounds having Formula (III):

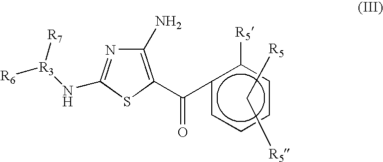

wherein:

R₃, R₅, R₅′, R₅″, R₆ and R₇ are as defined above.

In a preferred embodiment, the invention relates to compounds having Formula (IV):

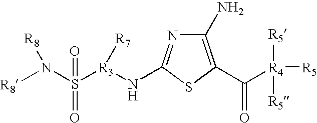

wherein:

R₃, R₅, R₅′, R₅″, R₇, R₈ and R_{8′} are defined above.

The preferred compounds of the invention are listed in Table 1.

TABLE 1

| Example | IUPAC Name | Compound Structure |
|---|---|---|
| Example A(1) | {4-Amino-2-[4-(piperazine-1- sulfonyl)-phenylamino]-thiazol- 5-yl}-(2,6-difluoro-phenyl)- methanone | 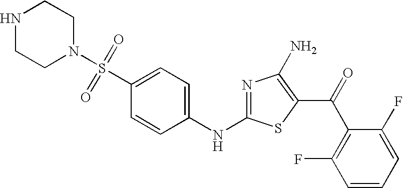 |
| Example A(2) | Example A(2): 4-[4-Amino-5- (2,6-difluoro-benzoyl)-thiazol- 2-ylamino]-N-(2- dimethylamino-ethyl)- benzenesulfonamide | 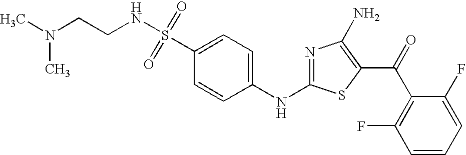 |
| Example A(3) | 4-[4-Amino-5-(2,6-difluoro- benzoyl)-thiazol-2-ylamino]-N- methoxymethyl- benzenesulfonamide | 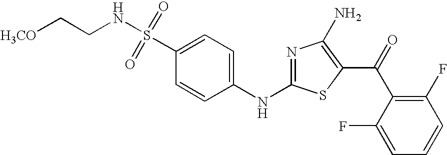 |
| Example A(4) | 4-[4-Amino-5-(2,6-difluoro- benzoyl)-thiazol-2-ylamino]-N- (2-hydroxy-ethyl)- benzenesulfonamide | 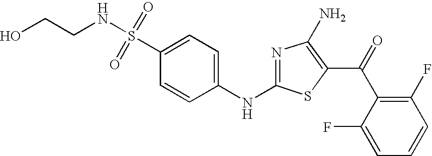 |
| Example A(5) | 4-[4-Amino-5-(2,6-difluoro- benzoyl)-thiazol-2-ylaminO]-N- (4-hydroxy-butyl)- benzenesulfonamide | 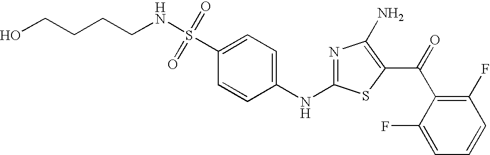 |
| Example A(6) | 4-[4-Amino-5-(2,6-difluoro- benzoyl)-thiazol-2-ylamino]-N- [2-(2-hydroxy-ethoxy)-ethyl]- benzenesulfonamide | 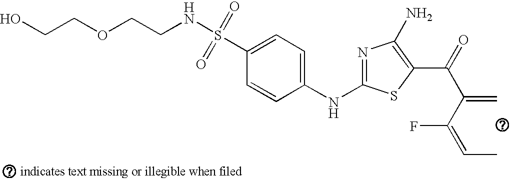 |
| Example A(7) | 4-[4-Amino-5-(2,6-difluoro- benzoyl)-thiazol-2-ylamino]-N- (2,5-dichloro-benzyl)- benzenesulfonamide | 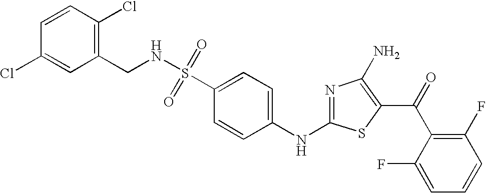 |
| Example A(8) | 4-[4-Amino-5-(2,6-difluoro- benzoyl)-thiazol-2-ylamino]-N- (3-pyrrolidin-1-yl-propyl)- benzenesulfonamide | 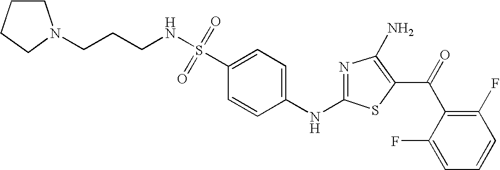 |
| Example A(9) | 4-[4-Amino-5-(2,6-difluoro- benzoyl)-thiazol-2-ylamino]-N- (2-phenylamino-ethyl)- benzenesulfonamide | 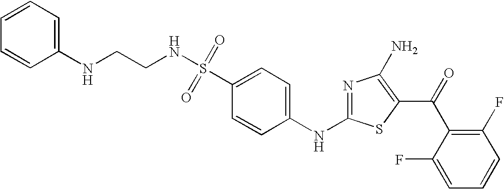 |
| Example A(10) | 4-(4-Amino-5-(2,6-difluoro- benzoyl)-thiazol-2-ylamino]-N- (3-isopropoxy-propyl)- benzenesulfonamide | 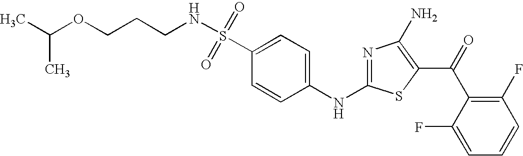 |
| Example A(11) | 4-[4-Amino-5-(2,6-difluoro- benzoyl)-thiazol-2-ylamino]-N- (5-methyl-furan-2-ylmethyl)- benzenesulfonamide | 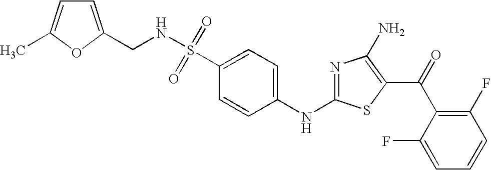 |
| Example A(12) | 4-[4-Amino-5-(2,6-difluoro- benzoyl)-thiazol-2-ylamino]-N- (5-hydroxy-1,5-dimethyl- hexyl)-benzenesulfonamide | 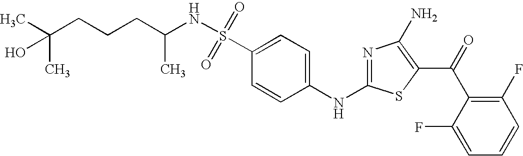 |
| Example A(13) | 4-[4-Amino-5-(2,6-difluoro- benzoyl)-thiazol-2-ylamino]-N- (3-diethylamino-propyl)- benzenesulfonamide | 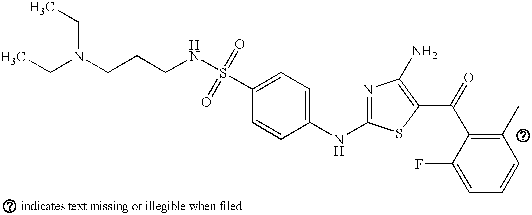 |
| Example A(14) | 4-[4-Amino-5-(2,6-difluoro- benzoyl)-thiazol-2-yiamino]-N- (3-piperidin-1-yl-propyl)- benzenesulfonamide | 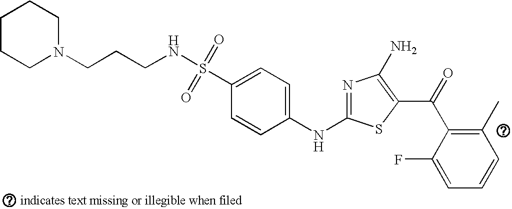 |
| Example A(15) | 4-(4-Amino-5-(2,6-difluoro- benzoyl)-thiazol-2-ylamino]-N- (3-(2RS-methyl-piperidin-1-yl)- propyl]-benzenesulfonamide | 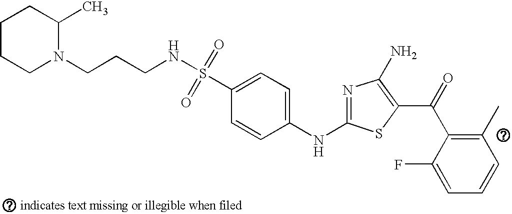 |
| Example A(16) | (4-Amino-2-{4-[4-(2-hydroxy- ethyl)-piperidine-1-sulfonyl]- phenylamino}-thiazol-5-yl)- (2,6-difluoro-phenyl)- methanone | 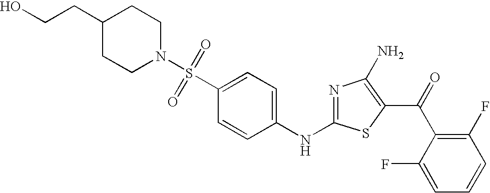 |
| Example A(17) | 4-[4-Amino-5-(2,6-difluoro- benzoyl)-thiazol-2-ylamino]-N- (2-isopropoxy-ethyl)- benzenesulfonamide | 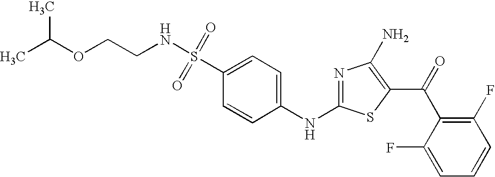 |
| Example A(18) | 4-[4-Amino-5-(2,6-difiuoro- benzoyl)-thiazol-2-ylamino]-N- (2-p-tolyl-ethyl)- benzenesulfonamide | 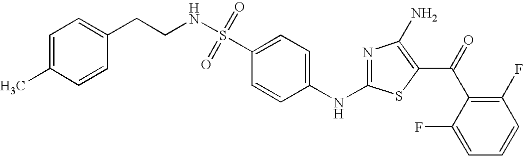 |
| Example A(19) | 4-[4-Amino-5-(2,6-difluoro- benzoyl)-thiazol-2-yiamino]-N- (2-ethylsulfanyl-ethyl)- benzenesulfonamide | 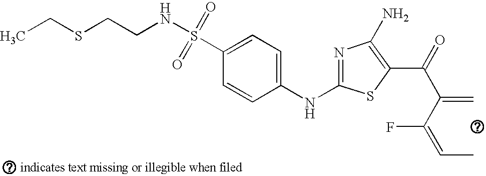 |
| Example A(20) | 4-[4-Amino-5-(2,6-difluoro- benzoyl)-thiazol-2-ylamino]-N- [2-(4-fluoro-phenyl)-ethyl]- benzenesulfonamide | 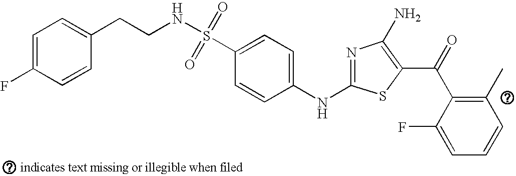 |
| Example A(21) | 4-[4-Amino-5-(2,6-difluoro- benzoyl)-thiazol-2-ylamino]-N- (3-dimethylamino-propyl)- benzenesulfonamide | 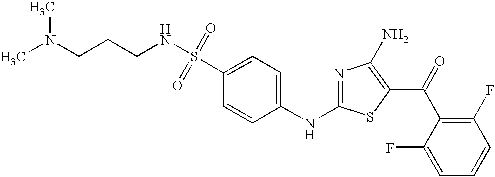 |
| Example A(22) | 4-[4-Amino-5-(2,6-difluoro- benzoyl)-thiazol-2-ylamino]-N- furan-2-ylmethyl- benzenesulfonamide | 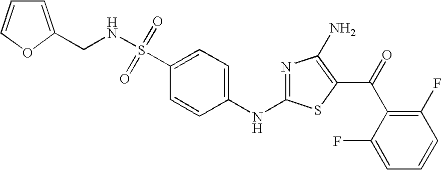 |
| Example A(23) | 4-[4-Amino-5-(2,6-difluoro- benzoyl)-thiazol-2-ylamino]-N- (2-thiophen-2-yl-ethyl)- benzenesulfonamide | 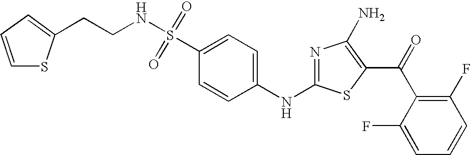 |
| Example A(24) | 4-[4-Amino-5-(2,6-difluoro- benzoyl)-thiazol-2-ylamino]-N- (2-pyridin-2-yl-ethyl)- benzenesulfonamide | 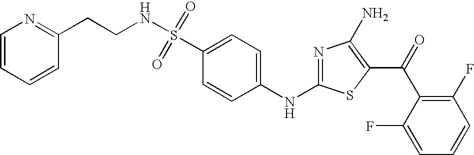 |
| Example A(25) | 4-[4-Amino-5-(2,6-difluoro- benzoyl)-thiazol-2-ylamino]-N- [3-(2-oxo-pyrrolidin-1-yl)- propyl]-benzenesulfonamide | 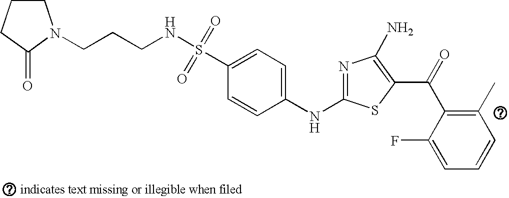 |
| Example A(26) | 4-[4-Amino-5-(2,6-difluoro- benzoyl)-thiazol-2-ylamino]-N- (4-diethylamino-butyl)- benzenesulfonamide | 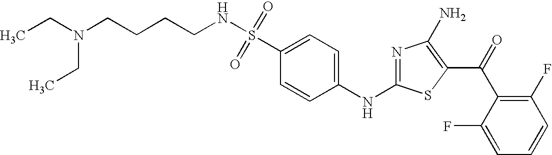 |
| Example A(27) | 4-[4-Amino-5-(2,6-difluoro- benzoyl)-thiazol-2-ylamino]-N- thiophen-2-ylmethyl- benzenesulfonamide | 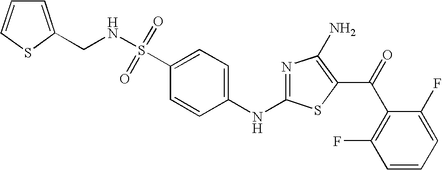 |
| Example A(28) | 4-[4-Amino-5-(2,6-difluoro- benzoyl)-thiazol-2-ylamino]-N- (5-hydroxy-pentyl)- benzenesulfonamide | 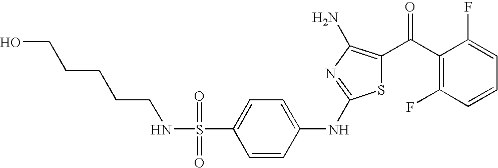 |
| Example A(29) | 4-[4-Amino-5-(2,6-difluoro- benzoyl)-thiazol-2-ylamino]-N- (5-methyl-thiophen-2- ylmethyl)-benzenesulfonamide | 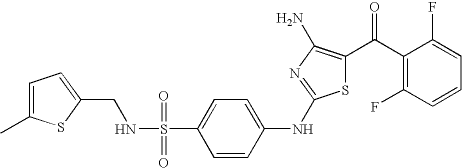 |
| Example A(30) | 4-[4-Amino-5-(2,6-difluoro- benzoyl)-thiazol-2-ylamino]-N- (1-(5-methyl-furan-2-yl)-ethyl]- benzenesulfonamide | 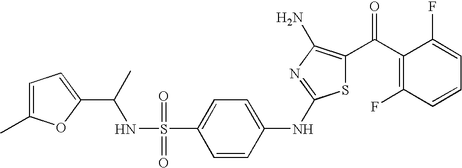 |
| Example A(31) | 4-[4-Amino-5-(2,6-difluoro- benzoyl)-thiazol-2-ylamino]-N- (2-propoxy-ethyl)- benzenesulfonamide | 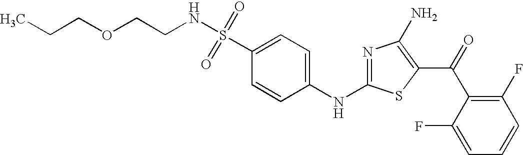 |
| Example A(32) | 4-[4-Amino-5-(2,6-difluoro- benzoyl)-thiazol-2-ylamino]-N- (3-phenyl-propyl)- benzenesulfonamide | 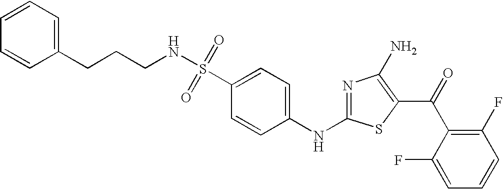 |
| Example A(33) | 4-[4-Amino-5-(2,6-difluoro- benzoyl)-thiazol-2-ylamino]-N- [2-(3-chloro-phenyl)-ethyl]- benzenesulfonamide | 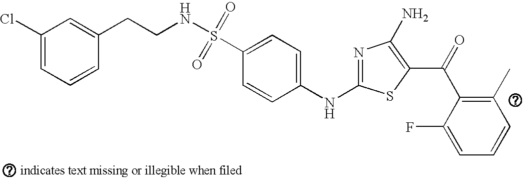 |
| Example A(34) | 4-[4-Amino-5-(2,6-difluoro- benzoyl)-thiazol-2-ylamino]-N- benzofuran-2-ylmethyl- benzenesulfonamide | 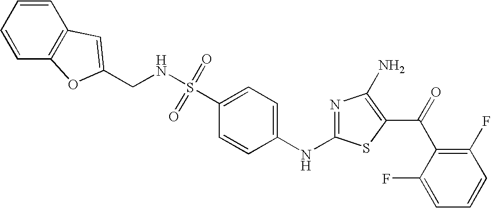 |
| Example A(35) | {4-[4-Amino-5-(2,6-difluoro- benzoyl)-thiazol-2-ylamino]- benzenesulfonylamino}-acetic Acid Ethyl Ester | 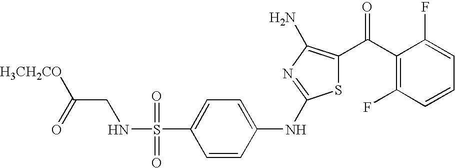 |
| Example A(36) | 4-(4-Amino-5-(2,6-difluoro- benzoyl)-thiazol-2-ylamino]-N- (5-hydroxy-5-methyl-hexyl)- benzenesulfonamide | 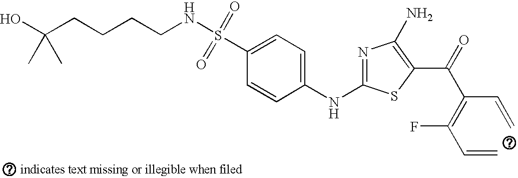 |
| Example A(37) | 4-[4-Amino-5-(2,6-difluoro- benzoyl)-thiazol-2-ylamino]-N- (5-methyl-hexyl)- benzenesulfonamide | 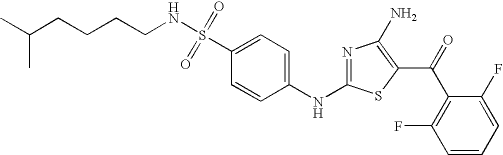 |
| Example A(38) | 4-[4-Amino-5-(2,6-difluoro- benzoyl)-thiazol-2-ylamino]-N- (1-methyl-1H-imidazol-5- ylmethyl)-benzenesulfonamide Trifluoroacetic Acid Salt | 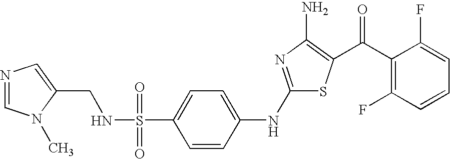 |
| Example B(1) | 4-[4-Amino-5-(2,4,6-trifluoro- benzoyl)-thiazol-2-ylamino]-N- (5-methyl-furan-2-ylmethyl)- benzenesulfonamide | 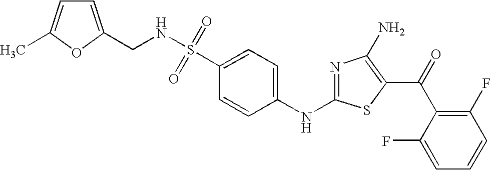 |
| Example B(2) | 4-[4-Amino-5-(2,4,6-trifluoro- benzoyl)-thiazol-2-ylamino]-N- (5-hydroxy-1,5-dimethyl- hexyl)-benzenesulfonamide | 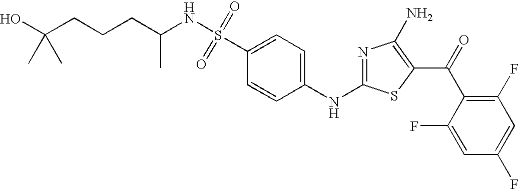 |
| Example C(1) | 4-[4-Amino-5-(2,6-difluoro- benzoyl)-thiazol-2-ylamino]-N- phenyl-benzenesulfonamide | 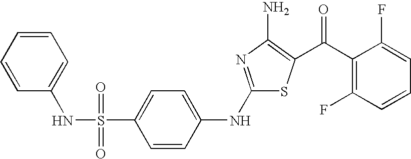 |
| Example D(1) | 4-[4-Amino-5-(2,6-difluoro- benzoyl)-thiazol-2-ylamino]-N- piperidin-3-ylmethyl- benzenesulfonamide | 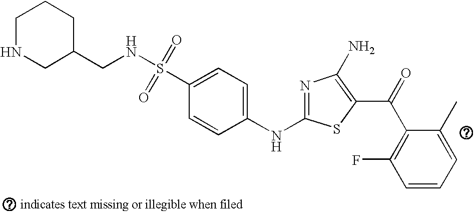 |
| Example D(2) | 4-[4-Amino-5-(2,6-difluoro- benzoyl)-thiazol-2-ylaminol-N- piperidin-2-ylmethyl- benzenesulfonamide | 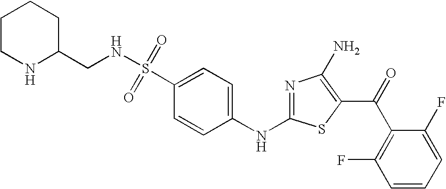 |
| Example D(3) | 4-[4-Amino-5-(2,6-difluoro- benzoyl)-thiazol-2-ylamino]-N- (2-methylamino-ethyl)- benzenesulfonamide | 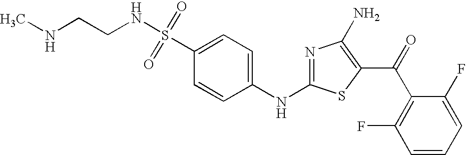 |
| Example E(1) | 4-(4-Amino-5-(2,6-difluoro- benzoyl)-thiazol-2-ylamino]-N- (4-methyl-thiazol-2-yl)- benzenesulfonamide | 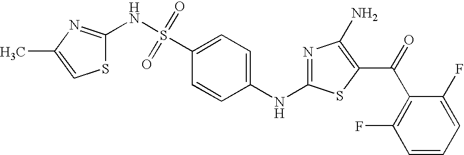 |
| Example R(2) | 4-Amino-5-(2,6-dichloro- benzoyl)-2-(4-methylthio- phenylamino)-thiazole | 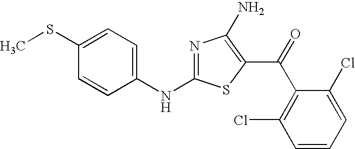 |
| Example R(3) | 4-Amino-5-(2,6-dichloro- benzoyl)-2-(3-methylthio- phenylamino)-thiazole | 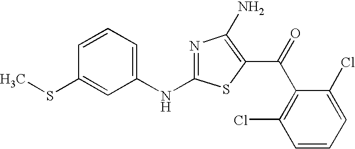 |
| Example W(1) | 4-Amino-5-(2,6-dichloro- benzoyl)-2-[4-(pyridin-4-ylthio)- phenylamino]-thiazole | 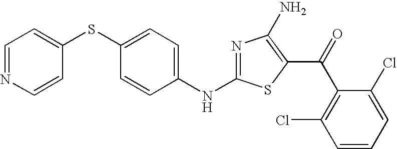 |
| Example W(2) | 4-Amino-5-(2,6-dichloro- benzoyl)-2-[4-pyridin-2-ylthio- phenylaminol-thiazole | 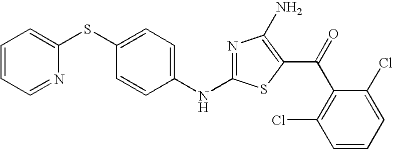 |
| Example X(1) | 4-Amino-5-(2,6-dichloro- benzoyl)-2-(4-mercapto- phenylamino)-thiazole | 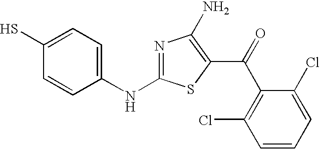 |
| Example X(2) | 3-Amino-5-(2,6-dichloro- benzoyl)-2-(4-mercapto- phenylamino)-thiazole | 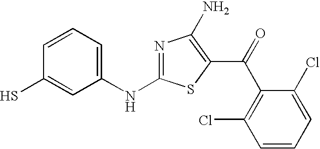 |
| Example Y(1) | 2-{4-[4-Amino-5-(26-dlchloro- benzoyl)-thiazol-2-ylamino]- phenylthio}-acetamide | 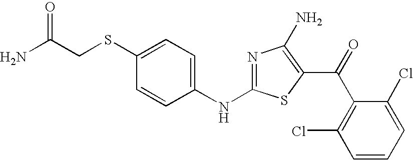 |
| Example Y(2) | 4-Amino-5-(2,6- dichlorobenzoyl)-2-[4-(2- hydroxy-ethylthio)- phenylamino]-thiazole | 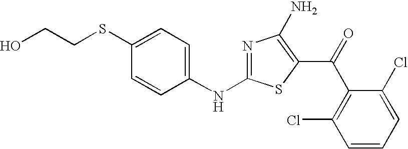 |
| Example Y(3) | 2-{3-[4-Amino-5-(2,6-dichloro- benzoyl)-thiazol-2-yiamino]- phenylthio}-acetamide | 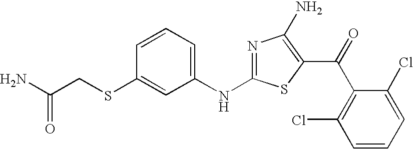 |
| Example Z(1) | 4-Amino-5-(2,6-dichloro- benzoyl)-2-(3-methanesulfinyl- phenylamino)-thiazole | 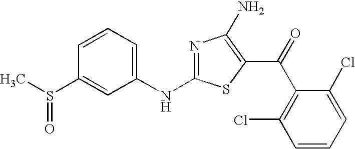 |
| Example Z(2) | 2-(4-{4-Amino-5-(2,6- dichlorobenzoyl)-thiazol-2- ylamino)-benzenesulfinyl)- acetamide | 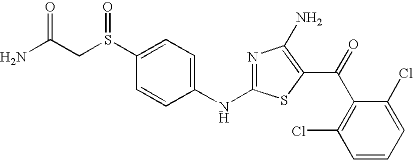 |
| Example Z(3) | 4-Amino-5-(2,6- dichlorobenzoyl)-2-[4-(2- hydroxy-ethanesulfinyl)- phenylamino]-thiazole | 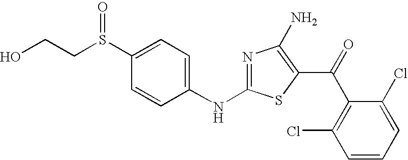 |
| Example Z(4) | 4-Amino-5-(2,6- dichlorobenzoyl)-2-(4- methanesulfinyl-phenylamino)- thiazole | 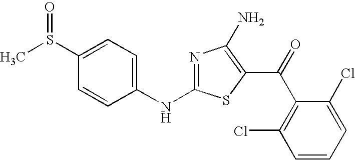 |
| Example Z(5) | 2-{3-[4-Amino-5-(2,6-dichloro- benzoyl)-thiazol-2-ylamino]- benzenesulfinyl}-acetamide | 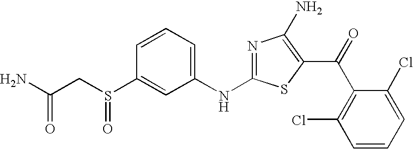 |
| Example AA(1) | 4-Amino-5-(2,6- dichloro- benzoyl)-2-(3- methanesulfonyl- phenylamino)-thiazole | 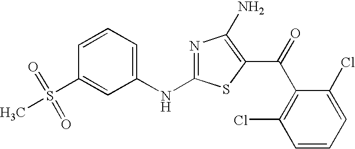 |
| Example AA(2) | 4-Amino-5-(2,6- dichlorobenzoyl)-2-(4- methanesulfonyl- phenylamino)-thiazole | 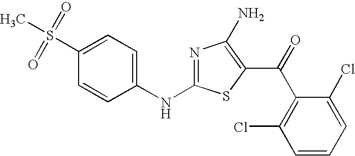 |
| Example AA(3) | 4-Amino-5-(2,6- dichlorobenzoyl)-2-[4- (pyridine-4-sulfonyl)- phenylamino]-thiazole | 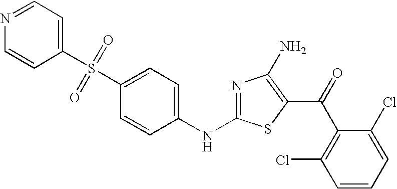 |
| Example BB(1) | 4-[4-Amino-5-(2,6-difluoro- benzoyl)-thiazol-2-ylamino]-N- piperidine-4-yl benzenesulfonamide | 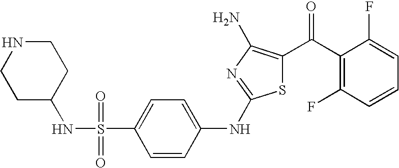 |
| Example CC(1) | 4-[4-Amino-5-(2,6-difluoro-4- methyl-benzoyl)-thiazol-2- ylamino]-N-(2-isopropoxy- ethyl)-benzenesulfonamide | 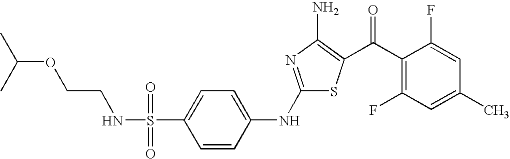 |

The inventive compounds of the present invention are potent anti-proliferative agents. The compounds are also useful for mediating the activity of protein kinases. More particularly, the compounds are useful as anti-angiogenesis agents and as agents for modulating and/or inhibiting the activity of various enzymes, for example protein kinases, thus providing treatments for cancer or other diseases associated with uncontrolled (or abnormal) cellular proliferation.

The diseases or disorders in association with uncontrolled (or abnormal) cellular proliferation include, but are not limited to, the following:

a variety of cancers, including, but not limited to, carcinoma, hematopoietic tumors of lymphoid lineage, hematopoietic tumors of myeloid lineage, tumors of mesenchymal origin, tumors of the central and peripheral nervous system and other tumors including melanoma, seminoma and Kaposi's sarcoma and the like.

a disease process which features abnormal cellular proliferation, e.g., benign prostate hyperplasia, familial adenomatosis polyposis, neuro-fibromatosis, atherosclerosis, pulmonary fibrosis, arthritis, psoriasis, glomerulonephritis, restenosis following angioplasty or vascular surgery, hypertrophic scar formation, inflammatory bowel disease, transplantation rejection, endotoxic shock, and fungal infections.

defective apoptosis-associated conditions, such as cancers (including but not limited to those types mentioned hereinabove), viral infections (including but not limited to herpervirus, poxyiru's, Epstein_barr virus, Sindbis virus and adenovirus), prevention of AIDS development in HIV-infected individuals, autoimmune diseases (including but not limited to systemic lupus, erythermatosus, rheumatoid arthritis, psoriasis, autoimmune mediated glomerulonephritis, inflammatory bowel disease and autoimmune diabetes mellitus), neurodegenerative disorders (including but not limited to Alzheimer's disease, amyotrophic lateral sclerosis, retinitis pigmentosa, Parkinson's disease, AIDS-related dementia, spinal muscular atrophy and cerebellar degeneration), myelodysplastic syndromes, aplastic anemia, ischemic injury associated with myocardial infarctions, stroke and reperfusion injury, arrhythmia, atherosclerosis, toxin-induced or alcohol related liver diseases, hematological diseases (including but not limited to chronic anemia and aplastic anemia), degenerative diseases of the musculoskeletal system (including but not limited to osteroporosis and arthritis), aspirin-sensitive rhinosinusitis, cystic fibrosis, multiple sclerosis, kidney diseases and cancer pain.

The active agents of the invention may also be useful in the inhibition of the development of invasive cancer, tumor angiogenesis and metastasis.

Moreover, the active agents of the invention, as inhibitors of the CDKs, can modulate the level of cellular RNA and DNA synthesis and therefore are expected to be useful in the treatment of viral infections such as HIV, human papilloma virus, herpesvirus, Epstein-Barr virus, adenovirus, Sindbis virus, poxvirus and the like.

Several terms employed throughout the present application are defined below.

In accordance with a convention used in the art,

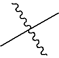

is used in structural formulae herein to depict the bond that is the point of attachment of the moiety or substituent to the core or backbone structure. Moreover,

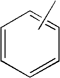

is used in structural formulae herein to depict that the point of attachment of the moiety or substituent to the core of the backbone aryl structure is unspecified. Where chiral carbons are included in chemical structures, unless a particular orientation is depicted, both stereoisomeric forms are intended to be encompassed. Further, the specific inhibitors of the present invention may exist as single stereoisomers, racemates, and/or mixtures of enantiomers and/or diastereomers. All such single stereoisomers, racemates, and mixtures thereof are intended to be within the broad scope of the present invention. The chemical formulae referred to herein may exhibit the phenomenon of tautomerism. Although the structural formulae depict one of the possible tautomeric forms, it should be understood that the invention nonetheless encompasses all tautomeric forms.

The terms “comprising” and “including” are used herein in their open, non-limiting sense.

The term “substituted” means that the specified group or moiety bears one or more substituents. The term “unsubstituted” means that the specified group bears no substituents. The term “optionally substituted” means that the specified group is unsubstituted or substituted by one or more substituents.

The term “alkyl” refers to a straight- or branched-chain alkyl group having from 1 to 12 carbon atoms in the chain. Exemplary alkyl groups include methyl (Me, which also may be structurally depicted by /), ethyl (Et), n-propyl, isopropyl, butyl, isobutyl, sec-butyl, tert-butyl (tBu), pentyl, isopentyl, tert-pentyl, hexyl, isohexyl, and the like. The term “C₃₋₁₄ alkyl” refers to a straight- or branched-chain alkyl group having from 3 to 14 atoms in the chain. The term “C₂₋₁₄ alkyl” refers to a straight- or branched-chain alkyl group having from 2 to 14 atoms in the chain.

The term “heteroalkyl” refers to a straight- or branched-chain alkyl group having from 2 to 12 atoms in the chain, one or more of which is a heteroatom selected from S, O, and N. Preferably, the hteroalkyls of the present invention have between 2 to 9 members. Exemplary heteroalkyls include alkyl ethers, secondary and tertiary alkyl amines, alkyl sulfides, alkoxyl, alcohols, esters and the like.

The term “alkenyl” refers to a straight- or branched-chain alkenyl group having from 2 to 12 carbon atoms in the chain. Illustrative alkenyl groups include prop-2-enyl, but-2-enyl, but-3-enyl, 2-methylprop-2-enyl, hex-2-enyl, ethenyl, pentenyl, and the like.

The term “heteroalkenyl” refers to a straight- or branched-chain alkenyl group having from 2 to 12 carbon atoms in the chain, with one or more of which is a heteroatom selected from S, O, and N. Preferably, the heteroalkenyls of the present invention have 2 to 9 members. Exemplary heteroalkyls include alkenyl ethers, secondary and tertiary alkenyl amines, alkenyl sulfides, alkenoxyl, alcohols, esters and the like.

The term “alkynyl” refers to a straight- or branched-chain alkynyl group having from 2 to 12 carbon atoms in the chain. Illustrative alkynyl groups include prop-2-ynyl, but-2-ynyl, but-3-ynyl, 2-methylbut-2-ynyl, hex-2-ynyl, ethynyl, propynyl, pentynyl and the like.

The term “aryl” (Ar) refers to a monocyclic, or fused polycyclic, aromatic carbocycle (ring structure having ring atoms that are all carbon) having from 6 ring atoms per ring. Illustrative examples of aryl groups include the following moieties:

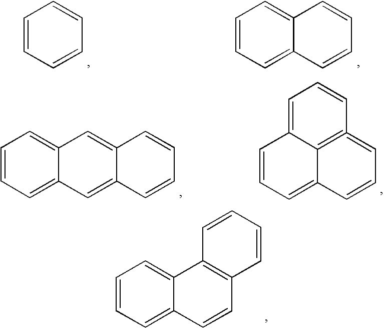

and the like.

The term “heteroaryl” (heteroAr) refers to a monocyclic, or fused polycyclic, aromatic heterocycle (ring structure having ring atoms selected from carbon atoms as well as nitrogen, oxygen, and sulfur heteroatoms) having from 3 to 10 ring atoms per ring. Illustrative examples of heteroaryl groups include moieties having 4 to 7 ring atoms per ring, such as the following moieties:

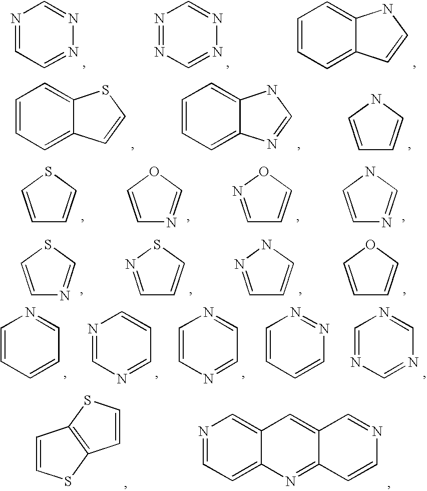

and the like.

The term “cycloalkyl” refers to a saturated or partially saturated, monocyclic or fused or spiro polycyclic, carbocycle having from 3 to 10 ring atoms per ring. Illustrative examples of cycloalkyl groups include cycloalkyl groups having 4 to 8 rings atoms per ring, such as the following moieties:

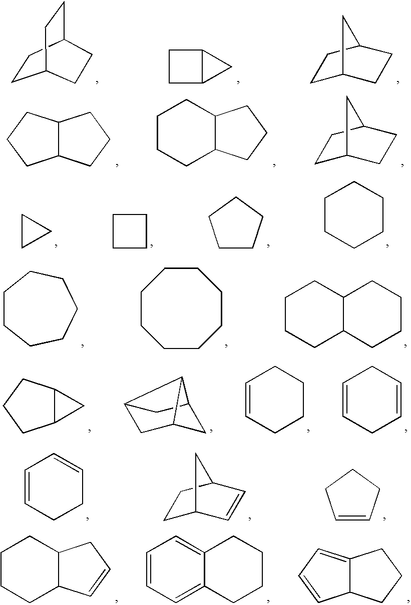

and the like.

A “heterocycloalkyl” refers to a monocyclic, or fused polycyclic, ring structure that is saturated or partially saturated and has from 3 to 10 ring atoms per ring selected from C atoms and N, O, and S heteroatoms. Illustrative examples of heterocycloalkyl groups include heterocycloalkyl groups having 4 to 8 ring atoms per ring, such as the following moieties:

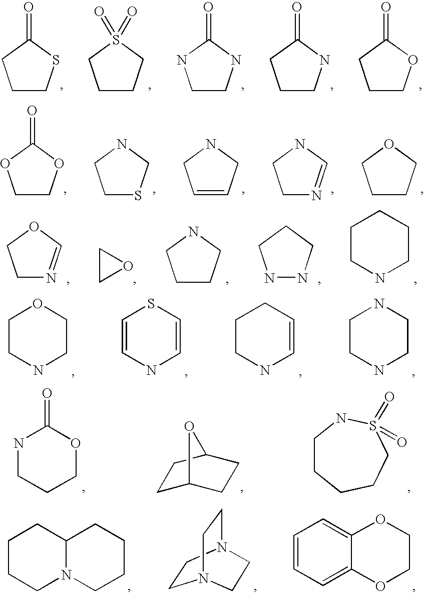

and the like.

An “alkoxyl group” is intended to mean the radical —ORₖ, where Rₖ is an alkyl group. Illustrative examples of alkoxyl groups include moieties having 1 to 14 carbon atoms, such as methoxy, ethoxy, propoxy and so on. “Lower alkoxy” refers to alkoxy groups wherein the alkyl portion has 1 to 4 carbon atoms.

A “hydroxy” group is intended to mean the radical —OH.

The term “amide” refers to the —C(O)NR_{d} radical, where R_{d} is H or C₁-C₆ alkyl.

The term “acetamido” represents —NR_{d}C(O)R_{b}, where R_{b} is selected from H, C₁-C₆ alkyl, —(CR_{d}Rₑ)ₜ(C₃-C₁₀ cycloalkyl), —(CR_{d}Rₑ)ₜ(aryl), and —(CR_{d}Rₑ)ₜ(4-10 membered heterocycloalkyl), —(CR_{d}Rₑ)ₜ(4-10 membered heteroaryl), wherein q and t are each independently an integer from 0 to 5, and R_{d} and Rₑ are as defined above.

The term “acyl” represents —C(O)H, —C(O)OH, —C(O)R_{d}, —C(O)OR_{d}, —C(O)NH₂, —C(O)NHR_{d}, NHR_{d}Rₑ, where R_{d} and Rₑ are independent H or C₁-C₆ alkyl. The term “carboxamide” refers to the radical —C(O)N(R′)(R″) where R′ and R″ are each independently selected from hydrogen, —OH and alkyl, alkenyl, alkynyl, alkoxy, cycloalkyl, heterocycloalkyl, heteroaryl, aryl groups as defined above; or R′ and R″ cyclize together with the nitrogen to form a heterocycloalkyl or heteroaryl as defined above.

The term “nitro” refers to —NO₂.

The term “amino” refers to —NH₂.

The term “halogen” represents chlorine, fluorine, bromine or iodine. The term “halo” represents chloro, fluoro, bromo or iodo.

Abbreviations that are used in the description of the invention include the following: MTBE is methyl tert-Butyl ether; DBU is 1,8-Diazabicyclo[5.4.0]undec-7-ene; EtoAc is ethyl acetate; hex is hexane; DMAP is 4-(N,N-dimethylamino)-pyridine; THF is tetrahydrofuran; TFA is trifluoroacetic acid; HATU is O-(7-azabenzotriazol-1-yl)-N,N,N′,N′-tetramethyluronium hexaflurophosphate; TBAF is tetrabutylammonium fluoride; TMS-OTF is trimethylsilyl triflate; conc. is concentrated; aq. is aqueous; sat. is saturated; DIEA is N,N-Disopropylethyl amine; NBS is N-bromosuccinimide; DMSO is dimethylsulfoxide; MTT is 3-(4,5-dimethylthiazol-2-yl)-2,5-[2H]-diphenyltetrazolium bromide and calcd. is calculated.

Some of the inventive compounds may exist in various stereoisomeric or tautomeric forms. The present invention encompasses all such CDK-inhibiting compounds, including active compounds in the form of single pure enantiomers (i.e., essentially free of other stereoisomers), racemates, mixtures of enantiomers and/or diastereomers, and/or tautomers. Preferably, the inventive compounds that are optically active are used in optically pure form.

As generally understood by those skilled in the art, an optically pure compound having one chiral center (i.e., one asymmetric carbon atom) is one that consists essentially of one of the two possible enantiomers (i.e., is enantiomerically pure), and an optically pure compound having more than one chiral center is one that is both diastereomerically pure and enantiomerically pure. Preferably, the compounds of the present invention are used in a form that is at least 90% optically pure, that is, a form that is at least 90% of a single isomer (80% enantiomeric excess (“e.e.”) or diastereomeric excess (“d.e.”)), more preferably at least 95% (90% e.e. or d.e.), even more preferably at least 97.5% (95% e.e. or d.e.), and most preferably at least 99% (98% e.e. or d.e.).

Additionally, the formulas are intended to cover solvated as well as unsolvated forms of the identified structures. For example, Formula (I) includes compounds of the indicated structure in both hydrated and non-hydrated forms. Other examples of solvates include the structures in combination with isopropanol, ethanol, methanol, DMSO, ethyl acetate, acetic acid, or ethanolamine.

Compositions in accordance with the invention inhibit the kinase activity of CDK/cyclin complexes, such as those active in the G₀ or G₁ stage of the cell cycle, e.g., CDK2, CDK4, and/or CDK6 complexes. Preferred compositions of the invention contain active agents having an inhibition constant against CDK4 or a CDK4/D-type cyclin complex of about 1 μM or less, more preferably of about 500 nM or less, even more preferably of about 200 nM or less, and most preferably of about 100 nM or less. Especially preferred compounds of the invention include those having a CDK4/cyclin D3 inhibition constant (Kᵢ CDK4/D3) of about 100 nM or less. Other preferred compositions of the invention contain active agents having an inhibition constant against CDK2 or a CDK2/E-type cyclin complex of about 1 μM or less, more preferably of about 500 nM or less, even more preferably of about 200 nM or less, and most preferably of about 100 nM or less.

In addition to compounds of Formulas (I-IV), the invention includes pharmaceutically acceptable prodrugs, multimeric forms, active metabolites, and pharmaceutically acceptable salts of such compounds of such compounds and metabolites.

The term “pharmaceutically acceptable” means pharmacologically acceptable and substantially non-toxic to the subject being administered the cell-cycle control agent.

A “prodrug” is a compound that may be converted under physiological conditions or by solvolysis to the specified compound or to a pharmaceutically acceptable salt of such compound. An “active metabolite” is a pharmacologically active product produced through metabolism in the body of a specified compound or salt thereof. Prodrugs and active metabolites of a compound may be identified using routine techniques known in the art. See, e.g., Bertolini et al., J. Med. Chem., (1997) 40:2011-2016; Shan et al., J. Pharm. Sci., 86 (7):765-767; Bagshawe, Drug Dev. Res., (1995) 34:220-230; Bodor, Advances in Drug Res., (1984) 13:224-331; Bundgaard, Design of Prodrugs (Elsevier Press 1985); Larsen, Design and Application of Prodrugs, Drug Design and Development (Krogsgaard-Larsen et al. eds., Harwood Academic Publishers, 1991); Dear et al., J. Chromatogr. B, (2000) 748:281-293; Spraul et al., J. Pharmaceutical & Biomedical Analysis, (1992) 10 (8):601-605; and Prox et al., Xenobiol, (1992) 3 (2):103-112.

A “solvate” is intended to mean a pharmaceutically acceptable solvate form of a specified compound that retains the biological effectiveness of such compound. Examples of solvates include compounds of the invention in combination with water, isopropanol, ethanol, methanol, DMSO, ethyl acetate, acetic acid, or ethanolamine.

The affinity of the compounds of the invention for a receptor may be enhanced by providing multiple copies of the ligand in close proximity, preferably using a scaffolding provided by a carrier moiety. Such multivalent or multimers of active forms of the compounds of the Formula (I) are referred to herein as “multimeric forms”. Multimers of various dimensions (i.e., bearing varying numbers of copies of an active compound) may be tested to arrive at a multimer of optimum size with respect to receptor binding. Provision of such multivalent forms of active receptor-binding compounds with optimal spacing between the receptor-binding moieties may enhance receptor binding (see, for example, Lee, R. T.; et al., Biochem., 1984, 23, 4255-4261). The artisan may control the multivalency and spacing by selection of a suitable carrier moiety or linker units. Useful moieties include molecular supports containing a multiplicity of functional groups that can be reacted with functional groups associated with the active compounds of the invention. A variety of carrier moieties may be used to build highly active multimers, including proteins such as BSA (bovine serum albumin) or HAS, peptides such as pentapeptides, decapeptides, pentadecapeptides, and the like, as well as non-biological compounds selected for their beneficial effects on absorbability, transport, and persistence within the target organism. Functional groups on the carrier moiety, such as amino, sulfhydryl, hydroxyl, and alkylamino groups, may be selected to obtain stable linkages to the compounds of the invention, optimal spacing between the immobilized compounds, and optimal biological properties.

A “pharmaceutically acceptable salt” is intended to mean a salt that retains the biological effectiveness of the free acids and bases of the specified compound and that is not biologically or otherwise undesirable. A compound of the invention may possess a sufficiently acidic, a sufficiently basic, or both functional groups, and accordingly react with any of a number of inorganic or organic bases, and inorganic and organic acids, to form a pharmaceutically acceptable salt. Exemplary pharmaceutically acceptable salts include those salts prepared by reaction of the compounds of the present invention with a mineral or organic acid or an inorganic base, such as salts including sulfates, pyrosulfates, bisulfates, sulfites, bisulfites, phosphates, monohydrogenphosphates, dihydrogenphosphates, metaphosphates, pyrophosphates, chlorides, bromides, iodides, acetates, propionates, decanoates, caprylates, acrylates, formates, isobutyrates, caproates, heptanoates, propiolates, oxalates, malonates, succinates, suberates, sebacates, fumarates, maleates, butyne-1,4-dioates, hexyne-1,6-dioates, benzoates, chlorobenzoates, methylbenzoates, dinitrobenzoates, hydroxybenzoates, methoxybenzoates, phthalates, sulfonates, xylenesulfonates, phenylacetates, phenylpropionates, phenylbutyrates, citrates, lactates, γ-hydroxybutyrates, glycolates, tartrates, methane-sulfonates, propanesulfonates, naphthalene-1-sulfonates, naphthalene-2-sulfonates, and mandelates.

If the inventive compound is a base, the desired pharmaceutically acceptable salt may be prepared by any suitable method available in the art, for example, treatment of the free base with an inorganic acid, such as hydrochloric acid, hydrobromic acid, sulfuric acid, sulfamic acid, nitric acid, phosphoric acid and the like, or with an organic acid, such as acetic acid, phenylacetic acid, propionic acid, stearic acid, lactic acid, ascorbic acid, maleic acid, hydroxymaleic acid, isethionic acid, succinic acid, mandelic acid, fumaric acid, malonic acid, pyruvic acid, oxalic acid, glycolic acid, salicylic acid, a pyranosidyl acid, such as glucuronic acid or galacturonic acid, an alpha-hydroxy acid, such as citric acid or tartaric acid, an amino acid, such as aspartic acid or glutamic acid, an aromatic acid, such as benzoic acid, 2-acetoxybenzoic acid or cinnamic acid, a sulfonic acid, such as p-toluenesulfonic acid, methansulfonic acid or ethanesulfonic acid, or the like.

If the inventive compound is an acid, the desired pharmaceutically acceptable salt may be prepared by any suitable method, for example, treatment of the free acid with an inorganic or organic base, such as an amine (primary, secondary or tertiary), an alkali metal hydroxide or alkaline earth metal hydroxide, or the like. Illustrative examples of suitable salts include organic salts derived from amino acids, such as glycine and arginine, ammonia, carbonates, bicarbonates, primary, secondary, and tertiary amines, and cyclic amines, such as benzylamines, pyrrolidines, piperidine, morpholine and piperazine, and inorganic salts derived from sodium, calcium, potassium, magnesium, manganese, iron, copper, zinc, aluminum and lithium.

Pharmaceutical compositions according to the invention may, alternatively or in addition to compounds of Formulas (I-IV), comprise as an active ingredient pharmaceutically acceptable prodrugs, multimeric forms, pharmaceutically active metabolites, and pharmaceutically acceptable salts of such compounds and metabolites. Such compounds, prodrugs, multimers, salts, and metabolites are sometimes referred to herein collectively as “active agents” or “agents.”

In the case of agents that are solids, it is understood by those skilled in the art that the inventive compounds and salts may exist in different crystal or polymorphic forms, all of which are intended to be within the scope of the present invention and specified formulas.

Therapeutically effective amounts of the active agents of the invention may be used to treat and/or prevent diseases mediated by modulation or regulation of various kinases, for example protein kinases or to treat and/or prevent cellular proliferative diseases. An “effective amount” is intended to mean that amount of an agent that significantly inhibits proliferation and/or prevents de-differentiation of a eukaryotic cell, e.g., a mammalian, insect, plant or fungal cell, and is effective for the indicated utility, e.g., specific therapeutic treatment.

The amount of a given agent that will correspond to such an amount will vary depending upon factors such as the particular compound, disease condition and its severity, the identity (e.g., weight) of the subject or host in need of treatment, but can nevertheless be routinely determined in a manner known in the art according to the particular circumstances surrounding the case, including, e.g., the specific agent being administered, the route of administration, the condition being treated, and the subject or host being treated. “Treating” is intended to mean at least the mitigation of a disease condition in a subject such as mammal (e.g., human), that is affected, at least in part, by the activity of one or more kinases, for example protein kinases such as tyrosine kinases, and includes: preventing the disease condition from occurring in a mammal, particularly when the mammal is found to be predisposed to having the disease condition but has not yet been diagnosed as having it; modulating and/or inhibiting the disease condition; and/or alleviating the disease condition.

### DETAILED DESCRIPTION OF THE INVENTION AND PREFERRED EMBODIMENTS

Agents that potently regulate, modulate, or inhibit the protein kinase activity associated with receptors CDK complexes, among others, and which inhibit angiogenesis and/or cellular proliferation are preferred. The present invention is further directed to methods of modulating or inhibiting protein kinase activity, for example in mammalian tissue, by administering an inventive agent. The activity of the inventive agents as modulators of protein kinase activity, such as the activity of kinases, may be measured by any of the methods available to those skilled in the art, including in vivo and/or in vitro assays. Examples of suitable assays for activity measurements include those described in WIPO International Publication No. WO 99/21845; Parast et al., Biochemistry, 37, 16788-16801 (1998); Jeffrey et al., Nature, 376, 313-320 (1995); WIPO International Publication No. WO 97/34876; and WIPO International Publication No. WO 96/14843. These properties may be assessed, for example, by using one or more of the biological testing procedures set out in the examples below.

The active agents of the invention may be formulated into pharmaceutical compositions as described below. Pharmaceutical compositions of this invention comprise an effective modulating, regulating, or inhibiting amount of a compound of Formula (I) and an inert, pharmaceutically acceptable carrier or diluent. In one embodiment of the pharmaceutical compositions, efficacious levels of the inventive agents are provided so as to provide therapeutic benefits involving modulation of protein kinases. By “efficacious levels” is meant levels in which the effects of protein kinases are, at a minimum, regulated. These compositions are prepared in unit-dosage form appropriate for the mode of administration, e.g., parenteral or oral administration.

An inventive agent can be administered in conventional dosage form prepared by combining a therapeutically effective amount of an agent (e.g., a compound of Formula (I)) as an active ingredient with appropriate pharmaceutical carriers or diluents according to conventional procedures. These procedures may involve mixing, granulating and compressing or dissolving the ingredients as appropriate to the desired preparation.

The pharmaceutical carrier employed may be either a solid or liquid. Exemplary of solid carriers are lactose, sucrose, talc, gelatin, agar, pectin, acacia, magnesium stearate, stearic acid and the like. Exemplary of liquid carriers are syrup, peanut oil, olive oil, water and the like. Similarly, the carrier or diluent may include time-delay or time-release material known in the art, such as glyceryl monostearate or glyceryl distearate alone or with a wax, ethylcellulose, hydroxypropylmethylcellulose, methylmethacrylate and the like.

A variety of pharmaceutical forms can be employed. Thus, if a solid carrier is used, the preparation can be tableted, placed in a hard gelatin capsule in powder or pellet form or in the form of a troche or lozenge. The amount of solid carrier may vary, but generally will be from about 25 mg to about 1 g. If a liquid carrier is used, the preparation will be in the form of syrup, emulsion, soft gelatin capsule, sterile injectable solution or suspension in an ampoule or vial or non-aqueous liquid suspension.

To obtain a stable water-soluble dose form, a pharmaceutically acceptable salt of an inventive agent is dissolved in an aqueous solution of an organic or inorganic acid, such as 0.3M solution of succinic acid or citric acid. If a soluble salt form is not available, the agent may be dissolved in a suitable cosolvent or combinations of cosolvents. Examples of suitable cosolvents include, but are not limited to, alcohol, propylene glycol, polyethylene glycol 300, polysorbate 80, gylcerin and the like in concentrations ranging from 0-60% of the total volume. In an exemplary embodiment, a compound of Formula (I) is dissolved in DMSO and diluted with water. The composition may also be in the form of a solution of a salt form of the active ingredient in an appropriate aqueous vehicle such as water or isotonic saline or dextrose solution.

It will be appreciated that the actual dosages of the agents used in the compositions of this invention will vary according to the particular complex being used, the particular composition formulated, the mode of administration and the particular site, host and disease being treated. Optimal dosages for a given set of conditions can be ascertained by those skilled in the art using conventional dosage-determination tests in view of the experimental data for an agent. For oral administration, an exemplary daily dose generally employed is from about 0.001 to about 1000 mg/kg of body weight, with courses of treatment repeated at appropriate intervals. Administration of prodrugs is typically dosed at weight levels which are chemically equivalent to the weight levels of the fully active form.

The compositions of the invention may be manufactured in manners generally known for preparing pharmaceutical compositions, e.g., using conventional techniques such as mixing, dissolving, granulating, dragee-making, levigating, emulsifying, encapsulating, entrapping or lyophilizing. Pharmaceutical compositions may be formulated in a conventional manner using one or more physiologically acceptable carriers, which may be selected from excipients and auxiliaries that facilitate processing of the active compounds into preparations which can be used pharmaceutically.

Proper formulation is dependent upon the route of administration chosen. For injection, the agents of the invention may be formulated into aqueous solutions, preferably in physiologically compatible buffers such as Hanks's solution, Ringers solution, or physiological saline buffer. For transmucosal administration, penetrants appropriate to the barrier to be permeated are used in the formulation. Such penetrants are generally known in the art.

For oral administration, the compounds can be formulated readily by combining the active compounds with pharmaceutically acceptable carriers known in the art. Such carriers enable the compounds of the invention to be formulated as tablets, pills, dragees, capsules, liquids, gels, syrups, slurries, suspensions and the like, for oral ingestion by a patient to be treated. Pharmaceutical preparations for oral use can be obtained using a solid excipient in admixture with the active ingredient (agent), optionally grinding the resulting mixture, and processing the mixture of granules after adding suitable auxiliaries, if desired, to obtain tablets or dragee cores. Suitable excipients include: fillers such as sugars, including lactose, sucrose, mannitol, or sorbitol; and cellulose preparations, for example, maize starch, wheat starch, rice starch, potato starch, gelatin, gum, methyl cellulose, hydroxypropylmethyl-cellulose, sodium carboxymethylcellulose, or polyvinylpyrrolidone (PVP). If desired, disintegrating agents may be added, such as crosslinked polyvinyl pyrrolidone, agar, or alginic acid or a salt thereof such as sodium alginate.

Dragee cores are provided with suitable coatings. For this purpose, concentrated sugar solutions may be used, which may optionally contain gum arabic, polyvinyl pyrrolidone, Carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active agents.

Pharmaceutical preparations which can be used orally include push-fit capsules made of gelatin, as well as soft, sealed capsules made of gelatin and a plasticizer, such as glycerol or sorbitol. The push-fit capsules can contain the active ingredients in admixture with fillers such as lactose, binders such as starches, and/or lubricants such as talc or magnesium stearate, and, optionally, stabilizers. In soft capsules, the active agents may be dissolved or suspended in suitable liquids, such as fatty oils, liquid paraffin, or liquid polyethylene glycols. In addition, stabilizers may be added. All formulations for oral administration should be in dosages suitable for such administration. For buccal administration, the compositions may take the form of tablets or lozenges formulated in conventional manner.

For administration intranasally or by inhalation, the compounds for use according to the present invention are conveniently delivered in the form of an aerosol spray presentation from pressurized packs or a nebuliser, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of gelatin for use in an inhaler or insufflator and the like may be formulated containing a powder mix of the compound and a suitable powder base such as lactose or starch.

The compounds may be formulated for parenteral administration by injection, e.g., by bolus injection or continuous infusion. Formulations for injection may be presented in unit-dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents.

Pharmaceutical formulations for parenteral administration include aqueous solutions of the active agents in water-soluble form. Additionally, suspensions of the agents may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions.

For administration to the eye, the active agent is delivered in a pharmaceutically acceptable ophthalmic vehicle such that the compound is maintained in contact with the ocular surface for a sufficient time period to allow the compound to penetrate the corneal and internal regions of the eye, including, for example, the anterior chamber, posterior chamber, vitreous body, aqueous humor, vitreous humor, cornea, iris/cilary, lens, choroid/retina and selera. The pharmaceutically acceptable ophthalmic vehicle may be an ointment, vegetable oil, or an encapsulating material. A compound of the invention may also be injected directly into the vitreous and aqueous humor.

Alternatively, the active agents may be in powder form for constitution with a suitable vehicle, e.g., sterile pyrogen-free water, before use. The compounds may also be formulated in rectal compositions such as suppositories or retention enemas, e.g., containing conventional suppository bases such as cocoa butter or other glycerides.

In addition to the formulations described above, the active agents also be formulated as a depot preparation. Such long-acting formulations may be administered by implantation (for example, subcutaneously or intramuscularly) or by intramuscular injection. Thus, for example, the compounds may be formulated with suitable polymeric or hydrophobic materials (for example, as an emulsion in an acceptable oil) or ion-exchange resins, or as sparingly soluble derivatives, for example, as a sparingly soluble salt.

An exemplary pharmaceutical carrier for hydrophobic compounds is a cosolvent system comprising benzyl alcohol, a nonpolar surfactant, a water-miscible organic polymer, and an aqueous phase. The cosolvent system may be a VPD co-solvent system. VPD is a solution of 3% w/v benzyl alcohol, 8% w/v of the nonpolar surfactant polysorbate 80, and 65% w/v polyethylene glycol 300, made up to volume in absolute ethanol. The VPD co-solvent system (VPD: 5W) contains VPD diluted 1:1 with a 5% dextrose in water solution. This co-solvent system dissolves hydrophobic compounds well, and itself produces low toxicity upon systemic administration. Naturally, the proportions of a co-solvent system may be varied considerably without destroying its solubility and toxicity characteristics. Furthermore, the identity of the co-solvent components may be varied: for example, other low-toxicity nonpolar surfactants may be used instead of polysorbate 80; the fraction size of polyethylene glycol may be varied; other biocompatible polymers may replace polyethylene glycol, e.g. polyvinyl pyrrolidone; and other sugars or polysaccharides may be substituted for dextrose.

Alternatively, other delivery systems for hydrophobic pharmaceutical compounds may be employed. Liposomes and emulsions are known examples of delivery vehicles or carriers for hydrophobic drugs. Certain organic solvents such as dimethylsulfoxide also may be employed, although usually at the cost of greater toxicity. Additionally, the compounds may be delivered using a sustained-release system, such as semipermeable matrices of solid hydrophobic polymers containing the therapeutic agent. Various sustained-release materials have been established and are known by those skilled in the art. Sustained-release capsules may, depending on their chemical nature, release the compounds for a few weeks up to over 100 days. Depending on the chemical nature and the biological stability of the therapeutic reagent, additional strategies for protein stabilization may be employed.

The pharmaceutical compositions also may comprise suitable solid- or gel-phase carriers or excipients. Examples of such carriers or excipients include calcium carbonate, calcium phosphate, sugars, starches, cellulose derivatives, gelatin, and polymers such as polyethylene glycols.

Some of the compounds of the invention may be provided as salts with pharmaceutically compatible counter ions. Pharmaceutically compatible salts may be formed with many acids, including hydrochloric, sulfuric, acetic, lactic, tartaric, malic, succinic, etc. Salts tend to be more soluble in aqueous or other protonic solvents than are the corresponding free-base forms.

The active agents of the invention may be useful in combination with known anti-cancer treatments such as, but not limited to, DNA interactive agents such as cisplatin or doxorubicin; topoisomerase II inhibitors such as etoposide, topoisomerase I inhibitors such as CPT-11 or topotecan; tubulin interacting agents such as paclitaxel, docetaxel or the epothilones; hormonal agents such as tamoxifen; thymidilate synthase inhibitors such as 5-fluorouracil; and anti-metabolites such as methotrexate. They may be administered together or sequentially, and when administered sequentially, the inventive agents may be administered either prior to or after administration of the known anticancer or cytotoxic agent.

The inventive agents may be prepared using the reaction routes and synthesis schemes as described below, employing the general techniques known in the art using starting materials that are readily available. The preparation of preferred compounds of the present invention is described in detail in the following examples, but the artisan will recognize that the chemical reactions described may be readily adapted to prepare a number of other protein kinase inhibitors of the invention. For example, the synthesis of non-exemplified compounds according to the invention may be successfully performed by modifications apparent to those skilled in the art, e.g., by appropriately protecting interfering groups, by changing to other suitable reagents known in the art, or by making routine modifications of reaction conditions. Alternatively, other reactions disclosed herein or generally known in the art will be recognized as having applicability for preparing other compounds of the invention.

General routes to the compounds of the invention are described as follows:

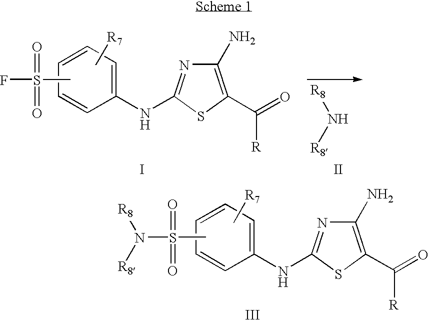

A direct approach to sulfonamide derivatives is described as Scheme I. Sulfonyl fluorides I and amines II provide corresponding sulfonamides III, with or without base catalysis or acid scavenging, in polar aprotic organic solvent, such as acetonitrile (MeCN), tetrahydrofuran (THF), or N,N-dimethylformamide (DMF). This method is amenable to parallel synthesis.

The starting material, sulfonyl fluorides 1, is available from standard methodology to form the 2,4-diaminothiazoles (see WO99/21845 and Gewald, et al, J. Prakt. Chem., 35, 97-104 (1967)), as depicted below as part of the route in Scheme 2. For example, if M is fluoride in Scheme 2, the sulfonyl fluoride survives defined reaction conditions wherein nitro V is reduced via catalytic hydrogenation to aniline VI, which in turn is converted with routine conditions, for example, with thiophosgene, under overall acidic conditions, to isothiocyanate VII. Subsequent condensation of cyanamide in the presence of a strong, but hindered tertiary base such as 1,8-diazabicyclo[5.4.0]undec-7-ene (DBU) provides the isothiourea anion VIII, which is S-alkylated in situ with a halocarbonyl IX to intermediate X. Many different halocarbonyl IX, are available commercially or from the literature. However, particularly poly-substituted 2-haloacetophenones IX (R=—Ar) were previously described in Patent Application WO 99/21845, and new additional preparations are enclosed herein. Base-promoted enolization of isothiourea X causes cyclization to furnish diaminothiazole XI (M=—F), bearing the preserved sulfonyl fluoride for Scheme 1.

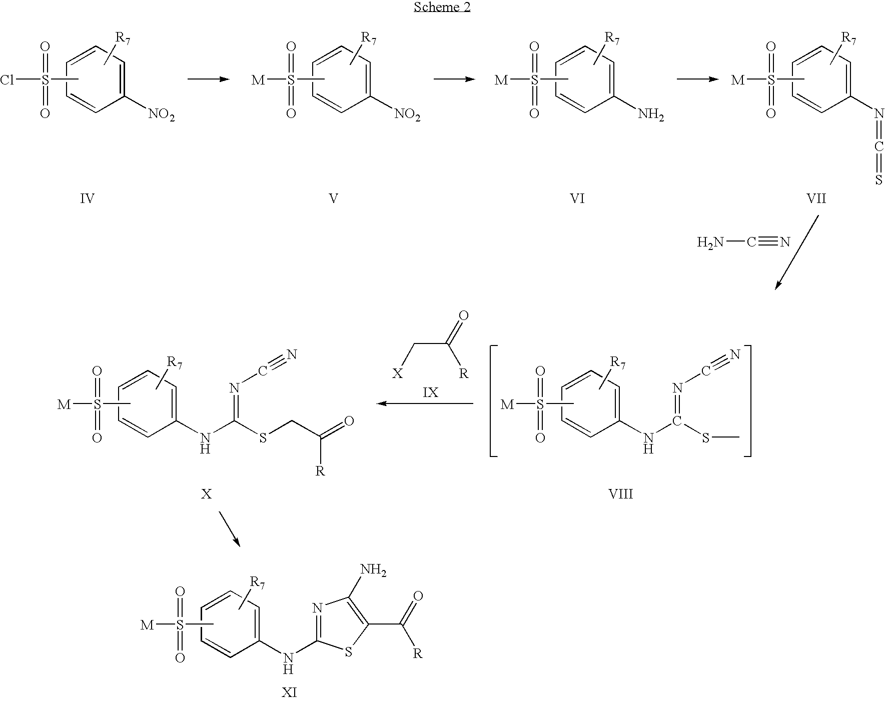

Alternatively, within Scheme 2, if M is an alkyl group (M=—R₇), or an alkylamino- (M=—NR₈R_{8′}) derived from a generic amine II—a sulfonyl moiety is installed at the outset, prior to thiazole formation, and carried through the sequence to arrive at either sulfones, XI (M=—R₇), or sulfonamides XI (M=—NR₈R_{8′}; or III), respectively. For sulfonamides, sulfonyl halides IV and amines II yield nitro-sulfonamides V (M=—NR₈R_{8′}), which are each subjected to the sequence outlined in Scheme 2 and as described for the sulfonyl fluoride above. For the sulfones, many starting materials are available at the later stage of aminophenylsulfones VI (M=—R₇), but likewise are suitable for the route in Scheme 2.

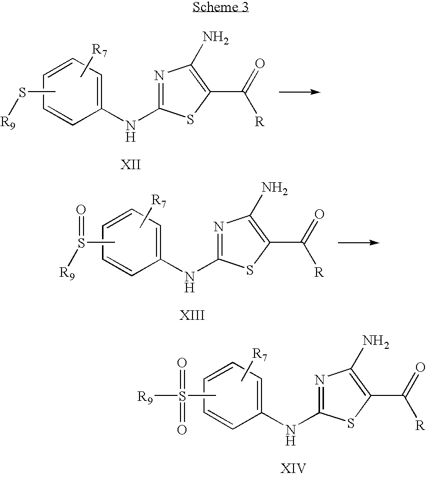

Sulfones are also available via another pathway, involving oxidation of 2,4-diaminothiazole sulfides XII as depicted in Scheme 3. Utilizing typical peracid oxidants, such as meta-chloroperbenzoic acid (MCPBA), stepwise oxidation of XII is also possible, and allows the preparation of sulfoxides XIII en route to sulfones XIV. The starting material XII for Scheme 3 are available from established methods for 2,4-diaminothiazole ring formation, see WO 99/21845, Gewald, et al, J. Prakt. Chem., 35, 97-104 (1967), and the route in Scheme 2, starting with thioalkyl-substituted phenyl isothiocyanate. Alternatively, the thiol XV can also be prepared and selectively S-alkylated to thioethers XII, as shown below. As is demonstrated herein, the sequence from thiols XV to sulfoxides XIII and subsequently to corresponding sulfones XIV, is adaptable to parallel synthesis.

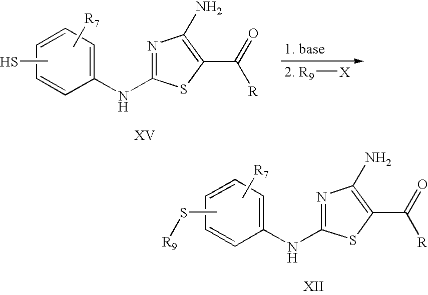

### EXAMPLES

In the examples described below, unless otherwise indicated, all temperatures are set forth in degrees Celsius and all parts and percentages are by weight. Reagents were purchased from commercial suppliers, such as Aldrich Chemical Company or Lancaster Synthesis Ltd. and were used without further purification unless otherwise indicated. Tetrahydrofuran (THF) and N,N-dimethylformamide (DMF) were purchased from Aldrich in Sure Seal bottles and used as received. All solvents were purified using standard methods known to those skilled in the art, unless otherwise indicated.

The reactions set forth below were done generally under a positive pressure of argon at an ambient temperature (unless otherwise stated) in anhydrous solvents, and the reaction flasks were fitted with rubber septa for the introduction of substrates and reagents via syringe. Glassware was oven dried and/or heat dried. Analytical thin layer chromatography (TLC) was performed on glass-backed silica gel 60 F 254 plates from Analtech (0.25 mm), eluted with the appropriate solvent ratios (v/v), and is denoted where appropriate. The reactions were assayed by TLC, HPLC, or ¹H NMR, and terminated as judged by the consumption of starting material.

Visualization of the TLC plates was done with iodine vapor, ultraviolet illumination, 2% Ce(NH₄)₄(SO₄)₄ in 20% aqueous sulfuric acid, 2% ninhydrin in ethanol, or p-anisaldehyde spray reagent, and activated with heat where appropriate. Work-ups were typically done by doubling the reaction volume with the reaction solvent or extraction solvent and then washing with the indicated aqueous solutions using 25% by volume of the extraction volume unless otherwise indicated. Product solutions were dried over anhydrous Na₂SO₄ and/or MgSO₄ prior to filtration and evaporation of the solvents under reduced pressure on a rotary evaporator and noted as solvents removed in vacuo. Flash column chromatography (Still et al., J. Org. Chem., 43, 2923 (1978)) was done using Merck silica gel (47-61 μm) with a silica gel crude material ratio of about 20:1 to 50:1, unless otherwise stated. Certain example compounds were purified via preparative high-performance liquid chromatography (HPLC), and unless otherwise indicated, refers to a Gilson 321 system, equipped with a C18 reversed-phase preparative column (Metasil AQ 10 micron, 120A, 250×21.2 mm, MetaChem) and elution with a gradient of 0.1% trifluoroacetic acid (TFA)/5% acetonitrile/water to 0.1% TFA/5% water/acetonitrile over 20 minutes and flow rate of 20 mL/minute. Hydrogenations were performed at ambient pressure unless otherwise indicated. All melting points (mp) are uncorrected.

¹H-NMR spectra were recorded on a Bruker or Varian instrument operating at 300 MHz and ¹³C-NMR spectra were recorded operating at 75 MHz. NMR spectra were obtained as CDCl₃ solutions (reported in ppm), using chloroform as the reference standard (7.27 ppm and 77.00 ppm) unless otherwise indicated. When peak multiplicities are reported, the following abbreviations are used: s (singlet), d (doublet), t (triplet), q (quartet), m (multiplet), bs (broad singlet), bm (broad multiplet), dd (doublet of doublets), ddd (doublet of doublet of doublets), dddd (doublet of doublet of doublet of doublets), dt (doublet of triplets). Coupling constants, when given, are reported in Hertz (Hz).

Infrared (IR) spectra were recorded on a Perkin-Elmer FT-IR Spectrometer as neat oils, KBr pellets, or CDCl₃ solutions, and when given are reported in wave numbers (cm⁻¹).

Mass spectrometry was conducted with various techniques. Matrix-Assisted Laser Desorption/Ionization Fourier Transform Mass Spectrometry (MALDI FTMS) was performed on an IonSpec FTMS mass spectrometer. Samples are irradiated with a nitrogen laser (Laser Science Inc.) operated at 337 nm and the laser beam is attenuated by a variable attenuator and focused on the sample target. The ions are then differentiated according to their m/z using an ion cyclotron resonance mass analyzer. The electrospray ionization (ESI) mass spectrometry experiments were performed on an API 100 Perkin Elmer SCIEX single quadrupole mass spectrometer. Electrospray samples are typically introduced into the mass analyzer at a rate of 4.0 μl/minute. The positive and negative ions, generated by charged droplet evaporation, enter the analyzer through an interface plate and a 100 mm orifice, while the declustering potential is maintained between 50 and 200V to control the collisional energy of the ions entering the mass analyzer. The emitter voltage is typically maintained at 4000V. The liquid chromatography (LC) electrospray ionization (ESI) mass spectrometry experiments were performed on a Hewlett-Packard (HP) 1100 MSD single quadrupole mass spectrometer. Electrospray samples are typically introduced into the mass analyzer at a rate of 100 to 1000 μl/minute. The positive and negative ions, generated by charged droplet evaporation, enter the analyzer through a heated capillary plate, while the declustering potential is maintained between 100 and 300 V to control the collisional energy of the ions entering the mass analyzer. The emitter voltage is typically maintained at 4000 V.

Compounds in accordance with the invention may be prepared in manners analogous to those specifically described below, with the lettered example prefixes (i.e., A, B, C, D, E, F, G, H, I, J, K, L, M, N, O, P, Q, R, S, T, U, V, W, X, Y, Z, M, BB, CC, DD, EE, and FF) designating general synthesis schemes.

### Example A(1)

### {4-Amino-2-[4-(piperazine-1-sulfonyl)-phenylamino]-thiazol-5-yl}-(2,6-difluoro-phenyl)-methanone

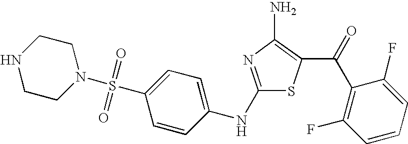

First 4-isothiocyanato-benzenesulfonyl fluoride, which has the structural formula

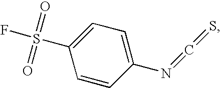

was prepared. According to a method from McKee et al., J. Am. Chem. Soc., 48 (1946), 2506-2507, sulfanilyl fluoride (6.00 g, 34.3 mmol, from Sigma-Aldrich Chemical—discontinued; see Krazer Helv. Chim. Acta. 43, 1513-1519 (1960)) was dissolved in water (60 mL) containing 38% HCl (14.4 mL). Thiophosgene (2.7 mL, 36.0 mmol) was added in one portion. The resultant mixture was stirred rapidly for a half hour, then diluted with water (200 mL). The resultant white precipitate was filtered off, washed with water, and dried under high vacuum to obtain 7.28 g (99%) of white powder, which was used without further purification.

¹H NMR (DMSO-d₆): δ 8.03 (2H, d, J=9.3 Hz), 7.44 (2H, d, J=9.3 Hz).

FABMS: (M−H⁺): 216.

2-Bromo-2′,6′-difluoroacetophenone, which has the structural formula

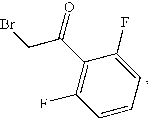

was prepared as follows. To a mechanically stirring solution of 2′,6′-difluoroacetophenone (100.0 g, 640.0 mmol; Melford Laboratories, Ltd.) in ethyl acetate (1300 mL) were added freshly milled copper(II) bromide (300 g, 1.35 mol) and bromine (1.6 mL, 32 mmol). The mixture was heated at reflux for 2.25 hours and allowed to cool to ambient temperature. The resultant green mixture was filtered and the solids rinsed with ethyl acetate (4×100 mL). The filtrate was concentrated with a rotary evaporator under reduced pressure, diluted with methyl t-butyl ether (MTBE; 650 mL), filtered through a pad of silica gel (230-400μ; 9.5 cm diam.×4 cm. ht.), and solids rinsed with MTBE (5×200 mL). Concentration of the filtrate gave a pale green oil, which was purified by fractional vacuum distillation to give 117 g of pale yellow oil, bp 88-97° C. (2.0 mm Hg) in 78% yield. Matched that previously described in World Patent Application WO99/21845 (in Example C(79)) and was used without any further purification or characterization.

¹H NMR: δ 7.48 (1H, ddd, J=6.3, 8.5, 14.8 Hz), 7.01 (2H, ddd, J=4.6, 5.8, 16.6 Hz), 4.37 (2H, t, J=0.7 Hz).

4-[4-Amino-5-(2,6-difluoro-benzoyl)-thiazol-2-ylamino]-benzenesulfonyl fluoride, which has the structural formula

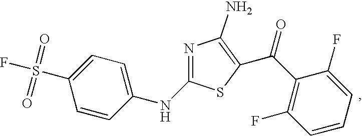

was then made as follows. To 4-isothiocyanato-benzenesulfonyl fluoride (4.00 g, 18.4 mmol) and cyanamide (851 mg, 20.3 mmol) in CH₃CN (20 mL), in a vessel placed in a cold-water bath, was added 1,8-diazabicyclo[5.4.0]undec-7-ene (DBU; 3.0 mL, 20 mmol). After 15 minutes, a solution of 2-bromo-2′,6′-difluoro-acetophenone (4.54 g, 19.3 mmol; from Example A(1)) in CH₃CN (1 mL) and DBU (3.0 mL, 20.3 mmol) were sequentially added. The mixture was stirred at ambient temperature for a half-hour, then partitioned between CH₂Cl₂ and water, and acidified to pH=4 with 1N HCl. The organic layer was separated, washed with brine, and dried over Na₂SO₄. The solvent was evaporated to give a hard foam, which was purified via column chromatography with 1:1 ethyl acetate (EtOAc) and hexane (hex) as eluant to afford 6.2 g (82% yield) of a yellow powder.

¹H NMR (DMSO-d₆): δ 11.50 (1H, s), 8.35 (2H, bm), 8.10 (2H, d, J=9.0 Hz), 7.96 (2H, d, J=9.0 Hz), 7.58 (1H, m), 7.24 (2H, dd, J=7.8, 8.2 Hz).

Anal. calcd. for C₁₆H₁₀F₂N₃O₃S₂.0.1 EtOAc: C, 46.65; H, 2.58; N, 9.95; S, 15.19. Found: C, 46.65; H, 2.55; N, 9.80; S, 15.02.

The title compound was prepared as follows. A mixture of 4-[4-amino-5-(2,6-difluoro-benzoyl)-thiazol-2-ylamino]-benzenesulfonyl fluoride (200 mg, 0.484 mmol), piperazine (125 mg, 1.45 mmol), CH₃CN (2 mL), and 4-(N,N-dimethylamino)-pyridine (DMAP; 5 mg) was refluxed for 2 hours. The solvent was removed under reduced pressure, the residue was taken up into MeOH (2 mL), then precipitated with water, filtered, and washed with water. Further purification with column chromatography gave 91 mg (43% yield) of a yellow powder.

¹H NMR (DMSO-d₈): δ 8.22 (2H, bs), 7.83 (2H, d, J=8.7 Hz), 7.68 (2H, d, J=8.7 Hz), 7.56 (1H, m), 7.22 (2H, dd, J=7.8, 8.2 Hz).

HRESIMS: calcd. for C₂₀H₂₀F₂N₅O₃S₂ (M+H⁺): 480.0976. Found: 480.0988.

Anal. calcd. for C₂₀H₁₉F₂N₅O₃S₂.0.7 MeOH: C, 49.53; H, 4.38; N, 13.95; S, 12.78. Found: C, 49.51; H, 4.39; N, 13.84; S, 12.93.

### Example A(2)

### 4-[4-Amino-5-(2,6-difluoro-benzoyl)-thiazol-2-ylamino]-N-(2-dimethylamino-ethyl)-benzenesulfonamide

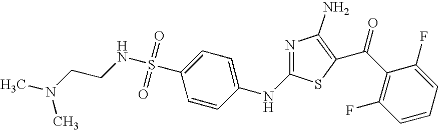

The title compound was prepared in a manner similar to that described for Example A(1). 4-[4-Amino-5-(2,6-difluoro-benzoyl)-thiazol-2-ylamino]-benzenesulfonyl fluoride (from Example A(1); 200 mg, 0.484 mmol) and N,N-dimethylethylenediamine (Sigma-Aldrich, 0.32 mL, 2.9 mmol) gave a yellow powder in 67% yield.

¹H NMR (DMSO-d₆): δ 11.50 (1H, s), 8.23 (2H, bs), 7.77 (4H, s), 7.56 (1H, m), 7.44 (1H, m), 7.21 (2H, dd, J=7.8, 8.1 Hz), 2.80 (2H, t, J=6.9 Hz), 2.24 (2H, t, J=6.9 Hz), 2.06 (6H, s).

HRESIMS: Calcd for C₂₀H₂₂F₂N₅O₃S₂ (M+H⁺): 482.1132. Found: 482.1174.

Anal. calcd. for C₂₀H₂₁F₂N₅O₃S₂.0.7 hexane.0.1H₂O: C, 50.87; H. 4.83; N, 13.99; S. 12.81. Found: C, 50.70; H, 4.88; N, 13.99; S, 12.82.

### Example A(3)

### 4-[4-Amino-5-(2,6-difluoro-benzoyl)-thiazol-2-ylamino]-N-methoxymethyl-benzenesulfonamide

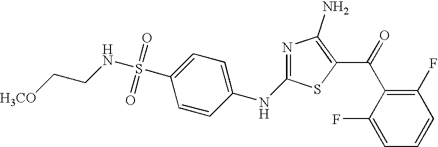

The title compound was prepared in a manner similar to that described for Example A(1). 4-[4-Amino-5-(2,6-difluoro-benzoyl)-thiazol-2-ylamino]-benzenesulfonyl fluoride (200 mg, 0.484 mmol) and 2-methoxyethylamine (0.25 mL, 2.9 mmol) gave a yellow powder in 80% yield.

¹H NMR (DMSO-d₆): δ 11.20 (1H, s), 8.25 (2H, bs), 7.78 (4H, s), 7.63 (1H, t, J=5.8 Hz), 7.56 (1H, m), 7.22 (2H, t, J=7.7 Hz), 3.29 (2H, t, J=5.5 Hz), 3.16 (3H, s), 2.89 (2H, q, J=5.5 Hz).

HRESIMS: calcd. for C₁₉H₁₉F₂N₄O₄S₂ (M+H⁺): 469.0816. Found: 469.0821.

Anal. calcd. for C₁₉H₁₈F₂N₄O₄S₂.0.2 hexane: C, 49.34; H, 4.10; N, 11.74; S. 13.44. Found: C, 49.46; H, 4.23; N, 11.56; S, 13.20.

### Example A(4)

### 4-[4-Amino-5-(2,6-difluoro-benzoyl)-thiazol-2-ylamino]-N-(2-hydroxy-ethyl)-benzenesulfonamide

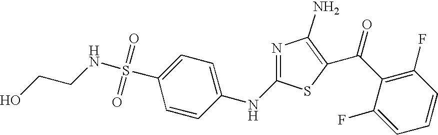

In a manner analogous to that of Example A(1), 4-[4-amino-5-(2,6-difluoro-benzoyl)-thiazol-2-ylamino]-benzenesulfonyl fluoride (150 mg, 0.36 mmol) and ethanolamine (0.046 mL, 1.1 mmol) gave a yellow solid in 31% yield.

¹H NMR (DMSO-d₆): δ 11.19 (1H, s), 8.25 (2H, bs), 7.80 (2H, d, J=9.3 Hz), 7.75 (2H, d, J=9.3 Hz), 7.55 (1H, m), 7.51 (1H, t, J=6.1 Hz), 7.22 (2H, dd, J=7.7, 8.2 Hz), 4.67 (1H, t, J=6.1 Hz), 3.35 (2H, q, J=6.1 Hz), 2.77 (2H, q, J=6.1 Hz).

HRESIMS: calcd. for C₁₈H₁₆F₂N₄O₄S₂Na (M+Na⁺): 477.0479. Found: 477.0472

Anal. calcd. for C₁₈H₁₆F₂N₄O₄S₂.1.0H₂O: C, 45.76; H, 3.84; N, 11.86; S. 13.57. Found: C, 46.08; H, 3.78; N, 11.59; S, 13.38.

### Example A(5)

### 4-[4-Amino-5-(2,6-difluoro-benzoyl)-thiazol-2-ylamino]-N-(4-hydroxy-butyl)-benzenesulfonamide

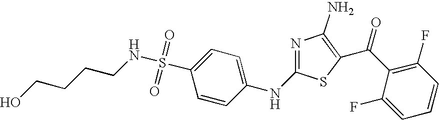

The title compound was prepared in a manner similar to that described for Example A(1). 4-[4-Amino-5-(2,6-difluoro-benzoyl)-thiazol-2-ylamino]-benzenesulfonyl fluoride (200 mg, 0.484 mmol) and 4-amino-1-butanol (0.13 mL, 1.4 mmol) gave a yellow powder in 39% yield.

¹H NMR (DMSO-d₆): δ 11.19 (1H, s), 8.25 (2H, bs), 7.79 (2H, d, J=9.1 Hz), 7.74 (2H, d, J=9.1 Hz), 7.56 (1H, m), 7.47 (1H, t, J=5.9 Hz), 7.22 (2H, dd, J=7.7, 8.2 Hz), 4.36 (1H, t, J=5.0 Hz), 3.33 (2H, q, J=5.9 Hz), 2.71 (2H, q, J=6.3 Hz), 1.44-1.32 (4H, m).

HRFABMS: calcd. for C₂₀H₂₁F₂N₄O₄S₂ (M+Na⁺): 483.0972. Found: 483.0976.

Anal. calcd. for C₂₀H₂₀F₂N₄O₄S₂.0.8H₂O.0.1 Hexane: C, 48.94; H, 4.59; N, 11.08; S, 12.69. Found: C, 48.96; H, 4.49; N, 10.92; S. 12.46.

### Example A(6)

### 4-[4-Amino-5-(2,6-difluoro-benzoyl)-thiazol-2-ylamino]-N-[2-(2-hydroxy-ethoxy)-ethyl]-benzenesulfonamide

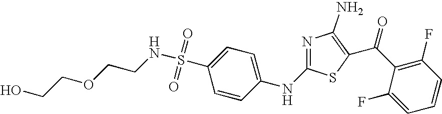

In a manner analogous to that of Example A(1), 4-[4-amino-5-(2,6-difluoro-benzoyl)-thiazol-2-ylamino]-benzenesulfonyl fluoride (150 mg, 0.36 mmol) and 2-(2-aminoethoxy) ethanol (0.29 mL, 2.9 mmol) gave a yellow solid in 57% yield.

¹H NMR (DMSO-d₆): δ 11.19 (1H, s), 8.25 (2H, bs), 7.79 (2H, d, J=9.5 Hz), 7.75 (2H, d, J=9.5 Hz), 7.58 (1H, t, J=6.1 Hz), 7.56 (1H, m), 7.22 (2H, dd, J=8.2, 7.8 Hz), 4.55 (1H, t, J=5.5 Hz), 3.43 (2H, q, J=5.0 Hz), 3.35 (4H, q, J=5.8 Hz), 2.89 (2H, q, J=5.8 Hz).

HRESIMS: calcd. for C₂₀H₂₁F₂N₄O₅S₂ (M+H⁺): 499.0921. Found: 499.0930.

Anal. calcd. for C₂₀H₂₀F₂N₄O₅S₂.0.5 CHCl₃: C, 44.11; H, 3.70; N, 10.04; S. 11.49. Found: C, 44.37; H, 3.72; N, 10.04; S. 11.64.

### Example A(7)

### 4-[4-Amino-5-(2,6-difluoro-benzoyl)-thiazol-2-ylamino]-N-(2,5-dichloro-benzyl)-benzenesulfonamide

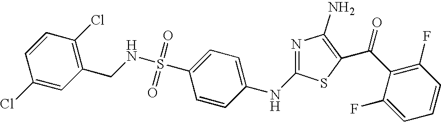

The title compound was prepared in a manner similar to that described for Example A(1). 4-[4-Amino-5-(2,6-difluoro-benzoyl)-thiazol-2-ylamino]-benzenesulfonyl fluoride (200 mg, 0.484 mmol) and 2,5-dichlorobenzylamine (256 mg, 1.4 mmol) gave a yellow powder in 40% yield.

¹H NMR (DMSO-d₆): δ 11.20 (1H, s), 8.25 (2H, bs), 8.22 (1H, t, J=6.2 Hz), 7.74 (4H, s), 7.56 (1H, m), 7.41 (1H, d, J=8.5 Hz), 7.35 (1H, d, J=2.4 Hz), 7.31(1H, dd, J=2.4, 8.5 Hz), 7.22 (1H, t, J=7.9 Hz), 4.08 (1H, d, J=6.2 Hz).

HRESIMS: calcd. for C₂₃H₁₇Cl₂F₂N₄O₃S₂ (M+H⁺): 569.0087. Found: 569.0112.

Anal. calcd. for C₂₃H₁₆Cl₂F₂N₄O₃S₂: C, 48.51; H, 2.83; N, 9.84; S, 11.26. Found: C, 48.81; H, 3.03; N, 9.80; S, 10.97.

### Example A(8)

### 4-[4-Amino-5-(2,6-difluoro-benzoyl)-thiazol-2-ylamino]-N-(3-pyrrolidin-1-yl-propyl)-benzenesulfonamide

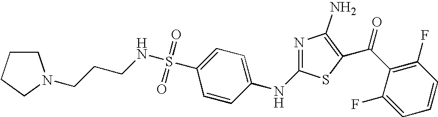

The title compound was prepared in a manner similar to that described for Example A(1). 4-[4-Amino-5-(2,6-difluoro-benzoyl)-thiazol-2-ylamino]-benzenesulfonyl fluoride (200 mg, 0.484 mmol) and 1-(3-aminopropyl)pyrrolidine (0.18 mL, 1.4 mmol) gave a yellow powder in 36% yield.

¹H NMR (DMSO-d₆): δ 11.10 (1H, bs), 8.25 (2H, bs), 7.79 (2H, d, J=9.0 Hz), 7.74 (2H, d, J=9.0 Hz), 7.55 (1H, m), 7.21 (1H, t, J=7.9 Hz).

HRESIMS: calcd. for C₂₃H₂₆F₂N₅O₃S₂ (M+H⁺): 522.1445. Found: 522.1458.

Anal. calcd. for C₂₃H₂₅F₂N₅O₃S₂.1.0H₂O: C, 51.19; H, 5.04; N, 12.98; S, 11.88. Found: C, 51.30; H, 5.00; N, 12.85; S, 11.66.

### Example A(9)

### 4-[4-Amino-5-(2,6-difluoro-benzoyl)-thiazol-2-ylamino]-N-(2-phenylamino-ethyl)-benzenesulfonamide

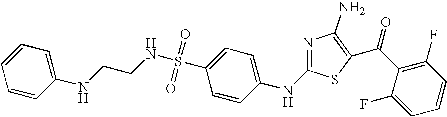

In a manner analogous to that of Example A(1), 4-[4-Amino-5-(2,6-difluoro-benzoyl)-thiazol-2-ylamino]-benzenesulfonyl fluoride (300 mg, 0.73 mmol) and N-phenylethylenediamine (0.28 mL, 2.2 mmol) gave a yellow solid in 65% yield.

¹H NMR (DMSO-d₆): δ 11.10 (1H, bs), 8.24 (2H, bs), 7.76 (4H, s), 7.65 (1H, bs), 7.56 (1H, m), 7.22 (2H, t, J=7.8 Hz), 7.04 (2H, t, J=7.8 Hz), 5.52 (1H, t, J=5.7 Hz), 3.07 (2H, q, J=6.3 Hz), 2.86 (2H, q, J=5.7 Hz).

HRESIMS: calcd. for C₂₄H₂₂F₂N₅O₃S₂ (M+H⁺): 530.1132. Found: 530.1129.

Anal. calcd. for C₂₄H₂₁F₂N₅O₃S₂.0.4 EtOH: C, 54.35; H, 4.30; N, 12.78; S. 11.70. Found: C, 54.14; H, 4.47; N, 12.91; S, 11.54.

### Example A(10)

### 4-[4-Amino-5-(2,6-difluoro-benzoyl)-thiazol-2-ylamino]-N-(3-isopropoxy-propyl)-benzenesulfonamide

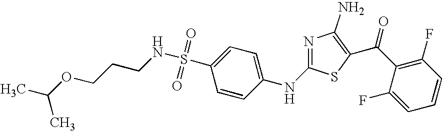

The title compound was prepared in a manner similar to that described for Example A(1). 4-[4-Amino-5-(2,6-difluoro-benzoyl)-thiazol-2-ylamino]-benzenesulfonyl fluoride (303 mg, 0.74 mmol) and 3-isopropoxypropylamine (0.31 mL, 2.2 mmol) gave a yellow powder in 56% yield.

¹H NMR (CD₃OD): δ 7.87 (2H, d, J=9.0 Hz), 7.81 (2H, d, J=9.0 Hz), 7.49 (1H, m), 7.07 (2H, dd, J=7.5, 8.3 Hz), 3.51 (1H, heptet, J=6.1 Hz), 3.43 (2H, q, J=6.1 Hz), 2.93 (2H, t, J=6.8 Hz), 1.67 (2H, quintet, J=6.3 Hz), 1.10 (6H, d, J=6.1 Hz).

HRFABMS: calcd. for C₂₀H₂₁F₂N₄O₄S₂ (M+Na⁺): 483.0972. Found: 483.0976.

Anal. calcd. for C₂₂H₂₄F₂N₄O₄S₂: C, 51.75; H, 4.74; N, 10.97; S. 12.56. Found: C, 51.77; H, 4.72; N, 10.99; S. 12.44.

### Example A(11)

### 4-[4-Amino-5-(2,6-difluoro-benzoyl)-thiazol-2-ylamino]-N-(5-methyl-furan-2-ylmethyl)-benzenesulfonamide

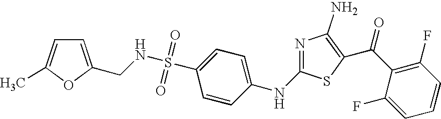

The title compound was prepared in a manner similar to that described for Example A(1). 4-[4-Amino-5-(2,6-difluoro-benzoyl)-thiazol-2-ylamino]-benzenesulfonyl fluoride (300 mg, 0.726 mmol) and 5-methyl-2-furfurylamine (2.2 mmol) gave a yellow powder in 84% yield.

¹H NMR (DMSO-d₆): δ 11.18 (1H, bs), 8.22 (2H, bs), 8.00 (1H, t, J=5.8 Hz), 7.71 (4H, s), 7.55 (1H, m), 7.22 (2H, dd, J=7.8, 8.0 Hz), 6.01 (1H, d, J=2.9 Hz), 5.86 (1H, q, J=2.9 Hz), 3.92 (2H, d, J=5.8 Hz), 2.09 (3H, s).

HRESIMS: calcd. for C₂₂H₁₉F₂N₄O₄S₂ (M+H⁺): 505.0816. Found: 505.0820.

Anal. calcd. for C₂₂H₁₈F₂N₄O₄S₂.0.2H₂O-0.3 n-C₆H₁₄: C, 53.53; H, 4.27; N, 10.49; S, 12.01. Found: C, 53.49; H, 4.23; N, 10.56; S, 11.94.

### Example A(12)

### 4-[4-Amino-5-(2,6-difluoro-benzoyl)-thiazol-2-ylamino]-N-(5-hydroxy-1,5-dimethyl-hexyl)-benzenesulfonamide

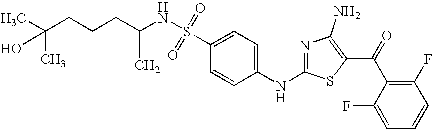

The title compound was prepared in a manner similar to that described for Example A(1). 4-[4-Amino-5-(2,6-difluoro-benzoyl)-thiazol-2-ylamino]-benzenesulfonyl fluoride (300 mg, 0.726 mmol) and 6-amino-2-methyl-2-heptanol (2.2 mmol) gave a white powder in 75% yield.

¹H NMR (DMSO-d₆): δ 11.18 (1H, bs), 8.22 (2H, bs), 7.76 (4H, s), 7.56 (1H, m), 7.43 (1H, d, J=7.8 Hz), 7.22 (2H, dd, J=8.0, 7.8 Hz), 4.00 (1H, s), 3.09 (1H, quintet, J=6.1 Hz), 0.97 (6H, d, J=1.2 Hz), 0.87 (3H, d, J=6.5 Hz).

HRFABMS: calcd. for C₂₀H₂₁F₂N₄O₄S₂ (M+Na⁺): 483.0972. Found: 483.0976.

Anal. calcd. for C₂₄H₂₈F₂N₄O₄S₂.0.4H₂O: C, 52.81; H, 5.32; N, 10.26; S. 11.75. Found: C, 53.08; H, 5.47; N, 10.13; S, 11.42.

### Example A(13)

### 4-[4-Amino-5-(2,6-difluoro-benzoyl)-thiazol-2-ylamino]-N-(3-diethylamino-propyl)-benzenesulfonamide

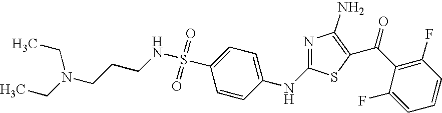

The title compound was prepared in a manner similar to that described for Example A(1). 4-[4-Amino-5-(2,6-difluoro-benzoyl)-thiazol-2-ylamino]-benzenesulfonyl fluoride (300 mg, 0.726 mmol) and 3-(diethylamino)propylamine (2.2 mmol) gave a yellow powder in 71% yield.

¹H NMR (DMSO-d₆): δ 11.10 (1H, bs), 8.22 (2H, bs), 7.79 (2H, d, J=9.0 Hz), 7.73 (2H, d, J=9.0 Hz), 7.56 (1H, bs), 7.55 (1H, m), 7.21 (2H, dd, J=7.8, 8.0 Hz), 2.76 (2H, q, J=3.8 Hz).

HRESIMS: calcd. for C₂₃H₂₈F₂N₅O₃S₂ (M+H⁺): 524.1602. Found: 524.1591.

Anal. calcd. for C₂₃H₂₇F₂N₅O₃S₂.1.0H₂O.0.9 Hexane: C, 55.09; H, 6.77; N, 11.31; S, 10.36. Found: C, 55.03; H, 6.55; N, 11.28; S, 10.27.

### Example A(14)

### 4-[4-Amino-5-(2,6-difluoro-benzoyl)-thiazol-2-ylamino]-N-(3-piperidin-1-yl-propyl)-benzenesulfonamide

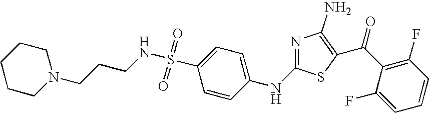

The title compound was prepared in a manner similar to that described for Example A(1). 4-[4-Amino-5-(2,6-difluoro-benzoyl)-thiazol-2-ylamino]-benzenesulfonyl fluoride (300 mg, 0.726 mmol) and 3-(1-piperidinyl)-propylamine (2.2 mmol) gave a yellow powder in 69% yield.

¹H NMR (DMSO-d₆): δ 11.05 (1H, bs), 8.22 (2H, bs), 7.79 (2H, d, J=9.0 Hz), 7.73 (2H, d, J=9.0 Hz), 7.56 (2H, m), 7.22 (2H, t, J=7.9 Hz).

HRESIMS: calcd. for C₂₄H₂₈F₂N₅O₃S₂ (M+H⁺): 536.1602. Found: 536.1583.

Anal. calcd. for C₂₄H₂₇F₂N₅O₃S₂.1.0 MeOH.0.2 Hexane: C, 53.80; H, 5.82; N, 11.97; S, 10.96. Found: C, 53.78; H, 5.78; N, 11.68; S, 12.62.

### Example A(15)

### 4-[4-Amino-5-(2,6-difluoro-benzoyl)-thiazol-2-ylamino]-N-[3-(2RS-methyl-piperidin-1-yl)-propyl]-benzenesulfonamide

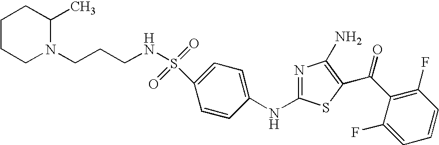

The title compound was prepared in a manner similar to that described for Example A(1). 4-[4-Amino-5-(2,6-difluoro-benzoyl)-thiazol-2-ylamino]-benzenesulfonyl fluoride (300 mg, 0.726 mmol) and 1-(3-aminopropyl)-2-pipecoline (0.38 mL, 2.2 mmol) gave a yellow powder in 52% yield.

¹H NMR (DMSO-d₆): δ 11.15 (1H, bs), 8.20 (2H, bs), 7.79 (2H, d, J=9.3 Hz), 7.73 (2H, d, J=9.3 Hz), 7.55 (1H, m), 7.53 (1H, bs), 7.22 (2H, dd, J=7.8, 8.2 Hz), 0.93 (3H, d, J=6.2 Hz).

HRESIMS: calcd. for C₂₅H₃₀F₂N₅O₃S₂ (M+H⁺): 550.1758. Found: 550.1751.

Anal. calcd. for C₂₅H₂₉F₂N₅O₃S₂.1.0H₂O.0.3 Hexane: C, 54.23; H, 5.98; N, 11.80; S, 10.81. Found: C, 54.53; H, 5.64; N, 11.67; S, 10.72.

### Example A(16)

### (4-Amino-2-{4-[4-(2-hydroxy-ethyl)-piperidine-1-sulfonyl]-phenylamino}thiazol-5-yl)-(2,6-difluoro-phenyl)-methanone

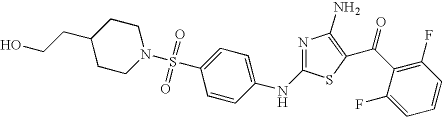

The title compound was prepared in a manner similar to that described for Example A(1). 4-[4-Amino-5-(2,6-difluoro-benzoyl)-thiazol-2-ylamino]-benzenesulfonyl fluoride (300 mg, 0.726 mmol) and 4-piperidineethanol (2.2 mmol) gave a yellow powder in 62% yield.

¹H NMR (DMSO-d₆): δ 11.22 (1H, s), 8.21 (2H, bs), 7.83 (2H, d, J=8.7 Hz), 7.69 (2H, d, J=8.7 Hz), 7.56 (1H, m), 7.21 (2H, dd, J=7.9, 8.0 Hz), 4.28 (1H, bs), 3.58 (2H, d, J=11.4 Hz), 3.38 (2H, d, J=3.7 Hz), 2.19 (2H, dd, J=10.4, 11.4 Hz), 1.68 (2H, d, J=12.0 Hz).

HRESIMS: calcd. for C₂₃H₂₅F₂N₄O₄S₂ (M+H⁺): 523.1285. Found: 523.1288.

Anal. calcd. for C₂₃H₂₄F₂N₄O₄S₂.1.0 MeOH: C, 51.97; H, 5.09; N, 10.10; S, 11.56. Found: C, 51.79; H, 4.94; N, 9.94; S, 11.28.

### Example A(17)

### 4-[4-Amino-5-(2,6-difluoro-benzoyl)-thiazol-2-ylamino]-N-(2-isopropoxy-ethyl)-benzenesulfonamide

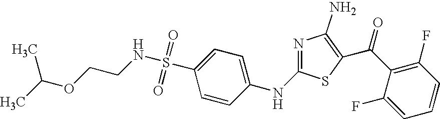

The title compound was prepared in a manner similar to that described for Example A(1). 4-[4-Amino-5-(2,6-difluoro-benzoyl)-thiazol-2-ylamino]-benzenesulfonyl fluoride (300 mg, 0.726 mmol) and 2-aminoethyl isopropyl ether (2.2 mmol) gave a yellow powder in 75% yield.

¹H NMR (DMSO-d₆): δ 11.07 (1H, s), 8.20 (2H, bs), 7.77 (4H, s), 7.56 (1H, bs), 7.55 (1H, m), 7.22 (2H, dd, J=8.0, 7.8 Hz), 3.45 (1H, heptet, J=6.0 Hz), 2.86 (2H, q, J=6.0 Hz), 1.01 (6H, d, J=6.0 Hz).

HRESIMS: calcd. for C₂₁H₂₃F₂N₄O₄S₂ (M+H⁺): 497.1129. Found: 497.1132.

Anal. calcd. for C₂₁H₂₂F₂N₄O₄S₂.0.7 MeOH.0.1 Hexane: C, 50.77; H, 5.01; N, 10.62; S, 12.16. Found: C, 50.96; H, 4.82; N, 10.67; S. 12.26.

### Example A(18)

### 4-[4-Amino-5-(2,6-difluoro-benzoyl)-thiazol-2-ylamino]-N-(2-p-tolyl-ethyl)-benzenesulfonamide

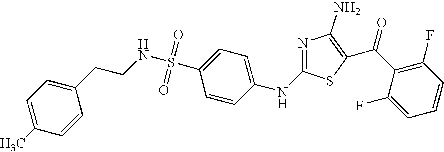

The title compound was prepared in a manner similar to that described for Example A(1). 4-[4-Amino-5-(2,6-difluoro-benzoyl)-thiazol-2-ylamino]-benzenesulfonyl fluoride (304 mg, 0.736 mmol) and 2-(p-tolyl)ethylamine (0.32 mL, 2.2 mmol) gave a white powder in 59% yield.

¹H NMR (DMSO-d₆): δ 11.19 (1H, s), 8.24 (2H, bs), 7.76 (2H, d, J=8.9 Hz), 7.71 (2H, d, J=8.9 Hz), 7.59 (1H, bs), 7.56 (1H, m), 7.22 (2H, dd, J=7.9, 8.1 Hz), 7.06 (2H, d, J=8.4 Hz), 7.01 (2H, q, J=8.4 Hz), 2.91 (2H, q, J=6.7 Hz), 2.61 (1H, t, J=7.7 Hz), 2.24 (3H, s).

Anal. calcd. for C₂₅H₂₂F₂N₄O₃S₂: C, 56.80; H, 4.20; N, 10.60; S, 12.13. Found: C, 56.73; H, 4.31; N, 10.66; S, 12.00.

### Example A(19)

### 4-[4-Amino-5-(2,6-difluoro-benzoyl)-thiazol-2-ylamino]-N-(2-ethylsulfanyl-ethyl)-benzenesulfonamide

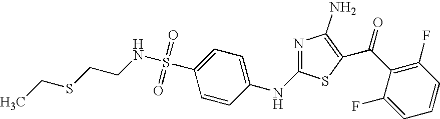

The title compound was prepared in a manner similar to that described for Example A(1). 4-[4-Amino-5-(2,6-difluoro-benzoyl)-thiazol-2-ylamino]-benzenesulfonyl fluoride (300 mg, 0.726 mmol) and 2-(ethylthio)ethylamine (0.23 mL, 2.2 mmol) gave a yellow powder in 42% yield.

¹H NMR (DMSO-d₆): δ 11.20 (1H, s), 8.25 (2H, bs), 7.80 (2H, d, J=9.3 Hz), 7.75 (2H, d, J=9.3 Hz), 7.71 (1H, t, J=5.7 Hz), 7.56 (1H, m), 7.22 (2H, dd, J=7.8, 7.9 Hz), 2.90 (2H, q, J=7.3 Hz), 2.44 (2H, q, J=7.3 Hz), 1.11 (3H, t, J=7.3 Hz).

Anal. calcd. for C₂₀H₂₀F₂N₄O₃S₃: C, 48.18; H, 4.04; N, 11.24; S, 19.29. Found: C, 48.01; H, 4.15; N, 11.23; S, 19.50.

### Example A(20)

### 4-[4-Amino-5-(2,6-difluoro-benzoyl)-thiazol-2-ylamino]-N-[2-(4-fluoro-phenyl)-ethyl]-benzenesulfonamide

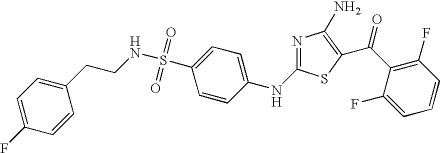

The title compound was prepared in a manner similar to that described for Example A(1). 4-[4-Amino-5-(2,6-difluoro-benzoyl)-thiazol-2-ylamino]-benzenesulfonyl fluoride (308 mg, 0.745 mmol) and 4-fluorophenethylamine (0.29 mL, 2.2 mmol) gave a yellow powder in 58% yield.

¹H NMR (DMSO-d₆): δ 8.25 (2H, bs), 7.77 (2H, d, J=9.1 Hz), 7.72 (2H, d, J=9.1 Hz), 7.60 (1H, t, J=5.5 Hz), 7.56 (1H, m), 7.07 (2H, dd, J=8.9, 8.7 Hz), 2.99 (2H, q, J=6.5 Hz), 2.66 (2H, t, J=7.3 Hz).

Anal. calcd. for C₂₄H₁₉F₃N₄O₃S₂: C, 54.13; H, 3.60; N, 10.52; S, 12.04. Found: C, 54.12; H, 3.66; N, 10.46; S, 11.96.

### Example A(21)

### 4-[4-Amino-5-(2,6-difluoro-benzoyl)-thiazol-2-ylamino]-N-(3-dimethylamino-propyl)-benzenesulfonamide

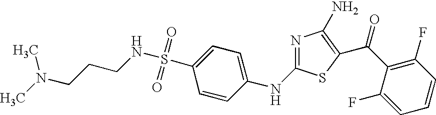

The title compound was prepared in a manner similar to that described for Example A(1). 4-[4-Amino-5-(2,6-difluoro-benzoyl)-thiazol-2-ylamino]-benzenesulfonyl fluoride (203 mg, 0.49 mmol) and 3-dimethylaminopropylamine (0.19 mL, 1.5 mmol) gave a yellow powder in 60% yield.

¹H NMR (DMSO-d₆): δ 11.15 (1H, bs), 8.21 (2H, bs), 7.79 (2H, d, J=9.2 Hz), 7.72 (2H, d, J=9.2 Hz), 7.55 (1H, m), 7.50 (1H, bs), 7.21 (2H, dd, J=7.8, 8.4 Hz), 2.74 (2H, t, J=6.9 Hz), 2.15 (2H, t, J=6.9 Hz), 2.05 (5H, s), 1.14 (2H, quintet, J=6.9 Hz).

HRESIMS: Calcd. For C₂₁H₂₄F₂N₅O₃S₂ (M+H⁺): 496.1289. Found: 496.1301.

Anal. calcd. for C₂₁H₂₃F₂N₅O₃S₂.0.2H₂O.0.7 CHCl₃: C, 44.73; H, 4.17; N, 12.02; S, 11.01. Found: C, 44.76; H, 4.36; N, 12.30; S, 11.38.

### Example A(22)

### 4-[4-Amino-5-(2,6-difluoro-benzoyl)-thiazol-2-ylamino]-N-furan-2-ylmethyl-benzenesulfonamide

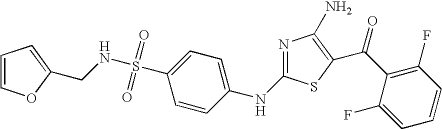

The title compound was prepared in a manner similar to that described for Example A(1). 4-[4-Amino-5-(2,6-difluoro-benzoyl)-thiazol-2-ylamino]-benzenesulfonyl fluoride (250 mg, 0.60 mmol) and furfurylamine (1.8 mmol) gave a yellow powder in 78% yield.

¹H NMR (DMSO-d₆): δ 11.18 (1H, s), 8.25 (2H, bs), 8.07 (1H, t, J=6.0 Hz), 7.77 (2H, d, J=9.5 Hz), 7.72 (2H, d, J=9.5 Hz), 7.56 (1H, m), 7.48 (1H, dd, J=0.8, 1.8 Hz), 7.22 (2H, dd, J=7.7, 8.2 Hz), 6.29 (1H, dd, J=1.8, 3.2 Hz), 6.16 (1H, dd, J=0.8, 3.2 Hz), 3.99 (2H, d, J=6.0 Hz).

HRESIMS: calcd. for C₂₁H₂₃F₂N₄O₄S₂ (M+H⁺): 491.0659. Found: 491.0647.

Anal. calcd. for C₂₁H₁₆F₂N₄O₄S₂.0.6H₂O: C, 50.31; H, 3.46; N, 11.18; S, 12.79. Found: C, 50.29; H, 3.49; N, 11.11; S. 12.75.

### Example A(23)

### 4-[4-Amino-5-(2,6-difluoro-benzoyl)-thiazol-2-ylamino]-N-(2-thiophen-2-yl-ethyl)-benzenesulfonamide

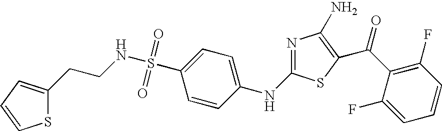

The title compound was prepared in a manner similar to that described for Example A(1). 4-[4-Amino-5-(2,6-difluoro-benzoyl)-thiazol-2-ylamino]-benzenesulfonyl fluoride (300 mg, 0.726 mmol) and 2-thiophenethylamine (2.2 mmol) gave a yellow powder in 69% yield.

¹H NMR (DMSO-d₆): δ 11.09 (1H, bs), 8.22 (2H, bs), 7.75 (4H, s), 7.55 (1H, m), 7.32 (1H, d, J=5.0 Hz), 7.21 (2H, dd, J=7.8, 8.0 Hz), 6.93 (1H, dd, J=3.1, 5.0 Hz), 6.86 (1H, d, J=3.1 Hz).

HRESIMS: calcd. for C₂₂H₁₉F₂N₄O₃S₃ (M+H⁺): 521.0587. Found: 521.0590.

Anal. calcd. for C₂₂H₁₈F₂N₄O₃S₃.1.2H₂O: C, 48.73; H, 3.79; N, 10.33; S. 17.74. Found: C, 48.66; H, 3.50; N, 10.14; S, 17.80.

### Example A(24)

### 4-[4-Amino-5-(2,6-difluoro-benzoyl)-thiazol-2-ylamino]-N-(2-pyridin-2-yl-ethyl)-benzenesulfonamide

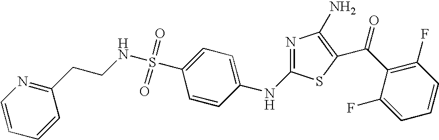

The title compound was prepared in a manner similar to that described for Example A(1). 4-[4-Amino-5-(2,6-difluoro-benzoyl)-thiazol-2-ylamino]-benzenesulfonyl fluoride (300 mg, 0.726 mmol) and 2-(2-aminoethyl)pyridine (0.26 mL, 2.2 mmol) gave a yellow powder in 53% yield.

¹H NMR (DMSO-d₆): δ 11.15 (1H, s), 8.44 (1H, d, J=4.7 Hz), 8.20 (2H, bs), 7.77 (2H, d, J=9.1 Hz), 7.03 (2H, d, J=9.1 Hz), 7.67 (1H, td, J=1.8, 7.7 Hz), 7.61 (1H, s), 7.56 (1H, m), 3.10 (2H, q, J=5.0 Hz), 2.83 (2H, t, J=7.4 Hz).

HRESIMS: calcd. for C₂₃H₂₀F₂N₅O₃S₂ (M+H⁺): 516.0976. Found: 516.0970.

Anal. calcd. for C₂₃H₁₉F₂N₅O₃S₂.1.2H₂O: C, 51.43; H, 4.02; N, 13.04; S, 11.94. Found: C, 51.23; H, 3.82; N, 12.84; S, 11.78.

### Example A(25)

### 4-[4-Amino-5-(2,6-difluoro-benzoyl)-thiazol-2-ylamino]-N-[3-(2-oxo-pyrrolidin-1-yl)-propyl]-benzenesulfonamide

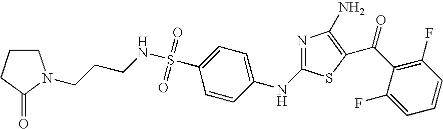

The title compound was prepared in a manner similar to that described for Example A(1). 4-[4-Amino-5-(2,6-difluoro-benzoyl)-thiazol-2-ylamino]-benzenesulfonyl fluoride (200 mg, 0.484 mmol) and N-(3′-aminopropyl)-2-pyrrolidinone (0.20 mL, 1.4 mmol) gave a yellow powder in 34% yield.

¹H NMR (DMSO-d₆): δ 11.14 (1H, s), 8.16 (2H, bs), 7.75 (2H, d, J=9.1 Hz), 7.70 (2H, d, J=9.1 Hz), 7.51 (1H, m), 7.43 (1H, t, J=5.7 Hz), 7.17 (2H, dd, J=7.6, 8.2 Hz), 3.19 (2H, t, J=7.0 Hz), 3.08 (2H, t, J=7.0 Hz), 2.65 (2H, q, J=6.6 Hz), 2.12 (2H, dd, J=7.3, 8.3 Hz), 1.82 (2H, quintet, J=7.8 Hz), 1.50 (2H, quintet, J=7.0 Hz).

HRESIMS: calcd. for C₂₃H₂₄F₂N₅O₄S₂ (M+H⁺): 536.1238. Found: 536.1220.

Anal. calcd. for C₂₃H₂₃F₂N₅O₄S₂.1.2H₂O: C, 49.58; H, 4.59; N, 12.57; S, 11.51. Found: C, 49.62; H, 4.34; N, 12.30; S. 11.25.

### Example A(26)

### 4-[4-Amino-5-(2,6-difluoro-benzoyl)-thiazol-2-ylamino]-N-(4-diethylamino-butyl)-benzenesulfonamide

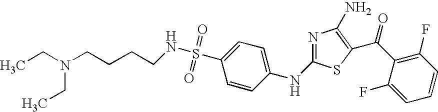

The title compound was prepared in a manner similar to that described for Example A(1). 4-[4-Amino-5-(2,6-difluoro-benzoyl)-thiazol-2-ylamino]-benzenesulfonyl fluoride (200 mg, 0.484 mmol) and N. N-diethyl-butane-1,4-diamine (1.4 mmol) gave a yellow powder in 42% yield.

¹H NMR (DMSO-d₆): δ 8.20 (2H, bs), 7.79 (2H, d, J=8.8 Hz), 7.73 (2H, d, J=8.8 Hz), 7.56 (1H, s), 7.55 (1H, m), 7.22 (2H, dd, J=7.7, 8.2 Hz), 0.93 (6H, t, J=6.9 Hz).

HRESIMS: calcd. for C₂₄H₃₀F₂N₅O₃S₂ (M+H⁺): 538.1758. Found: 538.1757.

Anal. calcd. for C₂₄H₂₉F₂N₅O₃S₂.0.5H₂O-0.3 CHCl₃: C, 50.11; H, 5.24; N, 12.02; S, 11.01. Found: C, 50.21; H, 5.26; N, 12.09; S. 11.15.

### Example A(27)

### 4-[4-Amino-5-(2,6-difluoro-benzoyl)-thiazol-2-ylamino]-N-thiophen-2-ylmethyl-benzenesulfonamide

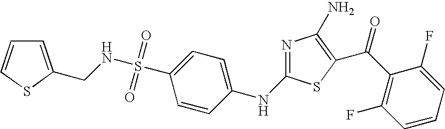

The title compound was prepared in a manner analogous to that described for Example A(1). Thiophene-2-methylamine and 4-{4-amino-5-[2,6-difluoro-benzoyl]-thiazol-2-ylamino}-benzenesulfonyl fluoride gave a product that was purified via column chromatography with 10% MeOH/CHCl₃ as eluant to provide a yellow foam in 56% yield.

¹H NMR (DMSO-d₆): δ 8.18 (1H, dd, J=6.1, 6.3 Hz), 7.74 (4H, s), 7.38 (1H, dd, J=3.0, 3.3 Hz), 7.26-7.18 (2H, dd, J=7.8, 8.0 Hz), 6.90 (1H, d, J=3.7 Hz), 4.17 (2H, d, J=6.2 Hz).

HRMALDIFTMS. Calcd for C₂₁H₁₇F₂N₄O₃S₃ (M+H⁺): 507.0431. Found: 507.0447.

Anal. calcd. for C₂₁H₁₆F₂N₄O₃S₃.0.8H₂O: C, 48.41; H, 3.41; N, 10.75; S, 18.07. Found: C, 48.59; H, 3.40; N, 10.38; S, 18.07.

### Example A(28)

### 4-[4-Amino-5-(2,6-difluoro-benzoyl)-thiazol-2-ylamino]-N-(5-hydroxy-pentyl)-benzenesulfonamide

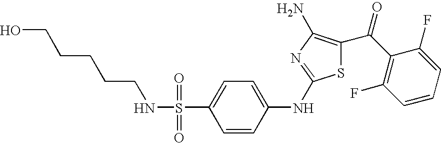

The title compound was prepared in a manner analogous to that described for Example A(1). 5-Amino-pentan-1-ol and 4-{4-amino-5-(2,6-difluoro-benzoyl)-thiazol-2-ylamino}benzenesulfonyl fluoride gave a product, which was purified via column chromatography with 10% MeOH/CHCl₃ as eluant to provide a yellow foam in 60% yield.

¹H NMR (DMSO-d₆): δ 7.92 (2H, d, J=9.6 Hz), 7.86 (2H, d, J=9.6 Hz), 7.72-7.64 (1H, m), 7.36 (2H, dd, J=7.7, 8.2 Hz).

HRMALDIFTMS C₂₁H₂₂F₂N₄O₄S₂Na (M+Na⁺): 519.0948. Found: 519.0964.

Anal. calcd. for C₂₁H₂₂F₂N₄O₄S₂.0.2MeOH.0.3CHCl₃: C, 47.93; H, 4.32; N, 10.40; S, 11.90. Found: C, 48.13; H, 4.50; N, 10.20; S, 11.52.

### Example A(29)

### 4-[4-Amino-5-(2,6-difluoro-benzoyl)-thiazol-2-ylamino]-N-(5-methyl-thiophen-2-ylmethyl)-benzenesulfonamide

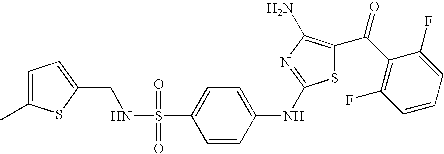

First (1)-(5-methyl-thiophen-2-yl)-methylamine, which has structural formula

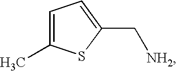

was prepared as follows. According to a procedure from Kuo, et al, Chem. Pharm. Bull., 39, 181-183 (1991), to a solution of 5-methyl-2-thiophenecarboxaldehyde (2.00 g, 15.9 mmol) in ethanol (20 mL) and H₂O (4 mL) were added hydroxylamine hydrochloride (1.65 g, 23.8 mmol) and NaOH (1.90 g, 47.6 mmol). The mixture was heated at reflux for 0.5 hour, allowed to cool to ambient temperature, and acidified to pH=4 with 2N HCl. The aqueous layer was extracted with ether (200 mL×2). The combined organic layers were dried over Na₂SO₄, filtered, and concentrated to give 1.44 g of cream-colored solid, of which a portion (1.00 g) was placed in a mixture with ethanol (16 mL) and conc. aq. NH₄OH (30 mL). Zn dust (3.47 g, 53.1 mmol) and ammonium acetate (437 mg, 5.66 mmol) were then added. The mixture was heated at reflux for 0.5 hour, allowed to cool to ambient temperature, and filtered. The filtrate was diluted with H₂O (25 mL) and extracted with 10% MeOH/CHCl₃ (50 mL). The organic layer was separated, dried over MgSO₄, and concentrated to afford 820 mg (61% yield from 5-methyl-2-thiophenecarboxaldehyde) of yellow oil, which was used without further purification.

The title compound was prepared in a manner analogous to that described for Example A(1). (5-Methyl-thiophen-2-yl)-methylamine (138 mg, 1.08 mmol) and 4-{4-amino-5-(2,6-difluoro-benzoyl)-thiazol-2-ylamino}-benzenesulfonyl fluoride (150 mg, 0.360 mmol) and purification via column chromatography with 8% MeOH/CHCl₃ as eluant provided a yellow foam in 67% yield.

¹H NMR (DMSO-d₆): δ 8.08 (1H, t, J=6.0 Hz), 7.72 (4H, s), 7.60-7.52 (1H, m), 7.22 (2H, dd, J=7.8, 8.0 Hz), 6.64 (1H, d, J=3.4 Hz), 6.54 (1H, dd, J=1.1, 3.3 Hz), 4.08 (2H, d, J=6.1 Hz), 2.32 (3H, s).

HRMALDIFTMS. calcd. for C₂₂H₁₈F₂N₄O₃S₃Na (M+Na⁺): 543.0407. Found: 543.0413.

Anal. calcd. for C₂₂H₁₈F₂N₄O₃S₃.0.4H₂O: C, 50.06; H, 3.59; N, 10.61; S, 18.23. Found: C, 49.71; H, 3.63; N, 10.50; S, 18.10.

### Example A(30)

### 4-[4-Amino-5-(2,6-difluoro-benzoyl)-thiazol-2-ylamino]-N-[1-(5-methyl-furan-2-yl)-ethyl]-benzenesulfonamide

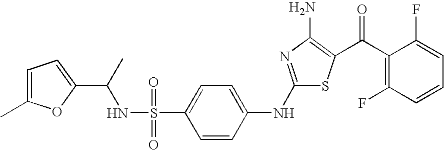

(1)-(5-Methyl-furan-2-yl)-ethylamine, which has the structural formula

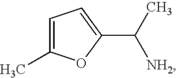

was prepared in a manner analogous to 1-(5-methyl-thiophen-2-yl)-methylamine, see Example A(29). 1-(5-Methyl-furan-2-yl)-ethanone (E/Z)-oxime (0.50 g, 3.6 mmol, from Kuo et al., Chem. Pharm. Bull., 39, 181-183 (1991)) was reduced to give 0.4 g of yellow oil, which displayed a ¹H NMR that matched literature (Kuo et al., Chem. Pharm. Bull., 39, 181-183, (1991)), and was used without further purification.

The title compound was prepared in a manner analogous to that described for Example A(1). 1-(5-Methyl-furan-2-yl)-ethylamine (272 mg, 2.17 mmol) and 4-{4-amino-5-(2,6-difluoro-benzoyl)-thiazol-2-ylamino}-benzenesulfonyl fluoride (300 mg, 0.720 mmol) and purification via column chromatography with 8% MeOH/CHCl₃ as eluant provided a yellow solid in 29% yield.

¹H NMR (CD₃OD): δ 7.52 (2H, d, J=9.3 Hz), 7.68 (2H, d, J=8.7 Hz), 7.52-7.44 (1H, m), 7.04 (2H, dd, J=7.4, 8.2 Hz), 5.88 (1H, d, J=3.2 Hz), 2.02 (3H, s), 1.38 (3H, d, J=7.0 Hz).

HRMALDIFTMS: calcd. for C₂₃H₂₁F₂N₄O₄S₂ (M+H⁺): 519.0972. Found: 519.0980.

Anal. calcd. for C₂₃H₂₀F₂N₄O₄S₂: C, 53.27; H, 3.89; N, 10.80; S, 12.37. Found: C, 53.09; H, 4.08; N, 10.57; S, 12.14.

### Example A(31)

### 4-[4-Amino-5-(2,6-difluoro-benzoyl)-thiazol-2-ylamino]-N-(2-propoxy-ethyl)-benzenesulfonamide

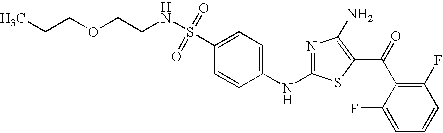

The title compound was prepared in a manner similar to that described for Example A(1). 4-[4-Amino-5-(2,6-difluoro-benzoyl)-thiazol-2-ylamino]-benzenesulfonyl fluoride (300 mg, 0.726 mmol) and 2-n-propoxyethylamine (224 mg, 2.2 mmol) gave a yellow powder in 83% yield.

¹H NMR (DMSO-d₆): δ 11.09 (1H, bs), 8.19 (2H, bs), 7.96 (4H, s), 7.55 (2H, m), 7.21 (2H, dd, J=8.0, 7.8 Hz), 1.43 (2H, hextet, J=7.1 Hz), 0.81 (3H, t, J=7.4 Hz).

HRESIMS: calcd. for C₂₁H₂₃F₂N₄O₄S₂ (M+H⁺): 497.1129. Found: 497.1126.

Anal. calcd. for C₂₁H₂₂F₂N₄O₄S₂.0.1H₂O.0.2 Hexane: C, 51.72; H, 4.89; N, 10.87; S, 12.44. Found: C, 51.52; H, 4.78; N, 11.13; S, 12.11.

### Example A(32)

### 4-[4-Amino-5-(2,6-difluoro-benzoyl)-thiazol-2-ylamino]-N-(3-phenyl-propyl)-benzenesulfonamide

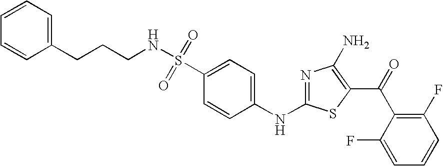

The title compound was prepared in a manner similar to that described for Example A(1). 4-[4-Amino-5-(2,6-difluoro-benzoyl)-thiazol-2-ylamino]-benzenesulfonyl fluoride (300 mg, 0.726 mmol) and 3-phenyl-1-propylamine (0.3 mL) gave a yellow powder in 84% yield.

¹H NMR (DMSO-d₆): δ 11.17 (1H, s), 8.20 (2H, bs), 7.79 (2H, d, J=9.0 Hz), 7.73 (2H, d, J=9.0 Hz), 7.10 (2H, d, J=8.2 Hz), 2.73 (2H, q, J=6.6 Hz), 1.64 (2H, quintet, J=7.4 Hz).

HRESIMS: calcd. for C₂₅H₂₃F₂N₄O₃S₂ (M+H⁺): 529.1180. Found: 529.1171.

Anal. calcd. for C₂₅H₂₂F₂N₄O₃S₂.0.1 Hexane: C, 57.24; H, 4.39; N, 10.43; S, 11.94. Found: C, 57.35; H, 4.45; N, 10.42; S, 11.73.

### Example A(33)

### 4-[4-Amino-5-(2,6-difluoro-benzoyl)-thiazol-2-ylamino]-N-[2-(3-chloro-phenyl)-ethyl]-benzenesulfonamide

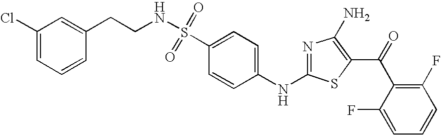

The title compound was prepared in a manner similar to that described for Example A(1). 4-[4-Amino-5-(2,6-difluoro-benzoyl)-thiazol-2-ylamino]-benzenesulfonyl fluoride (300 mg, 0.726 mmol) and 2-(3-chloro-phenyl)-ethylamine (0.3 mL) gave a yellow powder in 99% yield.

¹H NMR (DMSO-d₆): δ 11.07 (1H, s), 8.18 (2H, bs), 7.76 (2H, d, J=9.0 Hz), 7.71 (2H, d, J=9.0 Hz), 7.10 (2H, d, J=7.0 Hz), 2.98 (2H, q, J=6.7 Hz), 2.68 (2H, t, J=7.1 Hz).

HRESIMS: calcd. for C₂₄H₂₀ClF₂N₄O₃S₂ (M+H⁺): 549.0633. Found: 549.0636.

Anal. calcd. for C₂₄H₁₉ClF₂N₄O₃S₂.0.5H₂O.0.1 Hexane: C, 52.14; H, 3.81; N, 9.89; S, 11.32. Found: C, 52.50; H, 3.76; N, 9.89; S, 11.11.

### Example A(34)

### 4-[4-Amino-5-(2,6-difluoro-benzoyl)-thiazol-2-ylamino]-N-benzofuran-2-ylmethyl-benzenesulfonamide

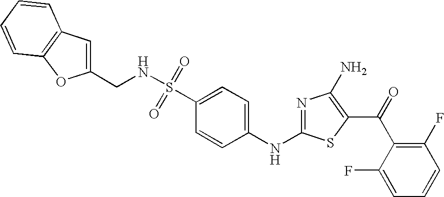

First C-benzofuran-2-yl-methylamine, which has the structural formula

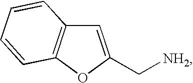

was made in a manner similar to that for 1-(5-methyl-thiophen-2-yl)-methylamine in Example A(29). Benzofuran-2-carboxaldehyde (3.00 g, 20.5 mmol) gave 2.48 g (82% overall yield) of a yellow oil, which was used without any further purification.

¹H NMR: δ 7.52 (1H, m), 7.44 (1H, m), 6.52 (2H, d, J=0.8 Hz), 3.98 (2H, d, J=0.8 Hz).

The title compound was prepared in a manner similar to that described for Example A(1). 4-[4-Amino-5-(2,6-difluoro-benzoyl)-thiazol-2-ylamino]-benzenesulfonyl fluoride (300 mg, 0.726 mmol) and C-benzofuran-2-yl-methylamine (320 mg, 2.20 mmol) gave a yellow powder in 33% yield.

¹H NMR (DMSO-d₆): δ 11.12 (1H, bs), 8.18 (1H, bs), 8.16 (2H, bs), 7.53 (2H, d, J=6.6 Hz), 7.42 (2H, d, J=7.7 Hz), 6.66 (1H, s), 4.17 (2H, s).

HRESIMS: calcd. for C₂₅H₁₉F₂N₄O₄S₂ (M+H⁺): 541.0816. Found: 541.0795.

Anal. calcd. for C₂₅H₁₈F₂N₄O₄S₂.0.5H₂O: C, 54.64; H, 3.48; N, 10.19; S, 11.67. Found: C, 54.60; H, 3.41; N, 10.30; S, 11.65.

### Example A(35)

### {4-[4-Amino-5-(2,6-difluoro-benzoyl)-thiazol-2-ylamino]-benzenesulfonylamino}acetic Acid Ethyl Ester

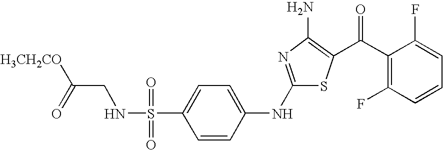

The title compound was prepared in a manner analogous to that described for Example A(1). Ethyl glycine hydrochloride and 4-{4-amino-5-(2,6-difluoro-benzoyl)-thiazol-2-ylamino}benzenesulfonyl fluoride gave a product which was purified via column chromatography with 10% MeOH/CHCl₃ as eluant to provide a yellow foam in 72% yield.

¹H NMR (DMSO-d₆): δ 8.08 (1H, t, J=6.2 Hz), 7.76 (4H, s), 7.60-7.50 (1H, m), 7.21 (2H, dd, J=7.8, 8.1 Hz), 3.96 (2H, q, J=7.1 Hz), 3.68 (2H, d, J=6.2 Hz), 1.10 (3H, t, J=7.1 Hz).

HRMALDIFTMS. calcd. for C₂₀H₁₉F₂N₄O₅S₂ (M+H⁺): 497.0765. Found: 497.0756.

Anal. calcd. for C₂₀H₁₈F₂N₄O₅S₂.0.1H₂O: C, 48.21; H, 3.68; N, 11.24; S, 12.87. Found: C, 47.91; H, 3.78; N, 11.20; S, 12.54.

### Example A(36)

### 4-[4-Amino-5-(2,6-difluoro-benzoyl)-thiazol-2-ylamino]-N-(5-hydroxy-5-methyl-hexyl)-benzenesulfonamide

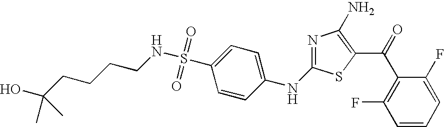

5-Hydroxy-5-methyl-hexyl mesylate, which has the structural formula

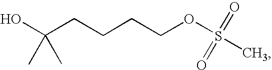

was prepared as follows. To a solution of 5-methyl-hexane-1,5-diol (500 mg, 3.78 mmol; Hernandez et al., J. Org. Chem., 62, 3153-3157 (1997)) in CH₂Cl₂ (5 mL) was added Et₃N (0.58 mL, 4.2 mmol). The resulting solution was cooled to −60° C. and methanesulfonyl chloride (0.323 mL, 4.16 mmol) was added. The mixture was allowed to warm to ambient temperature, stirred for 3 hours, then poured onto CH₂Cl₂ (50 mL). The organic layer was separated, washed with 0.5N HCl (25 mL), H₂O (25 mL), dried over Na₂SO₄, and concentrated to give a product, which was purified via column chromatography with 50% EtOAc/hexane as eluant to provide a white solid in 44% yield.

¹H NMR: δ 4.28 (2H, t, J=6.5 Hz), 3.02 (3H, s), 1.56 (1H, s), 1.26 (6H, s).

6-Azido-2-methyl-hexan-2-ol, which has the structural formula

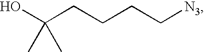

was prepared as follows. To a solution of 5-hydroxy-5-methyl-hexyl mesylate (350 mg, 1.66 mmol) in DMF (5 mL) was added NaN₃ (0.540 g, 8.30 mmol). The mixture was heated to 40° C. for 7 hours, then poured onto EtOAc (75 mL). The organic layer was separated, washed with H₂O (40 mL X 3), dried over Na₂SO₄, filtered, and concentrated to 0.250 g (96% yield) of colorless oil, which was used without any further purification.

¹H NMR: δ 3.16 (2H, t, J=6.8 Hz), 1.08 (6H, s).

6-Amino-2-methyl-hexan-2-ol, which has the structural formula

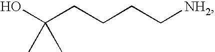

was prepared as follows. To a solution of 6-azido-2-methyl-hexan-2-ol (250 mg, 1.59 mmol) in a mixture of EtOAc (10 mL) and EtOH (2 mL) was added 10% Pd—C (75 mg). The resulting mixture was stirred under a H₂ balloon for 2 hours. The mixture was filtered through a pad of Celite and concentrated to 0.190 g (91% yield) of colorless oil, which was used without further purification.

¹H NMR: δ 1.00 (6H, s).

The title compound was prepared in a manner analogous to that described for Example A(1). 6-Amino-2-methyl-hexan-2-ol and 4-{4-amino-5-(2,6-difluoro-benzoyl)-thiazol-2-ylamino}-benzenesulfonyl fluoride gave a product, which was purified via column chromatography with 10% MeOH/CHCl₃ as eluant to provide a yellow foam in 83% yield.

¹H NMR (DMSO-d₆): δ 7.78 (2H, d, J=9.2 Hz), 7.72 (2H, d, J=9.1 Hz), 7.60-7.50 (1H, m), 7.46 (1H, t, J=6.0 Hz), 7.22 (2H, dd, J=7.9, 8.1 Hz), 4.0 (1H, s), 1.02 (6H, s).

HRMALDIFTMS. calcd. for C₂₃H₂₆F₂N₄O₄S₂Na (M+Na⁺): 547.1261. Found: 547.1241.

Anal. calcd. for C₂₃H₂₆F₂N₄O₄S₂.0.8H₂O: C, 51.25; H, 5.16; N, 10.39; S, 11.90. Found: C, 51.32; H, 5.19; N, 10.39; S, 11.81.

### Example A(37)

### 4-[4-Amino-5-(2,6-difluoro-benzoyl)-thiazol-2-ylamino]-N-(5-methyl-hexyl)-benzenesulfonamide

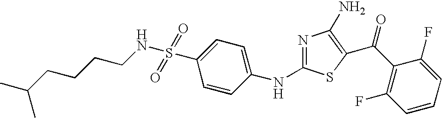

5-Methyl-hex-4-en-1-ol, which has the structural formula

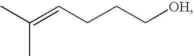

was prepared as follows. To a solution of δ-valerolactone (Sigma-Aldrich, 4.0 g, 40 mmol) in THF at −78° C. was added a solution of 1.5M MeLi in ether (66.6 mL, 99.9 mmol). The mixture was stirred for 0.5 hours at −78° C. and allowed to slowly warm to ambient temperature over 8 hours. The suspension was treated with HOAc (5.8 mL, 99.88 mmol) and stirred for 24 hours. The mixture was filtered and concentrated to give a colorless oil, which was distilled under reduced pressure to 1.5 g (28% in yield) of colorless oil, which was used further purification.

Methanesulfonic acid 5-methyl-hex-4-enyl ester, which has the structural formula

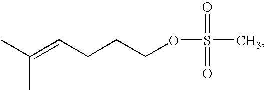

was prepared in a manner analogous to 5-hydroxy-5-methyl-hexyl mesylate, see Example A(38). 5-Methyl-hex-4-en-1-ol (200 mg, 1.72 mmol) provided 0.30 g of a yellow oil, which was used without further purification.

¹H NMR: δ 3.00 (3H, s), 1.72 (3H, s), 1.66 (3H, s).

6-Azido-2-methyl-hex-2-ene, which has the structural formula

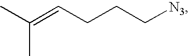

was prepared in a manner analogous to 6-azido-2-methyl-hexan-2-ol, see Example A(38). 5-methyl-hex-4-enyl mesylate (630 mg, 3.3 mmol) provided 400 mg of yellow oil, which was used without further purification.

¹H NMR: δ 1.72 (3H, s), 1.64 (3H, s).

5-Methyl-hexylamine, which has the structural formula

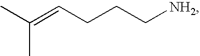

was prepared in a manner analogous to 6-amino-2-methyl-hexan-2-ol, see Example A(38). 6-Azido-2-methyl-hex-2-ene (400 mg, 2.87 mmol) provided 220 mg of colorless oil, which was used without further purification.

¹H NMR: δ 0.84 (3H, s), 0.78 (3H, s).

The title compound was prepared in a manner analogous to that described for Example A(1). 6-Amino-2-methyl-hexan-2-ol and 4-{4-amino-5-(2,6-difluoro-benzoyl)-thiazol-2-ylamino}-benzenesulfonyl fluoride gave a product, which was recrystallized from CH₃CN to provide a yellow solid in 79% yield.

¹H NMR (DMSO-d₆): δ 7.78 (2H, d, J=9.2 Hz), 7.72 (2H, d, J=9.2 Hz), 7.60-7.48 (1H, m), 7.44 (1H, t, J=5.8 Hz), 7.20 (2H, dd, J=7.8, 8.1 Hz), 2.73 (1H, d, J=6.8 Hz), 2.68 (1H, d, J=6.8 Hz), 0.82 (3H, s), 0.78 (3H, s).

MALDIFTMS (M+H⁺): 509.

Anal. calcd. for C₂₃H₂₆F₂N₄O₃S₂.0.5H₂O.0.5MeOH: C, 52.89; H, 5.48; N, 10.50; S, 12.02. Found: C, 53.02; H, 5.50; N, 10.75; S, 11.64.

### Example A(38)

### 4-[4-Amino-5-(2,6-difluoro-benzoyl)-thiazol-2-ylamino]-N-(1-methyl-1H-imidazol-5-ylmethyl)-benzenesulfonamide Trifluoroacetic Acid Salt

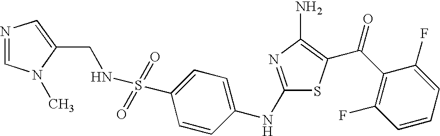

First the starting material, C-(1-methyl-1H-imidazol-5-yl)-methylamine, which has the structural formula

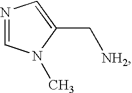

was prepared as follows. To a solution of 1-methylimidazole-5-carboxamide (931 mg, 7.44 mmol; Maybridge) in THF (15 mL) at 0° C. was carefully added lithium aluminum hydride (480 mg, 12.6 mmol). The mixture was heated at reflux overnight, cooled to 0° C., quenched with sat. aq. Na₂CO₃ (1.5 mL), diluted with ether (100 mL) and CH₂Cl₂ (100 mL), and filtered. The filtrate was concentrated in vacuo to give 713 mg (86% yield) of yellow oil, which was used without any further purification.

¹H NMR: δ 7.39 (1H, s), 6.89 (1H, s), 3.85 (2H, d, J=0.6 Hz), 3.66 (3H, s).

The title compound was prepared in a manner analogous to that described for Example A(1). Condensation of C-(1-methyl-1H-imidazol-5-yl)-methylamine and 4-{4-amino-5-(2,6-difluoro-benzoyl)-thiazol-2-ylamino}-benzenesulfonyl fluoride and subsequent purification via preparative HPLC provided 168 mg (57% yield) of a yellow powder.

¹H NMR (DMSO-d₆): δ 11.18 (1H, s), 8.90 (1H, s), 8.35-8.05 (3H, m), 7.73 (4H, dd, J=9.3, 11.6 Hz), 7.51 (1H, ddd, J=7.1, 8.1, 8.1 Hz), 7.40 (1H, s), 7.17 (2H, t, J=8.1 Hz), 4.08 (2H, d, J=5.8 Hz), 3.71 (3H, s).

Anal. calcd. for C₂₁H₁₈F₂N₆O₃S₂.1.4 TFA.1.0H₂O: C, 41.90; H, 3.16; N, 12.32; S, 9.40. Found: C, 41.99; H, 3.26; N, 12.31; S, 9.44.

### Example B(1)

### 4-[4-Amino-5-(2,4,6-trifluoro-benzoyl)-thiazol-2-ylamino]-N-(5-methyl-furan-2-ylmethyl)-benzenesulfonamide

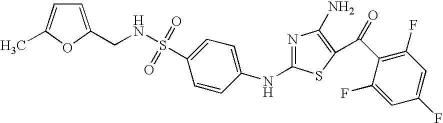

First 4-≡4-amino-5-(2,4,6-trifluoro-benzoyl)-thiazol-2-ylamino-}-benzenesulfonyl fluoride, which has the structural formula

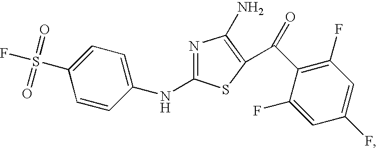

was made in the manner analogous 4-[4-amino-5-(2,6-difluoro-benzoyl)-thiazol-2-ylamino]-benzenesulfonyl fluoride from Example A(1). 4-isothiocyanato-benzenesulfonyl fluoride (547 mg, 2.52 mmol), cyanamide (116 mg, 2.77 mmol) and 2-bromo-2′,4′,6′-trifluoro-acetophenone (525 mg, 2.52 mmol, see World Patent Publication, WO 99/21845), afforded 750 mg (69% yield) of a yellow powder, which was used without further purification.

¹H NMR (DMSO-d₆): δ 11.55 (1H, s), 8.40 (2H, b), 8.12 (2H, d, J=9.0 Hz), 7.98 (2H, d, J=9.0 Hz), 7.38 (2H, dd, J=7.8, 7.8 Hz).

The title compound was prepared in a manner similar to that described for Example A(1). 4-[4-Amino-5-(2,6-difluoro-benzoyl)-thiazol-2-ylamino]-benzenesulfonyl fluoride (250 mg, 0.580 mmol) and 5-methyl-furfurylamine (0.20 mL, 1.7 mmol) gave a yellow powder in 48% yield.

¹H NMR (DMSO-d₆): δ 11.22 (1H, b), 8.25 (2H, b), 8.02 (1H, t, J=5.9 Hz), 7.75 (2H, d, J=9.4 Hz), 7.69 (2H, d, J=9.4 Hz), 7.35 (2H, dd, J=8.9, 8.1 Hz), 6.02 (1H, d, J=2.9 Hz), 5.86 (1H, q, J=2.9 Hz), 3.93 (2H, d, J=5.9 Hz), 2.09 (3H, s).

HRFABMS: calcd. for C₂₂H₁₈F₃N₄O₄S₂ (MH⁺): 523.0722. Found: 523.0710.

Anal. calcd. for C₂₂H₁₇F₃N₄O₄S₂.1.0 MeOH: C, 49.81; H, 3.82; N, 10.10; S, 11.56. Found: C, 49.91; H, 3.57; N, 10.06; S, 11.55.

### Example B(2)

### 4-[4-Amino-5-(2,4,6-trifluoro-benzoyl)-thiazol-2-ylamino]-N-(5-hydroxy-1,5-dimethyl-hexyl)-benzenesulfonamide

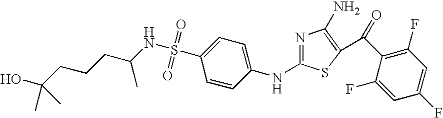

The title compound was prepared in a manner similar to that described for Example B(1). 4-[4-Amino-5-(2,4,6-trifluoro-benzoyl)-thiazol-2-ylamino]-benzenesulfonyl fluoride (200 mg, 0.464 mmol) and 6-amino-2-methyl-hepten-2-ol (1.4 mmol) gave a yellow powder in 56% yield.

¹H NMR (DMSO-d₆): δ 11.22 (1H, s), 8.28 (2H, bs), 7.77 (4H, s), 7.44 (1H, d, J=7.9 Hz), 7.34 (2H, dd, J=9.1, 7.9 Hz), 4.00 (1H, s), 0.97 (6H, d, J=1.4 Hz), 0.87 (3H, d, J=6.5 Hz).

HRESIMS: calcd. for C₂₄H₂₈F₃N₄O₄S₂ (M+H⁺): 557.1504. Found: 557.1482.

Anal. calcd. for C₂₄H₂₇F₃N₄O₄S₂.0.7H₂O: C, 50.64; H, 5.03; N, 9.84; S, 11.27. Found: C, 50.81; H, 5.07; N, 9.82; S, 11.17.

### Example C(1)

### 4-[4-Amino-5-(2,6-difluoro-benzoyl)-thiazol-2-ylamino]-N-phenyl-benzenesulfonamide

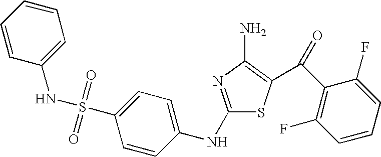

The title compound was made as follows. To 4-[4-amino-5-(2,6-difluoro-benzoyl)-thiazol-2-ylamino]-benzenesulfonyl fluoride (200 mg, 0.484 mmol) and aniline (132 μl, 1.45 mmol) in pyridine (1 mL), was added DMAP (5 mg). The mixture was heated at 100° C. for 48 hours. The mixture was partitioned between CHCl₃ and 1N HCl, the organic layer was washed with brine, dried over Na₂SO₄ and concentrated to a crude residue, which was purified via column chromatography to give 75 mg (32%) of a yellow solid.

¹H NMR (DMSO-d₆): δ 11.07 (1H, s), 10.20 (1H, s), 8.23 (2H, bs), 7.74 (2H, d, J=9.4 Hz), 7.69 (2H, d, J=9.4 Hz), 7.55 (1H, m), 7.08 (2H, dd, J=1.2, 7.5 Hz), 7.01 (2H, tt, J=1.2, 7.3 Hz).

HRESIMS: calcd. for C₂₂H₁₇F₂N₄O₃S₂ (M+H⁺): 487.0710. Found: 487.0706.

Anal. calcd. for C₂₂H₁₆F₂N₄O₃S₂.0.2 hexane-0.4H₂O: C, 54.54; H, 3.87; N, 10.96; S, 12.55. Found: C, 54.72; H, 3.67; N, 10.87; S, 12.39.

### Example D(1)

### 4-[4-Amino-5-(2,6-difluoro-benzoyl)-thiazol-2-ylamino]-N-piperidin-3-ylmethyl-benzenesulfonamide

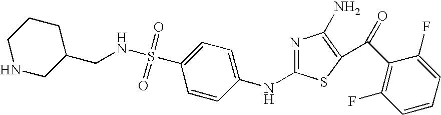

First, 3-[(4-{4-amino-5-(2,6-difluoro-benzoyl)-thiazol-2-ylamino}-benzenesulfonylamino)-methyl]-N-butoxycarbonyl-piperidine, which has the structural formula

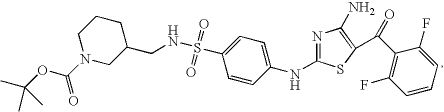

was prepared in a manner like that described for Example A(1). 4-[4-Amino-5-(2,6-difluoro-benzoyl)-thiazol-2-ylamino]-benzenesulfonyl fluoride (296 mg, 0.72 mmol) and 3-(aminomethyl)-1-N-BOC-piperidine (0.3 mL; Astatech, Inc.) gave a yellow solid that was used immediately in the next step without any further purification.

¹H NMR (DMSO-d₆): δ 11.32 (1H, s), 8.25 (2H, bs), 7.82 (2H, d, J=9.0 Hz), 7.75 (2H, d, J=9.0 Hz), 7.62 (1H, t, J=6.0 Hz), 7.57 (1H, m), 7.25 (2H, dd, J=7.68.3 Hz), 2.60 (2H, t, J=6.5 Hz), 1.39 (9H, s).

The title compound was made as follows. A solution of crude 3-[(4-{4-amino-5-(2,6-difluoro-benzoyl)-thiazol-2-ylamino}-benzenesulfonylamino)-methyl]-N-butoxycarbonyl-piperidine (0.72 mmol) in trifluoroacetic acid (TFA; 3 mL) at 0° C. stirred for a half hour, then concentrated under reduced pressure. The residue was taken up into MeOH (3 mL), and basified with sat. aq. Na₂CO₃ to pH=10, whereupon the resultant precipitate was filtered off, washed with water, and dried under vacuum to give 290 mg (80% for two steps) of a yellow solid.

¹H NMR (DMSO-d₆): δ 8.09 (2H, bs), 7.69 (2H, d, J=8.9 Hz), 7.63 (2H, d, J=8.9 Hz), 7.51 (1H, bs), 7.50 (1H, m), 7.17 (2H, dd, J=7.8, 8.0 Hz), 2.95 (1H, d, J=11.9 Hz), 2.87 (1H, d, J=11.9 Hz), 2.57 (2H, d, J=6.5 Hz), 2.18 (1H, dd, J=10.3, 11.9 Hz), 1.65 (2H, d, J=12.8 Hz),

EISMS: calcd. for C₂₂H₂₄F₂N₅O₃S₂ (M+H⁺): 508.1289, found: 508.1295.

Anal. calcd. for C₂₂H₂₃F₂N₅O₃S₂ 1.0 MeOH: C, 51.19; H, 5.04; N, 12.98; S. 11.88. Found: C, 51.50; H, 4.97; N, 12.85; S, 11.62.

### Example D(2)

### 4-[4-Amino-5-(2,6-difluoro-benzoyl)-thiazol-2-ylamino]-N-piperidin-2-ylmethyl-benzenesulfonamide

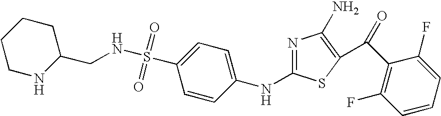

2-[(4-(4-Amino-5-(2,6-difluoro-benzoyl)-thiazol-2-ylamino)-benzenesulfonylamino)-methyl]-N-butoxycarbonyl-piperidine, which has the structural formula

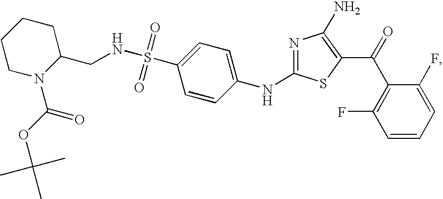

was prepared in a manner similar to Example A(1). 4-[4-Amino-5-(2,6-difluoro-benzoyl)-thiazol-2-ylamino]-benzenesulfonyl fluoride (400 mg, 0.968 mmol) and 2-(aminomethyl)-1-N-BOC-piperidine (622 mg, Astatech, Inc.) gave a yellow solid which was used immediately in the next step without any further purification.

The title compound was made in a manner analogous to 4-[4-amino-5-(2,6-difluoro-benzoyl)-thiazol-2-ylamino]-N-piperidin-3-ylmethyl-benzenesulfonamide in Example D(1). 2-[(4-{4-Amino-5-(2,6-difluoro-benzoyl)-thiazol-2-ylamino}-benzenesulfonylamino)-methyl]-N-butoxycarbonyl-piperidine gave 210 mg (43% for two steps) of a yellow solid.

¹H NMR (DMSO-d₆): δ 8.14 (2H, bs), 7.76 (2H, d, J=8.9 Hz), 7.70 (2H, d, J=8.9 Hz), 7.52 (1H, m), 7.19 (2H, dd, J=7.7, 8.1 Hz).

HRESIMS: calcd. for C₂₂H₂₄F₂N₅O₃S₂: 508.1289. Found: 508.1278.

Anal. calcd. for C₂₂H₂₃F₂N₅O₃S₂.0.5H₂O.0.2 TFA: C, 49.88; H, 4.52; N, 12.98; S, 11.89. Found: C, 49.93; H, 4.48; N, 12.81; S, 11.97.

### Example D(3)

### 4-[4-Amino-5-(2,6-difluoro-benzoyl)-thiazol-2-ylamino]-N-(2-methylamino-ethyl)-benzenesulfonamide

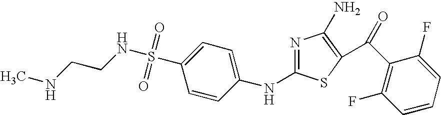

4-[4-Amino-5-(2,6-difluoro-benzoyl)-thiazol-2-ylamino]-N-(N-butoxycarbonyl-2-methylamino-ethyl)-benzenesulfonamide, which has the structural formula

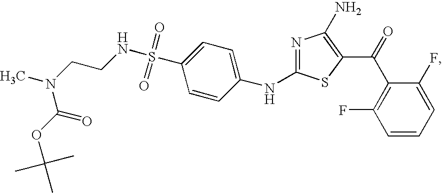

was prepared in a manner similar to Example A(1). 4-[4-Amino-5-(2,6-difluoro-benzoyl)-thiazol-2-ylamino]-benzenesulfonyl fluoride (300 mg, 0.726 mmol) and N-BOC-N-methyl-ethylenediamine (349 mg, 2.2 mmol; Fluka) gave a brown hard foam which was used immediately in the next step without any further purification.

The title compound was made in a manner analogous to 4-[4-amino-5-(2,6-difluoro-benzoyl)-thiazol-2-ylamino]-N-piperidin-3-ylmethyl-benzenesulfonamide in Example D(1). 4-[4-Amino-5-(2,6-difluoro-benzoyl)-thiazol-2-ylamino]-N-(N-butoxycarbonyl-2-methylamino-ethyl)-benzenesulfonamide gave 210 mg (62% for two steps) of a yellow solid.

¹H NMR (DMSO-d₆): δ 8.21 (2H, bs), 7.41 (4H, s), 7.54 (1H, m), 7.21 (2H, dd, J=7.7, 8.1 Hz), 2.80 (2H, t, J=6.6 Hz), 2.50 (2H, t, J=6.6 Hz), 2.20 (3H, s).

HRESIMS: calcd. for C₁₉H₂₀F₂N₅O₃S₂: 468.0976. Found: 469.0985.

Anal. calcd. for C₁₉H₁₉F₂N₅O₃S₂.0.2H₂O: C, 48.44; H, 4.15; N, 14.87; S, 13.61. Found: C, 48.45; H, 4.14; N, 14.72; S, 13.41.

### Example E(1)

### 4-[4-Amino-5-(2,6-difluoro-benzoyl)-thiazol-2-ylamino]-N-(4-methyl-thiazol-2-yl)-benzenesulfonamide

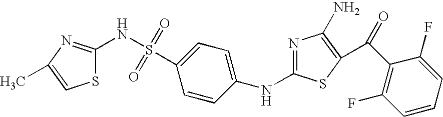

The title compound was made as follows. A mixture of 4-[4-amino-5-(2,6-difluoro-benzoyl)-thiazol-2-ylamino]-benzenesulfonyl fluoride (300 mg, 0.726 mmol), 2-amino-4-methylthiazole (249 mg, 2.2 mmol), pyridine (1.5 mL), and DMAP (6 mg) was heated at 100° C. for 3 days. The mixture was partitioned between 20% CH₃OH/CHCl₃ and 1N HCl, the organic layer was washed with brine, dried over Na₂SO₄, and concentrated to a residue, which was purified via preparative TLC to give 84 mg (23%) of a yellow solid.

¹H NMR (DMSO-d₆): 12.56 (1H, s), 11.13 (1H, s), 8.18 (2H, bs), 7.77 (2H, d, J=9.1 Hz), 7.71 (2H, d, J=9.1 Hz), 7.21 (2H, dd, J=7.6, 7.8 Hz), 2.08 (3H, s).

HRESIMS: calcd. for C₂₀H₁₆F₂N₅O₃S₃ (M+H⁺): 508.0383. Found: 508.0395.

Anal. calcd. for C₂₀H₁₅F₂N₅O₃S₃.0.3H₂O: C, 48.03; H, 3.50; N, 13.21; S, 18.14. Found: C, 47.84; H, 3.43; N, 13.03; S, 18.27.

### Example F(1)

### 4-[4-Amino-5-(2,6-difluoro-3-nitro-benzoyl)-thiazol-2-ylamino]-benzenesulfonamide

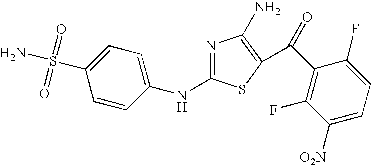

2′,6′-Difluoro-3′-nitro-acetophenone, which has the structural formula

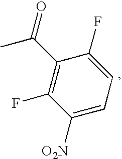

was first prepared as follows. To conc. H₂SO₄ (3 mL) and conc. HNO₃ (3 mL) at −40° C. was added 2,6-difluoroacetophenone (500 mg, 3.20 mmol). The mixture was allowed to slowly warm to 0° C. over 90 minutes, then dumped onto crushed ice and extracted with CH₂Cl₂. The organic layer was separated, washed with water and sat. aq. NaHCO₃, dried over Na₂SO₄, and concentrated to give 640 mg (100%) of yellow oil, which was used without further purification.

¹H NMR: δ 8.20 (1H, ddd, J=5.6, 8.3, 9.3 Hz), 7.12 (1H, ddd, J=1.8, 8.3, 9.3 Hz), 2.65 (3H, t, J=1.6 Hz).

2-Bromo-2′,6′-difluoro-3′-nitro-acetophenone, which has the structural formula

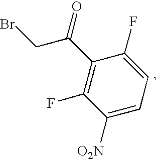

was made with a procedure from King et al., J. Org. Chem, 29, 3459-3461 (1964). To a solution of 2′,6′-difluoro-3′-nitro-acetophenone (3.91 g, 19.4 mmol) in EtOAc (25 mL) was added copper (II) bromide (8.70 g, 38.9 mmol). The resultant mixture was heated at reflux for 3 hours, allowed to cool, and the solid was filtered off and rinsed with ether. The filtrate was passed through a pad of silica gel and concentrated in vacuo to provide 5.37 g (99% yield) of a yellow solid, which was used without any further purification.

¹H NMR: δ 8.27 (1H, ddd, J=5.6, 8.4, 9.3 Hz), 7.17 (1H, ddd, J=1.8, 8.4, 9.3 Hz), 4.34 (2H, t, J=0.8 Hz).

The title compound was made as follows. To a mixture of 4-isothiocyanato-benzenesulfonamide (557 mg, 2.60 mmol), cyanamide (131 mg, 3.12 mmol), and MeCN (3 mL) was added a solution of potassium t-butoxide (321 mg, 2.86 mmol) in t-butanol (3 mL). After a half-hour, 2-bromo-2′,6′-difluoro-3′-nitro-acetophenone (800 mg, 2.86 mmol) was added. After one hour, water (20 mL) was added, allowed to stir for half hour, then acidified to pH=6 with 1N HCl. The resultant solid was filtered, washed with water and ether (2×3 mL), recrystallized from methanol, and dried under vacuum to furnish a yellow powder in 43% yield.

¹H NMR (DMSO-d₆): δ 11.08 (1H, s), 8.25 (2H, bs), 7.62 (2H, d, J=9.0 Hz), 7.56 (2H, d, J=9.0 Hz), 7.33 (2H, dd, J=8.1, 8.8 Hz), 7.09 (2H, s).

ESIMS (M+H⁺): 456.

Anal. calcd. for C₁₆H₁₁F₂N₅O₅S₂.0.6 MeOH: C, 42.01; H, 2.85; N, 14.75; S, 13.51. Found: C, 41.73; H, 2.57; N, 14.48; S, 13.45.

### Example F(2)

### 4-[4-Amino-5-(2-fluoro-benzoyl)-thiazol-2-ylamino]-benzenesulfonamide

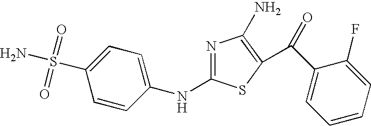

First the starting material 2-bromo-2′-fluoro-acetophenone, which has the structural formula

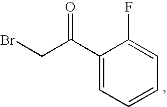

was made in a manner similar to that for 2-bromo-2′,6′-difluoro-3′-nitro-acetophenone in Example F(1). 2′-Fluoro-acetophenone (2.41 g, 17.4 mmol) and CuBr₂ (7.79 g, 34.9 mmol) gave 3.40 g (90%) of green oil, which was used without any further purification.

¹H NMR: δ 7.94 (1H, ddd, J=1.8, 7.6, 7.6 Hz), 7.59 (1H, ddd, J=1.8, 5.2, 9.3 Hz), 7.28 (1H, t, J=7.7 Hz), 7.17 (1H, dd, J=8.4, 11.4 Hz), 4.52 (2H, d, J=2.3 Hz).

The title compound was made in a manner analogous to that for 4-[4-amino-5-(2,6-difluoro-benzoyl)-thiazol-2-ylamino]-benzenesulfonyl fluoride from Example A(1). 4-Isothiocyanato-benzenesulfonamide (214 mg, 1.00 mmol) and 2-bromo-2′-fluoro-acetophenone (239 mg, 1.10 mmol), precipitation, ether rinse, and drying gave 167 mg (43% yield) of yellow powder.

¹H NMR (DMSO-d₆): δ 11.07 (1H, s), 8.15 (2H, bs), 7.78 (4H, ddd, J=3.1, 6.5, 9.5 Hz), 7.50 (2H, dd, J=6.3, 7.3 Hz), 7.18 (2H, s).

Anal. calcd. for C₁₆H₁₃FN₄O₃S₂: C, 48.97; H, 3.34; N, 14.28; S. 16.34. Found: C, 49.17; H, 3.51; N, 14.01; S. 16.11.

### Example G(1)

### 4-[4-Amino-5-(3-amino-2,6-difluoro-benzoyl)-thiazol-2-ylamino]-benzenesulfonamide

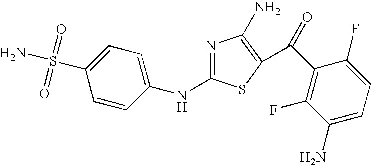

The title compound was made as follows. 4-[4-Amino-5-(2,6-difluoro-3-nitro-benzoyl)-thiazol-2-ylamino]-benzenesulfonamide (Example F(1); 333 mg, 0.73 mmol) and SnCl₂.2H₂O (495 mg, 2.19 mmol) in dioxane (5 mL) and EtOH (1.25 mL) refluxed for one hour and was then allowed to cool. A small amount of Celite and MeOH (5 mL) was added, basified to pH=10 with sat. aq. Na₂CO₃, and a solid cake filtered off and rinsed. The filtrate was concentrated in vacuo and the resultant residue purified via column chromatography to afford 171 mg (55% yield) of a light brown solid.

¹H NMR (DMSO-d₆): δ 11.14 (1H, s), 8.07 (2H, bs), 7.73 (4H, s), 7.21 (2H, s).

HREISMS: calcd. for C₁₆H₁₄F₂N₅O₃S₂: 426.0506, Found: 426.0518.

Anal. calcd. for C₁₆H₁₃F₂N₅O₃S₂.0.6H₂O: C, 44.05; H, 3.28; N, 16.05; S, 14.70. Found: C, 44.30; H, 3.31; N, 15.82; S. 14.81.

### Example G(2)

### 2-Amino-4-[4-amino-5-(2,6-difluoro-benzoyl)-thiazol-2-ylamino]-benzenesulfonamide

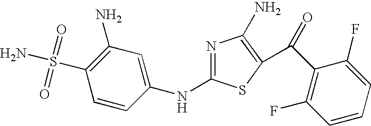

The title compound was prepared in a similar fashion to Example G(1): 4-[4-Amino-5-(2,6-difluoro-benzoyl)-thiazol-2-ylamino]-2-nitro-benzenesulfonamide {0.24 g, 0.52 mmol; Example S(3)} gave a yellow solid in 72% yield.

¹H NMR (CD₃OD): δ7.61 (1H, d, J=8.8 Hz), 7.52-7.42 (1H, m), 7.21 (1H, d, J=2.1 Hz), 7.06 (2H, dd, J=7.4, 8.5 Hz), 6.82 (1H, dd, J=2.2, 8.7 Hz)

HRMALDIFTMS (MH⁺): calcd.: 426.0501. Found: 426.0490.

Anal. calcd. for C₁₆H₁₃F₂N₅O₅S₂.0.9MeOH: C, 44.68; H, 3.68; N, 15.42; S, 14.12. Found: C, 44.94; H, 3.30; N, 15.11; S, 14.04.

### Example H(1)

### N-{3-[4-Amino-2-(4-sulfamoyl-phenylamino)-thiazole-5-carbonyl]-2,4-difluoro-phenyl}-acetamide

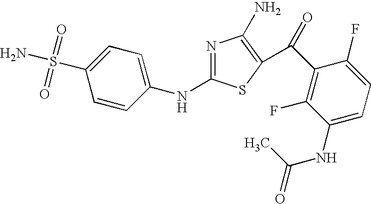

3′-Amino-2′,6′-difluoro-acetophenone, which has the structural formula

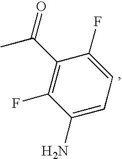

was first made as follows. 2′,6′-Difluoro-3′-nitro-acetophenone (from Example F(1); 527 mg, 2.61 mmol) and 10% Pd/C (53 mg) stirred in ethyl acetate (5 mL) under an atmosphere of H₂ overnight. The catalyst was filtered off and the filtrate concentrated in vacuo to obtain 450 mg (100% yield) of brown oil, which was submitted to the next step without any further purification.

¹H NMR: δ 6.81 (1H, td, J=5.7, 9.0 Hz), 6.76 (1H, td, J=1.0, 9.0 Hz), 3.67 (2H, bs), 2.57 (2H, t, J=1.8 Hz).

N-(3-Acetyl-2,4-difluoro-phenyl)-acetamide, which has the structural formula

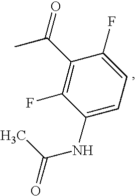

was prepared as follows. 3′-Amino-2′,6′-difluoro-acetophenone (from Example H(1); 450 mg, 2.60 mmol) and acetic anhydride (1.27 mL) in acetic acid (1.8 mL) stirred at 70° C. for a half hour. The mixture was partitioned between ether and water, the organic layer separated, washed with sat. aq. Na₂CO₃, dried over MgSO₄, and concentrated to give 452 mg (81% yield) of brown oil, which was submitted to the next step without any further purification.

¹H NMR: δ 8.35 (1H, td, J=5.7, 9.3 Hz), 6.95 (1H, td, J=1.0, 9.3 Hz), 2.60 (3H, dd, J=0.5, 1.5 Hz), 2.23 (3H, s).

N-[3-(2-Bromoacetyl)-2,4-difluoro-phenyl]-acetamide, which has the structural formula

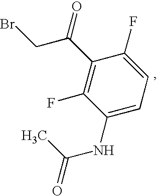

was then made in a manner similar to 2-bromo-2′,6′-difluoro-3′-nitro-acetophenone from Example F(1)). N-(3-Acetyl-2,4-difluoro-phenyl)-acetamide (452 mg, 2.12 mmol) and CuBr₂ (947 mg, 4.24 mmol) afforded 584 mg (95% yield) of a yellow solid, which was used without further purification.

¹H NMR: δ 8.45 (1H, td, J=5.8, 9.3 Hz), 6.99 (1H, td, J=1.9, 9.3 Hz), 4.35 (2H, t, J=0.8 Hz), 2.24 (3H, s).

The title compound was made in a manner analogous to that for 4-[4-amino-5-(2,6-difluoro-3-nitro-benzoyl)-thiazol-2-ylamino]-benzenesulfonamide (Example F(1). 4-Isothiocyanato-benzenesulfonamide (98 mg, 0.46 mmol) and N-[3-(2-bromoacetyl)-2,4-difluoro-phenyl]-acetamide (140 mg, 0.479 mmol) gave a yellow solid in 84% yield.

¹H NMR (DMSO-d₆): δ 11.17 (1H, s), 9.83 (1H, s), 8.26 (2H, bs), 7.89 (1H, m), 7.80 (2H, d, J=8.5 Hz), 7.75 (2H, d, J=8.5 Hz), 7.79 (2H, s), 7.16 (1H, dd, J=7.7, 8.6 Hz), 2.08 (3H, s).

ESIMS (MH⁺): 468.

Anal. calcd. for C₁₈H₁₅F₂N₅O₄S₂.1.1H₂O.0.3 t-BuOH: C, 45.26; H, 4.00; N, 13.74; S, 12.59. Found: C, 45.16; H, 3.62; N, 13.39; S, 12.58.

### Example 1(1)

### Thiophene-2-carboxylic Acid {3-[4-Amino-2-(4-sulfamoyl-phenylamino)-thiazole-5-carbonyl]-2,4-difluoro-phenyl}-amide

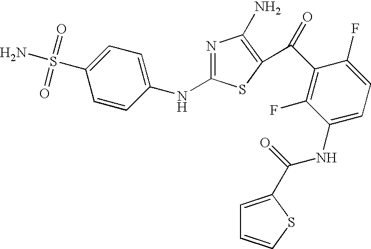

Thiophene-2-carboxylic acid (3-acetyl-2,4-difluoro-phenyl)-amide, which has the structural formula

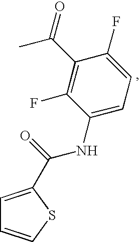

was first made as follows. To 3′-amino-2′,6′-difluoro-acetophenone (from Example H(1); 558 mg, 3.26 mmol) and 2-thiophenecarbonyl chloride (0.35 mL, 3.3 mmol) in CH₂Cl₂ (2 mL) at 0° C. was added dropwise pyridine (0.26 mL, 3.3 mmol). After 2 hours at ambient temperature, TLC showed starting material was still present, so DMAP (10 mg) was added. After 5 hours, the resultant mixture was partitioned between CH₂Cl₂ and 1N HCl, the organic layer was separated, washed with brine, dried over Na₂SO₄, and concentrated to obtain 905 mg (94% yield) of a light yellow solid, which was used without further purification.

¹H NMR: δ 8.47 (1H, td, J=5.8, 9.3 Hz), 7.83 (1H, bs), 7.65 (1H, dd, J=0.9, 3.7 Hz), 7.60 (1H, dd, J=0.9, 5.0 Hz), 7.16 (1H, dd, J=3.7, 5.0 Hz), 7.00 (1H, td, J=1.8, 9.3 Hz), 2.63 (3H, t, J=1.6 Hz).

Thiophene-2-carboxylic acid [3-(2-bromo-acetyl)-2,4-difluoro-phenyl]-amide, which has the structural formula

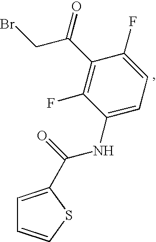

was made in a manner similar to that for 2-bromo-2′,6′-difluoro-3′-nitro-acetophenone from Example F(1). Thiophene-2-carboxylic acid (3-acetyl-2,4-difluoro-phenyl)-amide (903 mg, 3.21 mmol) and CuBr₂ (1.37 g, 6.13 mmol) gave a yellow solid in 80% yield, which was used without further purification.

¹H NMR: δ 8.56 (1H, td, J=5.8, 9.0 Hz), 7.65 (1H, dd, J=1.1, 3.8 Hz), 7.61 (1H, dd, J=1.1, 5.0 Hz), 7.17 (1H, dd, J=3.8, 5.0 Hz), 7.04 (1H, td, J=1.9, 9.0 Hz), 4.38 (2H, t, J=0.9 Hz).

The title compound was made similar to the procedure for 4-[4-amino-5-(2,6-difluoro-benzoyl)-thiazol-2-ylamino]-benzenesulfonyl fluoride in Example A(1). 4-Isothiocyanato-benzenesulfonamide (118 mg, 0.551 mmol) and thiophene-2-carboxylic acid [3-(2-bromo-acetyl)-2,4-difluoro-phenyl]-amide (208 mg, 0.578 mmol) gave 183 mg (62% yield) of a light yellow solid.

¹H NMR (DMSO-d₆): δ 11.19 (1H, s), 10.25 (1H, s), 8.25 (2H, bs), 8.00 (1H, dd. J=1.0, 3.8 Hz), 7.90 (1H, dd. J=1.0, 5.0 Hz), 7.84 (2H, d, J=9.3 Hz), 7.76 (2H, d, J=9.3 Hz), 7.65 (1H, td, J=6.1, 9.3 Hz), 7.29 (2H, bs), 7.24 (1H, dd, J=3.8, 5.0 Hz).

ESIMS: calcd. for C₂₁H₁₅F₂N₅O₄S₂Na: 558.0152; Found: 558.0164.

Anal. calcd. for C₂₁H₁₅F₂N₅O₄S₂.1.0 EtOH: C, 47.50; H, 3.64; N, 12.04; S, 16.54. Found: C, 47.42; H, 3.59; N, 11.94; S, 16.74.

### Example J(1)

### Thiazole-2-carboxylic Acid {3-[4-Amino-2-(4-sulfamoyl-phenylamino)-thiazole-5-carbonyl]-2,4-difluoro-phenyl}-amide

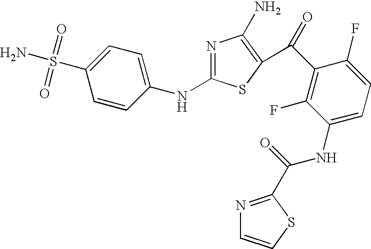

Thiazole-2-carboxylic acid (3-acetyl-2,4-difluoro-phenyl)-amide, which has the structural formula

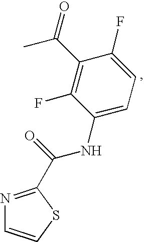

was made as follows. To thiazole-2-carboxylic acid (491 mg, 3.80 mmol; Metzger, et al., Bull. Soc. Chim. Fr., 708-709 (1953) and for ¹H NMR, see Borgen et al., Acta. Chem. Scand., 20; 2593-2600 (1966)) in THF (2 mL) was added O-(7-azabenzotriazol-1-yl)-N,N,N′,N′-tetramethyluronium hexafluorophosphate (HATU; 1.45 g, 3.81 mmol), followed by addition of 3′-amino-2′,6′-difluoro-acetophenone (from Example H(1); 542 mg, 3.36 mmol) and triethylamine (0.88 mL, 6.3 mmol). The mixture stirred under argon overnight, then partitioned between ethyl acetate and sat. aq. Na₂CO₃. The organic layer was separated, washed with 1N HCl, dried over Na₂SO₄, and concentrated to afford a residue that was purified via column chromatography to afford 823 mg (92% yield) of white solid, which was used without further purification.

¹H NMR: δ 9.33 (1H, bs), 8.54 (1H, td, J=5.7, 9.0 Hz), 7.96 (1H, d, J=3.1 Hz), 7.67 (1H, d, J=3.1 Hz), 7.02 (1H, td, J=1.8, 9.0 Hz), 2.64 (3H, t, J=1.8 Hz).

Thiazole-2-carboxylic acid [3-(2-bromoacetyl)-2,4-difluoro-phenyl]-amide, which has the structural formula

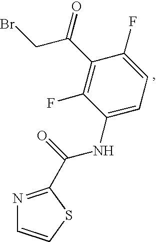

was made as follows. To thiazole-2-carboxylic acid [3-(2-acetyl)-2,4-difluoro-phenyl]-amide (530 mg, 1.88 mmol) in HOAc (5 mL) was added pyridinium tribromide (600 mg, 1.88 mmol). The mixture was heated at 70° C. for a half hour, allowed to cool, and partitioned between ether and water. The organic layer was separated, washed with water and sat. aq. NaHCO₃, dried over MgSO₄, and concentrated in vacuo to give 645 mg (95%) of white solid, which was used without further purification.

¹H NMR: δ 9.53 (1H, bs), 8.63 (1H, td, J=5.8, 9.0 Hz), 7.96 (1H, d, J=3.1 Hz), 7.69 (1H, d, J=3.1 Hz), 7.07 (1H, td, J=1.9, 9.0 Hz), 4.38 (2H, d, J=0.8 Hz).

The title compound was made analogously to 4-[4-amino-5-(2,6-difluoro-benzoyl)-thiazol-2-ylamino]-benzenesulfonyl fluoride from Example A(1). 4-Isothiocyanato-benzenesulfonamide (142 mg, 0.663 mmol) and thiazole-2-carboxylic acid [3-(2-bromoacetyl)-2,4-difluoro-phenyl]-amide (300 mg, 0.831 mmol) gave 245 mg (69% yield) of a yellow solid.

¹H NMR (DMSO-d₆): δ 11.19 (1H, s), 10.60 (1H, s), 8.45 (2H, bs), 8.17 (1H, d, J=3.1 Hz), 8.13 (1H, d, J=3.1 Hz), 7.80 (1H, d, J=9.2 Hz), 7.76 (1H, d, J=9.2 Hz).

HRESIMS: calcd. for C₂₀H₁₅F₂N₆O₃S₃: 537.0285. Found: 537.0272.

Anal. calcd. for C₂₀H₁₄F₂N₆O₄S₃.0.4H₂O.0.1 EtOH: C, 44.24; H, 2.83; N, 15.33; S, 17.54. Found: C, 44.23; H, 2.64; N, 15.16; S, 17.33.

### Example K(1)

### 4-[4-Amino-5-(2,6-difluoro-3-hydroxy-benzoyl)-thiazol-2-ylamino]-benzenesulfonamide

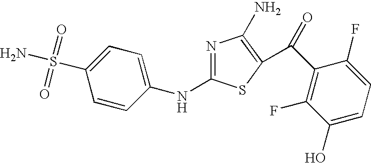

3′-(tert-Butyl-dimethyl-silyloxy)-2-chloro-2′,6′-difluoro-acetophenone, which has a structural formula

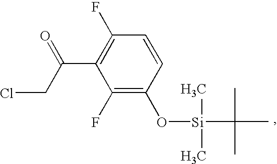

was first prepared. Conditions for aryl anion generation were adapted from Chen et al., J. Med. Chem.; 36; 3947-3955 (1993): To t-butyl-(2,4-difluoro-phenoxy)-dimethylsilane (2.03 g, 8.31 mmol; Chen, et al., J. Med. Chem.; 36; 3947-3955 (1993)) in ether (20 mL) at −78° C. was added dropwise n-BuLi (2.5 M in hexane, 3.7 mL) at a rate such that the internal temperature did not exceed −65° C. After 1 hour at −78° C., 2-chloro-N,N-dimethylacetamide (0.94 mL, 9.2 mmol) was added, and then allowed to slowly warm to ambient temperature overnight. The mixture was quenched with water and extracted with ether. The extracts were washed with brine, dried over MgSO₄, and concentrated in vacuo to provide a residue that was purified via column chromatography to give 516 mg (19% yield) of clear oil, which was used without further purification.

¹H NMR: δ 7.01 (1H, td, J=5.4, 9.1 Hz), 6.84 (1H, td, J=1.8, 9.1 Hz), 4.52 (2H, t, J=1.0 Hz), 1.00 (9H, s), 0.19 (6H, d, J=0.8 Hz).

4-(4-Amino-5-[3-(tert-butyl-dimethyl-silyloxy)-2,6-difluoro-benzoyl]-thiazol-2-ylamino)-benzenesulfonamide, which has the structural formula

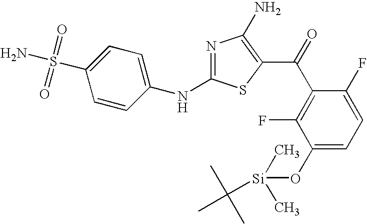

was made in a similar fashion to 4-[4-amino-5-(2,6-difluoro-benzoyl)-thiazol-2-ylamino]-benzenesulfonyl fluoride from Example A(1). 4-Isothiocyanato-benzenesulfonamide (177 mg, 0.826 mmol) and 3′-(tert-butyl-dimethyl-silyloxy)-2-chloro-2′,6′-difluoro-acetophenone (258 mg, 0.804 mmol) gave a yellow solid that was used in the next step without any further purification.

¹H NMR (DMSO-d₆): δ 11.53 (1H, s), 7.98 (2H, bs), 7.66 (2H, d, J=8.8 Hz), 7.58 (2H, d, J=8.8 Hz), 7.11 (2H, s), 0.77 (9H, s), −0.32 (6H, s).

The title compound was made as follows. To 4-(4-amino-5-[3-(tert-butyl-dimethyl-silanyloxy)-2,6-difluoro-benzoyl]-thiazol-2-ylamino)-benzenesulfonamide (462 mg, 0.854 mmol) in THF (10 mL) at 0° C. was added a solution of 1M TBAF in THF (0.94 mL). The mixture was allowed to stir at ambient temperature for a half-hour, solvent evaporated in vacuo, and the resultant residue diluted with water. The resultant yellow solid was filtered off and purified via column chromatography to provide 266 mg (71% yield for two steps) of a yellow solid.

¹H NMR (DMSO-d₆): δ 11.15 (1H, s), 10.08 (1H, s), 8.20 (2H, bs), 7.85 (2H, d, J=9.0 Hz), 7.80 (2H, d, J=9.0 Hz), 7.31 (2H, s).

Anal. calcd. for C₁₆H₁₂F₂N₄O₄S₂.1.0H₂O: C, 43.24; H, 3.18; N, 12.61; S, 14.43. Found: C, 43.50; H, 3.04; N, 12.38; S, 14.13.

### Example L(1)

### 4-[4-Amino-5-(2,6-difluoro-4-methoxy-benzoyl)-thiazol-2-ylamino]-benzenesulfonamide

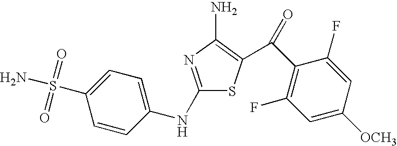

2-Chloro-2′,6′-difluoro-4-methoxy-acetophenone, which has the structural formula

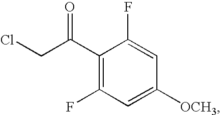

was made in a manner similar to that for 3′-(tert-butyl-dimethyl-silyloxy)-2-chloro-2′,6′-difluoro-acetophenone from Example K(1). 3,5-Difluoroanisole (5.00 g, 34.7 mmol) and 2-chloro-N,N-dimethylacetamide (3.92 mL, 38.2 mmol) gave 623 mg (8% yield) of a white powder, which was used without any further purification.

¹H NMR: δ 6.51 (2H, d, J=10.5 Hz), 4.51 (2H, t, J=2.3 Hz), 3.85 (3H, s).

The title compound was made in a manner similar to that for 4-[4-amino-5-(2,6-difluoro-benzoyl)-thiazol-2-ylamino]-benzenesulfonyl fluoride from Example A(1). 4-Isothiocyanato-benzenesulfonamide (84 mg, 0.39 mmol) and 2-chloro-2′,6′-difluoro-4′-methoxy-acetophenone (95 mg, 0.43 mmol) gave 78 mg (44% yield) of a yellow solid.

¹H NMR (DMSO-d₆): δ 11.10 (1H, s), 8.15 (2H, bs), 7.80 (2H, d, J=9.1 Hz), 7.74 (2H, d, J=9.1 Hz), 7.26 (2H, s), 6.83 (2H, d, J=9.7 Hz), 3.84 (3H, s).

Anal. calcd. for C₁₇H₁₄F₂N₄O₄S₂.1.0H₂O: C, 44.54; H, 3.52; N, 12.22; S, 13.99. Found: C, 44.59; H, 3.43; N, 11.91; S, 13.74.

### Example M(1)

### 4-[4-Amino-5-(2-hydroxy-2-methyl-propionyl)-thiazol-2-ylamino]-benzenesulfonamide

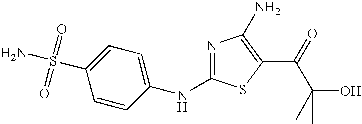

1-Bromo-3-methyl-3-trimethylsilyloxy-butan-2-one, which has the structural formula

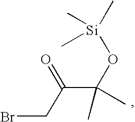

was made as follows. To 3-hydroxy-3-methyl-2-butanone (2.0 g, 19.6 mmol) in CH₂Cl₂ (200 mL) at 0° C. was added sequentially triethylamine (8.2 mL, 58.7 mmol) and trimethylsilyl triflate (TMS-OTf; 7.8 mL, 43.1 mmol). After 1 hour at 0° C., the mixture was partitioned between CH₂Cl₂ and sat. aq. NaHCO₃. The organic layer was separated, washed with brine, dried over Na₂SO₄, and concentrated to give a yellow oil, which was placed in THF (100 mL) and cooled to 0° C. NaHCO₃ (4.9 g, 58.8 mmol) and N-bromosuccinimide (NBS; 6.96 g, 39.2 mmol) were added sequentially. After 1 hour at ambient temperature, the mixture was extracted with ether. The organic extracts were washed with brine, dried over Na₂SO₄, and concentrated in vacuo. The resultant oil was passed through a pad of silica gel with hexane, and the filtrate was concentrated to afford 4.35 g (88% yield for two steps) of a yellow oil, which was used without further purification.

¹H NMR: δ 4.40 (2H, s), 1.41 (6H, s), 0.16 (9H, s).

The title compound was made in a manner analogous to that for 4-[4-amino-5-(2,6-difluoro-3-nitro-benzoyl)-thiazol-2-ylamino]-benzenesulfonamide (Example F(1)). 4-Isothiocyanato-benzenesulfonamide (269 mg, 1.26 mmol) and 1-bromo-3-methyl-3-trimethylsilanyloxy-butan-2-one (350 mg, 1.38 mmol), recrystallization from ethanol (5 mL), and vacuum drying furnished 145 mg (31% yield) of a yellow powder.

¹H NMR (DMSO-d₆): δ 10.83 (1H, s), 8.05 (2H, bs), 7.81 (2H, d, J=9.1 Hz), 7.76 (2H, d, J=9.2 Hz), 7.25 (2H, s), 5.69 (1H, bs), 1.26 (6H, s).

Anal. calcd. for C₁₃H₁₆N₄O₄S₂: C, 43.81; H, 4.52; N, 15.72; S, 17.99. Found: C, 43.81; H, 4.60; N, 15.55; S, 17.82.

### Example N (1)

### 4-(4-Amino-5-isobutyryl-thiazol-2-ylamino)-benzenesulfonamide

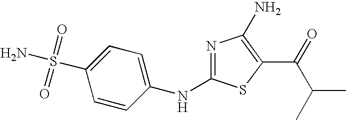

The title compound was made analogously to 4-[4-amino-5-(2,6-difluoro-benzoyl)-thiazol-2-ylamino]-benzenesulfonyl fluoride from Example A(1). 4-Isothiocyanato-benzenesulfonamide (500 mg, 2.33 mmol) and 1-bromo-3-methyl-butan-2-one (423 mg, 2.60 mmol; McMorris et al., J. Chem. Soc. Perkin Trans. I, 295-302 (1996)) gave 288 mg (37% yield) of a yellow solid.

¹H NMR (DMSO-d₆): δ 11.04 (1H, s), 7.80 (2H, d, J=9.6 Hz), 7.76 (2H, d, J=7.6 Hz), 7.27 (2H, s), 2.59 (1H, hept., J=6.8 Hz), 1.06 (3H, d, J=6.8 Hz).

Anal. calcd. for C₁₃H₁₆F₂N₄O₃S₂: C, 45.87; H, 4.74; N, 16.46; S, 18.84. Found: C, 46.05; H, 4.80; N, 16.46; S, 18.83.

### Example O(1)

### 1-H-Pyrrole-2-carboxylic acid (3-{1-[4-amino-2-(4-sulfamoyl-phenylamino)-thiazol-5-yl]-methanoyl}-2,4-difluoro-phenyl)-amide

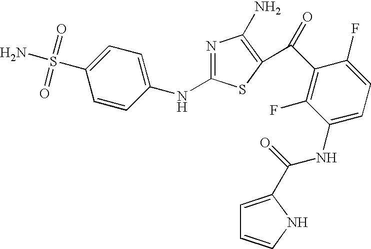

The title compound was made as follows. To 4-[4-amino-5-(3-amino-2,6-difluoro-benzoyl)-thiazol-2-ylamino]-benzenesulfonamide {(200 mg, 0.47 mmol, from Example G(1)} in THF (8 mL) at 0° C. was added sequentially triethylamine (0.16 mL) and 1-H-pyrrole-2-carbonyl chloride hydrochloride salt (86 mg, 0.52 mmol; Annoura et al., Tetrahedron Lett., 36; 413-416 (1995)). After 30 minutes at ambient temperature, TLC showed a small amount of remaining starting material, so more 1-H-pyrrole-2-carbonyl chloride hydrochloride salt (0.2 equiv.) was added. The mixture stirred for another half hour, and the solvent was evaporated in vacuo. The residue was taken upon into MeOH (3 mL), diluted with water, and filtered. The isolated yellow solid was purified via column chromatography to afford 90 mg (37% yield) of a yellow solid.

¹H NMR (DMSO-d₆): δ 11.68 (1H, bs), 11.19 (1H, s), 9.70 (1H, s), 8.27 (2H, bs), 7.80 (2H, d. J=9.2 Hz), 7.75 (2H, d. J=9.2 Hz), 7.68 (1H, td, J=6.2, 8.9 Hz), 7.29 (2H, s), 7.22 (1H, td, J=1.2, 8.9 Hz), 7.04 (1H, m), 6.97 (1H, m), 6.17 (1H, m).

HRESIMS: calcd. for C₂₁H₁₅F₂N₅O₄S₂Na (M+Na): 558.0152. Found: 558.0164.

Anal. calcd. for C₂₁H₁₆F₂N₆O₄S₂.1.0H₂O.0.3 MeOH: C, 46.84; H, 3.54; N, 15.39; S, 11.74. Found: C, 46.66; H, 3.30; N, 15.31; S, 11.58.

### Example P(1)

### 3-{4-Amino-5-[1-(2,6-difluoro-phenyl)-methanoyl]-thiazol-2-ylamino}-benzenesulfonamide

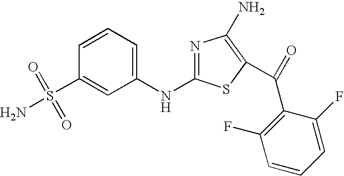

First the starting material, 3-isothiocyanato-benzenesulfonamide, which has the structural formula

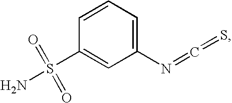

was prepared as follows. To a solution of 3-amino-benzenesulfonamide (Maybridge Chemical Co., 1.00 g, 5.81 mmol) in acetone (15 mL) at 0° C. was added sequentially thiophosgene (0.503 mL, 6.39 mmol) and fresh 25% aq. Na₂CO₃ (5.8 mL). The mixture was allowed to warm to ambient temperature. After 20 minutes, the acetone was removed under reduced pressure. The resultant suspension was adjusted to pH=7 with 10% aq. HCl and filtered to isolate a light tan solid, 1.24 g (quantitative yield), that matched previous (mp 146-149° C.; French patent application FR 1528249; Chem. Abs., 71, 30206 (1969)) and was used without further purification.

¹H NMR (DMSO-d₆): δ 7.83-7.75 (1H, m), 7.69-7.62 (1H, m), 7.52 (1H, s).

The title compound was prepared in a manner similar to that for 4-[4-amino-5-(2-hydroxy-2-methyl-propionyl)-thiazol-2-ylamino]-benzenesulfonamide (Example M(1)). 3-Isothiocyanato-benzenesulfonamide (212 mg, 0.989 mmol) furnished a yellow solid, 432 mg, that precipitated from iPrOH/hex to give 171 mg of orange-brown solid. Furthermore, the mother liquor was purified via column chromatography with a 5-10% MeOH/CHCl₃ stepwise gradient eluant to provide 120 mg of yellow solid that decomposed above 240° C. The total yield was 291 mg (73%).

¹H NMR (CD₃OD): δ 8.36 (1H, dd, J=1.8, 1.8 Hz), 7.78 (1H, ddd, J=1.0, 2.2, 8.1 Hz), 7.62 (1H, ddd, J=1.1, 1.6, 7.8 Hz), 7.50 (1H, t, J=8.1 Hz), 7.05 (2H, t, J=7.5 Hz).

FTIR (KBr): 3309, 3076, 1620, 1546, 1527, 1465, 1429, 1156 cm⁻¹.

HRFABMS: Calcd for C₁₆H₁₃F₂N₄O₃S₂ (M+H⁺) 411.0406. Found: 411.0406.

Anal. calcd. for C₁₆H₁₂F₂N₄O₃S₂.0.5H₂O: C, 45.82; H, 3.12; N, 13.36; S, 15.29. Found: C, 45.78; H, 3.12; N, 13.18; S, 15.50.

### Example Q(1)

### 1-[4-Amino-2-(4-methanesulfonyl-phenylamino)-thiazol-5-yl]-1-(2,6-difluoro-phenyl)-methanone

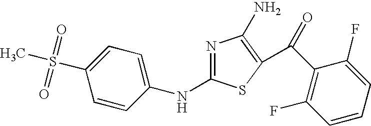

First 1-isothiocyanato-4-methanesulfonyl-benzene, which has the structural formula

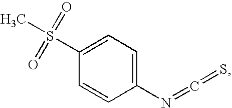

was prepared in a manner similar to that for 3-isothiocyanato-benzenesulfonamide in Example P(1). 1-Amino-4-methanesulfonyl-benzene (Maybridge Chemical Co., 256 mg, 1.50 mmol) provided 292 mg (91% yield) of a brown solid, which matched previous (mp 56° C.; Uher; et al. Chem. Zvesti, 21, 44-56, Chem. Abs., 67, 43495 (1967)) and was used without further purification.

¹H NMR: δ 7.97 (2H, ddd, J=2.2, 2.2, 8.6 Hz), 7.40 (2H, ddd, J=2.2, 2.2, 8.6 Hz), 3.08 (3H, s).

FTIR (KBr): 2096, 1586, 1306, 1286 1143 cm⁻¹.

The title compound was prepared in a manner similar to that for 4-[4-amino-5-(2-hydroxy-2-methyl-propionyl)-thiazol-2-ylamino]-benzenesulfonamide (Example M(1)). 1-Isothio-cyanato-4-methanesulfonyl-benzene and purification via column chromatography with 3% MeOH/CHCl₃ as eluant gave a yellow solid, 78 mg (41%), mp 225-230° C. (decomp).

¹H NMR (CD₃OD): δ 7.91 (2H, ddd, J=0.7, 0.7, 9.6 Hz), 7.89 (2H, ddd, J=1.0, 1.0, 4.8 Hz), 7.48 (1H, ddd, J=6.8, 8.4, 15.0 Hz), 7.07 (2H, ddd, J=0.7, 1.9, 8.2 Hz), 3.10 (3H, s).

FTIR (KBr): 1618, 1595, 1547, 1523, 1464, 1426, 1144 cm⁻¹.

HRFABMS. calcd. for C₁₇H₁₄F₂N₃O₃S₂ (M+H⁺): 410.0445. Found: 410.0429.

Anal. calcd. for C₁₇H₁₃F₂N₃O₃S₂.0.99 MeOH.0.2 CHCl₃: C, 46.98; H, 3.72; N, 9.04; S, 13.79. Found: C, 47.14; H, 3.32; N, 8.69; S, 13.39.

### Example R(1)

### 4-[4-Amino-5-(2,6-dichloro-benzoyl)-thiazol-2-ylamino]-benzenesulfonamide

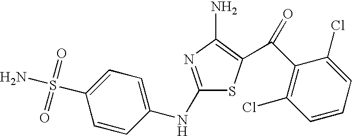

The title compound was prepared in a manner similar to 4-[4-amino-5-(2,6-difluoro-benzoyl)-thiazol-2-ylamino]-benzenesulfonyl fluoride from Example A(1): 2-Bromo-2′,6′-dichloro-acetophenone (World Patent Application WO 99/21845 and Mlotkowska, et al., Pol. J. Chem., 55, 631-642 (1981)) and 4-isothiocyanato-benzenesulfonamide provided a yellow solid in 12% yield.

¹H NMR (DMSO-d₆): δ 7.79 (2H, d, J=9.2 Hz), 7.74 (2H, d, J=9.2 Hz), 7.28 (2H, s).

HRFABMS. calcd. for C₁₆H₁₃Cl₂N₄O₃S₂ (MH⁺): 442.9806. Found: 442.9814.

Anal. calcd. for C₁₆H₁₂Cl₂N₄O₃S₂.0.3H₂O: C, 42.83; H, 2.83; N, 12.49; S, 14.29; Cl, 15.80. Found: C, 42.45; H, 2.99; N, 12.38; S, 14.10; Cl, 15.65.

### Example R(2)

### 4-Amino-5-(2,6-dichlorobenzoyl)-2-(4-methylthio-phenylamino)-thiazole

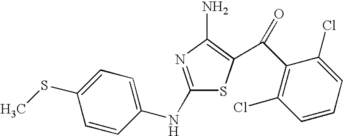

The title compound was prepared in a manner similar to 4-[4-amino-5-(2,6-difluoro-benzoyl)-thiazol-2-ylamino]-benzenesulfonyl fluoride from Example A(1): 4-(Methylthio)phenyl isothiocyanate (Lancaster, 362 mg, 2.00 mmol) and 2-bromo-2′,6′-dichloro-acetophenone (562 mg, 2.10 mmol; from Example R(1)) gave 372 mg (76% yield) of a yellow solid.

¹H NMR: δ 8.61 (s, 1H), 7.38-7.21 (m, 7H), 7.04 (s, 2H), 2.47 (s, 3H).

HRESIMS: calcd. for C₁₇H₁₄Cl₂N₃OS₂ (M+H⁺): 409.9955. Found: 409.9970.

Anal. calcd. for C₁₇H₁₃Cl₂N₃OS₂.0.29 EtOAc: C, 49.95; H, 3.56; N, 9.62; S, 14.69. Found: C, 50.13; H, 3.56; N, 9.58; S, 14.82.

### Example R(3)

### 4-Amino-5-(2,6-dichlorobenzoyl)-2-(3-methylthio-phenylamino)-thiazole

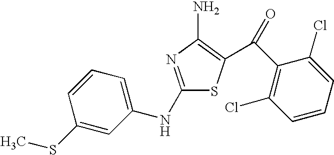

The title compound was prepared in a manner similar to 4-[4-amino-5-(2,6-difluoro-benzoyl)-thiazol-2-ylamino]-benzenesulfonyl fluoride from Example A(1): 3-Methylthio-phenyl isothiocyanate (TransWorld Chemical) and 2-bromo-2′,6′-dichloro-acetophenone (from Example R(1)) gave 607 mg (49%) of a yellow solid.

¹H NMR: δ 7.36-7.04 (m, 7H), 2.48 (s, 3H).

Anal. calcd. for C₁₇H₁₃Cl₂N₃OS₂: C, 49.76; H, 3.19; N, 10.24; S, 15.63. Found: C, 49.96; H, 3.16; N, 10.08; S, 15.85.

### Example R(4)

### 4-[4-Amino-5-(2,2-dimethyl-propionyl)-thiazol-2-ylamino]-benzenesulfonamide

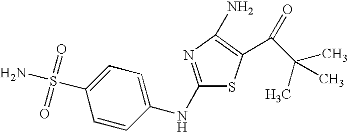

Title compound was made in a manner similar 4-[4-amino-5-(2,6-difluoro-benzoyl)-thiazol-2-ylamino]-benzenesulfonyl fluoride in Example A(1). 1-Bromopinacolone and 4-isothiocyanato-benzenesulfonamide gave 75 mg (39%) of a yellow solid.

¹H NMR (DMSO-d₆): δ 10.88 (s, 1H), 8.06 (br, 2H), 7.84-7.78 (m, 4H), 7.29 (s, 2H), 1.24 (s, 9H).

HRESIMS: calcd. for C₁₄H₁₉N₄O₃S₂ (M+H⁺): 355.0899. Found: 355.0908.

Anal. calcd. for C₁₄H₁₈N₄O₃S₂: C, 52.67; H, 4.91; N, 13.65; S, 7.81. Found: C, 52.72; H, 4.95; N, 13.64; S, 7.72.

### Example S(1)

### 4-[4-Amino-5-(2,6-difluoro-benzoyl)-thiazol-2-ylamino]-2-methyl-benzenesulfonamide

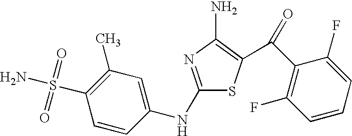

First N-(3-methyl-4-sulfamoyl-phenyl)-acetamide, which has structural formula

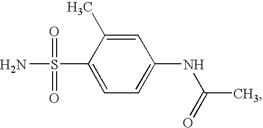

was prepared as follows. To a suspension of 4-acetamido-2-methyl-phenylsulfonic acid pyridinium salt (2.11 g, 6.84 mmol; Pieper, et al., Arzneim. Forsch., 39(11), 1073-1080 (1989)) in DMF (4 mL) at ambient temperature was added SOCl₂ (0.549 mL, 7.52 mmol). The mixture stirred until it formed a clear solution, then poured into a mixture of EtOAc: H₂O (100 mL, 1:1). The organic layer was separated, dried over Na₂SO₄, and concentrated to a yellow oil, which was treated with conc. aq. NH₄OH (20 mL) and stirred for 24 hours. The solution was concentrated in vacuo. The resultant solid was suspended in H₂O (20 mL) and filtered to provide a white solid in 53% yield.

¹H NMR (DMSO-d₆): δ 10.14 (1H, s), 7.72 (1H, d, J=8.9 Hz), 7.22 (2H, s), 2.52 (3H, s), 2.04 (3H, s).

4-Amino-2-methyl-benzenesulfonamide hydrochloride, which has structural formula

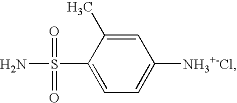

was prepared as follows. To a suspension of N-(3-methyl-4-sulfamoyl-phenyl)-acetamide (500 mg, 2.19 mmol) in ethanol (5 mL) was added 6N HCl (5 mL). The mixture was heated to reflux for 3 hours and concentrated to afford 0.45 g (93% yield) of white solid, which was used without further purification.

¹H NMR (CD₃OD): δ 8.08 (1H, d, J=8.9 Hz), 2.68 (3H, s).

4-Isothiocyanato-2-methyl-benzenesulfonamide, which has structural formula

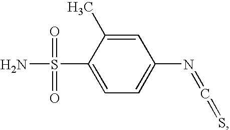

was prepared as follows. To a solution of 4-amino-2-methyl-benzenesulfonamide hydrochloride (0.45 g, 2.02 mmol) in THF (4 mL) and 10% HCl (2 mL) at ambient temperature was added thiophosgene (0.17 mL, 2.2 mmol). The mixture stirred for 2 hours, and then concentrated in vacuo to provide a white solid in 95% yield, which was used without any further purification.

¹H NMR: δ 8.02 (1H, d, J=8.9 Hz), 2.66 (3H, s).

The title compound was prepared in the manner described for 4-[4-amino-5-(2,6-difluoro-benzoyl)-thiazol-2-ylamino]-benzenesulfonyl fluoride from Example A(1): 2-Bromo-2′,6′-difluoro-acetophenone (from Example A(1)) and 4-isothiocyanato-2-methyl-benzenesulfonamide provided yellow powder in 65% yield.

¹H NMR (DMSO-d₆): δ 7.80 (1H, d, J=8.7 Hz), 7.30 (2H, s), 7.21 (2H, dd, J=7.8, 8.1 Hz), 2.57 (3H, s).

HRMALDIFTMS. calcd. for C₁₇H₁₅F₂N₄O₃S₂ (MH⁺): 425.0554. Found: 425.0546.

Anal. calcd. for C₁₇H₁₄F₂N₄O₃S₂.0.2H₂O: C, 47.70; H, 3.39; N, 13.09; S, 14.98. Found: C, 48.04; H, 3.65; N, 13.20; S, 14.58.

### Example S(2)

### 4-[4-Amino-5-(2,6-difluoro-benzoyl)-thiazol-2-ylamino]-2-trifluoromethyl-benzenesulfonamide

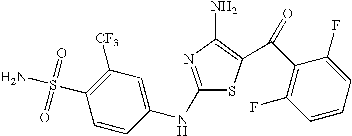

First 4-amino-2-trifluoromethyl-benzenesulfonamide, which has structural formula

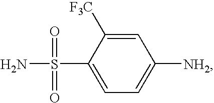

was prepared as follows. To a warm solution of SnCl₂.2H₂O (750 mg, 3.30 mmol) in a mixture of EtOH (2 mL) and conc. HCl (2 mL) was added 4-nitro-2-trifluoromethyl-benzenesulfonamide (200 mg, 0.740 mmol; Jones, et al., J. Med. Chem., 39 (1996), 904-917). The mixture was heated to 55° C. for 0.5 hours, concentrated in vacuo, adjusted to pH=6.5 with 2N NaOH, and extracted with EtOAc (3×25 mL). The combined organic layers were dried over Na₂SO₄, filtered, and concentrated to 0.18 g (100% crude yield) of white solid, which was uesd immediately.

4-isothiocyanato-2-trifluoromethyl-benzenesulfonamide, which has structural formula

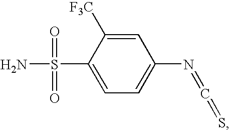

was prepared under similar conditions to that for the preparation of 4-isothiocyanato-2-methyl-benzenesulfonamide in Example S(1). The crude white solid provided 0.16 g (77% yield) of cream-colored powder, which was used without further purification.

¹H NMR (CD₃OD): δ 8.15 (1H, d, J=8.6 Hz), 8.08 (1H, d, J=8.8 Hz), 7.56 (1H, dd, J=2.2, 8.5 Hz).

The title compound was prepared essentially in the manner described for 4-[4-amino-5-(2,6-difluoro-benzoyl)-thiazol-2-ylamino]-benzenesulfonyl fluoride from Example A(1): 2-Bromo-2′,6′-difluoro-acetophenone (from Example A(1)) and 4-isothiocyanato-2-trifluoromethyl-benzenesulfonamide provided a yellow solid in 66% yield.

¹H NMR (DMSO-d₆): δ 7.63 (2H, s), 7.61-7.50 (1H, m), 7.24 (2H, t, J=8.0 Hz).

HRESIMS. Calcd for C₁₇H₁₂F₅N₄O₃S₂ (M+H⁺): 479.0270. Found: 479.0264.

Anal. calcd. for C₁₇H₁₁F₅N₄O₃S₂.0.3 CHCl₃: C, 39.72; H, 2.18; N, 10.65; S. 12.19. Found: C, 39.65; H, 2.38; N, 10.66; S, 12.13.

### Example S(3)

### 4-[4-Amino-5-(2,6-difluoro-benzoyl)-thiazol-2-ylamino]-2-nitro-benzenesulfonamide

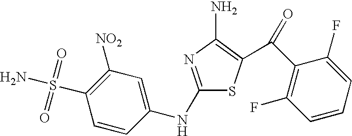

First 4-amino-2-nitro-benzenesulfonamide hydrochloride, which has structural formula

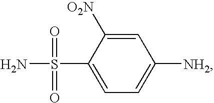

was prepared in a manner analogous to that for 4-amino-2-methyl-benzenesulfonamide hydrochloride from Example S(1). N-(3-Nitro-4-sulfamoyl-phenyl)-acetamide (720 mg, 2.77 mmol; Topliss et al., J. Med. Chem., 6, 122-127 (1963)) provided 520 mg (76% yield) of yellow solid, which was used without further purification.

¹H NMR (CD₃OD): δ 7.72 (1H, d, J=8.7 Hz), 6.86 (1H, d, J=2.3 Hz), 6.80 (1H, dd, J=2.3, 8.7 Hz).

4-Isothiocyanato-2-nitro-benzenesulfonamide, which has structural formula

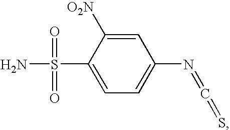

was prepared in a manner analogous to that for 4-isothiocyanato-2-methyl-benzenesulfonamide in Example S(1). 4-Amino-2-nitro-benzenesulfonamide hydrochloride (500 mg, 2.18 mmol) provided 0.564 g (100% yield) of cream solid, which was used without further purification.

¹H NMR (CD₃OD): δ 8.16 (1H, d, J=8.5 Hz), 8.06 (1H, d, J=8.7 Hz), 7.82 (1H, d, J=2.0 Hz), 7.68 (1H, dd, J=2.1, 8.5 Hz).

The title compound was prepared essentially in the manner described for 4-[4-amino-5-(2,6-difluoro-benzoyl)-thiazol-2-ylamino]-benzenesulfonyl fluoride from Example A(1): 2-Bromo-2′,6′-difluoro-acetophenone (from Example A(1)) and 4-isothiocyanato-2-nitro-benzenesulfonamide provided a yellow solid in 57% yield.

¹H NMR (DMSO-d₆): δ 8.35 (1H, d, J=5.1 Hz), 7.98 (1H, d, J=8.8 Hz), 7.80 (1H, dd, J=2.1, 8.7 Hz), 7.74 (2H, s), 7.62 (1H, m), 7.22 (2H, dd, J=7.9, 8.0 Hz).

HRESIMS: calcd. for C₁₆H₁₂F₂N₅O₅S₂ (M+H⁺): 456.274. Found: 456.0241.

Anal. calcd. for C₁₆H₁F₂N₅O₅S₂.0.7H₂O.0.7 EtOH: C, 41.77; H, 3.34; N, 14.00, S, 12.82. Found: C, 41.67; H, 3.32; N, 13.74; S, 14.08.

### Example T(1)

### 4-[4-Amino-5-(2,6-difluoro-benzoyl)-thiazol-2-ylamino]-2-methoxy-benzenesulfonamide

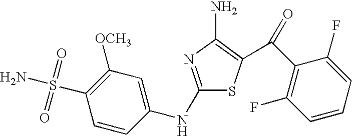

First 4-benzylsulfanyl-3-methoxy-nitrobenzene, which has structural formula

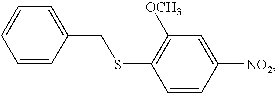

was prepared as follows. To a suspension of benzyl mercaptan (2.50 mL, 21.3 mmol) in H₂O was sequentially added a solution of 1-chloro-2-methoxy-4-nitro-benzene (2.00 g, 10.7 mmol) in ethanol (20 mL) and Na₂CO₃ (2.26 g, 21.3 mmol). The mixture was heated at reflux for 3 hours, allowed to cool to ambient temperature, diluted with H₂O, and filtered to isolate 2.95 g (100% yield) of green solid, which was used without any further purification.

¹H NMR (CD₃OD): δ 4.24 (2H, s), 3.61 (3H, s).

2-Methoxy-4-nitro-benzenesulfonamide, which has structural formula

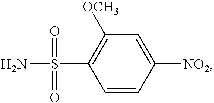

was prepared as follows. To a suspension of 4-benzylsulfanyl-3-methoxy-nitrobenzene (1.86 g, 6.75 mmol) in a mixture of HOAc (15 mL) and H₂O (2 mL) at 0° C. was bubbled Cl₂ (9) for 0.5 hours. The clear solution was allowed to warm to ambient temperature and diluted with CHCl₃ (100 mL). The organic layer was separated, dried over Na₂SO₄, and concentrated to a yellow residue, which was cooled to 0° C., treated with conc. aq. NH₄OH (30 mL), and allowed to warm to ambient temperature. After 24 hours, removal of solvent in vacuo led to 1.1 g (71% yield) of a cream-colored solid, which was used without further purification.

¹H NMR (CD₃OD): δ 8.08 (1H, d, J=8.5 Hz, 7.98 (1H, d, J=2.0 Hz), 7.94 (1H, dd, J=2.1, 8.5 Hz).

4-Amino-2-methoxy-benzenesulfonamide, which has structural formula

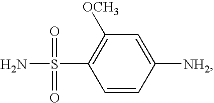

was prepared in a manner analogous to 4-amino-2-trifluoromethyl-benzenesulfonamide from Example S(2). 2-Methoxy-4-nitro-benzenesulfonamide (500 mg, 2.15 mmol) provided 330 mg (76% yield) of yellow oil, which was used without further purification.

¹H NMR (DMSO-d₆): δ 6.58 (2H, s), 5.82 (2H, s), 3.78 (3H, s).

4-Isothiocyanato-2-methoxy-benzenesulfonamide, which has structural formula

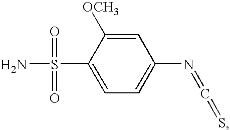

was prepared was prepared in a manner analogous to 4-isothiocyanato-2-trifluoromethyl benzenesulfonamide in Example S(2). 4-Amino-2-methoxy-benzenesulfonamide (300 mg, 1.48 mmol) provided 320 mg (88% yield) of yellow solid, which was used without further purification.

¹H NMR (DMSO-d₆): δ 7.75 (1H, d, J=8.3 Hz), 7.32 (1H, d, J=1.9 Hz), 7.21 (2H, s), 7.10 (1H, dd, J=1.9, 8.3 Hz), 3.46 (3H, s).

The title compound was prepared in the manner described for 4-[4-amino-5-(2,6-difluoro-benzoyl)-thiazol-2-ylamino]-benzenesulfonyl fluoride from Example A(1): 2-Bromo-2′,6′-difluoro-acetophenone (from Example A(1)) and 4-isothiocyanato-2-methoxy-benzenesulfonamide provided a yellow solid in 65% yield.

¹H NMR (DMSO-d₆): δ 7.67 (1H, d, J=8.6 Hz), 7.62-7.50 (1H, m), 7.22 (2H, dd, J=7.7, 8.2 Hz), 7.13 (2H, dd, J=1.9, 8.6 Hz), 6.99 (2H, s), 3.91 (3H, s).

HRESIMS: calcd. for C₁₇H₁₅F₂N₄O₄S₂ (MH⁺): 441.0502. Found: 441.0488.

Anal. calcd. for C₁₇H₁₄F₂N₄O₄S₂.0.5H₂O: C, 45.43; H, 3.36; N, 12.47: S, 14.27. Found: C, 45.55; H, 3.32; N, 12.17; S, 13.93.

### Example T(2)

### 4-[4-Amino-5-(2,6-difluoro-benzoyl)-thiazol-2-ylamino]-2-chloro-benzenesulfonamide

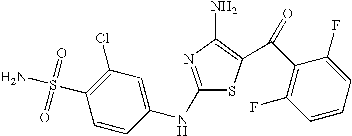

4-Benzylsulfanyl-3-chloro-nitrobenzene, which has structural formula

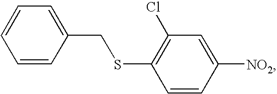

was prepared in a manner analogous to 4-benzylsulfanyl-3-methoxy-nitrobenzene in Example T(1). 2-Chloro-1-fluoro-4-nitro-benzene (2.00 g, 11.4 mmol) provided 1.5 g (47%) of yellow solid, which was used without further purification.

¹H NMR (CD₃OD): δ8.24 (1H, d, J=2.4 Hz), 8.08 (1H, dd, J=2.4, 8.8 Hz), 7.56 (1H, d, J=8.8 Hz), 4.32 (2H, s).

2-Chloro-4-nitro-benzenesulfonamide, which has structural formula

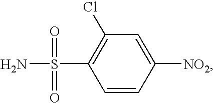

was prepared in a manner analogous to 2-methoxy-4-nitro-benzenesulfonamide in Example T(1). 4-Benzylsulfanyl-3-chloro-nitrobenzene (1.50 g, 5.36 mmol) provided 1.0 g (79% yield) of brown solid, which was used without further purification.

¹H NMR (CD₃OD): δ 8.46 (1H, dd, J=1.2, 1.4 Hz), 7.44 (2H, s).

4-Amino-2-chloro-benzenesulfonamide, which has structural formula

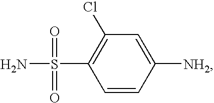

was prepared in a manner analogous to 4-amino-2-trifluoromethyl-benzenesulfonamide in Example S(2). 2-Chloro-4-nitro-benzenesulfonamide (0.500 g, 2.11 mmol) provided 0.31 g (71% yield) of yellow oil, which was used without further purification.

¹H NMR: δ 7.86 (1H, d, J=8.6 Hz), 6.74 (1H, d, J=2.3 Hz), 6.58 (1H, dd, J=2.3, 8.6 Hz).

The title compound was prepared as follows. 4-Amino-2-chloro-benzenesulfonamide (370 mg, 1.79 mmol) was subjected to the conditions that were described for 4-isothiocyanato-2-trifluoromethyl-benzenesulfonamide in Example S(2) to provide 0.17 g of yellow oil, which in turn was employed with 2-bromo-2′,6′-difluoro-acetophenone (from Example A(1)) in the manner that was described for 4-[4-amino-5-(2,6-difluoro-benzoyl)-thiazol-2-ylamino]-benzenesulfonyl fluoride in Example A(1) to furnish a yellow solid in 20% overall yield.

¹H NMR (DMSO-d₆): δ 8.06 (1H, d, J=2.9 Hz), 7.82 (1H, d, J=8.8 Hz), 7.39 (2H, s), 7.22 (2H, dd, J=7.7, 8.2 Hz).

Anal. calcd. for C₁₆H₁₁F₂ClN₄O₃S₂.0.35 CHCl₃: C, 40.35; H, 2.70; N, 11.50: S, 13.18; Cl, 14.93. Found: C, 40.66; H, 2.70; N, 11.47; S, 13.12; Cl, 14.55.

### Example U(1)

### N-{4-[4-Amino-2-(4-sulfamoyl-phenylamino)-thiazole-5-carbonyl]-3,5-difluoro-phenyl}-acetamide

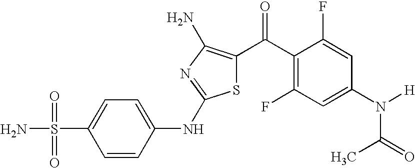

First 4′-amino-2′,6′-difluoro-acetophenone, which has structural formula

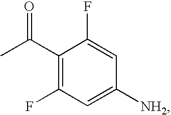

was prepared as follows. To a solution of 2′,4′,6′-trifluoro-acetophenone (1.00 g, 5.74 mmol; Joshi, et al., J. Indian. Chem. Soc., 59, 293-294 (1982)) in acetonitrile (8 mL) was added a solution of NaN₃ (0.467 g, 7.17 mmol) in H₂O (4 mL). The mixture was heated at reflux for 72 hours, allowed to cool to ambient temperature, and extracted with EtOAc (75 mL). The separated organic layer was washed with H₂O (25 mL), dried over Na₂SO₄, and concentrated to a red oil, which was placed in EtOAc (25 mL) with 10% Pd/C (0.15 g) under an atmosphere of H₂ (balloon). After 12 hours at ambient temperature, the catalyst was filtered onto a pad of Celite, and the filtrate concentrated to a brown solid, which was purified via column chromatography with 30% EtOAc/hexane as eluant to afford 330 mg (34% yield) of white solid and was used without further purification.

¹H NMR: δ 2.61 (3H, s).

N-(4-Acetyl-3,5-difluoro-phenyl)-acetamide, which has structural formula

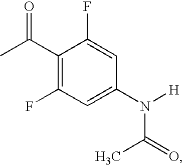

was prepared as follows. A mixture of 4′-amino-2′,6′difluoro-acetophenone (100 mg, 0.580 mmol), HOAc (2 mL) and acetic anhydride (0.276 mL, 2.92 mmol) was heated at reflux for 0.5 hours, allowed to cool to ambient temperature, and concentrated to give 124 mg (100% yield) of colorless solid, which was used without further purification.

¹H NMR: δ 7.21 (2H, d, J=10.4 Hz), 2.58 (3H, t, J=2.10 Hz), 2.30 (3H, s).

N-[4-(2-Bromoacetyl)-3,5-difluoro-phenyl]-acetamide, which has structural formula

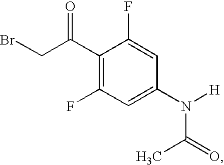

was prepared in a similar manner as 2-bromo-2′,6′-difluoro-3′-nitro-acetophenone in Example F(1). N-(4-Acetyl-3,5-difluoro-phenyl)-acetamide (430 mg, 2.01 mmol) and CuBr₂ (0.901 g, 4.03 mmol) gave 500 mg (85% yield) of a yellow solid, which was used without further purification.

¹H NMR: δ 4.36 (2H, t, J=0.9 Hz), 2.20 (3H, s).

The title compound was prepared in the same manner that was described for 4-[4-amino-5-(2,6-difluoro-benzoyl)-thiazol-2-ylamino]-benzenesulfonyl fluoride from Example A(1): N-[4-(2-Bromocetyl)-3,5-difluoro-phenyl]-acetamide and 4-isothiocyanato-benzenesulfonamide provided a yellow solid in 39% yield.

¹H NMR (DMSO-d₆): δ 7.78 (2H, d, J=9.1 Hz), 7.74 (2H, d, J=9.2 Hz), 7.36 (2H, d, J=10.0 Hz), 7.25 (2H, s), 2.10 (3H, s).

HRESIMS: calcd. for C₁₈H₁₆F₂N₅O₄S₂ (M+H⁺): 468.0617. Found: 468.0657.

Anal. calcd. for C₁₈H₁₅F₂N₅O₄S₂.1.0H₂O: C, 44.53; H, 3.53; N, 14.43; S, 13.21. Found: C, 44.42; H, 3.54; N, 14.53; S, 13.36.

### Example V(1)

### 4-[4-Amino-5-(4-amino-2,6-difluoro-benzoyl)-thiazol-2-ylamino]-benzenesulfonamide

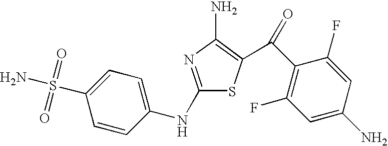

To a solution of N-{4-[4-amino-2-(4-sulfamoyl-phenylamino)-thiazole-5-carbonyl]-3,5-difluoro-phenyl}-acetamide (Example U(1); 100 mg, 0.214 mmol) in ethanol (5 mL) was added 6N HCl (5 mL) and heated at reflux for 2 hours. The ethanol was removed in vacuo, the aqueous layer neutralized to pH=7 with 2N aq. NaOH, and filtered to isolate a yellow solid in 90% yield.

¹H NMR (DMSO-d₆): δ 7.68 (2H, d, J=9.4 Hz), 7.64 (2H, d, J=9.4 Hz), 7.12 (2H, s), 6.08 (2H, d, J=10.6 Hz), 5.88 (2H, s).

HRESIMS: calcd. for C₁₆H₁₄F₂N₅O₃S₂ (MH⁺): 426.0506. Found: 426.0501.

Anal. calcd. for C₁₆H₁₃F₂N₅O₃S₂.0.5H₂O: C, 44.23; H, 3.25; N, 16.12; S. 14.76. Found: C, 44.30; H, 3.26; N, 15.79; S, 14.86.

### Example W(1)

### 4-Amino-5-(2,6-dichloro-benzoyl)-2-[4-(pyridin-4-ylthio)-phenylamino]-thiazole

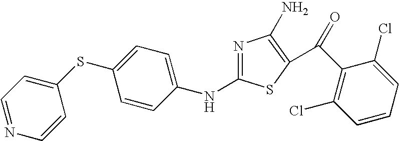

First, 4-(4-nitro-phenylthio)-pyridine, which has the structural formula

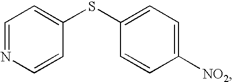

was prepared. 1-Iodo-4-nitro-benzene (Aldrich, 1.24 g, 5.00 mmol) and pyridine-4-thiol (Aldrich, 0.55 g, 5.0 mmol) in DMF was heated at 128° C. for 5 hours. The solvent was removed under reduced pressure and ethyl acetate added. The solution was washed with 0.1 N NaOH, dried over MgSO₄, and evaporated. Purification via column chromatography gave 0.62 g (54% yield) of a yellow solid, which was used without any further purification. ¹HNMR: δ 8.52 (2H, d, J=6.5 Hz), 8.22 (2H, d, J=8.6 Hz), 7.56 (2H, d, J=8.6 Hz), 7.20 (2H, d, J=6.5 Hz).

4-(4-Isothiocyanato-phenylthio)-pyridine, which has the structural formula

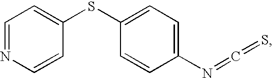

was prepared as follows. A solution of 4-(4-nitro-phenylthio)-pyridine (500 mg, 2.15 mmol) in concentrated HCl (0.5 mL) and methanol (50 mL) was hydrogenated at 20 psi in the presence of 10% Pd/C for 2 hours, then filtered through Celite. The filtrate was concentrated (free base previously known, mp 169-71° C., see Takahashi, et al, Pharm. Bull., 30 (1954)), immediately a portion (0.46 g, 2.0 mmol) was combined with Et₃N (0.5 g, 5 mmol) and CH₂Cl₂, and cooled to 0° C. Thiophosgene (0.26 g, 2.2 mmol) was added dropwise and the mixture allowed to warm to ambient temperature over 1 hours. More CH₂Cl₂ was added, washed with sat. aq. NaHCO₃ and brine, dried over MgSO₄, and concentrated in vacuo to give a crude product, which was purified by column chromatography to provide 0.20 g (40% yield) of a yellow solid, which was used without any further purification.

¹H NMR: δ 8.35 (2H, d, J=6.5 Hz), 7.54 (2H, d, J=8.6 Hz), 7.26 (2H, d, J=8.6 Hz), 6.95 (2H, d, J=6.5 Hz).

FTIR (KBr): 2180 (s) cm⁻¹.

The title compound was prepared in the same manner that was described for 4-[4-amino-5-(2,6-difluoro-benzoyl)-thiazol-2-ylamino]-benzenesulfonyl fluoride from Example A(1). 4-(4-Isothiocyanato-phenylthio)-pyridine and 2-bromo-2′,6′-dichloro-acetophenone (from World Patent Application WO 99/21845 and Mlotkowska, et al., Pol. J. Chem., 55, 631-642 (1981)) gave 10 mg (3%) of a yellow solid.

¹H NMR: δ 8.36 (2H, d, J=6.5 Hz), 7.50 (2H, d, J=8.6 Hz), 7.42 (2H, d, J=9.2 Hz), 7.32 (2H, d, J=8.6 Hz), 7.24 (1H, m), 6.92 (2H, d, J=6.5 Hz).

HRFABMS calcd. for C₂₁H₁₅N₄OS₂Cl₂ (M+H⁺): 473.0064. Found: 473.0070.

### Example W(2)

### 4-Amino-5-(2,6-dichloro-benzoyl)-2-[4-(pyridin-2-ylthio)-phenylamino]-thiazole

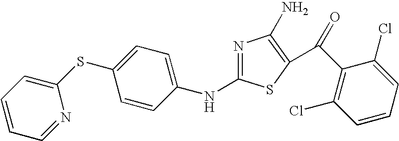

The title compound was prepared in the same manner that was described for 4-[4-amino-5-(2,6-difluoro-benzoyl)-thiazol-2-ylamino]-benzenesulfonyl fluoride from Example A(1). 2-(4-Isothiocyanato-phenylthio)-pyridine (Chuchani, et al, J. Chem. Soc. C, p.1436 (1969)) and 2-bromo-2′,6′-dichloro-acetophenone (World Patent Application WO 99/21845 and Mlotkowska, et al., Pol. J. Chem., 55, 631-642 (1981)) gave 180 mg (25%) of yellow solid.

¹H NMR (DMSO-d₆): δ 8.28 (2H, d, J=4.2 Hz), 7.60-7.30 (8H, m), 6.98 (1H, m), 6.76 (2H, d, J=8.6 Hz).

HRFABMS: calcd. for C₂₁H₁₅N₄OS₂Cl₂ (M+H⁺): 473.0064. Found: 473.0076.

### Example X(1)

### 4-Amino-5-(2,6-dichloro-benzoyl)-2-(4-mercapto-phenylamino)-thiazole

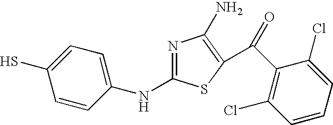

First 4-[1,1-bis-(4-methoxy-phenyl)-1-phenyl-methylthio]-phenylamine, which has structural formula

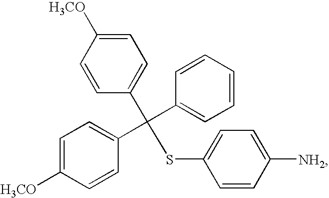

was prepared. To a solution of 4,4′-dimethoxytrityl chloride (3.39 g, 10.0 mmol) in CH₂Cl₂ (100 mL) was added a solution of 4-amino-thiophenol (2.50 g, 20.0 mmol) in CH₂Cl₂ (50 mL). After 2 hours, the resultant solution was washed with sat. aq. citric acid, sat. aq. NaHCO₃ and brine, dried over MgSO₄, and concentrated in vacuo to give a crude product, which was purified via column chromatography to give 3.21 g (37% yield) of a solid. Used without any further purification.

¹H NMR: δ 7.38-7.14 (m, 9H), 6.80-6.63 (m, 6H), 6.36 (d, 2H, J=8.6 Hz), 3.78 (s, 6H).

4-[1,1-Bis-(4-methoxy-phenyl)-1-phenyl-methylthio]-phenylisothiocyanate, which has structural formula

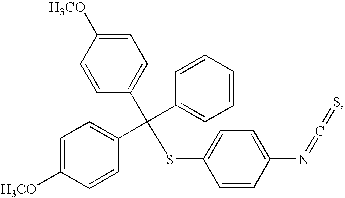

was prepared in a manner similar to that for 3-isothiocyanato-benzenesulfonamide in Example P(1). 4-[1,1-Bis-(4-methoxy-phenyl)-1-phenyl-methylthio]-phenylamine gave 1.87 g (53% yield) of a solid, which was used without any further purification.

¹H NMR: δ 7.35 (2H, m), 7.28-7.14 (7H, m), 6.95-6.84 (4H, m), 6.79-6.72 (4H, m), 3.79 (6H, s).

4-Amino-2-{4-[1,1-bis-(4-methoxy-phenyl)-1-phenyl-methylthio]-phenylamino}-5-(2,6-dichlorobenzoyl)-thiazole, with the structural formula

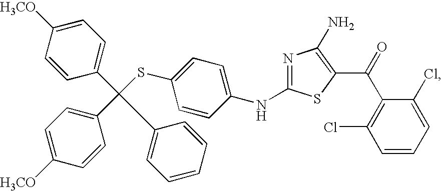

was prepared in a manner analogous to that for 4-[4-amino-5-(2,6-difluoro-benzoyl)-thiazol-2-ylamino]-benzenesulfonyl fluoride from Example A(1). 4-[1,1-Bis-(4-methoxy-phenyl)-1-phenyl-methylthio]-phenylisothiocyanate and 2-bromo-2′,6′-dichloro-acetophenone (World Patent Application WO 99/21845 and Mlotkowska, et al., Pol. J. Chem., 55, 631-642 (1981)) gave 1.39 g (63% yield) of a yellow solid, which was used without any further purification.

¹H NMR (DMSO-d₆): δ 10.81 (1H, s), 8.14 (2H, bs), 7.59-7.44 (3H, m), 7.32-7.14 (11H, m), 6.91-6.78 (6H, m), 3.83 (6H, s).

The title compound was prepared as follows. A solution of 4-amino-2-{4-[1,1-bis-(4-methoxy-phenyl)-1-phenyl-methylthio]-phenylamino}-5-(2,6-dichlorobenzoyl)-thiazole (1.50 g, 2.15 mmol) and triisobutylsilane (0.59 ml, 2.3 mmol) in 50% TFA/CH₂Cl₂ (30 ml) stirred at ambient temperature for 3 hours. After removal of solvent in vacuo, CH₂Cl₂ was added. The solution was washed with a sat. aq. NaHCO₃, brine, dried over MgSO₄, and evaporated. The crude solid was purified by column chromatography to give 720 mg (91% yield) of a yellow solid which was immediately used without any further purification or characterization.

¹H NMR (DMSO-d₆): δ 10.90 (1H, bs), 8.11 (2H, bs), 7.41-7.42 (4H, m), 7.32-7.12 (2H, m), 6.90-6.80 (1H, m).

FABMS (MH⁺): 398.

### Example X(2)

### 3-Amino-5-(2,6-dichlorobenzoyl)-2-(4-mercapto-phenylamino)-thiazole

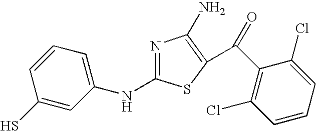

First 3-[1,1-bis-(4-methoxy-phenyl)-1-phenyl-methylthio]-phenylamine, which has the structural formula

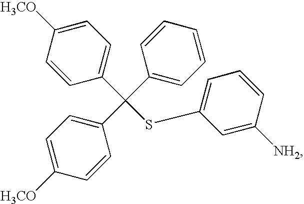

was prepared in a manner analogous to that for 4-[1,1-bis-(4-methoxy-phenyl)-1-phenyl-methylthio]-phenylamine from Example X(1). 3-Aminothiophenol provided 4.50 g (53% yield) of a yellow solid, which was used without any further purification.

¹H NMR: δ 7.38-7.14 (m, 9H), 6.80-6.63 (m, 6H), 6.36 (d, 2H, J=8.6 Hz), 3.78 (s, 6H).

3-[1,1-Bis(4-methoxy-phenyl)-1-phenyl-methylthio]-phenylisothiocyanate, which has the structural formula

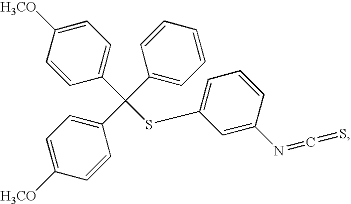

was prepared in a manner analogous to that for 3-isothiocyanato-benzenesulfonamide in Example P(1). 3-[1,1-Bis-(4-methoxy-phenyl)-1-phenyl-methylthio]-phenylamine led to 3.55 g (65% yield) of yellow solid, which was used without any further purification.

¹H NMR: δ 7.35 (2H, m), 7.28-7.14 (7H, m), 6.95-6.84 (4H, m), 6.79-6.72 (4H, m), 3.79 (6H, s).

4-Amino-2-{3-[1,1-bis-(4-methoxy-phenyl)-1-phenyl-methylthio]-phenylamino-}-5-(2,6-dichlorobenzoyl)-thiazole, which has the structural formula

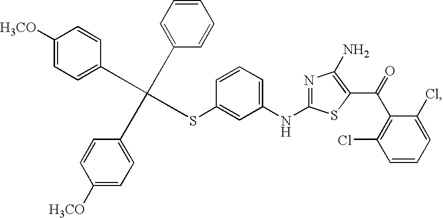

was prepared in a manner analogous to that for 4-[4-amino-5-(2,6-difluoro-benzoyl)-thiazol-2-ylamino]-benzenesulfonyl fluoride in Example A(1). 3-[1,1-Bis-(4-methoxy-phenyl)-1-phenyl-methylthio]-phenylisothiocyanate and 2-bromo-2′,6′-dichloro-acetophenone (World Patent Application WO 99/21845 and Mlotkowska, et al., Pol. J. Chem., 55, 631-642 (1981)) gave 2.52 g (47% yield) of a yellow solid, which was used without any further purification.

¹H NMR (DMSO-d₆): δ 10.81 (1H, s), 8.14 (2H, bs), 7.59-7.44 (3H, m), 7.32-7.14 (11H, m), 6.91-6.78 (6H, m), 3.83 (6H, s).

The title compound was prepared in a manner similar to that used to prepare 4-amino-5-(2,6-benzoyl)-2-(4-mercapto-phenylamino)-thiazole (Example X(1)). 4-Amino-2-{3-[1,1-bis-(4-methoxy-phenyl)-1-phenyl-methylthio]-phenylamino-}-5-(2,6-dichlorobenzoyl)-thiazole gave 1.19 g (83% yield) of a yellow solid, which was used without any further purification.

¹H NMR (DMSO-d₆): δ 10.55 (1H, s), 7.91 (2H, bs), 7.38-7.25 (4H, m), 7.18-7.00 (2H, m), 6.85 (1H, d, J=7.6 Hz), 5.30 (1H, s).

HRESIMS: calcd. for C₁₆H₁₂Cl₂N₃OS₂ (M+H⁺): 395.9799. Found: 395.9813.

Anal. calcd. for C₁₆H₁₁Cl₂N₃OS₂.0.45 EtOAc: C, 49.04; H, 3.38; N, 9.64; S, 14.71. Found: C, 48.97; H, 3.12; N, 9.59; S, 14.84.

### Example Y(1)

### 2-{4-[4-Amino-5-(2,6-dichloro-benzoyl)-thiazol-2-ylamino]-phenylthio}-acetamide

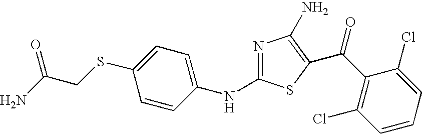

A mixture of 4-amino-5-(2,6-benzoyl)-2-(4-mercapto-phenylamino)-thiazole (Example X(1); 297 mg, 0.749 mmol), 2-bromo-acetamide (124 mg, 0.899 mmol) and N,N-diisopropylethylamine (DIEA; 156 μL, 0.896 mmol) in DMF (10 mL) was stirred at ambient temperature for 30 minutes. The solvent was evaporated under reduced pressure. Ethyl acetate was added, then washed with sat. aq. citric acid, sat. aq. NaHCO₃, and brine, dried over MgSO₄, concentrated, and to give 361 mg (76%) of a yellow solid.

¹H NMR (DMSO-d₆): 10.88 (1H, s), 8.24 (2H, bs), 7.56-7.28 (8H, m), 7.11 (1H, bs), 3.54 (2H, s).

HRESIMS: calcd. for C₁₈H₁₅Cl₂N₄O₂S₂ (M+H⁺): 453.0013. Found: 453.0022.

Anal. calcd. for C₁₈H₁₄Cl₂N₄O₂S₂.0.18 CH₃OH.0.25 CHCl₃: C, 45.27; H, 3.09; N, 11.46; S, 13.12. Found: C, 45.14; H, 3.28; N, 11.46; S, 13.38.

### Example Y(2)

### 4-Amino-5-(2,6-dichlorobenzoyl)-2-[4-(2-hydroxy-ethylthio)-phenylamino]-thiazole

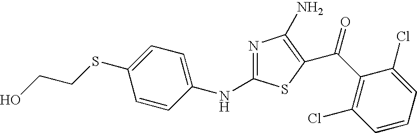

The title compound was prepared in a manner similar to that used to prepare 2-{4-[4-amino-5-(2,6-dichloro-benzoyl)-thiazol-2-ylamino]-phenylthio}-acetamide (Example Y(1). 4-Amino-2-(4-mercapto-phenylamino)-5-(2,6-benzoyl)-thiazole (Example X(1)) and 2-bromo-ethanol and purification via column chromatography with EtOAc: CH₂Cl₂ (1:1) as eluant afforded 92 mg (28% yield) of yellow solid.

¹H NMR (DMSO-d₆): δ 10.85 (1H, s), 8.12 (2H, bs), 7.58-7.42 (5H, m), 7.34 (2H, d, J=8.8 Hz), 4.91 (1H, t, J=5.6 Hz), 3.53 (2H, m), 3.01 (2H, t, J=6.9 Hz).

FABMS (MH⁺): 442.

### Example Y(3)

### 2-{3-[4-Amino-5-(2,6-dichloro-benzoyl)-thiazol-2-ylamino]-phenylthio) acetamide

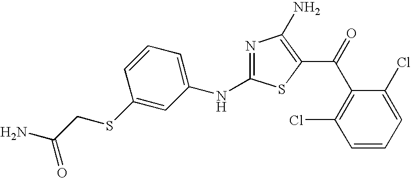

The title compound was prepared in a manner similar to that used to prepare 2-(4-[4-amino-5-(2,6-dichloro-benzoyl)-thiazol-2-ylamino]-phenylthio}-acetamide (Example Y(1). 4-Amino-2-(3-mercapto-phenylamino)-5-(2,6-dichlorobenzoyl)-thiazole (Example R(2)) and 2-bromo-acetamide and purification via column chromatography with EtOAc: hex (1:1) as eluant afforded 63.7 mg (56% yield) of yellow solid.

¹H NMR (DMSO-d₆): δ 10.88 (1H, bs), 8.24 (2H, bs), 7.71 (1H, bs), 7.64-7.50 (4H, m), 7.35-7.31 (2H, m), 7.25 (1H, m), 7.11 (1H, m), 3.70 (2H, s).

HRESIMS: calcd. for C₁₈H₁₅Cl₂N₄O₂S₂ (M+H⁺): 413.0013. Found: 413.0024.

### Example Z(1)

### 4-Amino-5-(2,6-benzoyl)-2-(3-methanesulfinyl-phenylamino)-thiazole

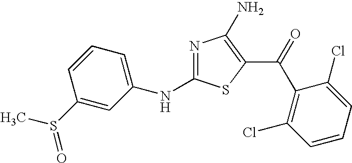

To a solution of 4-amino-5-(2,6-benzoyl)-2-(3-methylthio-phenylamino)-thiazole (Example R(3)); 100 mg, 0.250 mmol) in THF was added 32% peracetic acid (60 μL, 0.25 mmol). After 30 minutes, CH₂Cl₂ was added. The organic layer was washed with a sat. aq. NaHCO₃ and brine, dried over MgSO₄, and concentrated to give a crude solid, which was purified by column chromatography to give 81 mg (76% yield) of a yellow solid.

¹H NMR: δ 7.75 (m, 2H), 7.50 (m, 1H), 7.30 (m, 4H), 2.78 (s, 3H).

HRFABMS: calcd. for C₁₇H₁₄Cl₂N₃O₂S₂ (M+H⁺): 425.9905. Found: 425.9913.

### Example Z(2)

### 2-(4-{4-Amino-5-(2,6-dichlorobenzoyl)-thiazol-2-ylamino}-benzenesulfinyl)-acetamide

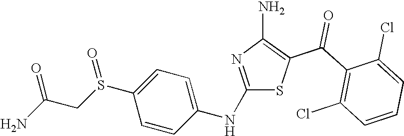

The title compound was prepared in manner similar to that used to prepare 4-amino-5-(2,6-benzoyl)-2-(3-methanesulfinyl-phenylamino)-thiazole (Example Z(1)): 2-{4-[4-Amino-5-(2,6-dichloro-benzoyl)-thiazol-2-ylamino]-phenylthio}-acetamide (Example Y(1)) gave 365 mg (76% yield) of a yellow solid.

¹H NMR (DMSO-d₆): δ 11.05 (1H, s), 8.18 (2H, br), 7.84 (2H, d, J=8.8 Hz), 7.76 (2H, d, J=8.8 Hz), 7.58-7.42 (3H, m), 7.28 (1H, bs), 3.69 (2H, q, J=19.4 Hz).

HRFABMS. Calcd for C₁₈H₁₄Cl₂N₄O₃S₂Na (M+Na⁺): 490.9782. Found: 490.9768.

Anal. calcd. for C₁₈H₁₄Cl₂N₄O₃S₂.0.7 CH₃OH: C, 45.67; H, 3.44; N, 11.39; S, 13.04. Found: C, 45.92; H, 3.58; N, 11.11; S, 13.21.

### Example Z(3)

### 4-Amino-5-(2,6-dichlorobenzoyl)-2-[4-(2-hydroxy-ethanesulfinyl)-phenylamino]-thiazole

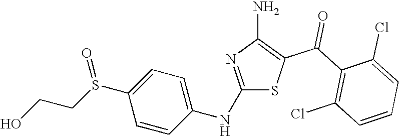

The title compound was prepared in manner similar to that used to prepare 4-amino-5-(2,6-benzoyl)-2-(3-methanesulfinyl-phenylamino)-thiazole (Example Z(1)): 4-Amino-5-(2,6-dichlorobenzoyl)-2-[4-(2-hydroxy-ethylthio)-phenylamino]-thiazole (Example Y(2)) and purification via column chromatography with MeOH:CH₂Cl₂:EtOAc (0.2:1:2) as eluant gave 23 mg (32% yield) of a yellow solid.

¹H NMR (DMSO-d₆): δ 7.81 (2H, d, J=8.7 Hz), 7.71 (2H, d, J=8.7 Hz), 7.48-7.38 (3H, m), 4.00 (1H, m), 3.82 (1H, m), 3.08 (2H, m).

HRFABMS: calcd for C₁₈H₁₅Cl₂N₃O₃S₂Na (M+Na⁺): 477.9830. Found: 477.9816.

### Example Z(4)

### 4-Amino-5-(2,6-dichlorobenzoyl)-2-(4-methanesulfinyl-phenylamino)-thiazole

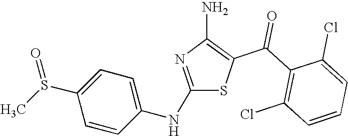

The title compound was prepared in manner similar to that used to prepare 4-amino-5-(2,6-benzoyl)-2-(3-methanesulfinyl-phenylamino)-thiazole (Example Z(1)): 4-Amino-5-(2,6-benzoyl)-2-(4-methylthio-phenylamino)-thiazole (Example (R(2)) gave 26 mg (31% yield) of a yellow solid.

¹H NMR (CD₃OD): δ 7.90 (2H, d, J=8.8 Hz), 7.72 (2H, d, J=8.8 Hz), 7.51-7.38 (3H, m), 2.80 (3H, s).

HRESIMS: calcd. for C₁₇H₁₄Cl₂N₃O₂S₂ (M+H⁺): 425.9905. Found: 425.9920.

### Example Z(5)

### 2-{3-[4-Amino-5-(2,6-dichloro-benzoyl)-thiazol-2-ylamino]-benzenesulfinyl}-acetamide

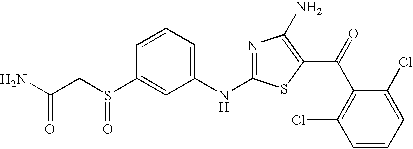

The title compound was prepared in manner similar to that used to prepare 4-amino-5-(2,6-dichloro-benzoyl)-2-(3-methanesulfinyl-phenylamino)-thiazole (Example Z(1)): 2-{3-[4-Amino-5-(2,6-dichloro-benzoyl)-thiazol-2-ylamino]-phenylthio}-acetamide (Example R(3)) purification via column chromatography with MeOH:EtOAc (0.5:4) as eluant gave 76 mg (62% yield) of a yellow solid.

¹H NMR (DMSO-d₆): δ 11.04 (1H, s), 8.18 (2H, bs), 7.88 (2H, m), 7.64-7.35 (7H, m), 3.70 (2H, q, J=17.3 Hz).

HRESIMS: calcd. for C₁₈H₁₅Cl₂N₄O₃S₂ (M+H⁺): 468.9963. Found: 468.9981.

Anal. calcd. for C₁₈H₁₄Cl₂N₄O₃S₂ 0.43 CHCl₃: C, 42.51; H, 2.79; N, 10.76; S, 12.32. Found: C, 42.47; H, 2.95; N, 10.69; S, 12.28.

### Example AA(1)

### 4-Amino-5-(2,6-dichloro-benzoyl)-2-(3-methanesulfonyl-phenylamino)-thiazole

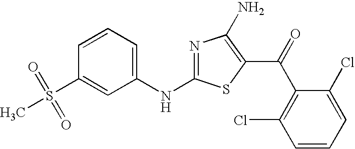

To a solution of 4-amino-2-(3-methylsulfinyl-phenylamino)-5-(2,6-benzoyl)-thiazole (Example Z(1); 100 mg, 0.235 mmol) in THF was added 32% peracetic acid (180 μL, 0.75 mmol). After 30 minutes, CH₂Cl₂ was added. The organic layer was washed with a sat aq NaHCO₃ and brine, dried over MgSO₄, and concentrated. The crude product was purified by column chromatography to give 74 mg (67% yield) of a yellow solid.

¹H NMR: δ 7.94 (1H, s), 7.78 (1H, m), 7.64 (2H, m), 7.30 (3H, m), 3.08 (3H, s).

HRFABMS: calcd for C₁₇H₁₄Cl₂N₃O₃S₂ (M+H⁺): 441.9854. Found: 441.9841.

### Example AA(2)

### 4-Amino-5-(2,6-dichlorobenzoyl)-2-(4-methanesulfonyl-phenylamino)-thiazole

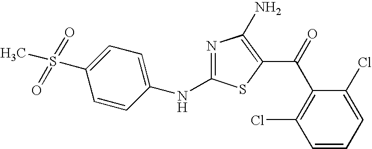

The title compound was prepared in manner similar to that used to prepare 4-amino-5-(2,6-dichloro-benzoyl)-2-(3-methanesulfonyl-phenylamino)-thiazole (Example AA(1)): 4-Amino-5-(2,6-dichloro-benzoyl)-2-(4-methylthio-phenylamino)-thiazole (Example R(2)) gave 14 mg (17% yield) of a yellow solid.

¹H NMR (DMSO-d₆): δ 7.94-7.88 (4H, s), 7.57-7.34 (3H, m), 3.10 (3H, s).

HRFABMS: calcd. for C₁₇H₁₄Cl₂N₃O₃S₂ (M+H⁺): 441.9854. Found: 441.9856.

### Example AA(3)

### 4-Amino-5-(2,6-dichlorobenzoyl)-2-[4-(pyridine-4-sulfonyl)-phenylamino]-thiazole

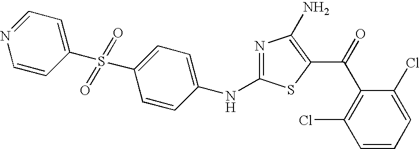

The title compound was prepared in manner similar to that used to prepare 4-amino-5-(2,6-dichlorobenzoyl)-2-(3-methanesulfonyl-phenylamino)-thiazole (Example AA(1)): 4-Amino-5-(2,6-dichlorobenzoyl)-2-[4-(pyridin-4-ylthio)-phenylamino]-thiazole (Example W(1)) gave 5 mg (5% yield) of a yellow solid.

¹H NMR (DMSO-d₆): δ 8.86 (2H, d, J=8.0 Hz), 7.97 (2H, d, J=8.0 Hz), 7.88-7.81 (4H, m), 7.56-7.47 (3H, m).

HRFABMS: calcd. for C₂₁H₁₅N₄O₃S₂Cl₂ (M+H⁺): 504.9963. Found: 504.9955.

### Example BB(1)

### 4-[4-Amino-5-(2,6-difluoro-benzoyl)-thiazol-2-ylamino]-N-piperidin-4-yl-benzenesulfonamide

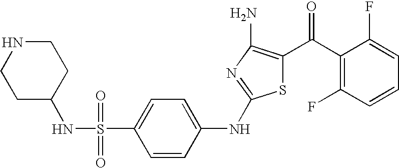

First the starting material, 4-(4-acetylamino-benzenesulfonylamino)-piperidine-1-carboxylic acid ethyl ester, which has the structural formula

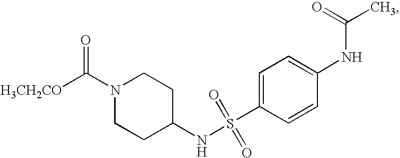

was prepared as follows. To a suspension of ethyl 4-amino-1-piperidinecarboxylate (5.00 g, 29.0 mmol) and sodium acetate (5.95 g, 72.6 mmol) in ethanol (58 mL) at 0° C. was added N-acetylsulfanilyl chloride (6.10 g, 26.1 mmol). The mixture stirred at ambient temperature for one hour, then was diluted with water (400 mL) and filtered. The isolated white solid washed with water, dried under vacuum, and used without any further purification.

¹H NMR (CD₃OD): δ 7.82 (2H, d, J=8.8 Hz), 7.76 (2H, d, J=8.8 Hz), 4.08 (2H, q, J=7.1 Hz), 3.91 (1H, dt, J=3.0, 13.8 Hz), 3.34-3.30 (2H, m), 3.23 (1H, tt, J=4.1, 10.3 Hz), 2.88 (2H, t, J=10.3 Hz), 1.73-1.63 (2H, m), 1.40-1.27 (2H, m), 1.23 (3H, t, J=7.1 Hz).

4-Amino-N-piperidin-4-yl-benzenesulfonamide, which has the structural formula

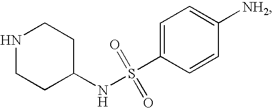

was prepared as follows. 4-(4-Acetylamino-benzenesulfonylamino)-piperidine-1-carboxylic acid ethyl ester was dissolved in conc. HCl (60 mL), heated at reflux for 7 hours, allowed to cool, concentrated in vacuo, and dissolved in water (20 mL). Basified to pH=11 with 4N NaOH and extracted with 30% iPrOH/CHCl₃. The organic layer was dried over Na₂SO₄ and concentrated to give 2.56 g of white solid (38% for two steps, from N-acetylsulfanilyl chloride), which was used without any further purification.

¹H NMR (DMSO-d₆): δ 7.42 (2H, d, J=8.7 Hz), 7.16 (1H, d, J=7.2 Hz), 6.58 (2H, d, J=8.7 Hz), 5.87 (2H, s), 3.32 (1H, bs), 2.78 (2H, dt, J=3.9, 12.6 Hz), 2.28 (2H, td, J=2.1, 11.6 Hz), 1.45 (2H, dd, J=2.8, 12.6 Hz), 1.15 (2H, qd, J=3.9, 11.6 Hz).

FABMS. (MH⁺): 256.

4-(4-Amino-benzenesulfonylamino)-piperidine-1-carboxylic acid t-butyl ester, which has the structural formula

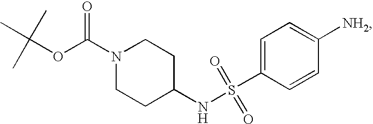

was prepared as follows. Triethylamine (0.66 mL, 4.7 mmol) and di t-butyl dicarbonate (1.13 g, 5.17 mmol) were sequentially added to a solution of 4-amino-N-piperidin-4-yl-benzenesulfonamide (1.20 g, 4.70 mmol) in THF (16 mL) and CH₂Cl₂ (16 mL) at 0° C. The mixture was allowed to warm to ambient temperature and stir overnight. The resultant mixture was extracted with CH₂Cl₂. The organic layer was separated, washed with 0.5 N HCl, dried over Na₂SO₄, and concentrated to give 1.37 g (82% yield) of white solid, which was used without any further purification.

¹H NMR (DMSO-d₆): δ 7.43 (2H, d, J=8.7 Hz), 7.25 (1H, d, J=7.3 Hz), 6.59 (2H, d, J=8.7 Hz), 3.69 (2H, bd, J=13.4 Hz), 3.02 (1H, m), 2.76 (2H, bs), 1.52 (2H, dd, J=3.6, 13.4 Hz), 1.36 (9H, s), 1.16 (2H, qd, J=4.2, 10.3 Hz).

4-(4-Isothiocyanato-benzenesulfonylamino)-piperidine-1-carboxylic acid t-butyl ester, which has the structural formula

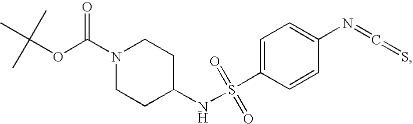

was prepared as follows. Thiophosgene (121 mL) was added in one portion to a solution of 4-(4-amino-benzenesulfonylamino)-piperidine-1-carboxylic acid t-butyl ester (562 mg, 1.58 mmol) in 1N HCl (4 mL) and THF (4 mL). The mixture stirred for 20 minutes, then partitioned between ether and water. The organic layer was separated, washed with water and brine, dried over Na₂SO₄, and evaporated to give 578 mg (92% yield) of yellow powder.

¹H NMR (DMSO-d₆): δ 7.91 (1H, d, J=7.4 Hz), 7.86 (2H, d, J=8.7 Hz), 7.62 (2H, d, J=8.7 Hz), 3.71 (2H, bd, J=13.2 Hz), 3.17 (1H, m), 2.76 (2H, bs), 1.56-1.48 (2H, m), 1.36 (9H, s), 1.18 (2H, qd, J=4.1, 11.2 Hz).

4-(4-{4-Amino-5-(2,6-difluoro-benzoyl)-thiazol-2-ylamino}-benzenesulfonylamino)-piperidine-1-carboxylic acid t-butyl ester, which has the structural formula

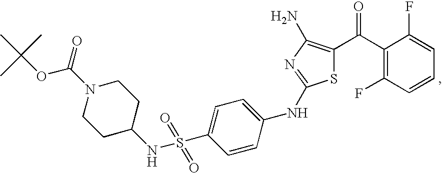

was prepared in a manner similar to that for 4-[4-amino-5-(2-hydroxy-2-methyl-propionyl)-thiazol-2-ylamino]-benzenesulfonamide (Example M(1)). 4-(4-Isothiocyanato-benzenesulfonylamino)-piperidine-1-carboxylic acid t-butyl ester (1.43 g, 3.60 mmol) led to 1.52 g (80% yield) of a yellow solid, which was used without further purification.

¹H NMR (DMSO-d₆): δ 11.21 (1H, s), 8.25 (2H, bs), 7.80 (4H, s), 7.72 (1H, d, J=7.3 Hz), 7.58 (1H, m), 7.25 (2H, dd, J=7.8, 8.1 Hz), 3.71 (2H, bd, J=13.2 Hz), 3.18 (1H, m), 2.80 (2H, bs), 1.55 (2H, dd, J=3.3, 13.2 Hz), 1.38 (9H, s), 1.21 (2H, qd, J=3.9, 10.5 Hz).

The title compound was prepared in a manner similar to that for Example D(1). 4-(4-{4-Amino-5-(2,6-difluoro-benzoyl)-thiazol-2-ylamino}-benzenesulfonylamino)-piperidine-1-carboxylic acid t-butyl ester (1.50 g, 2,8 mmol) furnished 0.80 g (59% yield) of a yellow solid.

¹H NMR (DMSO-d₆): δ 8.13 (2H, bs), 7.73 (2H, d, J=8.9 Hz), 7.66 (2H, d, J=8.9 Hz), 7.61 (1H, b), 7.52 (1H, m), 7.19 (2H, dd, J=7.7, 8.2 Hz), 3.00 (1H, m), 2.84 (2H, bd, J=12.5 Hz), 2.40 (2H, t, J=11.0 Hz), 1.51 (2H, d, J=12.5 Hz), 1.23 (2H, qd, J=3.9, 11.0 Hz).

HRFABMS: calcd. for C₂₁H₂₂N₅O₃F₂S₂ (M+H⁺): 494.1132. Found: 494.1114.

Anal. calcd. for C₂₁H₂₁N₅O₃F₂S₂.0.6H₂O-0.3 EtOH: C, 50.07; H, 4.67; N, 13.52; S, 12.38. Found: C, 50.19; H, 4.71; N, 13.44; S, 12.47.

### Example CC(1)

### 4-[4-Amino-5-(2,6-difluoro-4-methyl-benzoyl)-thiazol-2-ylamino]-N-(2-isopropoxy-ethyl)-benzenesulfonamide

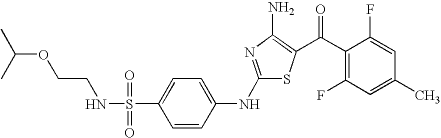

First the starting material (2,6-difluoro-4-methyl-phenyl)-trimethylsilane, which has the structural formula

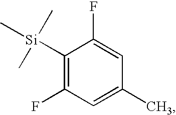

was made as follows. To a solution of (4-bromo-2,6-difluoro-phenyl)-trimethylsilane (2.52 g, 9.50 mmol; from Example FF(2)) in ether (25 mL) at −60° C. was added n-BuLi (7.10 mL of 1.6 M in hex). The mixture was allowed to warm to 0° C. over 35 minutes, then recooled to 60° C., iodomethane (0.89 mL, 14 mmol) added, and allowed to warm to ambient temperature. After 1 hour, quenched with water and extracted with ether. The separated organic layer was washed with water and brine, dried over MgSO₄, and carefully concentrated under reduced pressure on a rotary evaporator below 30° C. to give 1.90 g (100%) of yellow oil, which was immediately used in the next step without any further purification.

¹H NMR: δ 6.61 (2H, d, J=8.1 Hz), 0.35 (9H, dd, J=1.2, 1.3 Hz).

2′,6′-Difluoro-4′-methyl-acetophenone, which has the structural formula

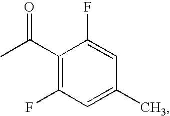

was made in a similar manner to that for 2-bromo-2′,6′-difluoro-acetophenone in Example A(1), with a procedure described by Bennetau, et al., Tetrahedron, 49, 10843-10845 (1993). (2,6-Difluoro-4-methyl-phenyl)-trimethylsilane (1.90 g, 9.50 mmol) provided 1.56 g (97% yield) of yellow oil, which was used without any further purification.

¹H NMR: δ 6.76 (2H, d, J=9.3 Hz), 2.57 (3H, t, J=1.9 Hz), 2.36 (3H, s).

2-Bromo-2′,6′-difluoro-4′-methyl-acetophenone, which has the structural formula

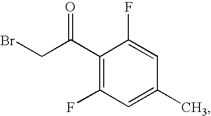

was made in a manner similar to that for 2-bromo-2′,6′-difluoro-3′-nitro-acetophenone in Example F(1). 2′,6′-Difluoro-4′-methyl-acetophenone (1.25 g, 7.35 mmol) and CuBr₂ (3.28 g, 14.7 mmol) gave 1.75 g (96% yield) of yellow oil, which was used without any further purification.

¹H NMR: δ 7.21 (2H, d, J=9.4 Hz), 4.35 (2H, s), 2.40 (3H, s).

4-[4-Amino-5-(2,6-difluoro-4-methyl-benzoyl)-thiazol-2-ylamino]-benzenesulfonyl fluoride, which has the structural formula

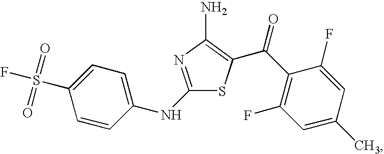

was made in a manner analogous to that for 4-[4-amino-5-(2,6-difluoro-benzoyl)-thiazol-2-ylamino]-benzenesulfonyl fluoride from Example A(1). 4-Isothiocyanato-benzenesulfonyl fluoride (793 mg, 3.65 mmol; from Example A(1)) and 2-bromo-2′,6′-difluoro-4′-methyl-acetophenone (1.00 g, 4.02 mmol) gave 1.61 g a yellow powder, which was used without any further purification.

¹H NMR (DMSO-d₆): δ 11.57 (1H, s), 8.22 (2H, bs), 8.09 (2H, d, J=8.9 Hz), 7.98 (2H, d, J=8.9 Hz), 7.06 (2H, d, J=8.7 Hz), 2.38 (3H, s).

FABMS. (MH⁻): 426.

The title compound was prepared in a manner analogous to that for Example A(1). 4-[4-Amino-5-(2,6-difluoro-4-methyl-benzoyl)-thiazol-2-ylamino]-benzenesulfonyl fluoride (200 mg, 0.54 mmol) and 2-aminoethyl isopropyl ether (0.20 mL, 1.63 mmol; TCl) and purification via preparative HPLC gave 234 mg (85% yield) of a green solid.

¹H NMR (DMSO-d₆): δ 11.15 (1H, s), 8.17 (2H, bs), 7.76 (4H, s), 7.55 (1H, t, J=6.0 Hz), 7.04 (2H, d, J=8.5 Hz), 3.45 (1H, heptet, J=6.1 Hz), 3.31 (2H, t, J=6.0 Hz), 2.86 (2H, q, J=5.8 Hz), 2.37 (3H, s), 1.01 (6H, d, J=6.1 Hz).

FABMS. (MH⁺): 511.

Anal. calcd. for C₂₂H₂₄F₂N₄O₄S₂.0.4 TFA.1.0H₂O: C, 47.67; H, 4.63; N, 9.76; S, 11.17. Found: C, 47.88; H, 4.50; N, 9.67; S, 10.96.

### Examples DD(1)-DD(240)

Collections of compounds were made in parallel and the assumed structures are provided in Table 3. For the first subset of alkylated thiols, reaction conditions analogous to that for 2-{4-[4-amino-5-(2,6-dichloro-benzoyl)-thiazol-2-ylamino]-phenylthio}-acetamide (Example Y(1)) were employed—adapted for parallel synthesis apparatus and workup—for the subset designated plates 1 and 4: a volume of stock solution corresponding to 15 μmol of either 4-amino-5-(2,6-dichloro-benzoyl)-2-(4-mercapto-phenylamino)-thiazole (Example X(1)) or 4-amino-2-(3-mercapto-phenylamino)-5-(2,6-dichloro-benzoyl)-thiazole (Example X(2)), respectively, in 5% DIEA/DMF was distributed into each well of two 96 deep-well plates. Then, various halides (15 μmol) were added into individual wells of each plates 1 and 4. After 1 hour at ambient temperature, solvent was removed in vacuo with a Genevac HT-4 Evaporator and then THF (600 μL) was added into each well.

Distribution of plates 1 and 4 and further processing led to subsequent sets of derivatives: For the plates 2 and 3, 200 μL of each well of plate 1 was each transferred into a corresponding well on each of plates 2 and 3, respectively. For plates 5 and 6, 200 μL of each well from plate 4 was placed into a respective corresponding well on each of the plates 5 and 6.

For a set of sulfoxides, conditions analogous to the preparation of [4-amino-2-(2-(3-methanesulfinyl-phenylamino)-5-(2,6-dichloro-benzoyl)-thiazole (Example Z(1) was adapted: To each of the wells in plates 2 and 5 was added a solution of peracetic acid (5.5 mmol) in THF.

For a set of sulfones, conditions analogous to the preparation of [4-amino-2-(3-methanesulfonyl-phenylamino)-5-(2,6-dichloro-benzoyl)-thiazole (Example M(1) were adapted: to each of the wells in plates 3 and 6, was added peracetic acid (15 μmol) in THF.

For plates 2, 3, 5, and 6, after 2 hours of agitation, 20% aqueous Na₂S₂O₃ (50 μL) was added to each well, allowed to agitate for another hour, and all solvent removed in vacuo.

For all plates, random wells were examined by HPLC to ensure appropriate processing. Crude residues in the wells were submitted for bioassay without further purification, and results are tabulated in Table 2.

### Examples EE(1)-EE(120)

To a mixture of 4-{[4-amino-5-(2,6-difluorobenzoyl)-1,3-thiazol-2-yl]amino}benzenesulfonyl fluoride (from Example A(1); 2 mg, 10 μmol) and anhydrous DMSO (10 μL) in each well of 1 mL deep-well plates were added corresponding commercially available amines (30 μmol). The plates were each sealed with a BECKMAN CAPMA™ and heated (alongside a 1 L beaker with deionized water (500 mL) as a heatsink) in a microwave oven (1100 W, 1.8 cu. ft.) at high power for three 20 minute intervals. After each interval, the water was replaced with deionized water at ambient temperature. The plates were allowed to cool and each well monitored by LCFIMS and LCMS (positive mode). These crude wells were submitted for bioassay without further purification, and the results are tabulated in Table 4.

### Example FF(1)

### 4-[4-Amino-5-(2,6-difluoro-3-iodo-benzoyl)-thiazol-2-ylamino]-benzenesulfonamide

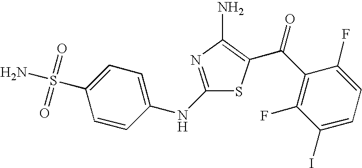

(2,6-Difluoro-3-iodo-phenyl)-trimethyl-silane, which has the structural formula

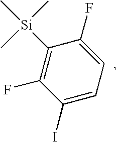

was made according to a procedure described by Akama et al., Synthesis; 1446-1450 (1997): To diisopropylamine (10.3 mL, 66.9 mmol) in THF (120 mL) at −78° C. under argon was added slowly n-BuLi (29.4 mL of 2.5 M in hex). The mixture stirred at 0° C. for 20 minutes and then recooled to −78° C., whereupon 2,4-difluoro-1-iodobenzene (8.0 mL, 66.9 mmol) was added at such a rate that the temperature never exceeded −60° C. The solution stirred at −78° C. for 1 hour, chlorotrimethylsilane (11.0 mL, 87.0 mmol) was added, and then allowed to warm to ambient temperature over 1 hour, then quenched with water, and extracted with ether. The separated organic layers were washed with brine and concentrated in vacuo to give a yellow oil, which was used in the next step without any further purification.

¹H NMR: δ 7.53 (1H, m), 6.48 (1H, td, J=8.8, 0.8 Hz), 0.21 (9H, t, J=1.6 Hz).

2′,6′-Difluoro-3′-iodo-acetophenone, which has the structural formula

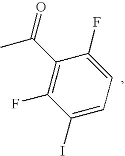

was made according to a procedure described by Bennetau et al., Tetrahedron, 49; 10843-10845 (1993). To a mixture of AlCl₃ (3.74 g, 28.0 mmol) in CH₂Cl₂ (40 mL) at 0° C. was added acetyl chloride (1.99 mL, 28.0 mmol). After 15 minutes at 0° C., (2′,6′-difluoro-3′-iodo-phenyl)-trimethyl-silane (22.4 mmol) in CH₂Cl₂ (25 mL) was added slowly, then allowed to warm to ambient temperature overnight. The mixture was cooled to 0° C., sat. aq. NH₄Cl (10 mL) added, stirred at 0° C. for 10 minutes, and partitioned between ether and 1N HCl. The ether layer was separated, washed with brine, dried over MgSO₄, and concentrated to give an oil, which was purified via column chromatography to provide 4.81 g (76% yield for two steps) of a yellow oil, which was used without any further purification.

¹H NMR: δ 7.79 (1H, m), 6.80 (1H, td, J=8.9, 1.4 Hz), 2.60 (3H, t, J=1.7 Hz).

2-Bromo-2′,6′-difluoro-3′-iodo-acetophenone, which has the structural formula

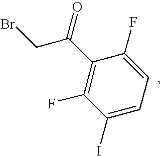

was made in a manner similar to that for 2-bromo-2′,6′-difluoro-3′-nitro-acetophenone in Example F(1). 2′,6′-Difluoro-3′-iodo-acetophenone (2.0 g, 7.1 mmol) and CuBr₂ (3.2 g, 14.2 mmol) gave a yellow solid in quantitative yield, which was used without any further purification.

¹H NMR: δ 7.86 (1H, m), 6.85 (1H, td, J=8.9, 1.4 Hz), 4.34 (2H, t, J=0.8 Hz).

The title compound was made in a manner analogous to that for 4-[4-amino-5-(2,6-difluoro-benzoyl)-thiazol-2-ylamino]-benzenesulfonyl fluoride from Example A(1). 4-Isothiocyanato-benzenesulfonamide (648 mg, 3.02 mmol) and 2-bromo-2′,6′-difluoro-3′-iodo-acetophenone (1.20 g, 3.32 mmol) gave 730 mg (45% yield) of a yellow solid.

¹H NMR (DMSO-d₆): δ 11.16 (1H, s), 8.25 (2H, bs), 7.99 (1H, m), 7.81 (2H, d, J=9.0 Hz), 7.75 (2H, d, J=9.0 Hz), 7.27 (2H, s), 7.12 (1H, dd, J=8.8, 8.6 Hz).

Anal. calcd. for C₁₆H₁₁F₂1N₄O₃S₂: C, 35.83; H, 2.07; N, 10.45; S, 11.69. Found: C, 35.81; H, 2.22; N, 10.18; S, 11.69.

### Example FF(2)

### 4-[4-Amino-5-(4-bromo-2,6-difluoro-benzoyl)-thiazol-2-ylamino]-benzenesulfonamide

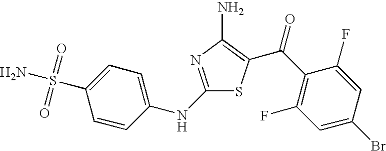

First the starting material (4-bromo-2,6-difluoro-phenyl)trimethylsilane, which has the structural formula

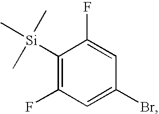

was made as follows. To diisopropylamine (1.73 mL, 12.4 mmol) in THF (30 mL) at −78° C. under argon was added slowly n-BuLi (7.73 mL of 1.6 M in hex). The mixture stirred at 0° C. for 20 minutes and then was recooled to −100° C. with a liquid nitrogen/ether slush bath, whereupon 1-bromo-3,5-difluorobenzene (2.17 g, 11.2 mmol) was added at such a rate that the temperature never exceeded −90° C. The solution stirred at −100° C. for 2 hours, chlorotrimethylsilane (1.86 mL, 14.6 mmol) was added dropwise at such a rate that the temperature kept below −85° C., allowed to warm to ambient temperature overnight, then quenched with water (2 mL), and extracted with ether. The separated organic layer was washed with brine and carefully concentrated under reduced pressure on a rotary evaporator below 30° C. to give 2.97 g (100%) of a colorless oil, which was used in the next step without any further purification.

¹H NMR: δ 7.00 (2H, ddd, J=2.6, 2.6, 7.9 Hz), 0.36 (9H, dd, J=1.4, 1.4 Hz).

4′-Bromo-2′,6′-difluoro-acetophenone, which has the structural formula

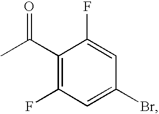

was made in a similar manner to that for 2-bromo-2′,6′-difluoro-acetophenone in Example A(1) with a procedure described by Bennetau, et al., Tetrahedron, 49; 10843-10845 (1993). 4′-Bromo-2′,6′-difluoro-phenyl)-trimethylsilane (11.2 mmol) provided 2.10 g (80% yield) of a yellow oil, which was used without any further purification.

¹H NMR: δ 7.16 (2H, ddd, J=2.3, 2.3, 10.2 Hz), 2.57 (3H, t, J=1.8 Hz).

2,4′-Dibromo-2′,6′-difluoro-acetophenone, which has the structural formula

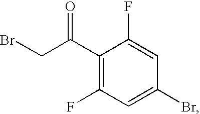

was made in a manner similar to that for 2-bromo-2′,6′-difluoro-3′-nitro-acetophenone in Example F(1). 4′-Bromo-2′,6′-difluoro-acetophenone (600 mg, 2.55 mmol) and CuBr₂ (1.14 g, 5.11 mmol) gave 796 mg (100%) of yellow oil, which was used without any further purification.

¹H NMR: δ 7.21 (2H, ddd, J=2.8, 2.8, 9.6 Hz), 4.31 (2H, bt, J=0.6 Hz).

The title compound was made in a manner analogous to that for 4-[4-amino-5-(2,6-difluoro-benzoyl)-thiazol-2-ylamino]-benzenesulfonyl fluoride from Example A(1). 4-Isothiocyanato-benzenesulfonamide (187 mg, 0.873 mmol) and 2,4′-dibromo-2′,6′-difluoro-acetophenone (300 mg, 0.962 mmol) and subsequent preparative HPLC purification gave 205 mg (48% yield) of yellow powder.

¹H NMR (DMSO-d₆): δ 11.10 (1H, s), 8.15 (2H, bs), 7.69 (4H, dd, J=8.8, 13.6 Hz), 7.53 (2H, d, J=6.9 Hz), 7.18 (2H, s).

Anal. calcd. for C₁₆H₁₁BrF₂N₄O₃S₂.0.3 TFA.0.8H₂O: C, 37.06; H, 2.42; N, 10.42; S, 11.92. Found: C, 37.15; H, 2.49; N, 10.26; S, 11.87.

### Example GG(1)

### 4-[4-Amino-5-(2-chloro-6-fluoro-benzoyl)-thiazol-2-ylamino]-benzenesulfonamide

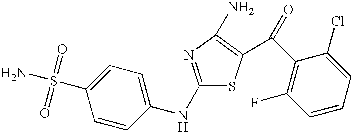

First 2-bromo-2′-chloro-6′-fluoro-acetophenone, which has the structural formula

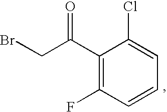

was made in a manner similar to that for 2-bromo-2′,6′-difluoro-3′-nitro-acetophenone in Example F(1). 2′-Chloro-6′-fluoro-acetophenone and CuBr₂ gave a colorless oil, which was used without any further purification.

¹H NMR: δ 7.45-7.32 (m, 1H), 7.12 (d, 1H, J=8.8 Hz), 7.07 (dd, 1H, J=4.2, 8.7 Hz), 4.38 (s, 2H).

The title compound was made in a manner analogous to that for 4-[4-amino-5-(2,6-difluoro-benzoyl)-thiazol-2-ylamino]-benzenesulfonyl fluoride from Example A(1). 4-Isothiocyanato-benzenesulfonamide (210 mg, 0.98 mmol) and 2-bromo-2′-chloro-6′-fluoro-acetophenone (259 mg, 1.03 mmol) and subsequent preparative PTLC purification with 1% (58% NH₄OH)/10% MeOH/CH₂Cl₂ gave 120 mg (27% yield) of brown powder.

¹H NMR (CD₃OD): δ 7.82 (4H, ddd, J=2.4, 6.7, 7.6 Hz), 7.43 (1H, ddd, J=5.9, 8.1, 10.5 Hz), 7.31 (1H, d, J=8.1 Hz), 7.16 (1H, ddd, J=0.9, 8.4, 8.6 Hz).

LCESIMS: (M+H⁺): 426.95.

Anal. calcd. for C₁₆H₁₂ClFN₄O₃S₂.0.1 hex.0.1 CH₂Cl₂: C, 44.74; H, 3.22; N, 12.54; S, 14.35; Cl: 8.73. Found: C, 44.82; H, 3.20; N, 12.40; S, 14.04; Cl: 8.84.

### Biochemical and Biological Evaluation

Cyclin-dependent kinase activity was measured by quantifying the enzyme-catalyzed, time-dependent incorporation of radioactive phosphate from [³²P]ATP or [³³P]ATP into a protein substrate. Unless noted otherwise, assays were performed in 96-well plates in a total volume of 50 μL, in the presence of 10 mM HEPES (N-[2-hydroxyethyl]piperazine-N′-[2-ethanesulfonic acid]) (pH 7.4), 10 mM MgCl₂, 25 μM adenosine triphosphate (ATP), 1 mg/mL ovalbumin, 5 μg/mL leupeptin, 1 mM dithiothreitol, 10 mM β-glycerophosphate, 0.1 mM sodium vanadate, 1 mM sodium fluoride, 2.5 mM ethylene glycol-bis(β-aminoethyl ether)-N,N,N′N′-tetraacetic acid (EGTA), 2% (v/v) dimethylsulfoxide, and 0.03-0.4 μCi [^{32/33}P]ATP per reaction. Reactions were initiated with enzyme, incubated at 30° C., and terminated after 20 minutes by the addition of ethylenediaminetetraacetic acid (EDTA) to 250 mM. The phosphorylated substrate was then captured on a nitrocellulose or phosphocellulose membrane using a 96-well filtration manifold, and unincorporated radioactivity was removed by repeated washing with 0.85% phosphoric acid. Radioactivity was quantified by exposing the dried membranes to a phosphorimager.

Compounds from combinatorial libraries were screened from 96-well plates for % inhibition of CDK activity at 100, 30, and/or 10 nM theoretical compound concentration. Inhibition was measured relative to control wells that contained all reaction components including 2% (v/v) DMSO but no compound, after subtraction of background radioactivity measured in the absence of enzyme. Apparent Ki values of discrete compounds were measured by assaying enzyme activity in the presence of different inhibitor compound concentrations and subtracting the background radioactivity measured in the absence of enzyme. The kinetic parameters (k_{cat}, Kₘ for ATP) were measured for each enzyme under the usual assay conditions by determining the dependence of initial rates on ATP concentration. Inhibition data were fit to an equation for competitive inhibition using Kaleidagraph (Synergy Software), or were fit to an equation for competitive tight-binding inhibition using the software KineTic (BioKin, Ltd.).

### Inhibition of CDK4/Cyclin D Retinoblastoma Kinase Activity

A complex of human CDK4 and genetically truncated (1-264) cyclin D3 was purified using traditional biochemical chromatographic techniques from insect cells that had been co-infected with the corresponding baculovirus expression vectors (see e.g., Meijer and Kim, “Chemical Inhibitors of Cyclin-Dependent Kinases,” Methods in Enzymol., 283, 113-128 (1997)). The enzyme complex (5 nM) was assayed with 0.3-0.5 μg of purified recombinant retinoblastoma protein fragment (Rb) as a substrate. The engineered Rb fragment (residues 386-928 of the native retinoblastoma protein; 62.3 kDa) contains the majority of the phosphorylation sites found in the native 106-kDa protein, as well as a tag of six histidine residues for ease of purification. Phosphorylated Rb substrate was captured by microfiltration on a nitrocellulose membrane and quantified using a phosphorimager as described above. For measurement of tight-binding inhibitors, the assay duration was extended to 60 minutes, during which the time-dependence of product formation was linear and initial rate conditions were met. Kᵢ values were measured as described above and shown in Table 2. Percent inhibition at 1 mM, 0.1 μM and 0.03 μM of test compounds were calculated as described above and shown in Table 3. Table 4 shows percent inhibition calculated at 0.01 μM and 0.03 μM of test compounds.

### Inhibition of CDK2/Cyclin A Retinoblastoma Kinase Activity

CDK2 was purified using published methodology (Rosenblatt et al., J. Mol. Biol., 230, 1317-1319 (1993)) from insect cells that had been infected with a baculovirus expression vector. Cyclin A was purified from E. coli cells expressing full-length recombinant cyclin A, and a truncated cyclin A construct was generated by limited proteolysis and purified as described previously (Jeffrey et al., Nature, 376, 313-320 (1995)). A complex of CDK2 and proteolyzed cyclin A was prepared and purified by gel filtration. The substrate for this assay was the same R_{b} substrate fragment used for the CDK4 assays, and the methodology of the CDK2/delta cyclin A and the CDK4/delta cyclin D3 assays was essentially the same, except that CDK2 was present at 10 nM or 19 nM. The duration of the assay was 60 or 75 minutes, during which the time-dependence of product formation was linear and initial rate conditions were met. Kᵢ values were measured as described above and shown in Table 2. Percent inhibition at 0.01 μM and 0.03 μM of test compounds were calculated as described above and shown in Table 4.

### Inhibition of CDK1(cdc2)/Cyclin B Histone H1 Kinase Activity

The complex of human CDK1 (cdc2) and cyclin B was purchased from New England Biolabs (Beverly Mass.). Alternatively, a CDK1/glutathione-S-transferase-cyclin B1 complex was purified using glutathione affinity chromatography from insect cells that had been co-infected with the corresponding baculovirus expression vectors. The assay was executed as described above at 30° C. using 2.5 units of cdc2/cyclin B, 10 μg Histone H1 protein, and 0.1-0.3 μCi [^{32/33}P]ATP per assay. Phosphorylated histone substrate was captured by microfiltration on a phosphocellulose P81 membrane and quantified using a phosphorimager as described above. Kᵢ values were measured using the described curve-fitting programs and are shown in Table 2.

### Inhibition of Cell Growth: Assessment of Cytotoxicity

Inhibition of cell growth was measured using the tetrazolium salt assay, which is based on the ability of viable cells to reduce 3-(4,5-dimethylthiazol-2-yl)-2,5-[2H]-diphenyltetrazolium bromide (MTT) to formazan (Mosmann, J. Immunol. Meth., 65, 55-63 (1983)). The water-insoluble purple formazan product was then detected spectrophotometrically. HCT-116 cells were grown in 96-well plates. Cells were plated in the appropriate medium at a volume of 135 μl/well in either McCoy's 5A Medium. Plates were incubated for four hours before addition of inhibitor compounds. Different concentrations of inhibitor compounds were added in 0.5% (v/v) dimethylsulfoxide (15 μL/well), and cells were incubated at 37° C. (5% CO₂) for four to six days (depending on cell type). At the end of the incubation, MTT was added to a final concentration of 0.2 mg/mL, and cells were incubated for 4 hours more at 37° C. After centrifugation of the plates and removal of medium, the absorbance of the formazan (solubilized in dimethylsulfoxide) was measured at 540 nm. The concentration of inhibitor compound causing 50%(IC₅₀) or 90%(IC₅₀) inhibition of growth was determined from the linear portion of a semi-log plot of inhibitor concentration versus percentage inhibition. All results were compared to control cells treated only with 0.5% (v/v) dimethylsulfoxide. The IC₅₀ and IC₉₀ are shown in Table 2.

TABLE 2

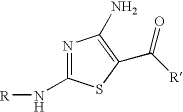

| | | | Ki | Ki | Ki | HCT116 |
|---|---|---|---|---|---|---|
| | | | CDK4/D3 | CDK2/A | CDK1/B | IC50,90 |
| Example | R | R' | (nM) | (nM) | (nM) | (μM) |
| C(48)* | 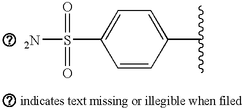 | 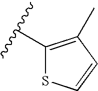 | 19.1 | 11 | 15 | 0.34 |
| C(50*) | 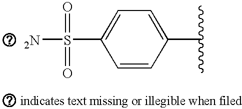 | 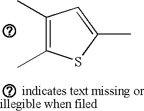 | 5.5 | 5.6 | 29 | 0.61 |
| C(85)* | 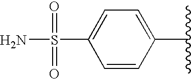 | 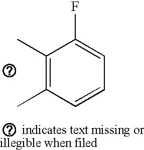 | 13 | 5.7 | 2.2 | 0.25, 0.56 |
| C(108)* | 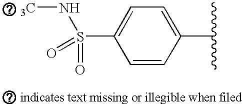 | 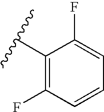 | 23 | 6.8 | 9 | 0.4, 1.4 |
| C(109)* | 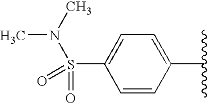 | 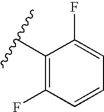 | 83 | 28 | 35 | 1.6, 3.0 |
| C(115)* | 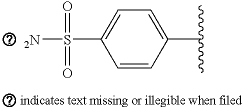 | 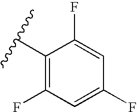 | 57 | 18 | 50 | 0.98, 2.1 |
| (116)* | 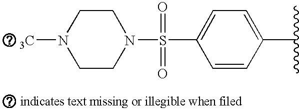 | 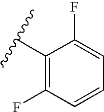 | 121 | 120 | 77 | ND |
| J(4)* | 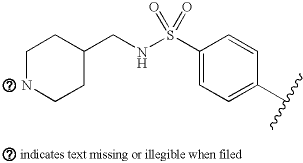 | 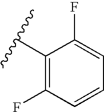 | 10.2 | 13 | 22 | >25 |
| A(1) | 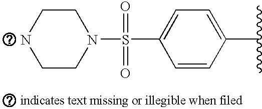 | 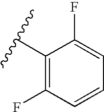 | 26 | 14 | ND | 1.0, 2.7 |
| A(2) | 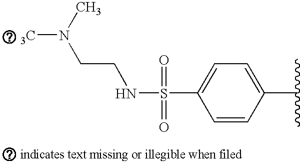 | 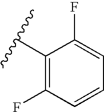 | 7.5 | 4.5 | ND | 0.36, 1.1 |
| A(3) | 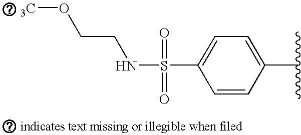 | 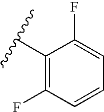 | 53 | 3.5 | ND | 0.34, 0.72 |
| A(4) | 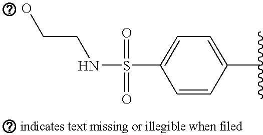 | 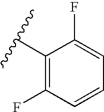 | 28 | 4.5 | ND | 1.0, 2.8 |
| A(5) | 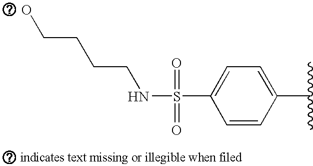 | 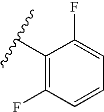 | 24 | 5.7 | ND | 1.3, 3.0 |
| A(6) | 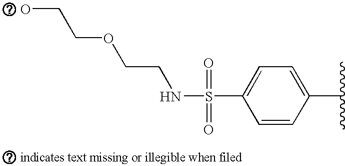 | 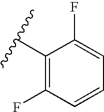 | 26 | 5.3 | ND | 1.9, 4.2 |
| A(7) | 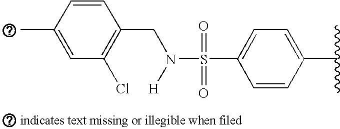 | 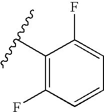 | 62 | 11 | ND | ND |
| A(8) | 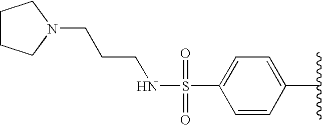 | 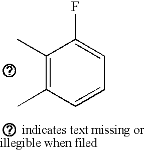 | 9.9 | 11 | ND | 2.4, 5.0 |
| A(9) | 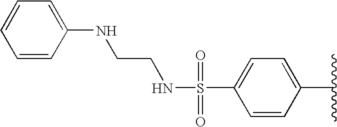 | 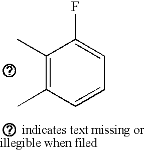 | <5 | <5 | ND | 0.14, 0.40 |
| A(10) | 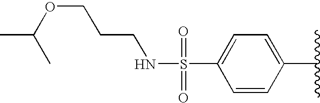 | 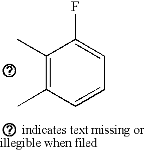 | 12 | 2.2 | ND | 0.9, 2.2 |
| A(11) | 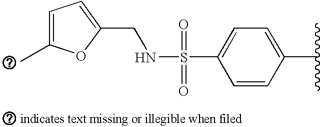 | 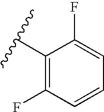 | 28 | <5 | 3.9 | 0.043, 0.14 |
| A(12) | 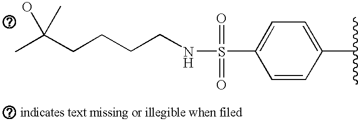 | 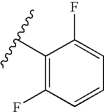 | 4.2 | <5 | ND | 0.15, 0.63 |
| A(13) | 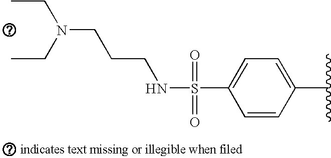 | 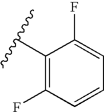 | 8.9 | 10 | ND | 0.73, 3 |
| A(14) | 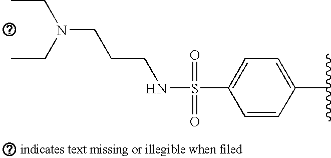 | 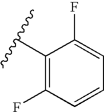 | 8.1 | 6 | ND | 0.42, 1.7 |
| A(15) | 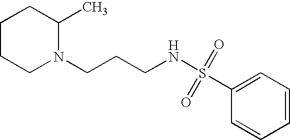 | 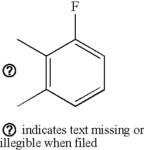 | 8.4 | 8.2 | ND | 0.46, 1.8 |
| A(16) | 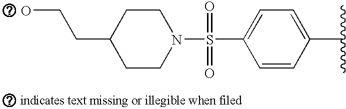 | 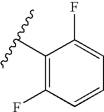 | 57 | 18 | ND | 1.5, 3.9 |
| A(17) | 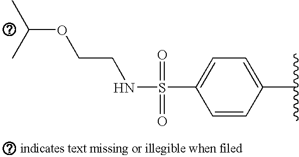 | 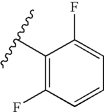 | 14 | 2 | ND | 0.14, 0.45 |
| A(18) | 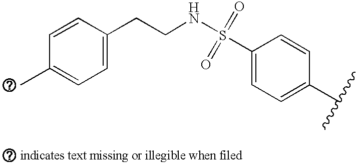 | 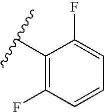 | 24 | 5.8 | ND | 1.8, 3.7 |
| A(19) | 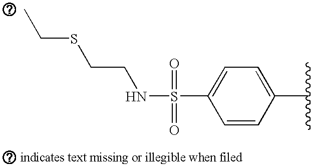 | 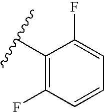 | 24 | 1.8 | ND | 0.4, 1.3 |
| A(20) | 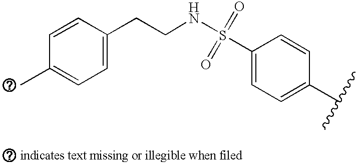 | 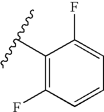 | 14 | <5 | ND | 0.9, 2.6 |
| A(21) | 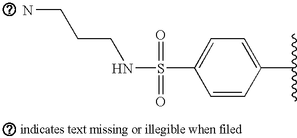 | 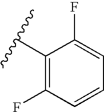 | 6.8 | 14 | ND | 1.8, 4.4 |
| A(22) | 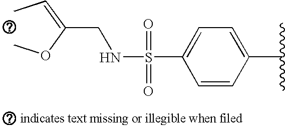 | 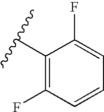 | 22 | ND | ND | 0.24, 0.62 |
| A(23) | 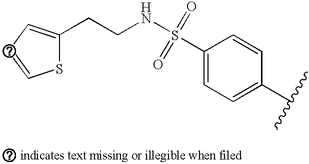 | 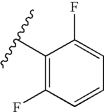 | 17 | 13 | ND | 0.44, 1.2 |
| A(24) | 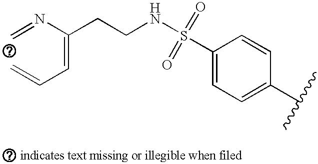 | 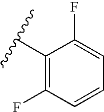 | 18 | 5 | ND | 1.2, 2.6 |
| A(25) | 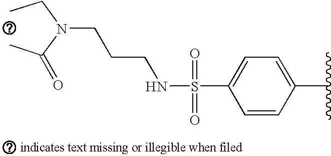 | 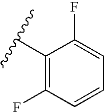 | 16 | 4.1 | ND | 3.4, 5.0 |
| A(26) | 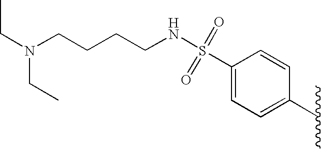 | 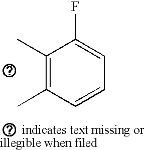 | 3 | 2.9 | ND | 0.85, 3.3 |
| A(27) | 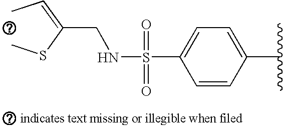 | 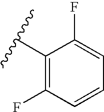 | 57 | 4.6 | ND | 0.40, 1.0 |
| A(28) | 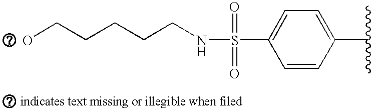 | 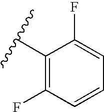 | 12 | 2.6 | ND | 1.2, 2.8 |
| A(29) | 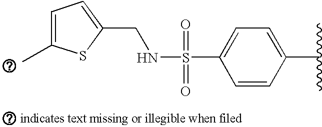 | 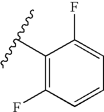 | 15 | <5 | ND | 0.19, 0.56 |
| A(30) | 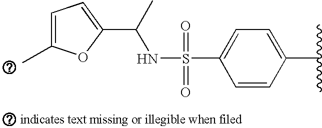 | 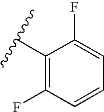 | 9 | <5 | ND | 0.31, 0.64 |
| A(32) | 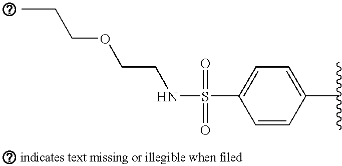 | 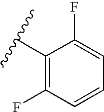 | 16 | <5 | ND | 0.14, 0.38 |
| A(33) | 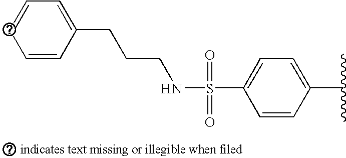 | 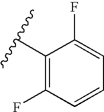 | 9.3 | 3.6 | ND | 0.55, 1.3 |
| A(34) | 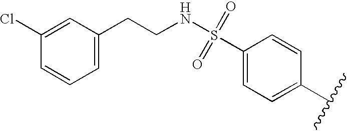 | 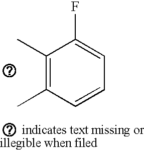 | 5.1 | <10 | ND | 0.78, 1.8 |
| A(36) | 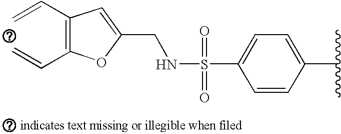 | 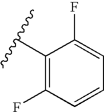 | 35 | 4.6 | ND | 0.51, 1.2 |
| A(37) | 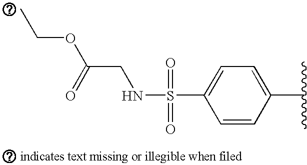 | 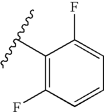 | 35 | 4.9 | ND | 1.2, 3.8 |
| A(38) | 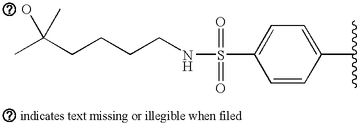 | 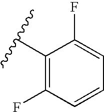 | 3.9 | 0.48 | ND | ND |
| A(39) | 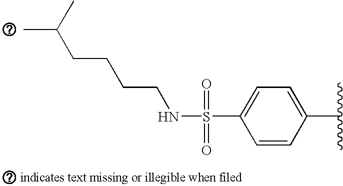 | 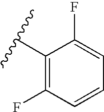 | 21 | 3.7 | ND | 0.9, 2.3 |
| A(40) | 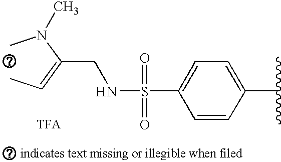 | 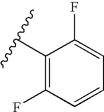 | 42 | 6.9 | ND | ND |
| B(1) | 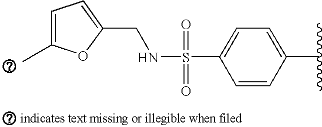 | 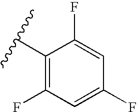 | 100 | <10 | ND | 0.90, 2.0 |
| B(2) | 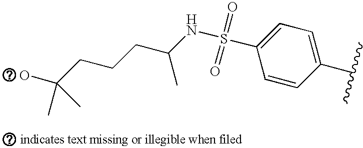 | 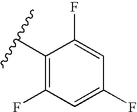 | 6.2 | 1.3 | ND | 0.98, 2.3 |
| C(1) | 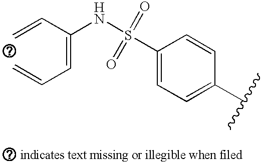 | 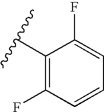 | 76 | 17 | ND | 0.87, 1.9 |
| D(1) | 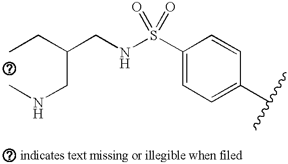 | 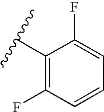 | 12 | 18 | ND | 17, >25 |
| D(2) | 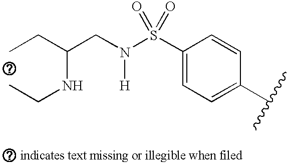 | 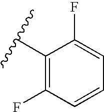 | 20 | 45 | ND | 4, 9.2 |
| D(3) | 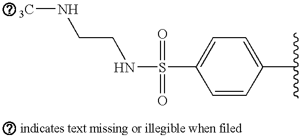 | 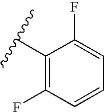 | 5.7 | 9.4 | ND | 2.9, >5.0 |
| E(1) | 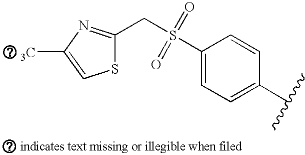 | 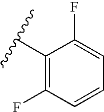 | 42 | 15 | ND | 7.0, 17 |
| Q(1) | 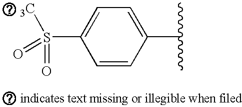 | 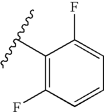 | 73 | 210 | ND | 1.3, 3.8 |
| R(2) | 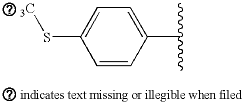 | 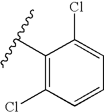 | 30 | 250 | ND | 12, >25 |
| R(3) | 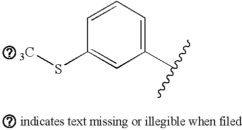 | 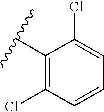 | 74 | ND | ND | ND |
| W(1) | 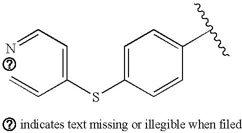 | 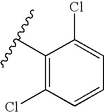 | 89 | ND | ND | ND |
| W(2) | 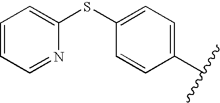 | 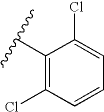 | 190 | 500 | ND | ND |
| X(1) | 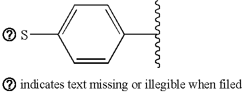 | 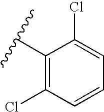 | ND | 520 | ND | ND |
| X(2) | 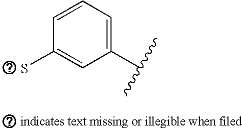 | 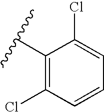 | ND | 1600 | ND | ND |
| Y(1) | 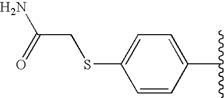 | 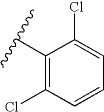 | 11 | 66 | ND | 2.3, 6 |
| Y(2) | 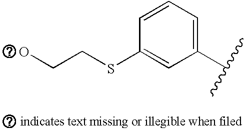 | 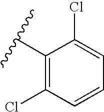 | 7.6 | 140 | ND | 3.9, 13 |
| Y(3) | 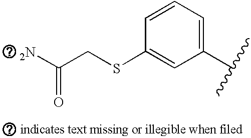 | 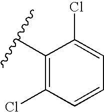 | 33 | 92 | ND | 11, >25 |
| Z(1) | 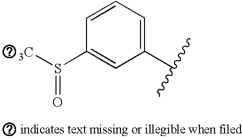 | 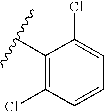 | ND | 450 | ND | ND |
| Z(2) | 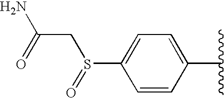 | 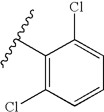 | 15 | 62 | ND | 18, >25 |
| Z(3) | 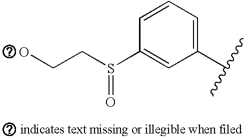 | 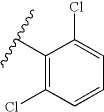 | 30 | 170 | ND | 6.8, 16 |
| Z(4) | 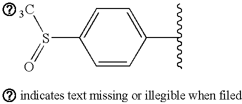 | 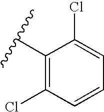 | 38 | 303 | 120 | 4, 12 |
| Z(5) | 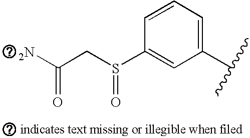 | 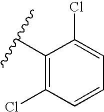 | 230 | 330 | ND | >25, >25 |
| AA(1) | 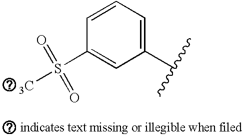 | 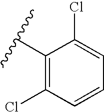 | ND | 430 | ND | ND |
| AA(2) | 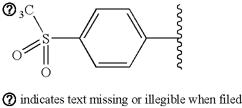 | 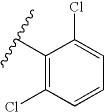 | 42 | 138 | 93 | 4.5, 18 |
| AA(3) | 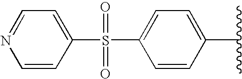 | 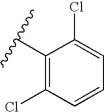 | ND | 190 | ND | ND |
| BB(1) | 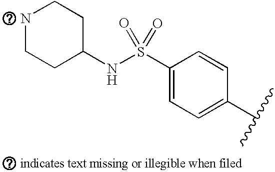 | 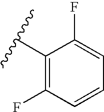 | 13 | 13 | ND | 22, >50 |
| CC(1) | 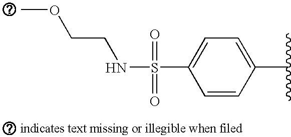 | 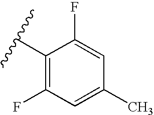 | 9.7 | 0.74 | ND | 0.28, 0.48 |

ND = not determined
*= previously disclosed in WO99/21845

| | | % Inhib | % Inhib | % Inhib |
|---|---|---|---|---|
| | | CDK4/D | CDK4/D | CDK4/D |
| | | @ 1 | @ 0.1 | @ 0.03 |
| Example | Structure mM | μM | μM | |
| DD(1) | 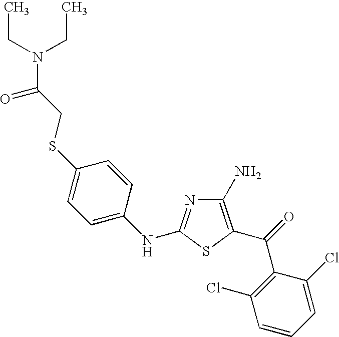 | 46.9 | −19 | −34 |
| DD(2) | 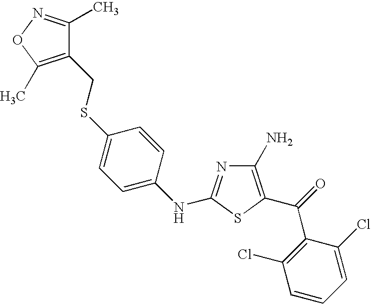 | 74 | 19 | 37 |
| DD(3) | 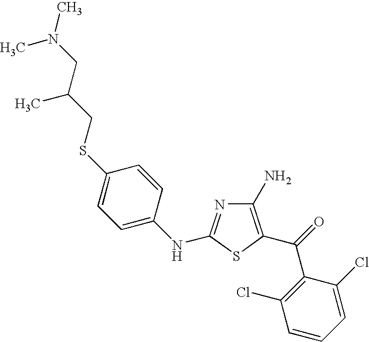 | 43.6 | 0.2 | 8.1 |
| DD(4) | 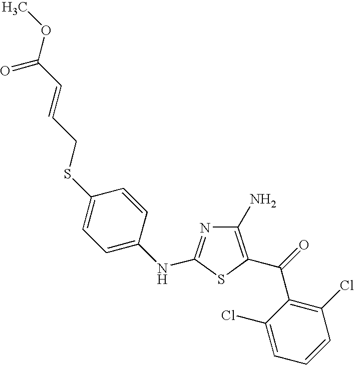 | 49.1 | 11 | 5.4 |
| DD(5) | 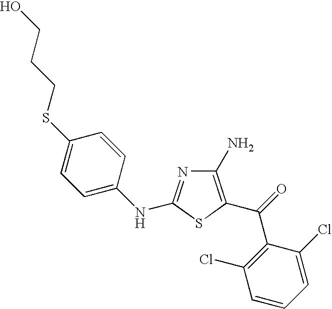 | 85.2 | 40 | 21 |
| DD(6) | 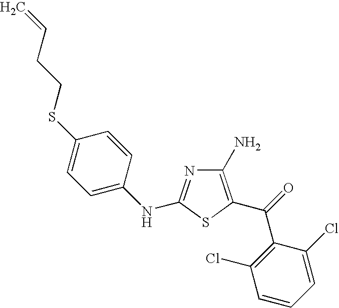 | 48 | 20 | 22 |
| DD(7) | 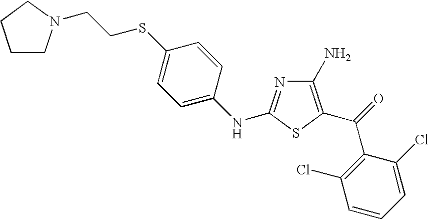 | 55 | 31 | 11 |
| DD(8) | 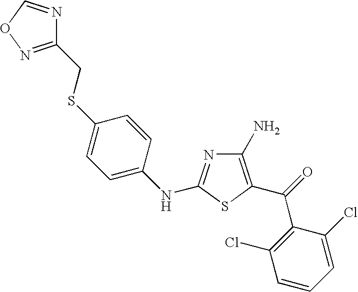 | 82.1 | 43 | 16 |
| DD(9) | 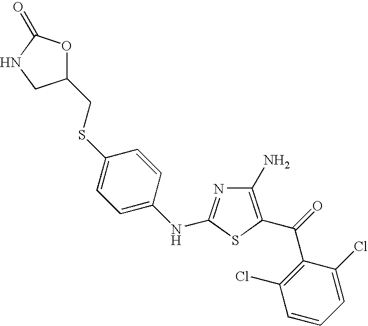 | 40.3 | −14 | −28 |
| DD(10) | 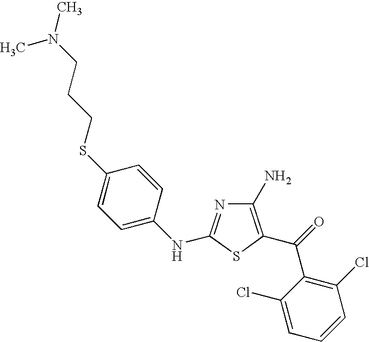 | 54.5 | 6.6 | 2.6 |
| DD(11) | 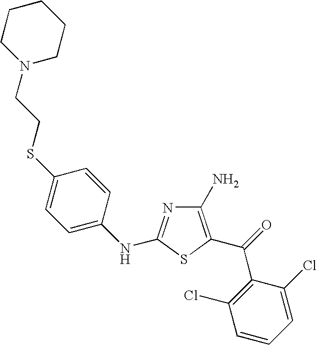 | 78.1 | 28 | 4.2 |
| DD(12) | 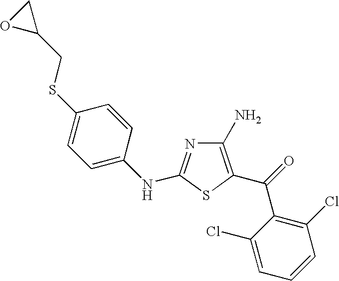 | 72 | 19 | 3.9 |
| DD(13) | 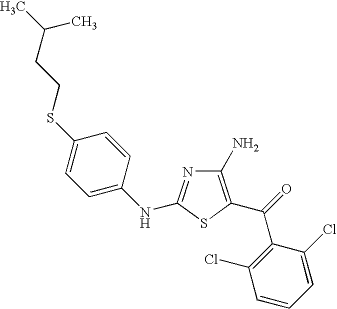 | 35.3 | 4.7 | 0.6 |
| DD(14) | 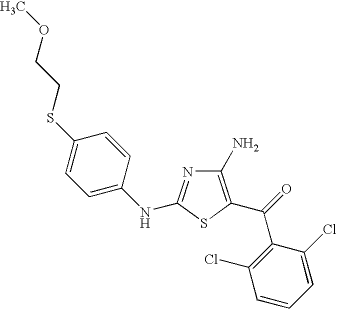 | 80 | 41 | 18 |
| DD(15) | 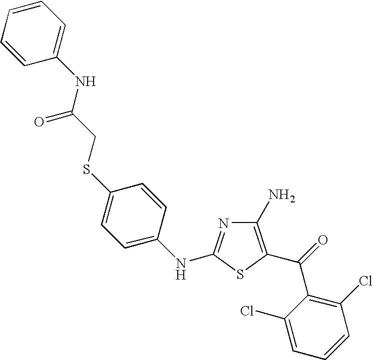 | 82 | 53 | 38 |
| DD(16) | 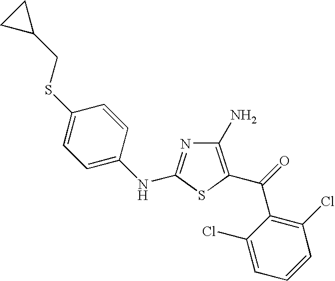 | 56.2 | −7 | −9.4 |
| DD(17) | 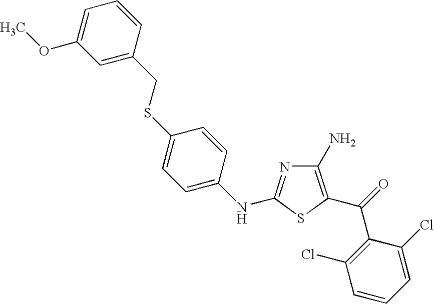 | 28.3 | −3 | −9.5 |
| DD(18) | 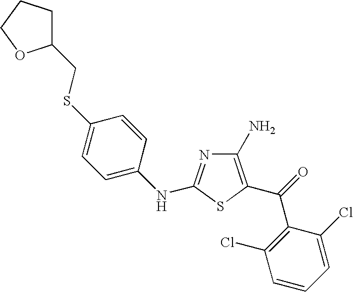 | 70.6 | 9.9 | 10 |
| DD(19) | 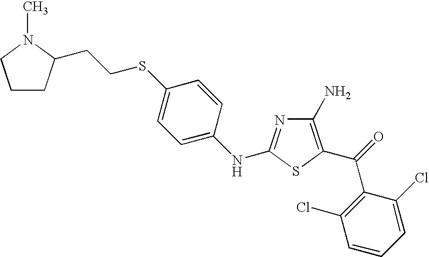 | 82.9 | 19 | −3 |
| DD(20) | 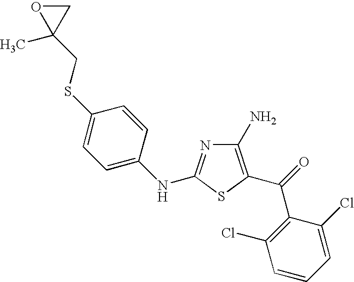 | 68.2 | 6.8 | 4.2 |
| DD(21) | 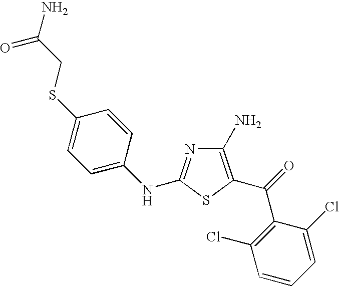 | 85.5 | 67 | 50 |
| DD(22) | 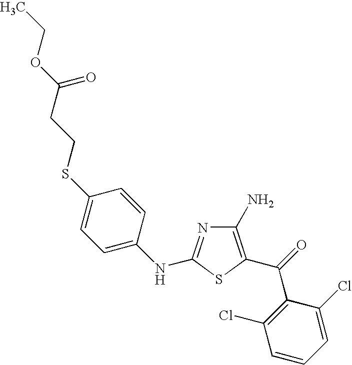 | 56.2 | 9.1 | 8.1 |
| DD(23) | 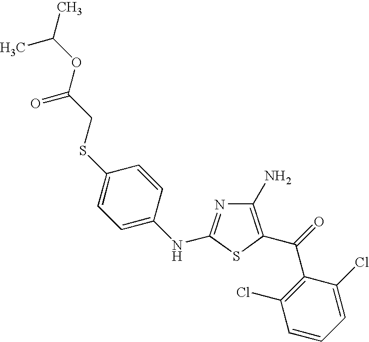 | 72.1 | 37 | 19 |
| DD(24) | 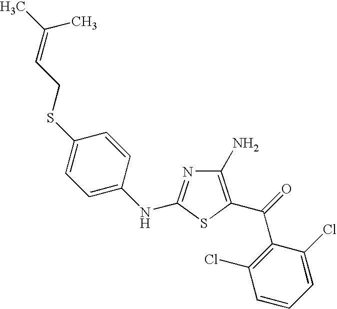 | 42.5 | −3 | −17 |
| DD(25) | 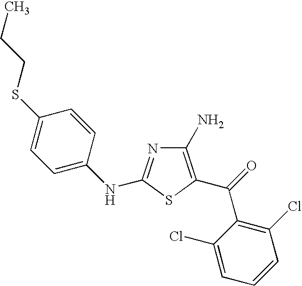 | 65.5 | 0.4 | −10 |
| DD(26) | 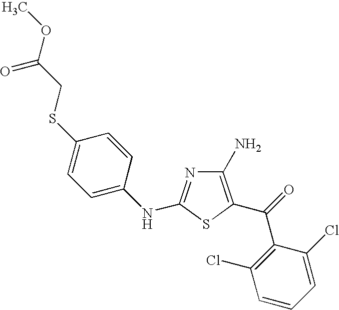 | 87.5 | 65 | 9 |
| DD(27) | 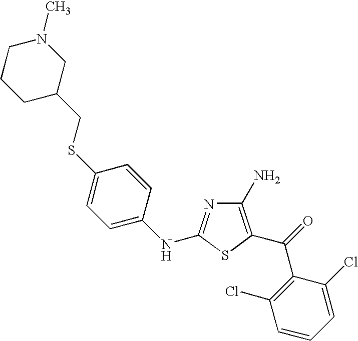 | 58.7 | 10 | −2.6 |
| DD(28) | 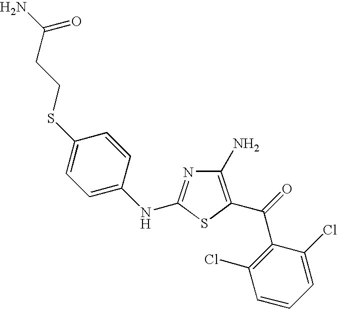 | 86.8 | 51 | 19 |
| DD(29) | 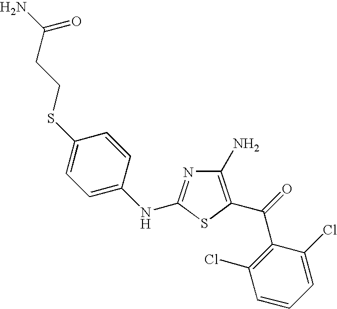 | 87.2 | 50 | 24 |
| DD(30) | 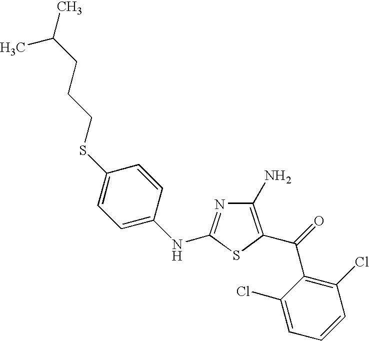 | 32.3 | 13 | 3.1 |
| DD(31) | 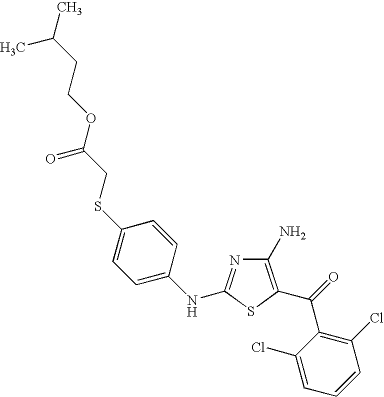 | 59.7 | 30 | 15 |
| DD(32) | 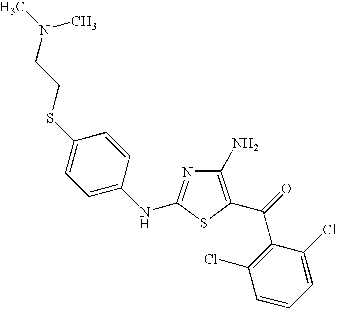 | 61.3 | 14 | 5.5 |
| DD(33) | 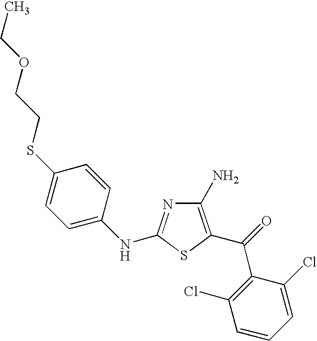 | 64.7 | 21 | 8.1 |
| DD(34) | 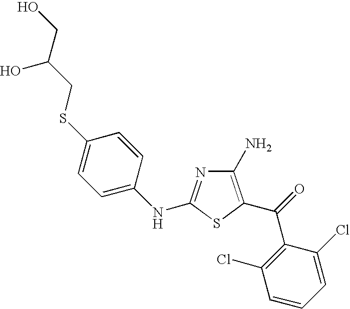 | 89.3 | 52 | 32 |
| DD(35) | 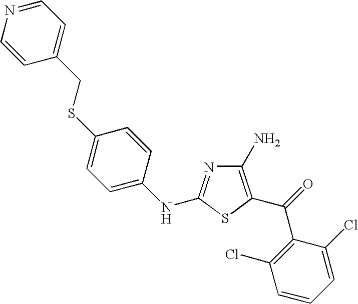 | 76.8 | 42 | 12 |
| DD(36) | 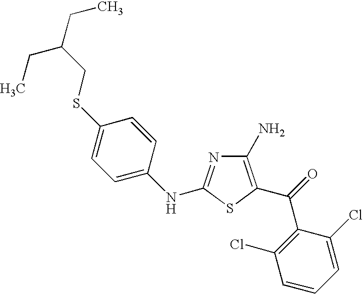 | 49.6 | 18 | 9 |
| DD(37) | 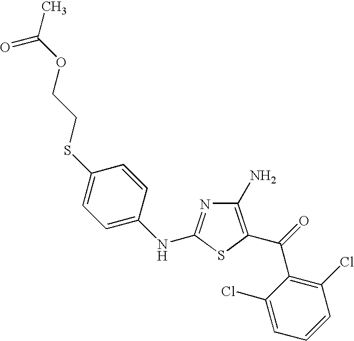 | 74.3 | 30 | 13 |
| DD(38) | 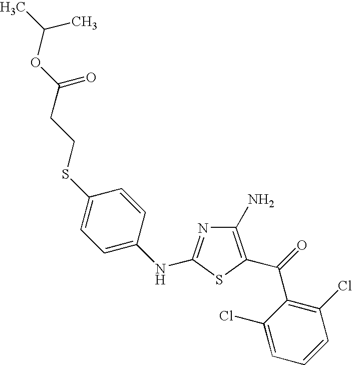 | 57.8 | 32 | 6.5 |
| DD(39) | 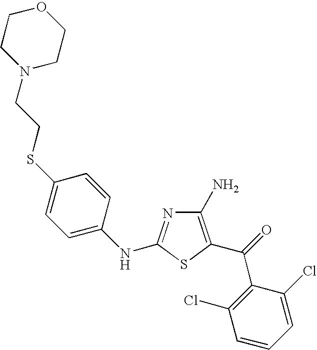 | 68.4 | 18 | 0.2 |
| DD(40) | 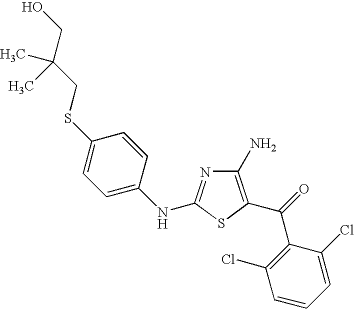 | 50.3 | 23 | −1.2 |
| DD(41) | 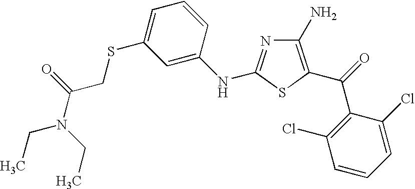 | 70.3 | 33 | 28 |
| DD(42) | 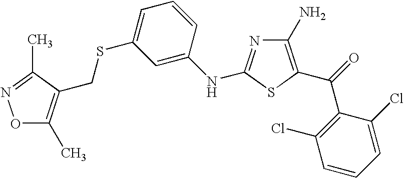 | 63.7 | 41 | 29 |
| DD(43) | 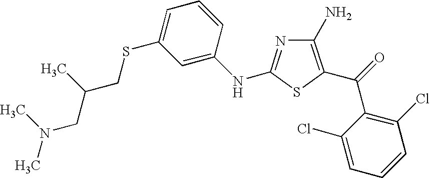 | 71.3 | 16 | 25 |
| DD(44) | 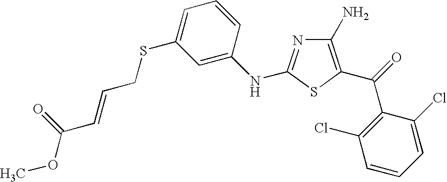 | 46 | 31 | 23 |
| DD(45) | 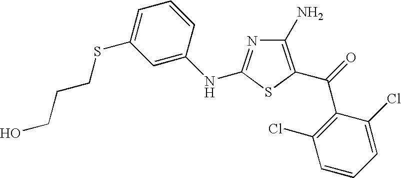 | 79.6 | 54 | 32 |
| DD(46) | 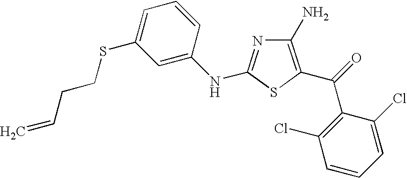 | 50.7 | 39 | 18 |
| DD(47) | 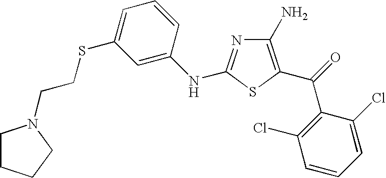 | 62.9 | 7.3 | 20 |
| DD(48) | 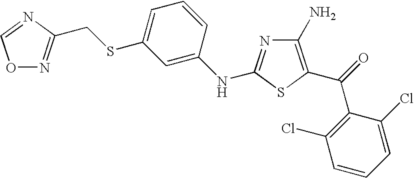 | 52.2 | 36 | 26 |
| DD(49) | 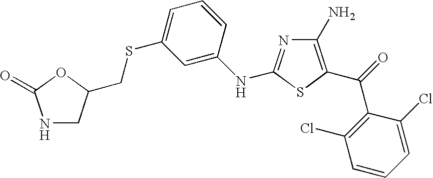 | 54.2 | 19 | 12 |
| DD(50) | 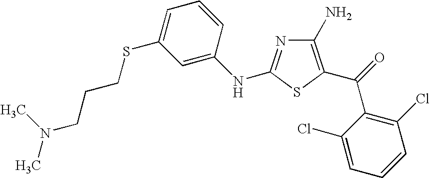 | 80.3 | 10 | 24 |
| DD(51) | 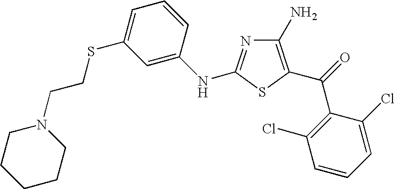 | 75.6 | 30 | 4.1 |
| DD(52) | 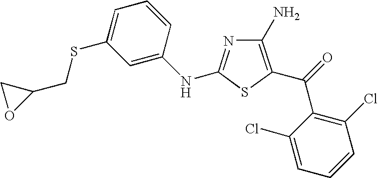 | 74.9 | 34 | 15 |
| DD(53) | 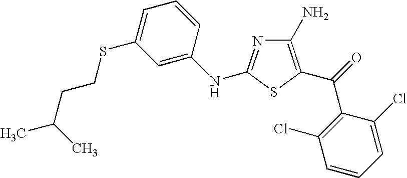 | 31.5 | 14 | 5.6 |
| DD(54) | 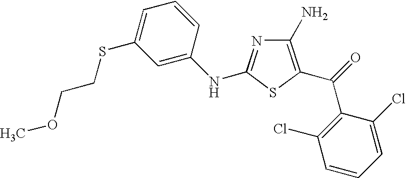 | 75.2 | 34 | 20 |
| DD(55) | 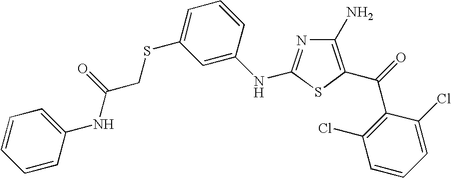 | 63.4 | 24 | 17 |
| DD(56) | 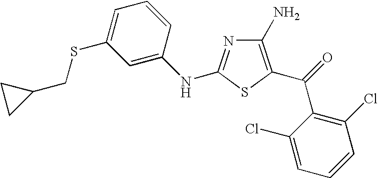 | 55.4 | 17 | 2.9 |
| DD(57) | 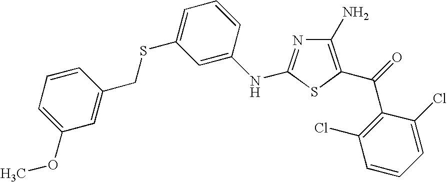 | 24.6 | −9 | −14 |
| DD(58) | 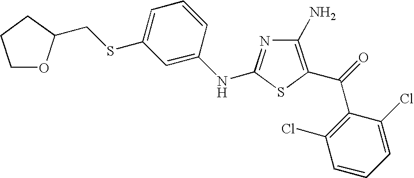 | 72.7 | 23 | 11 |
| DD(59) | 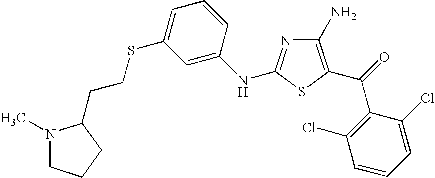 | 57.4 | 37 | 20 |
| DD(60) | 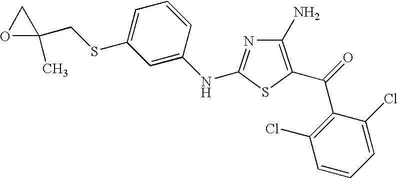 | 80.1 | 39 | 22 |
| DD(61) | 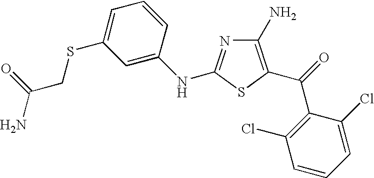 | 81.5 | 57 | 20 |
| DD(62) | 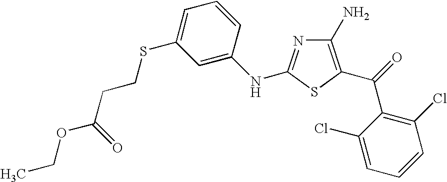 | 46.4 | 27 | 6.4 |
| DD(63) | 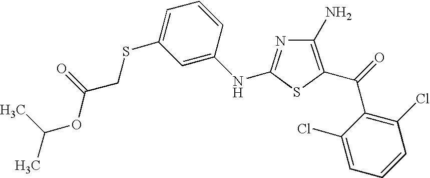 | 46.6 | 29 | −3.8 |
| DD(64) | 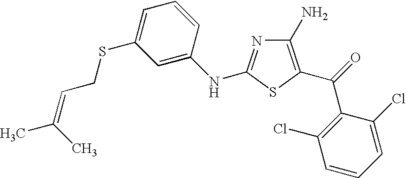 | 22.1 | 27 | −11 |
| DD(65) | 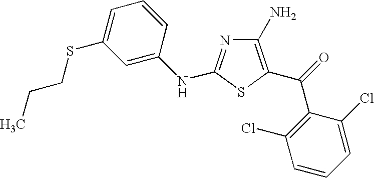 | 51.1 | 7 | 6.5 |
| DD(66) | 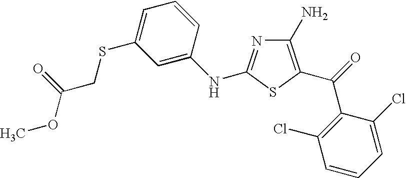 | 77.1 | 23 | 18 |
| DD(67) | 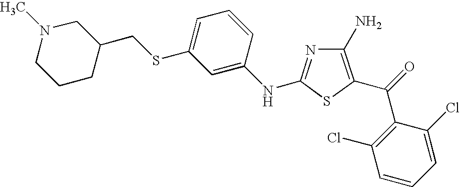 | 49.1 | 14 | 4.3 |
| DD(68) | 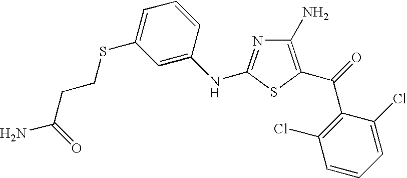 | 77.4 | 52 | 25 |
| DD(69) | 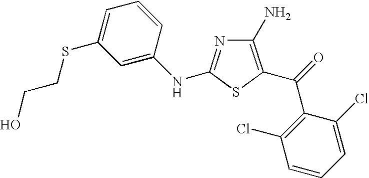 | 78.4 | 41 | 16 |
| DD(70) | 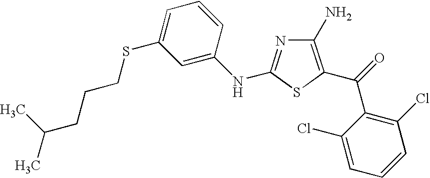 | 5.3 | 16 | 93 |
| DD(71) | 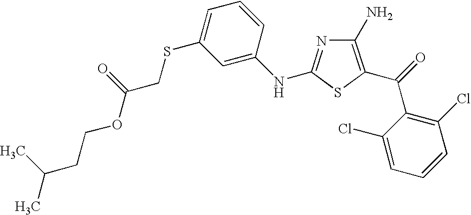 | 36 | 6.9 | 77 |
| DD(72) | 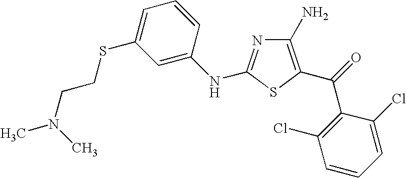 | 69 | 25 | −1.1 |
| DD(73) | 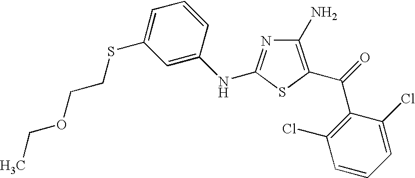 | 73.7 | 31 | 11 |
| DD(74) | 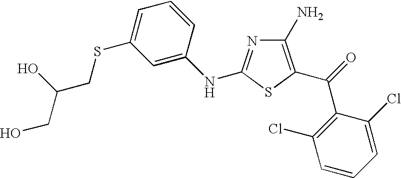 | 85.7 | 59 | 28 |
| DD(75) | 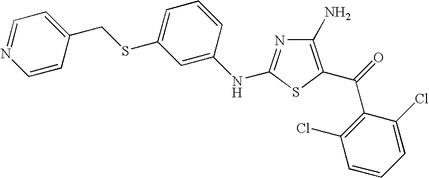 | 66.5 | 29 | 18 |
| DD(76) | 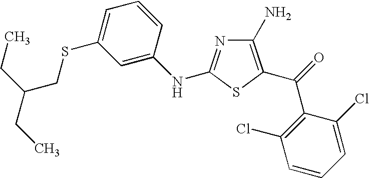 | 29.5 | 9.6 | 19 |
| DD(77) | 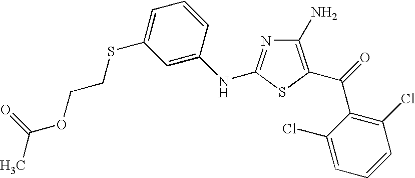 | 71.9 | 30 23 | |
| DD(78) | 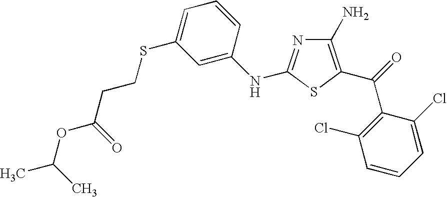 | 36.2 | 22 | 13 |
| DD(79) | 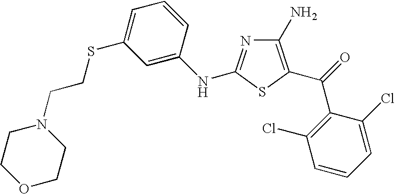 | 62.3 | 4.3 | 9.1 |
| DD(80) | 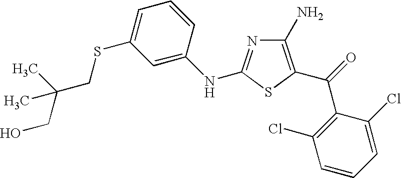 | 56 | 1.5 | 1.2 |
| DD(81) | 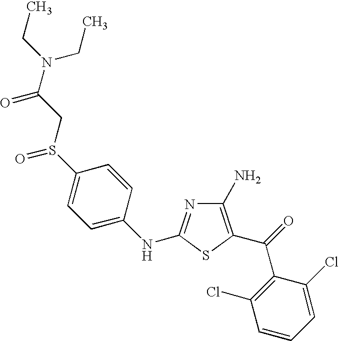 | 57.4 | 21 | −21 |
| DD(82) | 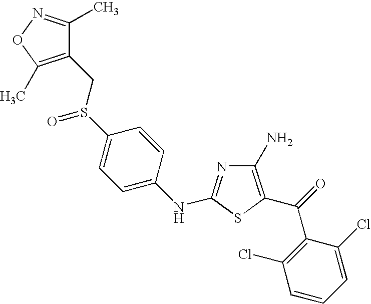 | 72.3 | −4 | −3.3 |
| DD(83) | 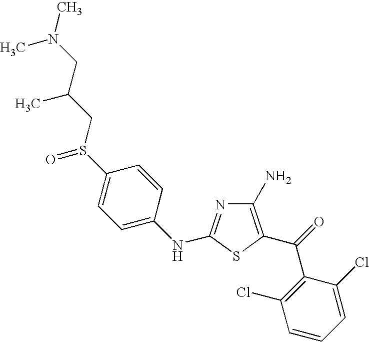 | 43 | 31 | −17 |
| DD(84) | 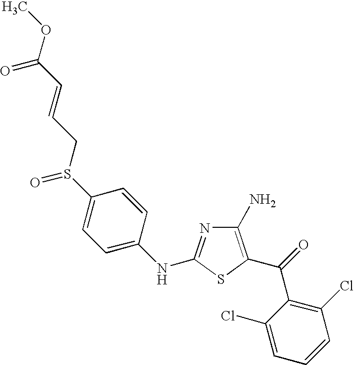 | 45.7 | 26 | −5.6 |
| DD(85) | 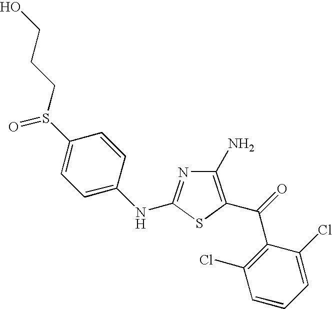 | 76.8 | 49 | 3.7 |
| DD(86) | 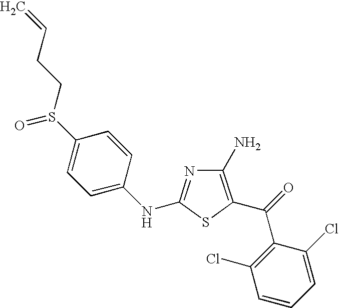 | 62.5 | 36 | −9.8 |
| DD(87) | 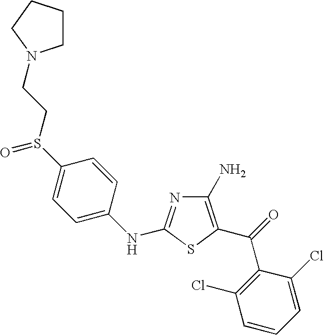 | 68.2 | 29 | −16 |
| DD(88) | 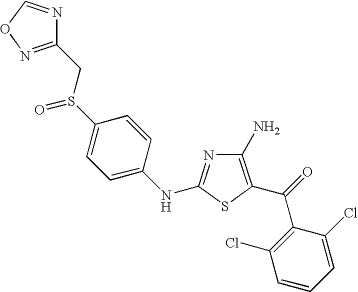 | 81.5 | 56 | 4.1 |
| DD(89) | 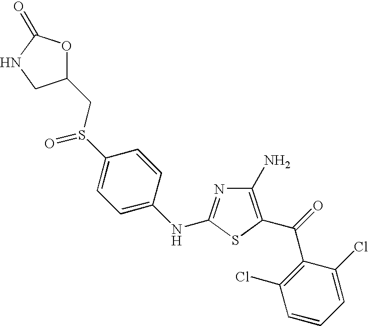 | 61.7 | 35 | −29 |
| DD(90) | 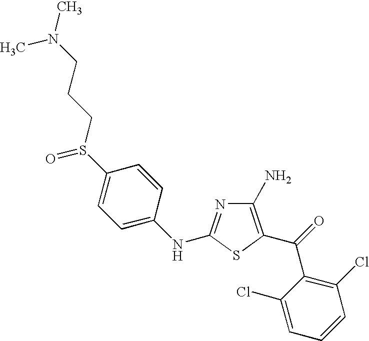 | 70.2 | 44 | −4.9 |
| DD(91) | 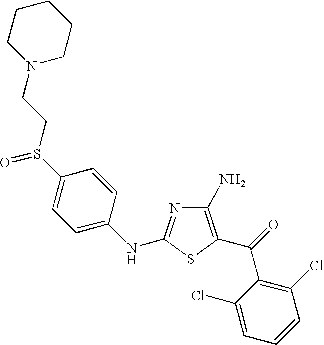 | 68.2 | 24 | −6.3 |
| DD(92) | 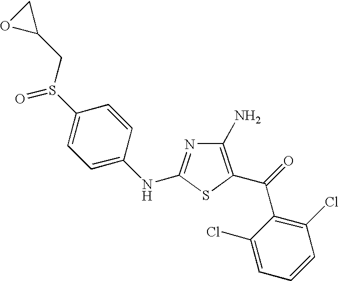 | 65.8 | 26 | −4.6 |
| DD(93) | 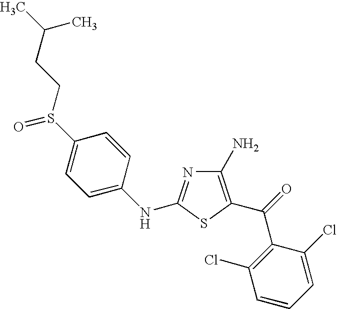 | 43 | 35 | −4.4 |
| DD(94) | 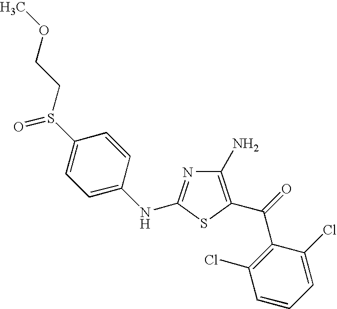 | 70.9 | 51 | 17 |
| DD(95) | 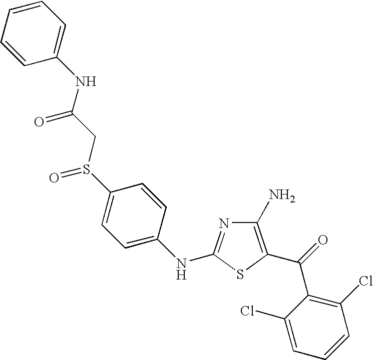 | 80.7 | 55 | 5.3 |
| DD(96) | 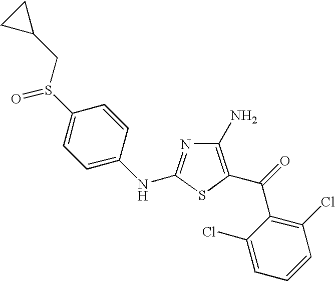 | 55.4 | 28 | −8.1 |
| DD(97) | 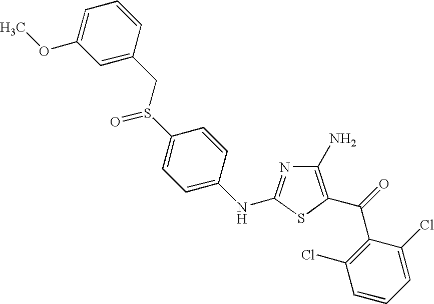 | 66.8 | 36 | −3.5 |
| DD(98) | 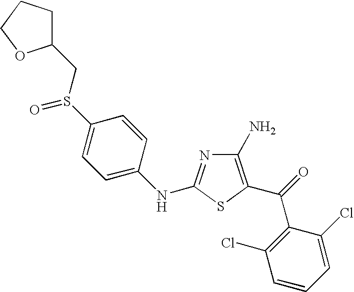 | 45.4 | 21 | −12 |
| DD(99) | 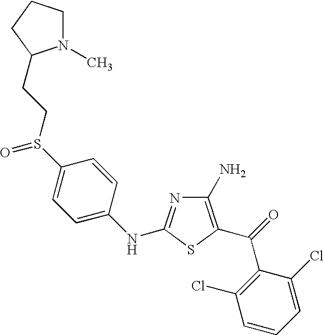 | 77.1 | 34 | −5.6 |
| DD(100) | 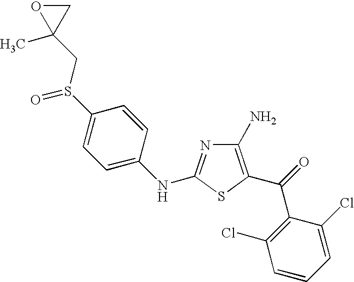 | 63.7 | 33 | 7.1 |
| DD(101) | 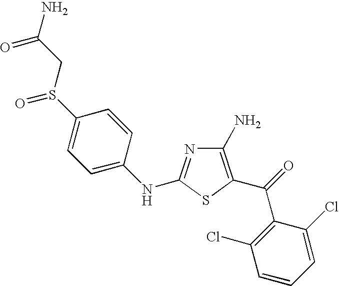 | 80.4 | 78 | 38 |
| DD(102) | 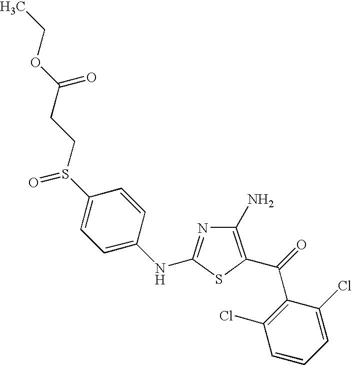 | 46.2 | 42 | 2.1 |
| DD(103) | 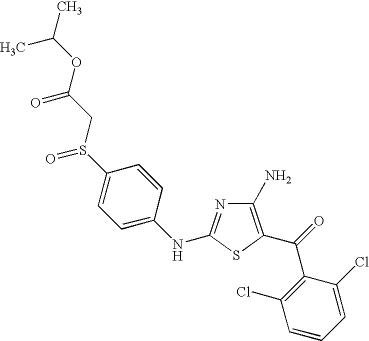 | 47.5 | 38 | −2.9 |
| DD(104) | 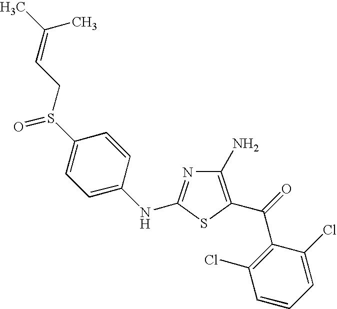 | 36.4 | 14 | −38 |
| DD(105) | 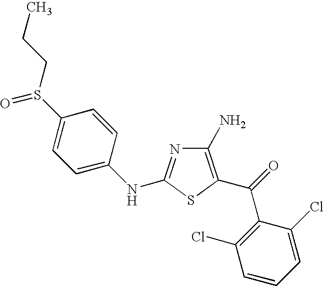 | 61.9 | 39 | 0.9 |
| DD(106) | 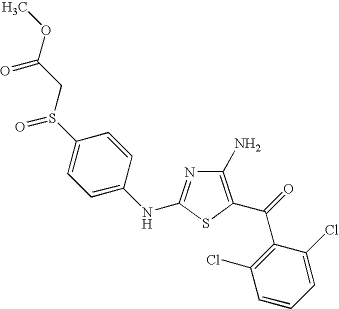 | 66.8 | 45 | 1.7 |
| DD(107) | 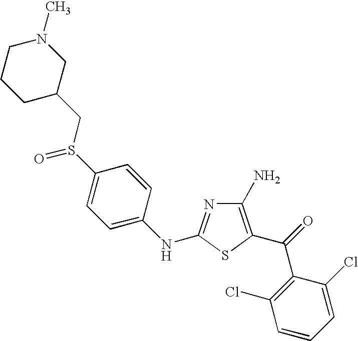 | 47.3 | 28 | −11 |
| DD(108) | 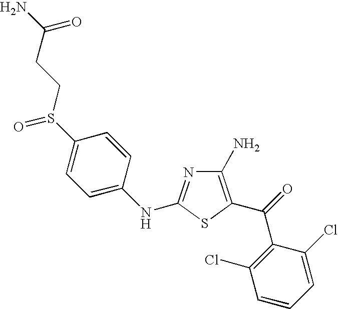 | 73.9 | 60 | 14 |
| DD(109) | 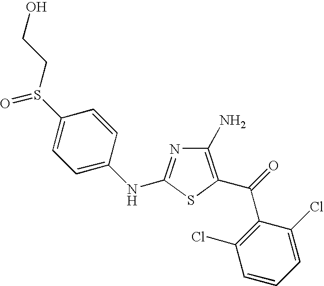 | 73.5 | 67 | 25 |
| DD(110) | 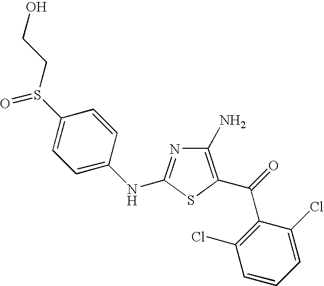 | 28.9 | 27 | 3.7 |
| DD(111) | 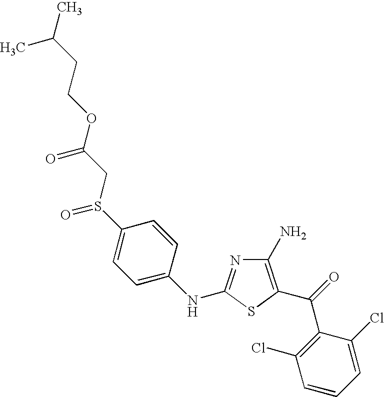 | 64.8 | 32 | −1.7 |
| DD(112) | 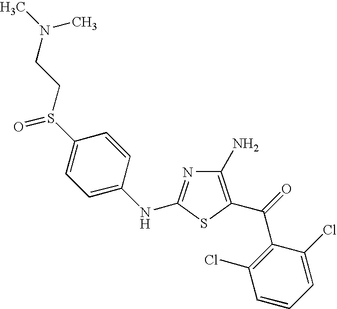 | 63.2 | 27 | −4.9 |
| DD(113) | 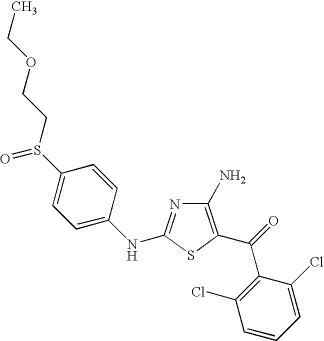 | 59.4 | 30 | 1.1 |
| DD(114) | 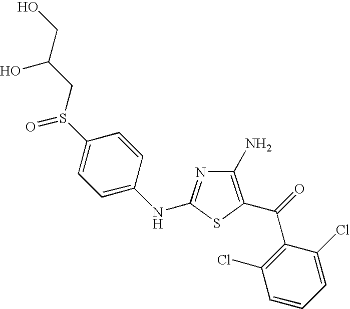 | 77.9 | 46 | 16 |
| DD(115) | 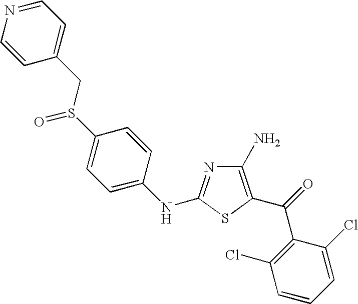 | 68.5 | 45 | 6.3 |
| DD(116) | 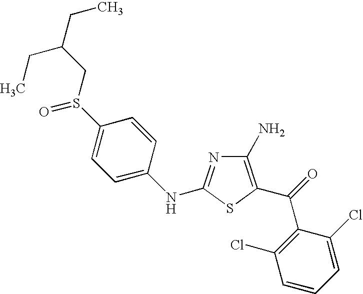 | 26.6 | 15 | −2.9 |
| DD(117) | 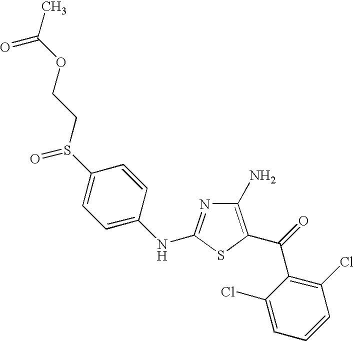 | 61.1 | 52 | 16 |
| DD(118) | 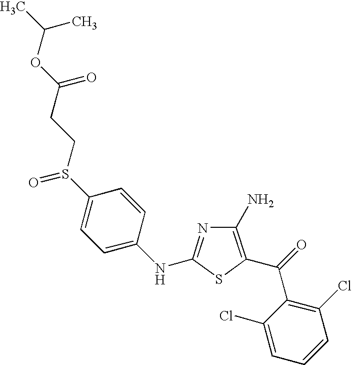 | 49.9 | 22 | −0.7 |
| DD(119) | 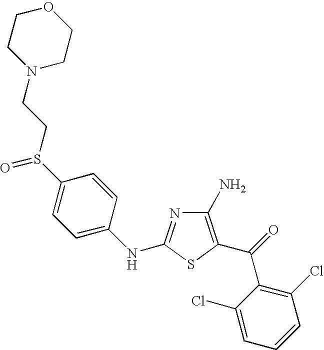 | 58 | 36 | 3.8 |
| DD(120) | 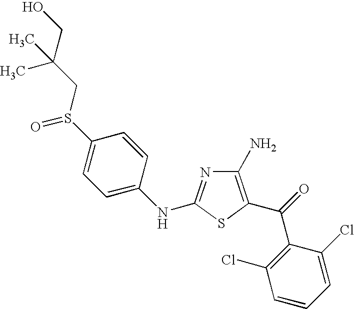 | 57.1 | 29 | −34 |
| DD(121) | 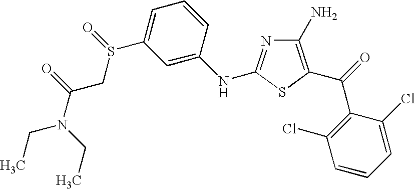 | 59.8 | 41 | −0.5 |
| DD(122) | 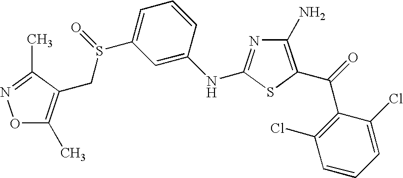 | 54.3 | 40 | 16 |
| DD(123) | 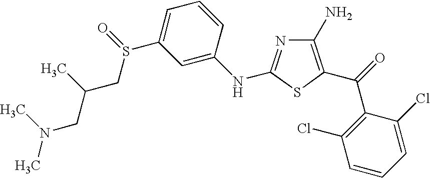 | 51.2 | 34 | 8 |
| DD(124) | 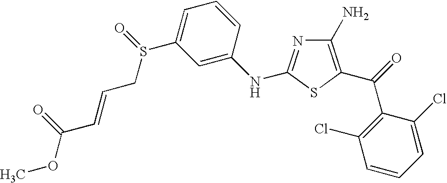 | 38.8 | 34 | 2.1 |
| DD(125) | 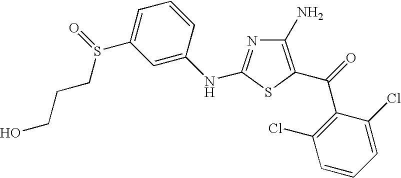 | 61.5 | 55 | 15 |
| DD(126) | 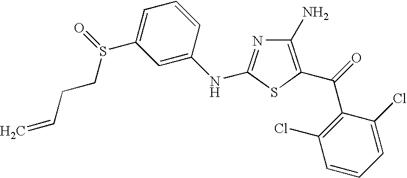 | 43.1 | 28 | 7.5 |
| DD(127) | 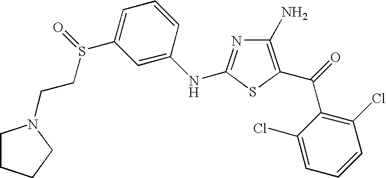 | 49.6 | 34 | −4.3 |
| DD(128) | 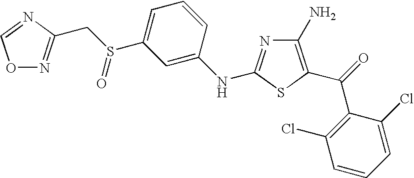 | 49.9 | 54 | 12 |
| DD(129) | 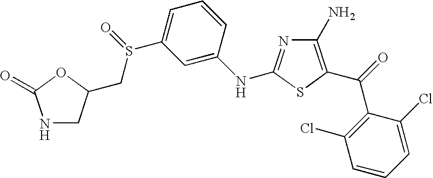 | 33.3 | 33 | 19 |
| DD(130) | 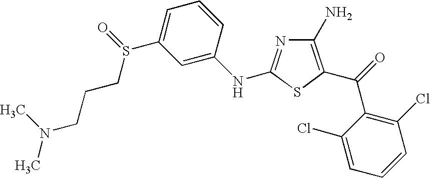 | 56 | 41 | 12 |
| DD(131) | 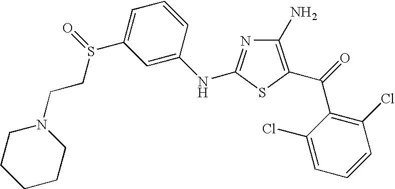 | 57 | 32 | 13 |
| DD(132) | 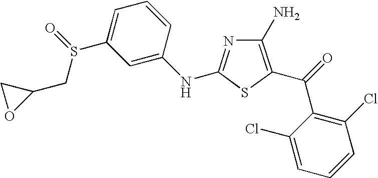 | 62 | 64 | 44 |
| DD(133) | 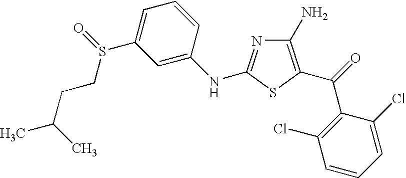 | 24.3 | 28 | 14 |
| DD(134) | 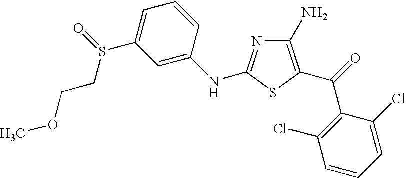 | 49.9 | 48 | 8.4 |
| DD(135) | 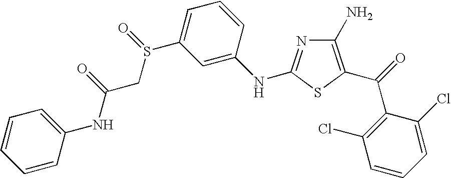 | 51.9 | 42 | 16 |
| DD(136) | 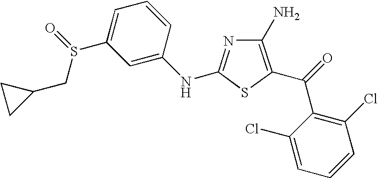 | 44.9 | 27 | 14 |
| DD(137) | 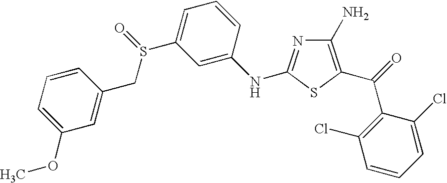 | 36.7 | 22 | −1.7 |
| DD(138) | 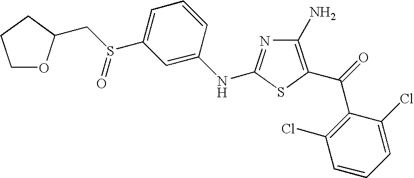 | 63.8 | 34 | 2.9 |
| DD(139) | 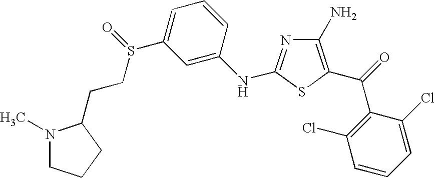 | 77.6 | 53 | 17 |
| DD(140) | 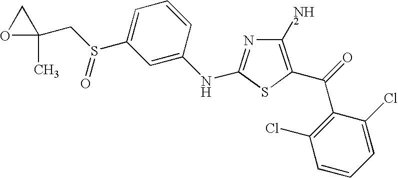 | 68 | 41 | 25 |
| DD(141) | 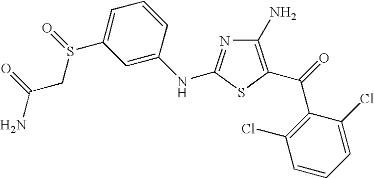 | 64.5 | 71 | 36 |
| DD(142) | 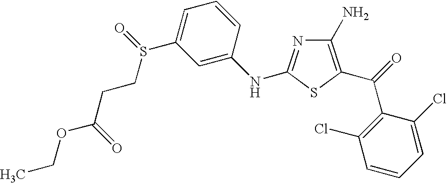 | 39.3 | 36 | −2.6 |
| DD(143) | 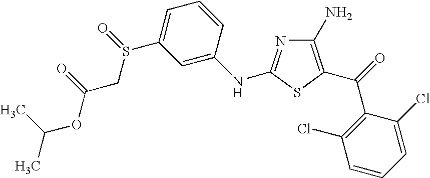 | 36.7 | 24 | 15 |
| DD(144) | 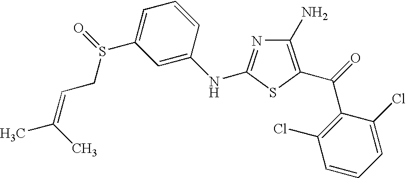 | 27.7 | 12 | −66 |
| DD(145) | 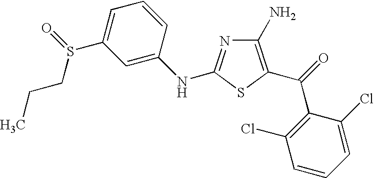 | 42.3 | 32 | 4.2 |
| DD(146) | 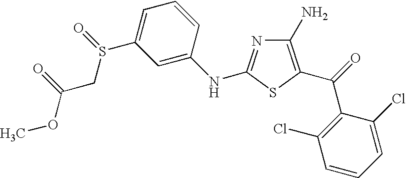 | 47.6 | 53 | 15 |
| DD(147) | 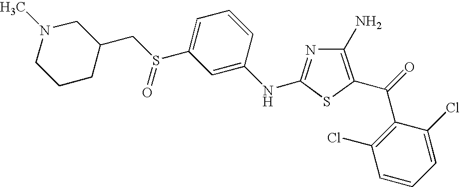 | 39.6 | 38 | 19 |
| DD(148) | 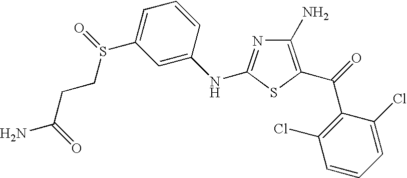 | 65.5 | 53 | 35 |
| DD(149) | 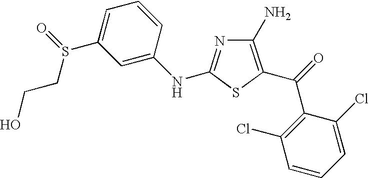 | 57.7 | 55 | 33 |
| DD(150) | 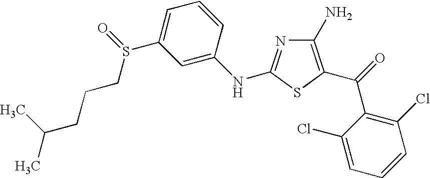 | 10.4 | 21 | 5.6 |
| DD(151) | 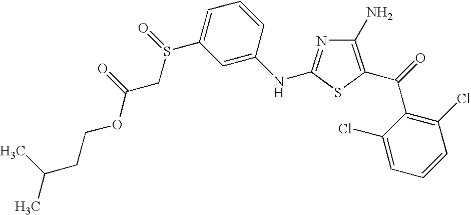 | 34.3 | 9.2 | 16 |
| DD(152) | 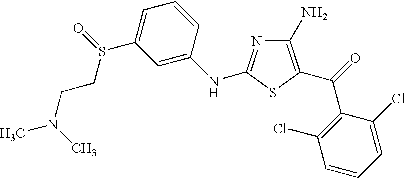 | 57.3 | 28 | 21 |
| DD(153) | 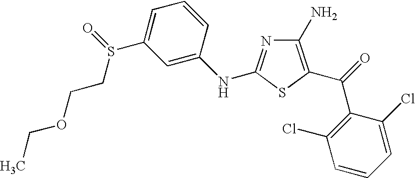 | 51.8 | 44 | 15 |
| DD(154) | 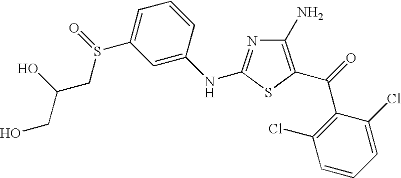 | 73.5 | 60 | 31 |
| DD(155) | 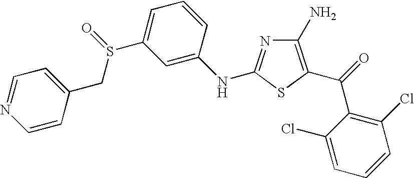 | 59.6 | 35 | 25 |
| DD(156) | 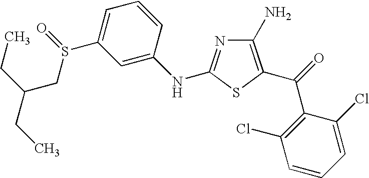 | 15.1 | 25 | 17 |
| DD(157) | 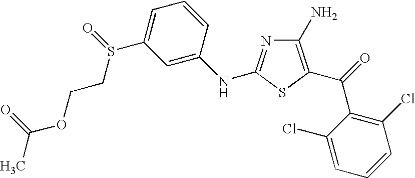 | 59.7 | 42 | 21 |
| DD(158) | 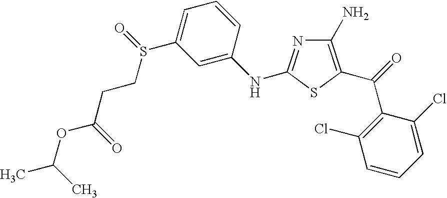 | 35 | 27 | 22 |
| DD(159) | 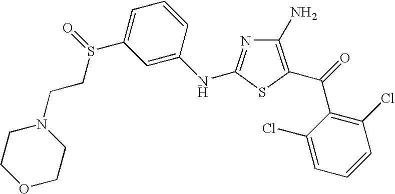 | 44 | 25 | 36 |
| DD(160) | 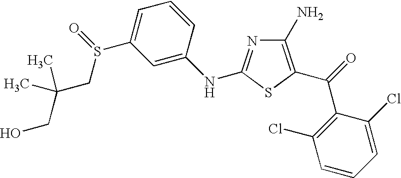 | 49 | 19 | 8.1 |
| DD(161) | 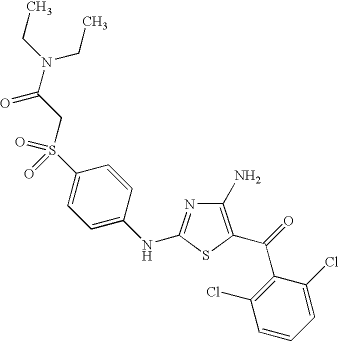 | 9.1 | −7 | −9.2 |
| DD(162) | 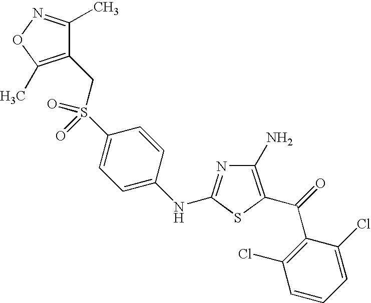 | 14 | 13 | 5.8 |
| DD(163) | 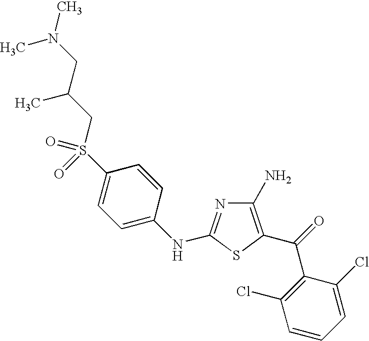 | 52.8 | 27 | −5.8 |
| DD(164) | 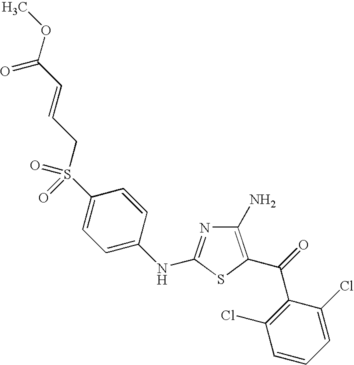 | −6.9 | 8.6 | −7.4 |
| DD(165) | 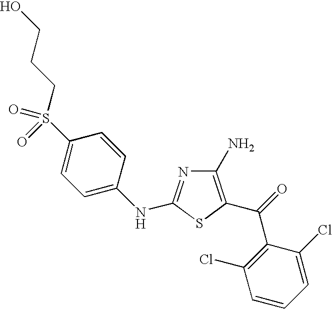 | 34.3 | 10 | −1.8 |
| DD(166) | 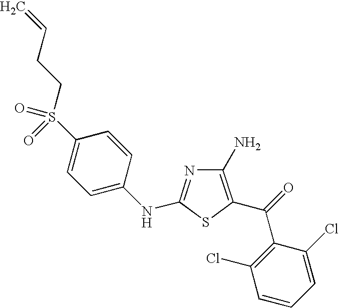 | 14.5 | 14 | 3.5 |
| DD(167) | 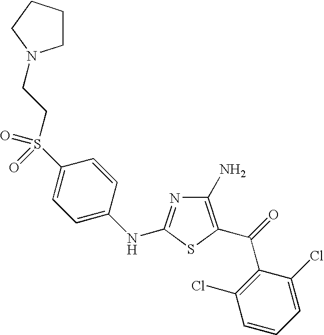 | 0.8 | 17 | −2.5 |
| DD(168) | 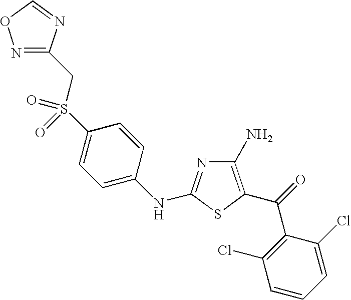 | 30.7 | 7.8 | 8.3 |
| DD(169) | 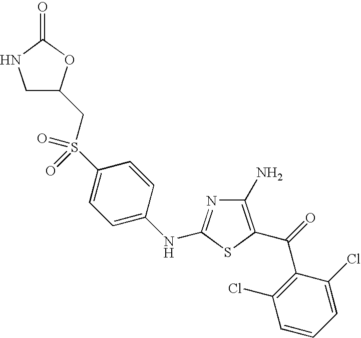 | 36.4 | 33 | 44 |
| DD(170) | 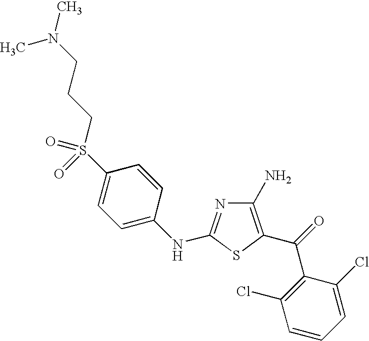 | 25.8 | 18 | 22 |
| DD(171) | 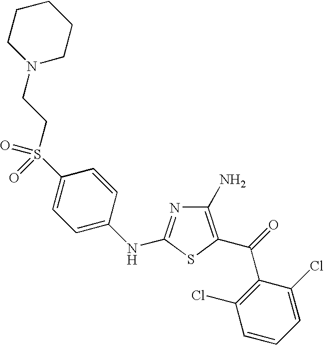 | 17.1 | 21 | −29 |
| DD(172) | 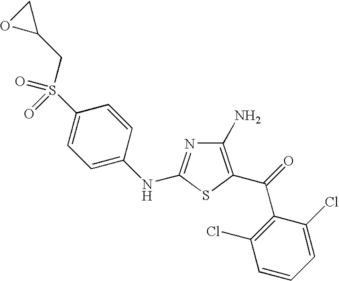 | 24.9 | 24 | −26 |
| DD(173) | 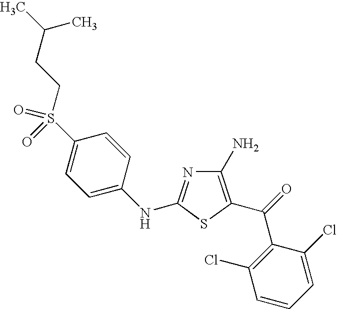 | 14.5 | 17 | −11 |
| DD(174) | 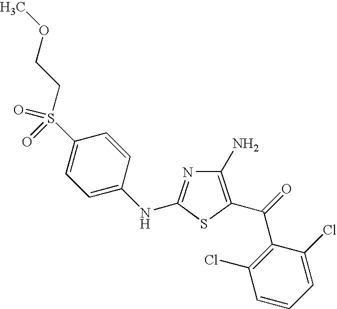 | 24.6 | 24 | −16 |
| DD(175) | 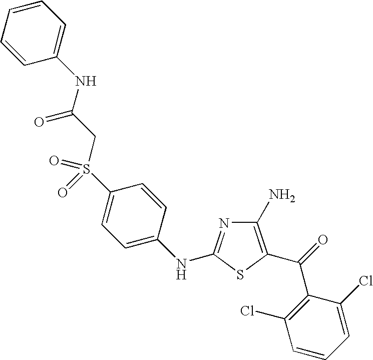 | 12.4 | 23 | −14 |
| DD(176) | 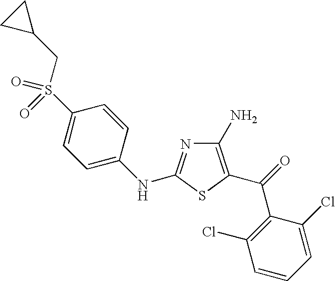 | 5.4 | 13 | 17 |
| DD(177) | 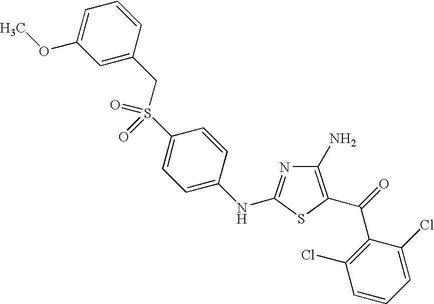 | 3.2 | 15 | −29 |
| DD(178) | 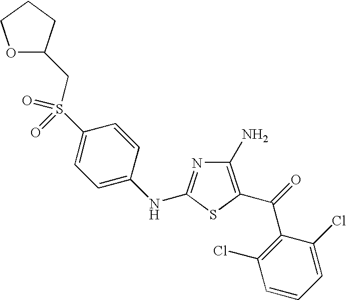 | 16.6 | 16 | −27 |
| DD(179) | 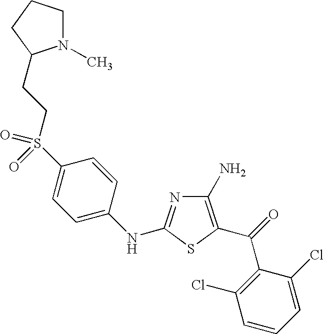 | 9.4 | 24 | −1.1 |
| DD(180) | 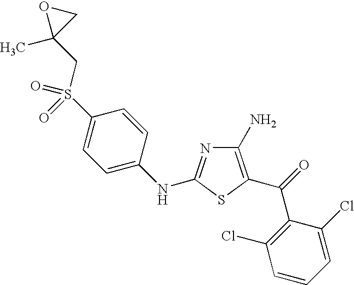 | 4.2 | 13 | −14 |
| DD(181) | 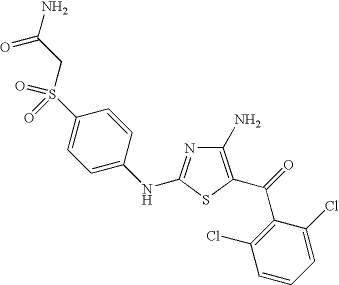 | 27.5 | 28 | −10 |
| DD(182)) | 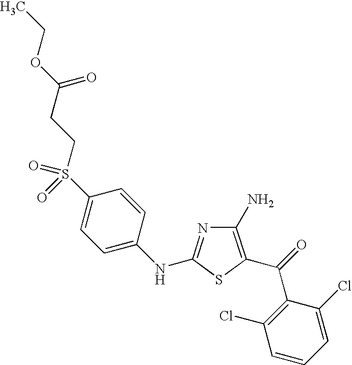 | 5.4 | 17 | −12 |
| DD(183) | 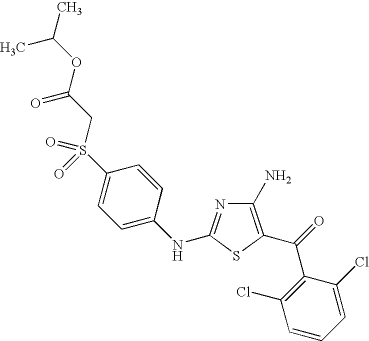 | 1.9 | 17 | 8.6 |
| DD(184) | 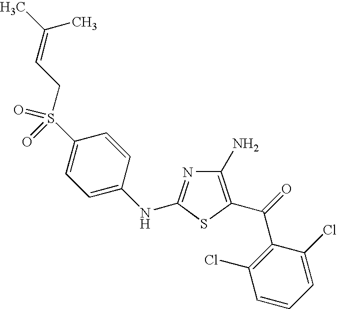 | −3.3 | 9 | 1.6 |
| DD(185) | 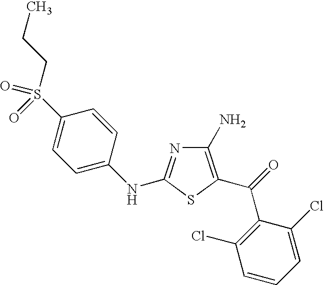 | 22.3 | 33 | 8.5 |
| DD(186) | 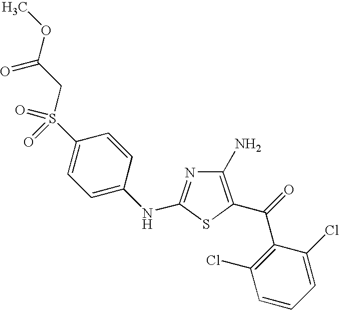 | 38.9 | 26 | 7 |
| DD(187) | 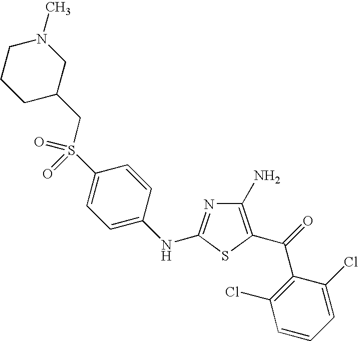 | −6.4 | 24 | 6 |
| DD(188) | 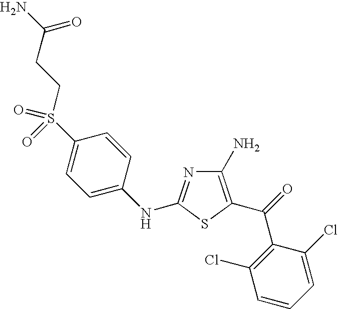 | 25.9 | 35 | −11 |
| DD(189) | 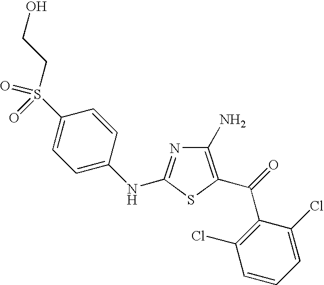 | 32.2 | 31 | −19 |
| DD(190) | 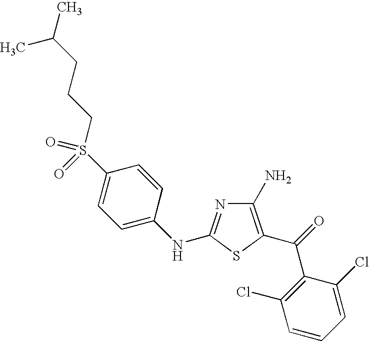 | 33.4 | 25 | −21 |
| DD(191) | 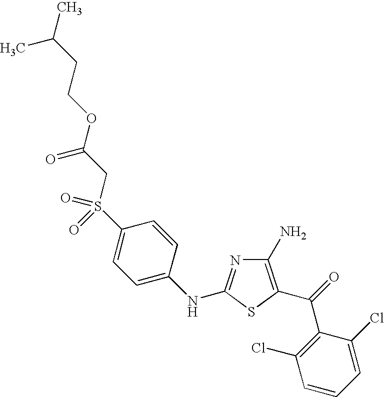 | 9.3 | 31 | −24 |
| DD(192) | 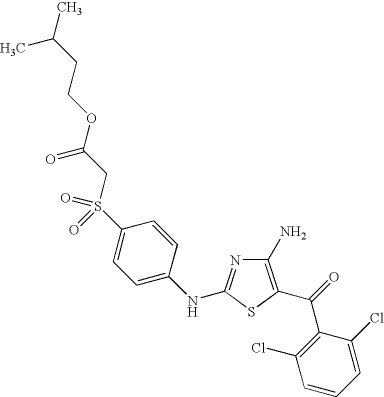 | −29 | 11 | 5.6 |
| DD(193) | 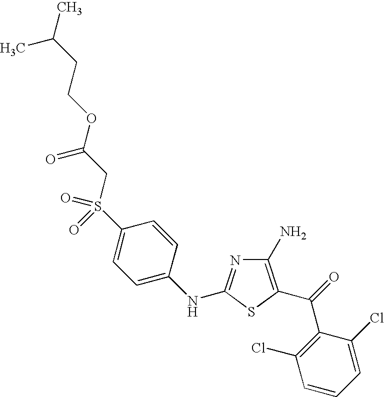 | 61.1 | 31 | −2.9 |
| DD(194) | 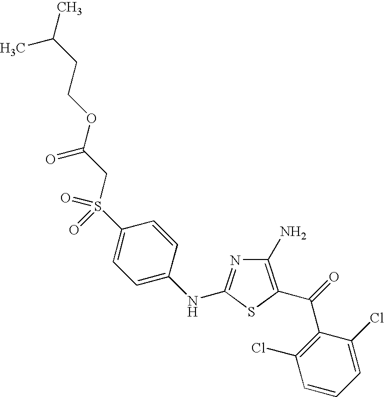 | 6.3 | 32 | −29 |
| DD(195) | 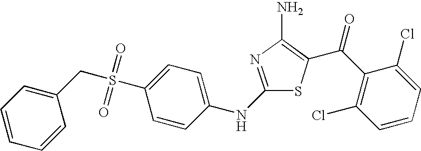 | 22.3 | 25 | 3.8 |
| DD(196) | 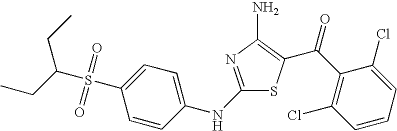 | 14.4 | 15 | 1.3 |
| DD(197) | 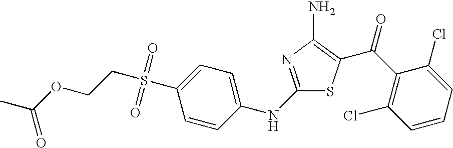 | 62.3 | 37 | 6.5 |
| DD(198) | 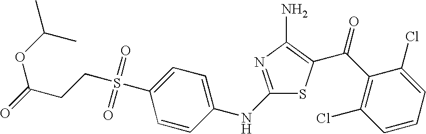 | 5.1 | 9.2 | −24 |
| DD(199) | 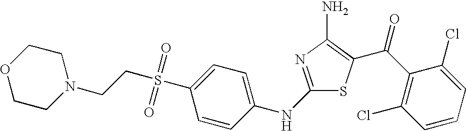 | 40.5 | 31 | −7.7 |
| DD(200) | 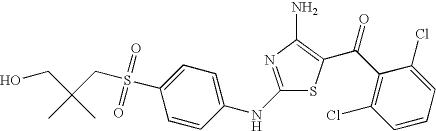 | 22.4 | 22 | −13 |
| DD(201) | 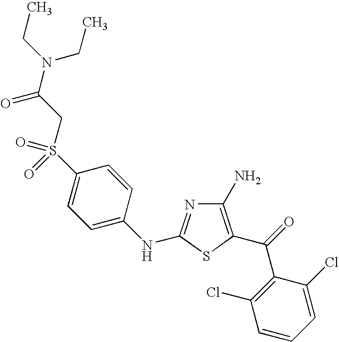 | 10.3 | 36 | 3.5 |
| DD(202) | 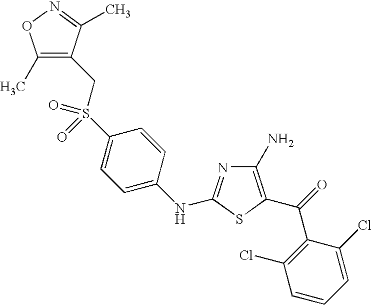 | 45.2 | 29 | 17 |
| DD(203) | 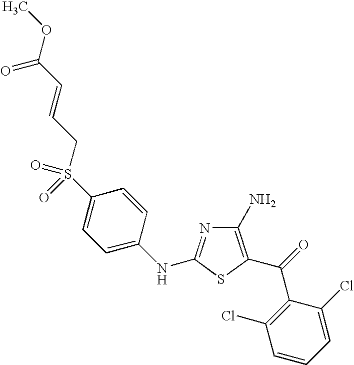 | 33.7 | 37 | −5.5 |
| DD(204) | 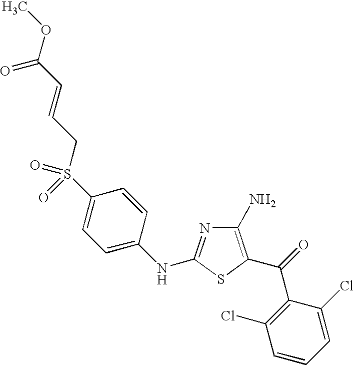 | 20.8 | 29 | −3.4 |
| DD(205) | 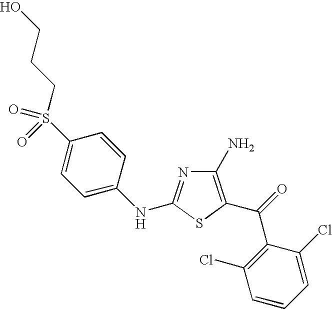 | 56.4 | 43 | −4.1 |
| DD(206) | 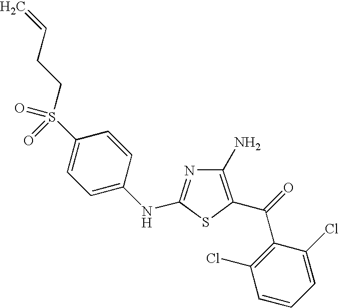 | 47.3 | 24 | −11 |
| DD(207) | 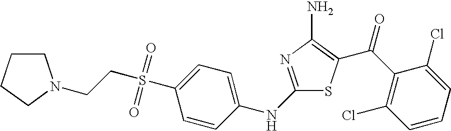 | 36.8 | 41 | −9.9 |
| DD(208) | 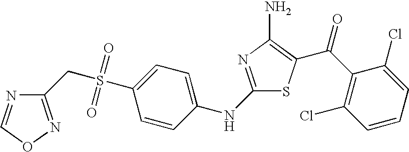 | 47.8 | 36 | 0.2 |
| DD(209) | 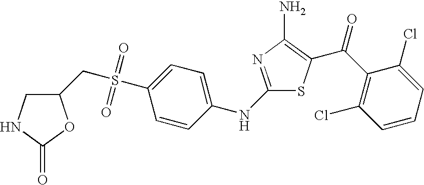 | −21 | 8 | −10 |
| DD(210) | 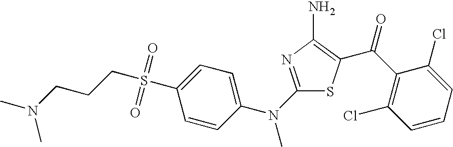 | −37 | 21 | −22 |
| DD(211) | 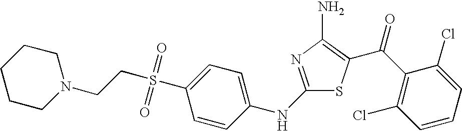 | −0.2 | 21 | −11 |
| DD(212) | 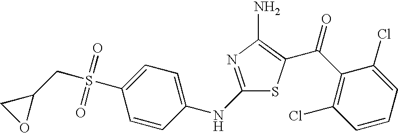 | −14 | 17 | −16 |
| DD(213) | 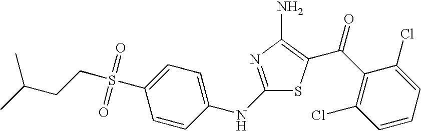 | −11 | 14 | −22 |
| DD(214) | 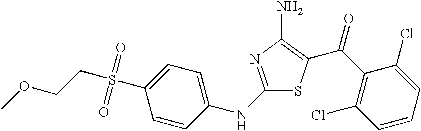 | 60.6 | 29 | −10 |
| DD(215) | 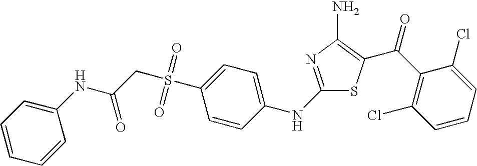 | 4.9 | 25 | −15 |
| DD(216) | 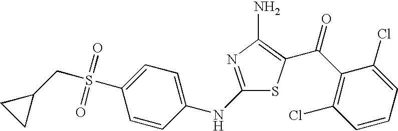 | 47.1 | 20 | 12 |
| DD(217) | 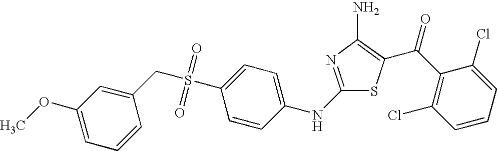 | −21 | 39 | −8 |
| DD(218) | 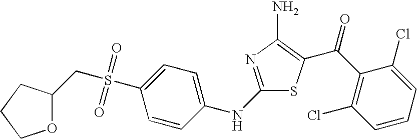 | 23.4 | 33 | −12 |
| DD(219) | 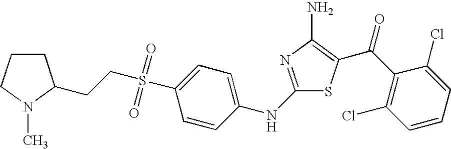 | 19.5 | 22 | 3.3 |
| DD(220) | 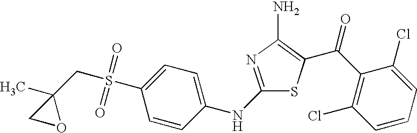 | 26.3 | 30 | −19 |
| DD(221) | 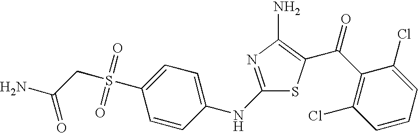 | 17.6 | 21 | −14 |
| DD(222) | 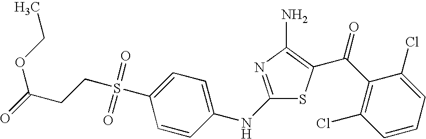 | −11 | −11 | −9 |
| DD(223) | 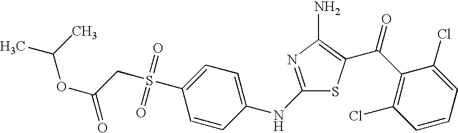 | 11.7 | 35 | −11 |
| DD(224) | 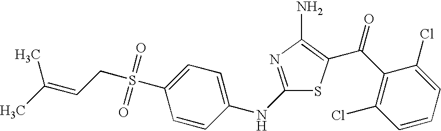 | 9.8 | 12 | −2.6 |
| DD(225) | 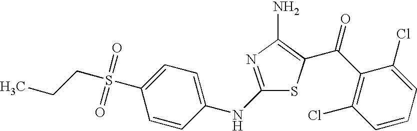 | 0.6 | 17 | −5 |
| DD(226) | 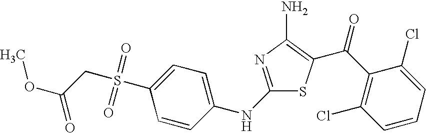 | −3.9 | 18 | −6.3 |
| DD(227) | 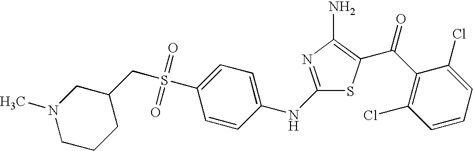 | 15.2 | 16 | −12 |
| DD(228) | 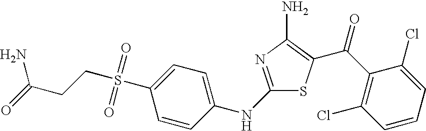 | 43 | 29 | 1 |
| DD(229) | 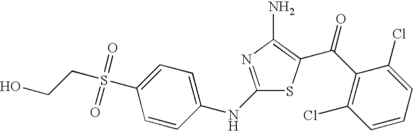 | 3.5 | 23 | −13 |
| DD(230) | 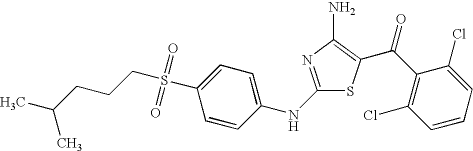 | −1.6 | 20 | −17 |
| DD(231) | 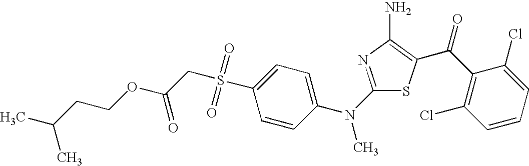 | 33.3 | 27 | −1.8 |
| DD(232) | 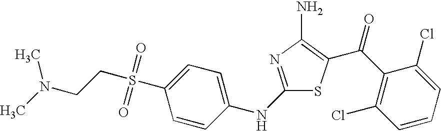 | 17.6 | 26 | 3.7 |
| DD(233) | 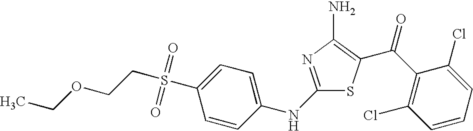 | −12 | 11 | 8.4 |
| DD(234) | 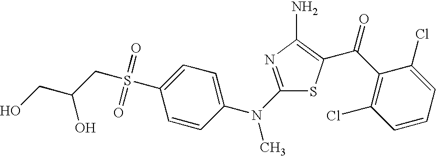 | 26.1 | 35 | 9.6 |
| DD(235) | 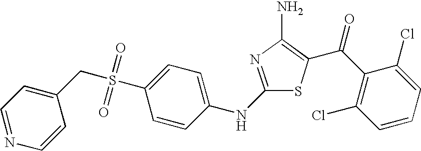 | 6.9 | 27 | 7.7 |
| DD(236) | 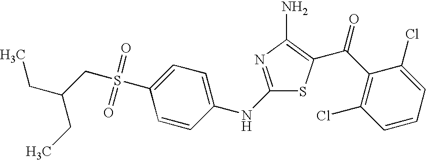 | 1.7 | 30 | −0.4 |
| DD(237) | 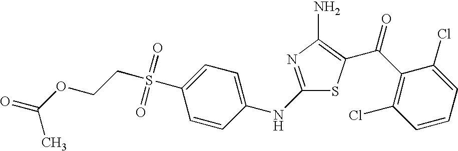 | 7.3 | 22 | −1.7 |
| DD(238) | 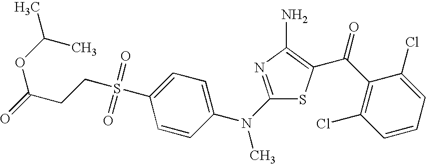 | 21.5 | 22 | −19 |
| DD(239) | 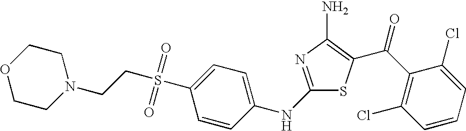 | 8 | 27 | 12 |
| DD(240) | 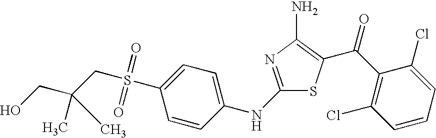 | 8.7 | 11 | 6.8 |

TABLE 4

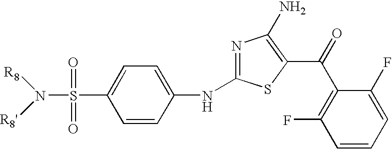

| | | | % inhib | % inhib | % inhib | % inhib |
|---|---|---|---|---|---|---|
| | | | CDK2/A @ | CDK2/A @ | CDK4/D @ | CDK4/D @ |
| Example | R₈ | R₈′ | 0.01 μM | 0.03 μM | 0.01 μM | 0.03 μM |
| EE(1) | —H | 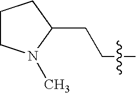 | −10 | 37 | 11 | 46 |
| EE(2) | —H | 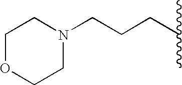 | 23 | 44 | 8 | 36 |
| EE(3) | —H | 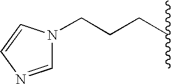 | 32 | 58 | 17 | 39 |
| EE(4) | —H | 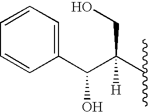 | −4 | 30 | 1 | 23 |
| EE(5) | —H | 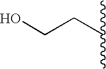 | 26 | 55 | 16 | 36 |
| EE(6) | —H | 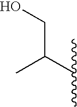 | 6 | 36 | 8 | 24 |
| EE(7) | —H | 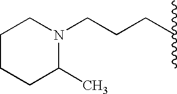 | 30 | 56 | 34 | 66 |
| EE(8) | —H | 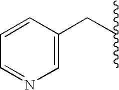 | 32 | 60 | 10 | 17 |
| EE(9) | —H | 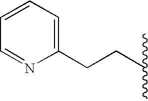 | 44 | 54 | 14 | 33 |
| EE(10) | —H | 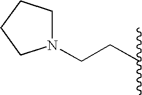 | 32 | 52 | 35 | 65 |
| EE(11) | —H | 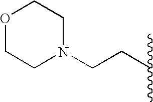 | 33 | 51 | 16 | 43 |
| EE(12) | —H | 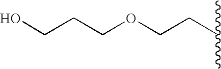 | 16 | 39 | 0 | 13 |
| EE(13) | —H | 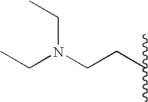 | 27 | 47 | 27 | 61 |
| EE(14) | —H | 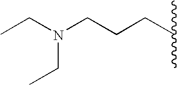 | 24 | 54 | 27 | 61 |
| EE(15) | —H | 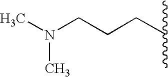 | 29 | 49 | 28 | 62 |
| EE(16) | —H | 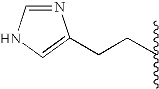 | −17 | 9 | −5 | 7 |
| EE(17) | —H | 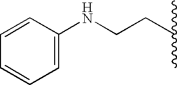 | 78 | 87 | 63 | 87 |
| EE(18) | —H | 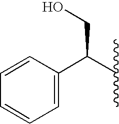 | 14 | 37 | −2 | 16 |
| EE(19) | —H | 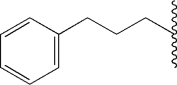 | 44 | 66 | 15 | 41 |
| EE(20) | —H | 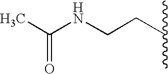 | 29 | 53 | 12 | 35 |
| EE(21) | —H | 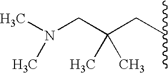 | 25 | 49 | 24 | 50 |
| EE(22) | —H | 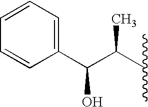 | 46 | 72 | 17 | 43 |
| EE(23) | —H | 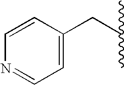 | −10 | 41 | −10 | 25 |
| EE(24) | —H | 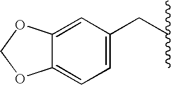 | 26 | 61 | −7 | 21 |
| EE(25) | —H | 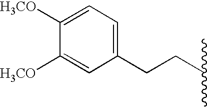 | 19 | 35 | −3 | 11 |
| EE(26) | —H | 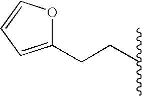 | 45 | 68 | 11 | 26 |
| EE(27) | —H | 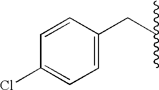 | 34 | 63 | −1 | 18 |
| EE(28) | —H | 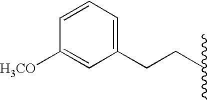 | 23 | 54 | 10 | 32 |
| EE(29) | —H | 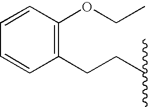 | 23 | 50 | −2 | 20 |
| EE(30) | —H | 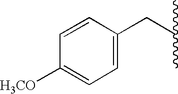 | 29 | 46 | 11 | 18 |
| EE(31) | —H | 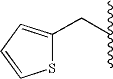 | 45 | 75 | 12 | 34 |
| EE(32) | —H | 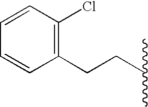 | 32 | 56 | 2 | 16 |
| EE(33) | —H | 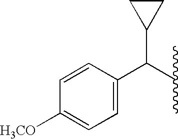 | 1 | 20 | 8 | 15 |
| EE(34) | —H | 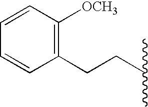 | 8 | 48 | −6 | 21 |
| EE(35) | —H | 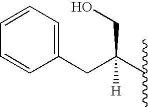 | −1 | 35 | −1 | 17 |
| EE(36) | —CH₃ | 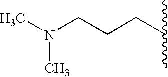 | 1 | 36 | 14 | 34 |
| EE(37) | —H | 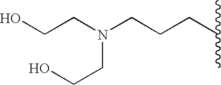 | 15 | 31 | 13 | 39 |
| EE(38) | —H | 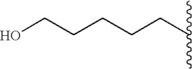 | 28 | 66 | 11 | 38 |
| EE(39) | —H | 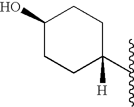 | 27 | 51 | 5 | 25 |
| EE(40) | —H | 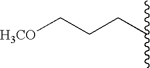 | 28 | 56 | 4 | 32 |
| EE(41) | —H | 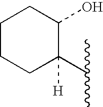 | 7 | 34 | 5 | 22 |
| EE(42) | —H | 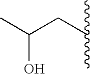 | 36 | 64 | 11 | 39 |
| EE(43) | —H | 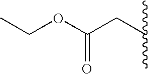 | 8 | 15 | 0 | 12 |
| EE(44) | —H | 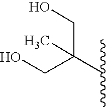 | 13 | 18 | 0 | 23 |
| EE(45) | —H | 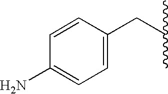 | 16 | 54 | −6 | 20 |
| EE(46) | —H | 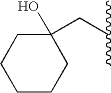 | −1 | 12 | −5 | 11 |
| EE(47) | —H | 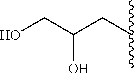 | 30 | 64 | 4 | 30 |
| EE(48) | —H | 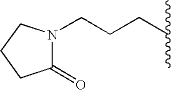 | 42 | 60 | 1 | 33 |
| EE(49) | —H | 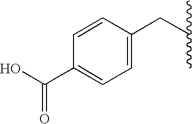 | 1 | 22 | −9 | 5 |
| EE(50) | —H | 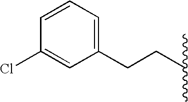 | 45 | 69 | 25 | 56 |
| EE(51) | —H | 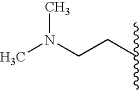 | 20 | 48 | 30 | 64 |
| EE(52) | —H | 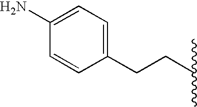 | 2 | 54 | 29 | 30 |
| EE(53) | —H | 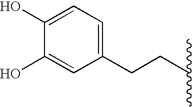 | −1 | 9 | 1 | 14 |
| EE(54) | —H | 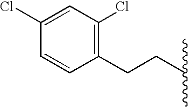 | 23 | 45 | 2 | 18 |
| EE(55) | —H | 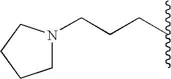 | 34 | 56 | 34 | 61 |
| EE(56) | —H | 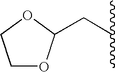 | −19 | 34 | −19 | 12 |
| EE(57) | —H | 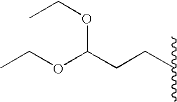 | 20 | 64 | 0 | 27 |
| EE(58) | —H | 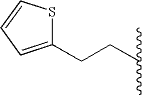 | 53 | 75 | 7 | 42 |
| EE(59) | —H | 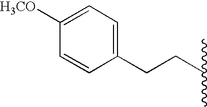 | 24 | 46 | 5 | 16 |
| EE(60) | —H | 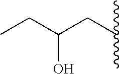 | 22 | 50 | 4 | 20 |
| EE(61) | —H | 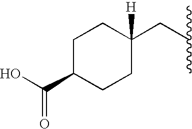 | 8 | 14 | 7 | 13 |
| EE(62) | —H | 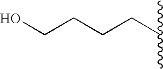 | 31 | 42 | 2 | 26 |
| EE(63) | —H | 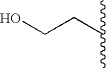 | 42 | 56 | 20 | 33 |
| EE(64) | —H | 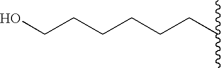 | 40 | 65 | 24 | 33 |
| EE(65) | —H | 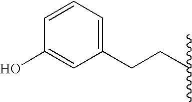 | 10 | 19 | 4 | 8 |
| EE(66) | —H | 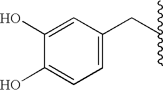 | 29 | 14 | 14 | 10 |
| EE(67) | —H | 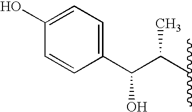 | −30 | −10 | −21 | 0 |
| EE(68) | —H | 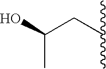 | 17 | 55 | 3 | 32 |
| EE(69) | —H | 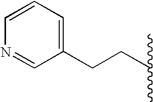 | 33 | 52 | −4 | 37 |
| EE(70) | —H | 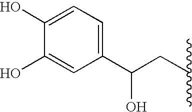 | 7 | 17 | 2 | 9 |
| EE(71) | —H | 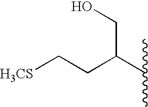 | 29 | 53 | 2 | 25 |
| EE(72) | —H | 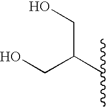 | 14 | 21 | 4 | 12 |
| EE(73) | —H | 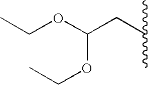 | 20 | 37 | −2 | 14 |
| EE(74) | —H | 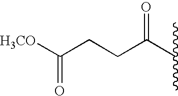 | −18 | −2 | −12 | 8 |
| EE(75) | —H | 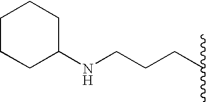 | 28 | 46 | 26 | 52 |
| EE(76) | —H | 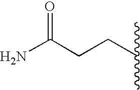 | 26 | 7 | 5 | 10 |
| EE(77) | —H | 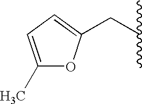 | 79 | 75 | 29 | 46 |
| EE(78) | —H | 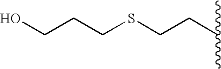 | 3 | 54 | −18 | 21 |
| EE(79) | —H | 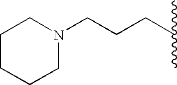 | 1 | 54 | 16 | 59 |
| EE(80) | —H | 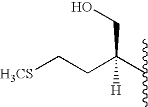 | 24 | 34 | −16 | 23 |
| EE(81) | —H | 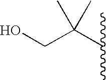 | 12 | 25 | −2 | 2 |
| EE(82) | —H | 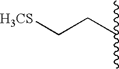 | 43 | 56 | 1 | 21 |
| EE(83) | —H | 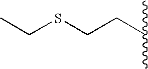 | 60 | 66 | 15 | 35 |
| EE(84) | —H | 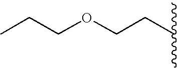 | 56 | 82 | 11 | 40 |
| EE(85) | —H | 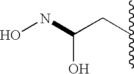 | 14 | 5 | 5 | 10 |
| EE(86) | —H | 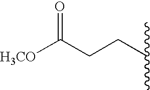 | 9 | 5 | −9 | 3 |
| EE(87) | —H | 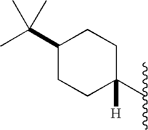 | 10 | 9 | 6 | 3 |
| EE(88) | —H | 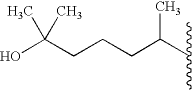 | 47 | 72 | 50 | 73 |
| EE(89) | −CH₃ | 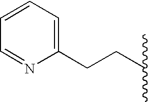 | ND | 16 | ND | 17 |
| EE(90) | — | 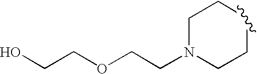 | ND | −1 | ND | 13 |
| EE(91) | —CH₃ | 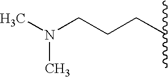 | ND | 22 | ND | 41 |
| EE(92) | —CH₃ | 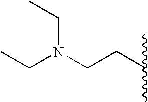 | ND | 27 | ND | 35 |
| EE(93) | — | 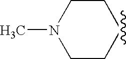 | ND | 18 | ND | 11 |
| EE(94) | — | 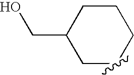 | ND | 30 | ND | 20 |
| EE(95) | | 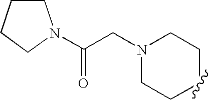 | ND | 14 | ND | 13 |
| EE(96) | | 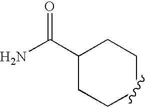 | ND | 40 | ND | 28 |
| EE(97) | —CH₃ | 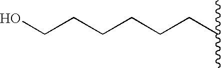 | ND | 28 | ND | 26 |
| EE(98) | — | 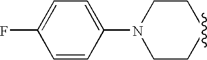 | ND | 37 | ND | 15 |
| EE(99) | —CH₃ | 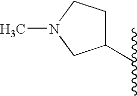 | ND | 22 | ND | 28 |
| EE(100) | —CH₃ | 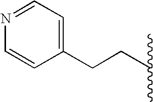 | ND | 19 | ND | 26 |
| EE(101) | 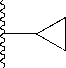 | 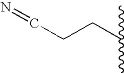 | ND | 8 | ND | 23 |
| EE(102) | —CH₃ | 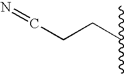 | ND | 27 | ND | 25 |
| EE(103) | — | 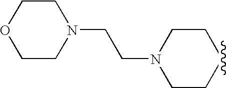 | ND | 11 | ND | 18 |
| EE(104) | — | 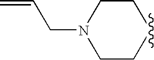 | ND | 6 | ND | 17 |
| EE(105) | 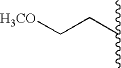 | 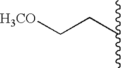 | ND | 8 | ND | 15 |
| EE(106) | 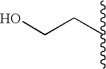 | 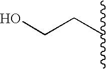 | ND | −1 | ND | 8 |
| EE(107) | — | 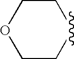 | ND | 27 | ND | 22 |
| EE(108) | —CH₃ | 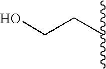 | ND | 28 | ND | 19 |
| EE(109) | —CH₃ | 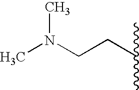 | ND | 26 | ND | 31 |
| EE(110) | −CH₂CH₃ | 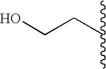 | ND | 24 | ND | 19 |
| EE(111) | —CH₃ | 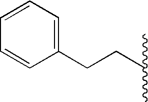 | ND | 9 | ND | 17 |
| EE(112) | — | 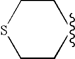 | ND | 23 | ND | 21 |
| EE(113) | —CH₃ | 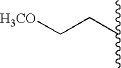 | ND | 4 | ND | 18 |
| EE(114) | —CH₃ | 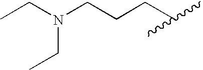 | ND | 21 | ND | 35 |
| EE(115) | —CH₃ | 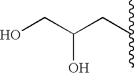 | ND | 15 | ND | 9 |
| EE(116) | —CH₃ | 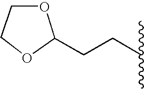 | ND | 20 | ND | 13 |
| EE(117) | —CH₃ | 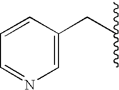 | ND | 12 | ND | 14 |
| EE(118) | —CH₃ | 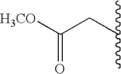 | ND | 18 | ND | 22 |
| EE(119) | —CH₃ | 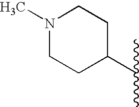 | ND | 9 | ND | 11 |
| EE(120) | — | 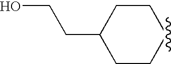 | ND | 21 | ND | 19 |

ND = Not determined.

What is claimed is:

1. A compound of Formula (I): 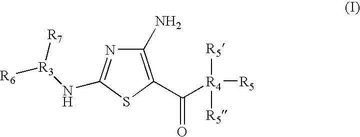
wherein:
R₃ is a monocycle selected from the group consisting of C₃-C₁₀ cycloalkyl, 3-10 membered heterocycloalkyl, aryl and 3-10 membered heteroaryl;
R₄ is a moiety selected from the group consisting of C₂-C₁₄ alkyl, C₃-C₁₀ cycloalkyl, 3-10 membered heterocycloalkyl, aryl and 3-10 membered heteroaryl, wherein R₄ is unsubstituted or substituted with 1 to 4 R₁₀ groups;
R₅ is a moiety selected from the group consisting of hydroxyl, halo, C₁-C₁₄ alkyl, C₁-C₁₄ alkoxyl, acyl, amide and nitro;
R₅′ and R₅″ are independently selected from hydrogen, hydroxyl, halo, C₁₋₁₄ alkyl, C₁-C₁₄ alkoxyl, acyl, amide, amino, acetamido and nitro;
R₆ is a group selected from the following formulae: 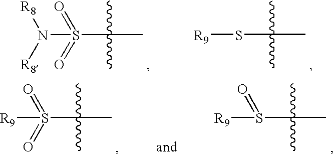
wherein:
R₈ is hydrogen, C₁-C₃ alkyl, C₃-C₁₀ cycloalkyl, or C₁-C₁₄ alkoxyl;
R_{8′} is an C₃-C₁₄ alkyl, 2 to 9 membered heteroalkyl, acyl, C₁-C₃ alkyl-nitrile, C₁-C₃ alkyl-carboxamide, C₁-C₄ alkyl-heterocycloalkyl, C₁-C₄ alkyl-aryl, C₁-C₄ alkyl-heteroaryl, C₃-C₁₀ cycloalkyl, 3-10 membered heterocycloalkyl, aryl or 3-10 membered heteroaryl, or together with R₈ cyclizes to form an unsubstituted or substituted C₃-C₁₀ cycloalkyl, 3-10 membered heterocycloalkyl, aryl or 3-10 membered heteroaryl, with the proviso that R₆ is not 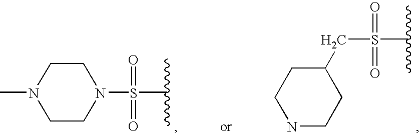
and wherein R_{8′} is unsubstituted or substituted with 1 to 4 R₁₀ groups;
R₉ is hydrogen, or a moiety selected from the group consisting of an C₁-C₉ alkyl, C₂-C₉ alkenyl, 2-9 membered heteroalkenyl, C₁-C₉ alkylamide, C₁-C₉ alkyl-carboxamide, 2-9 membered heteroalkyl, C₁-C₄ alkyl-cycloalkyl, C₁-C₄ alkyl-heterocycloalkyl, C₁-C₄ alkyl-aryl, C₁-C₄ alkyl-heteroaryl, C₃-C₁₀ cycloalkyl, 3-10 membered heterocycloalkyl, aryl and 3-10 membered heteroaryl, with the proviso that R₆ is not 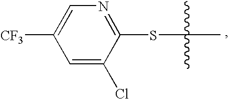
and wherein R₉ is unsubstituted or substituted with 1 to 4 R₁₀ groups;
R₇ is a moiety selected from the group consisting of hydrogen, hydroxyl, halo, C₁-C₁₄ alkyl, C₁-C₁₄ alkoxyl, acyl, amide and nitro;
wherein each R₁₀ is independently selected from halo, cyano, nitro, trifluoromethoxy, trifluoromethyl, azido, hydroxyl, C₁-C₆ alkoxyl, C, C₁₀ alkyl, C₂-C₆ alkenyl, C₂-C₆ alkynyl, —C(O)Rₐ, —C(O)OR_{b}, —OC(O)R_{b}, —NR_{b}C(O)R_{c}, —C(O)NR_{b}R_{c}, —NR_{b}R_{c}, —NR_{b}OR_{c}, —S(O)ⱼ(C₁-C₆ alkyl) wherein j is an integer from 0 to 2, —(CR_{d}Rₑ)ₜ(C₃-C₁₀ cycloalkyl), —(CR_{d}Rₑ)ₜ(aryl), —(CR_{d}Rₑ)ₜ(4-10 membered heterocycloalkyl), —(CR_{d}Rₑ)ₜ(4-10 membered heteroaryl), —(CR_{d}Rₑ)_{q}C(O)(CR_{d}Rₑ)ₜ(C₃-C₁₀ cycloalkyl), —(CR_{d}Rₑ)_{q}C(O)(CR_{d}Rₑ)ₜ(aryl), —(CR_{d}Rₑ)_{q}C(O)(CR_{d}Rₑ)ₜ(4-10 membered heterocycloalkyl), —(CR_{d}Rₑ)_{q}C(O)(CR_{d}Rₑ)ₜ(4-10 membered heteroaryl), —(CR_{d}Rₑ)ₜO(CR_{d}Rₑ)_{q}(C₃-C₁₀ cycloalkyl), —(CR_{d}Rₑ)ₜO(CR_{d}Rₑ)_{q}(aryl), —(CR_{d}Rₑ)ₜO(CR_{d}Rₑ)_{q}(4-10 membered heterocycloalkyl), —(CR_{d}Rₑ)ₜO(CR_{d}Rₑ)_{q}(4-10 membered heteroaryl), —(CR_{d}Rₑ)_{q}SO₂(CR_{d}Rₑ)ₜ(C₃-C₁₀ cycloalkyl), —(CR_{d}Rₑ)_{q}SO₂(CR_{d}Rₑ)ₜ(aryl), and —(CR_{d}Rₑ)_{q}SO₂(CR_{d}Rₑ)ₜ(4-10 membered heterocycloalkyl), —(CR_{d}Rₑ)_{q}SO₂(CR_{d}Rₑ)ₜ(4-10 membered heteroaryl), wherein Rₐ is selected from the group consisting of halo, hydroxyl, —NR_{d}Rₑ C₁-C₆ alkyl, trifluoromethyl, C₁-C₆ alkoxyl, and trifluoromethoxy, R_{b} and R_{c} are independently selected from H, C₁-C₆ alkyl, —(CR_{d}Rₑ)ₜ(C₃-C₁₀ cycloalkyl), —(CR_{d}Rₑ)ₜ(aryl), —(CR_{d}Rₑ)ₜ(4-10 membered heterocycloalkyl), and —(CR_{d}Rₑ)ₜ (4-10 membered heteroaryl), wherein q and t are each independently an integer from 0 to 5, R_{d} and Rₑ are independently H or C₁-C₆ alkyl, wherein 1 or 2 ring carbon atoms of the heterocyclic and heteroaryl moieties of the foregoing R₁₀ groups are unsubstituted or substituted with an oxo (═O) moiety, and the alkyl, alkenyl, alkynyl, aryl and heterocyclic and heteroaryl moieties of the foregoing R₁₀ groups are unsubstituted or substituted with 1 to 3 substituents independently selected from halo, cyano, nitro, trifluoromethyl, trifluoromethoxy, azido, —OR_{b}, —C(O)R_{b}, —C(O)OR_{b}, —NR_{b}C(O)R_{c}, —C(O)NR_{b}R_{c}, —NR_{b}R_{c}, —NR_{b}OR_{c}, C₁-C₆ alkyl, C₂-C₆ alkenyl, C₂-C₆ alkynyl, —(CR_{d}Rₑ)ₜ(C₃-C₁₀ cycloalkyl), —(CR_{d}Rₑ)ₜ(aryl), —(CR_{d}Rₑ)ₜ(4-10 membered heterocycloalkyl), and —(CR_{d}Rₑ)ₜ(4-10 membered heteroaryl);
and wherein any of the above-mentioned substituents comprising a CH₃ (methyl), CH₂ (methylene), or CH (methane) group which is not attached to a halogeno, SO or SO₂ group or to a N, O, or S is unsubstituted or substituted with a substituent from the group selected from hydroxyl, halo, C₁-C₄ alkyl, C₁-C₄ alkoxyl and —NR_{d}Rₑ wherein R_{d} and Rₑ are as defined above;
or a pharmaceutically acceptable salt of a compound of the Formula (I), or a multimer, prodrug or pharmaceutically active metabolite of a compound of the Formula (I) or pharmaceutically acceptable salt thereof.

2. A compound of Formula (II): 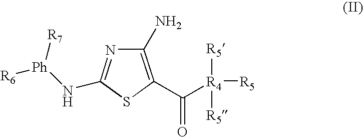
wherein:
R₄ is a moiety selected from the group consisting of C₂-C₁₄ alkyl, C₃-C₁₀ cycloalkyl, 3-10 membered heterocycloalkyl, aryl and 3-10 membered heteroaryl, wherein R₄ is unsubstituted or substituted with 1 to 4 R₁₀ groups;
R₅ is a moiety selected from the group consisting of hydroxyl, halo, C₁₋₁₄ alkyl, C₁-C₁₄ alkoxyl, acyl, amide and nitro;
R₅′ and R₅″ are independently selected from hydrogen, hydroxyl, halo, C₁₋₁₄ alkyl, C₁-C₁₄ alkoxyl, acyl, amide, amino, acetamido and nitro;
R₆ is a group selected from the following formulae: 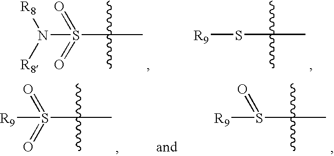
wherein:
R₈ is hydrogen, C₁₋₃ alkyl, C₃-C₁₀ cycloalkyl, or C₁-C₁₄ alkoxyl;
R_{8′} is an C₃-C₁₄ alkyl, 2-9 membered heteroalkyl, acyl, C₁-C₃ alkyl-nitrile, C₁-C₃ alkyl-carboxamide, C₁-C₄ alkyl-heterocycloalkyl, C₁-C₄ alkyl-aryl, C₁-C₄ alkyl-heteroaryl, C₃-C₁₀ cycloalkyl, 3-10 membered heterocycloalkyl, aryl or 3-10 membered heteroaryl, or together with R₈ cyclizes to form a C₃-C₁₀ cycloalkyl, 3-10 membered heterocycloalkyl, aryl or 3-10 membered heteroaryl, with the proviso that R₆ is not 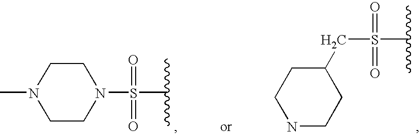
and wherein R_{8′} is unsubstituted or substituted with 1 to 4 R₁₀ groups;
R₉ is hydrogen, or a moiety selected from the group consisting of an C₁-C₉ alkyl, C₂-C₉ alkenyl, 2-9 membered heteroalkenyl, C₁-C₉ alkylamide, C₁-C₉ alkyl-carboxamide, 2-9 membered heteroalkyl, C₁-C₄ alkyl-cycloalkyl, C₁-C₄ alkyl-heterocycloalkyl, C₁-C₄ alkyl-aryl, C₁-C₄ alkyl-heteroaryl, C₃-C₁₀ cycloalkyl, 3-10 membered heterocycloalkyl, aryl and 3-10 membered heteroaryl, with the proviso that R₆ is not 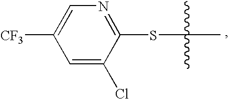
wherein R₉ is unsubstituted or substituted with 1 to 4 R₁₀ groups;
R₇ is a moiety selected from the group consisting of hydrogen, hydroxyl, halo, C₁-C₁₄ alkyl, C₁-C₁₄ alkoxyl, acyl, amide and nitro;
wherein each R₁₀ is independently selected from halo, cyano, nitro, trifluoromethoxy, trifluoromethyl, azido, hydroxyl, C₁-C₆ alkoxyl, C, C₁₀ alkyl, C₂-C₆ alkenyl, C₂-C₆ alkynyl, —C(O)Rₐ, —C(O)OR_{b}, —OC(O)R_{b}, —NR_{b}C(O)R_{c}, —C(O)NR_{b}R_{c}, —NR_{b}R_{c}, —NR_{b}OR_{c}, —S(O)ⱼ(C₁-C₆ alkyl) wherein j is an integer from 0 to 2, —(CR_{d}Rₑ)ₜ(C₃-C₁₀ cycloalkyl), —(CR_{d}Rₑ)ₜ(aryl), —(CR_{d}Rₑ)ₜ(4-10 membered heterocycloalkyl), —(CR_{d}Rₑ)ₜ(4-10 membered heteroaryl), —(CR_{d}Rₑ)_{q}C(O)(CR_{d}Rₑ)ₜ(C₃-C₁₀ cycloalkyl), —(CR_{d}Rₑ)_{q}C(O)(CR_{d}Rₑ)ₜ(aryl), —(CR_{d}Rₑ)_{q}C(O)(CR_{d}Rₑ)ₜ(4-10 membered heterocycloalkyl), —(CR_{d}Rₑ)_{q}C(O)(CR_{d}Rₑ)ₜ(4-10 membered heteroaryl), —(CR_{d}Rₑ)ₜO(CR_{d}Rₑ)_{q}(C₃-C₁₀ cycloalkyl), —(CR_{d}Rₑ)ₜO(CR_{d}Rₑ)_{q}(aryl), —(CR_{d}Rₑ)ₜO(CR_{d}Rₑ)_{q}(4-10 membered heterocycloalkyl), —(CR_{d}Rₑ)ₜO(CR_{d}Rₑ)_{q}(4-10 membered heteroaryl), —(CR_{d}Rₑ)_{q}SO₂(CR_{d}Rₑ)ₜ(C₃-C₁₀ cycloalkyl), —(CR_{d}Rₑ)_{q}SO₂(CR_{d}Rₑ)ₜ(aryl), and —(CR_{d}Rₑ)_{q}SO₂(CR_{d}Rₑ)ₜ(4-10 membered heterocycloalkyl), —(CR_{d}Rₑ)_{q}SO₂(CR_{d}Rₑ)ₜ(4-10 membered heteroaryl), wherein Rₐ is selected from the group consisting of halo, hydroxyl, —NR_{d}Rₑ C₁-C₆ alkyl, trifluoromethyl, C₁-C₆ alkoxyl, and trifluoromethoxy, R_{b} and R_{c} are independently selected from H, C₁-C₆ alkyl, —(CR_{d}Rₑ)ₜ(C₃-C₁₀ cycloalkyl), —(CR_{d}Rₑ)ₜ(aryl), —(CR_{d}Rₑ)ₜ(4-10 membered heterocycloalkyl), and
—(CR_{d}Rₑ)ₜ(4-10 membered heteroaryl), wherein q and t are each independently an integer from 0 to 5, R_{d} and Rₑ are independently H or C₁-C₆ alkyl, wherein 1 or 2 ring carbon atoms of the heterocyclic and heteroaryl moieties of the foregoing R₁₀ groups are unsubstituted or substituted with an oxo (═O) moiety, and the alkyl, alkenyl, alkynyl, aryl and heterocyclic and heteroaryl moieties of the foregoing R₁₀ groups are unsubstituted or substituted with 1 to 3 substituents independently selected from halo, cyano, nitro, trifluoromethyl, trifluoromethoxy, azido, —OR_{b}, —C(O)R_{b}, —C(O)OR_{b}, —NR_{b}C(O)R_{c}, —C(O)NR_{b}R_{c}, —NR_{b}R_{c}, —NR_{b}OR_{c}, C₁-C₆ alkyl, C₂-C₆ alkenyl, C₂-C₆ alkynyl, —(CR_{d}Rₑ)ₜ(C₃-C₁₀ cycloalkyl), —(CR_{d}Rₑ)ₜ(aryl), —(CR_{d}Rₑ)ₜ(4-10 membered heterocycloalkyl), and
—(CR_{d}Rₑ)ₜ(4-10 membered heteroaryl);
wherein any of the above-mentioned substituents comprising a CH₃ (methyl), CH₂ (methylene), or CH (methane) group which is not attached to a halogeno, SO or SO₂ group or to a N, O, or S is unsubstituted or substituted with a substituent from the group selected from hydroxyl, halo, C₁-C₄ alkyl, C₁-C₄ alkoxy and —NR_{d}Rₑ wherein R_{d} and Rₑ are as defined above;
and wherein Ph means phenyl;
or a pharmaceutically acceptable salt of a compound of the Formula (I), or a multimer, prodrug or pharmaceutically active metabolite of a compound of the Formula (I) or pharmaceutically acceptable salt thereof.

3. A compound according to claim 1 wherein R₄ is a phenyl;
R₃ is a monocycle selected from the group consisting of C₃-C₁₀ cycloalkyl, 3-10 membered heterocycloalkyl, aryl and 3-10 membered heteroaryl;
R₅ is a moiety selected from the group consisting of hydroxyl, halo, C₁-C₁₄ alkyl, C₁-C₁₄ alkoxyl, acyl, amide and nitro;
R₅′ and R₅″ are independently selected from hydrogen, hydroxyl, halo, C₁₋₁₄ alkyl, C₁-C₁₄ alkoxyl, acyl, amide, amino, acetamido and nitro;
R₆ is a group selected from the following formulae: 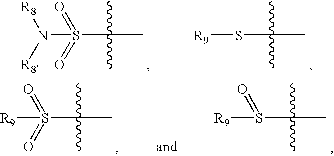
wherein:
R₈ is hydrogen, C₁-C₃ alkyl, C₃-C₁₀ cycloalkyl, or C₁-C₁₄ alkoxyl;
R_{8′} is an C₃₋₁₄ alkyl, 2-9 membered heteroalkyl, acyl, C₁₋₃ alkyl-nitrile, C₁₋₃ alkyl-carboxamide, C₁₋₄ alkyl-heterocycloalkyl, C₁₋₄ alkyl-aryl, C₁₋₄ alkyl-heteroaryl, C₃-C₁₀ cycloalkyl, 3-10 membered heterocycloalkyl, aryl or 3-10 membered heteroaryl, or together with R₈ cyclizes to form a C₃-C₁₀ cycloalkyl, 3-10 membered heterocycloalkyl, aryl or 3-10 membered heteroaryl, with the proviso that R₆ is not 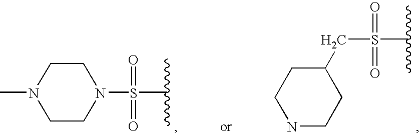
and wherein R₈ is unsubstituted or substituted with 1 to 4 R₁₀ groups;
R₉ is hydrogen, or a moiety selected from the group consisting of an C₁₋₉ alkyl, C₂₋₉ alkenyl, 2-9 membered heteroalkenyl, C₁₋₉ alkylamide, C₁₋₉ alkyl-carboxamide, 2-9 membered heteroalkyl, C₁₋₄ alkyl-cycloalkyl, C₁₋₄ alkyl-heterocycloalkyl, C₁₋₄ alkyl-aryl, C₁₋₄ alkyl-heteroaryl, C₃-C₁₀ cycloalkyl, 3-10 membered heterocycloalkyl, aryl and 3-10 membered heteroaryl, with the proviso that R₆ is not 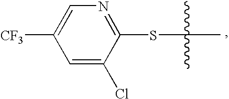
wherein R₉ is unsubstituted or substituted with 1 to 4 R₁₀ groups;
R₇ is a moiety selected from the group consisting of hydrogen, hydroxyl, halo, C₁-C₁₄ alkyl, C₁-C₁₄ alkoxyl, acyl, amide and nitro;
wherein each R₁₀ is independently selected from halo, cyano, nitro, trifluoromethoxy, trifluoromethyl, azido, hydroxyl, C₁-C₆ alkoxyl, C, C₁₀ alkyl, C₂-C₆ alkenyl, C₂-C₆ alkynyl, —C(O)Rₐ, —C(O)OR_{b}, —OC(O)R_{b}, —NR_{b}C(O)R_{c}, —C(O)NR_{b}R_{c}, —NR_{b}R_{c}, —NR_{b}OR_{c}, —S(O)ⱼ(C₁-C₆ alkyl) wherein j is an integer from 0 to 2, —(CR_{d}Rₑ)ₜ(C₃-C₁₀ cycloalkyl), —(CR_{d}Rₑ)ₜ(aryl), —(CR_{d}Rₑ)ₜ(4-10 membered heterocycloalkyl), —(CR_{d}Rₑ)ₜ(4-10 membered heteroaryl), —(CR_{d}Rₑ)_{q}C(O)(CR_{d}Rₑ)ₜ(C₃-C₁₀ cycloalkyl), —(C R_{d}Rₑ)_{q}C(O)(CR_{d}Rₑ)ₜ(aryl), —(CR_{d}Rₑ)_{q}C(O)(CR_{d}Rₑ)ₜ(4-10 membered heterocycloalkyl), —(CR_{d}Rₑ)_{q}C(O)(CR_{d}Rₑ)ₜ(4-10 membered heteroaryl), —(CR_{d}Rₑ)ₜO(CR_{d}Rₑ)_{q}(C₃-C₁₀ cycloalkyl), —(CR_{d}Rₑ)ₜO(CR_{d}Rₑ)_{q}(aryl), —(CR_{d}Rₑ)ₜO(CR_{d}Rₑ)_{q}(4-10 membered heterocycloalkyl), —(CR_{d}Rₑ)ₜO(CR_{d}Rₑ)_{q}(4-10 membered heteroaryl), —(CR_{d}Rₑ)_{q}SO₂(CR_{d}Rₑ)ₜ(C₃-C₁₀ cycloalkyl), —(CR_{d}Rₑ)_{q}SO₂(CR_{d}Rₑ)ₜ(aryl), and —(CR_{d}Rₑ)_{q}SO₂(CR_{d}Rₑ)ₜ(4-10 membered heterocycloalkyl), —(CR_{d}Rₑ)_{q}SO₂(CR_{d}Rₑ)ₜ(4-10 membered heteroaryl), wherein Rₐ is selected from the group consisting of halo, hydroxyl, —NR_{d}Rₑ C₁-C₆ alkyl, trifluoromethyl, C₁-C₆ alkoxyl, and trifluoromethoxy, R_{b} and R_{c} are independently selected from H, C₁-C₆ alkyl, —(CR_{d}Rₑ)ₜ(C₃-C₁₀ cycloalkyl), —(CR_{d}Rₑ)ₜ(aryl), —(CR_{d}Rₑ)ₜ(4-10 membered heterocycloalkyl), and
—(CR_{d}Rₑ)ₜ(4-10 membered heteroaryl), wherein q and t are each independently an integer from 0 to 5, R_{d} and Rₑ are independently H or C₁-C₆ alkyl, wherein 1 or 2 ring carbon atoms of the heterocyclic and heteroaryl moieties of the foregoing R₁₀ groups are unsubstituted or substituted with an oxo (═O) moiety, and the alkyl, alkenyl, alkynyl, aryl and heterocyclic and heteroaryl moieties of the foregoing R₁₀ groups are unsubstituted or substituted with 1 to 3 substituents independently selected from halo, cyano, nitro, trifluoromethyl, trifluoromethoxy, azido, —OR_{b}, —C(O)R_{b}, —C(O)OR_{b}, —NR_{b}C(O)R_{c}, —C(O)NR_{b}R_{c}, —NR_{b}R_{c}, —NR_{b}OR_{c}, C₁-C₆ alkyl, C₂-C₆ alkenyl, C₂-C₆ alkynyl, —(CR_{d}Rₑ)ₜ(C₃-C₁₀ cycloalkyl), —(CR_{d}Rₑ)ₜ(aryl), —(CR_{d}Rₑ)ₜ(4-10 membered heterocycloalkyl), and
—(CR_{d}Rₑ)ₜ(4-10 membered heteroaryl);
and wherein any of the above-mentioned substituents comprising a CH₃ (methyl), CH₂ (methylene), or CH (methane) group which is not attached to a halogeno, SO or SO₂ group or to a N, O, or S is unsubstituted or substituted with a substituent from the group selected from hydroxyl, halo, C₁-C₄ alkyl, C₁-C₄ alkoxy and —NR_{d}Rₑ wherein R_{d} and Rₑ are as defined above;
or a pharmaceutically acceptable salt of a compound of the Formula (I), or a multimer, prodrug or pharmaceutically active metabolite of a compound of the Formula (I) or pharmaceutically acceptable salt thereof.

4. A compound of Formula (IV): 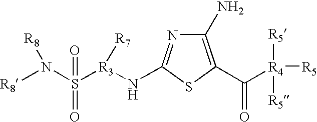
wherein:
R₃ is a monocycle selected from the group consisting of C₃-C₁₀ cycloalkyl, 3-10 membered heterocycloalkyl, aryl and 3-10 membered heteroaryl;
R₄ is a moiety selected from the group consisting of substituted or unsubstituted C₂-C₁₄ alkyl, C₃-C₁₀ cycloalkyl, 3-10 membered heterocycloalkyl, aryl and 3-10 membered heteroaryl;
R₅ is a moiety selected from the group consisting of hydroxyl, halo, C₁-C₁₄ alkyl, C₁-C₁₄alkoxyl, acyl, amide and nitro;
R₅′ and R₅″ are independently selected from hydrogen, hydroxyl, halo, C₁₋₁₄ alkyl, C₁-C₁₄ alkoxyl, acyl, amide, amino, acetamido and nitro;
R₇ is a moiety selected from the group consisting of hydrogen, hydroxyl, halo, C₁-C₁₄ alkyl, C₁-C₁₄ alkoxyl, acyl, amide and nitro;
R₈ is hydrogen, C₁-C₃ alkyl, C₃-C₁₀ cycloalkyl, or C₁-C₁₄ alkoxyl;
R_{8′} is an C₃₋₁₄ alkyl, 2-9 membered heteroalkyl, acyl, C₁₋₃ alkyl-nitrile, C₁₋₃ alkyl-carboxamide, C₁₋₄ alkyl-heterocycloalkyl, C₁₋₄ alkyl-aryl, CIA alkyl-heteroaryl, C₃-C₁₀ cycloalkyl, 3-10 membered heterocycloalkyl, aryl or 3-10 membered heteroaryl, or together with R₈ cyclizes to form a C₃-C₁₀ cycloalkyl, 3-10 membered heterocycloalkyl, aryl or 3-10 membered heteroaryl, with the proviso that R₆ is not 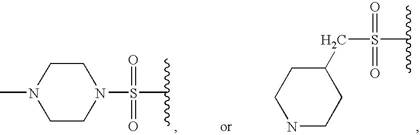
and wherein R is unsubstituted or substituted with 1 to 4 R₁₀ groups;
R₉ is hydrogen, or a moiety selected from the group consisting of an C₁₋₉ alkyl, C₂₋₉ alkenyl, 2-9 membered heteroalkenyl, C₁₋₉ alkylamide, C₁₋₉ alkyl-carboxamide, 2-9 membered heteroalkyl, C₁₋₄ alkyl-cycloalkyl, C₁₋₄ alkyl-heterocycloalkyl, C₁₋₄ alkyl-aryl, C₁₋₄ alkyl-heteroaryl, C₃-C₁₀ cycloalkyl, 3-10 membered heterocycloalkyl, aryl and 3-10 membered heteroaryl, with the proviso that R₆ is not 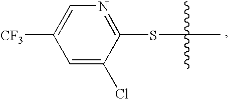
wherein R₉ is unsubstituted or substituted with 1 to 4 R₁₀ groups;
R₇ is a moiety selected from the group consisting of hydrogen, hydroxyl, halo, C₁-C₁₄ alkyl, C₁-C₁₄ alkoxyl, acyl, amide and nitro;
wherein each R₁₀ is independently selected from halo, cyano, nitro, trifluoromethoxy, trifluoromethyl, azido, hydroxyl, C₁-C₆ alkoxyl, C₁-C₁₀ alkyl, C₂-C₆ alkenyl, C₂-C₆ alkynyl, —C(O)R₈, —C(O)OR_{b}, —OC(O)R_{b}, —NR_{b}C(O)R_{c}, —C(O)NR_{b}R_{c}, —NR_{b}R_{c}, —NR_{b}OR_{c}, —S(O)ⱼ(C₁-C₆ alkyl) wherein j is an integer from 0 to 2, —(CR_{d}Rₑ)ₜ(C₃-C₁₀ cycloalkyl), —(CR_{d}Rₑ)ₜ(aryl), —(CR_{d}Rₑ)ₜ(4-10 membered heterocycloalkyl), —(CR_{d}Rₑ)ₜ(4-10 membered heteroaryl), —(CR_{d}Rₑ)_{q}C(O)(CR_{d}Rₑ)ₜ(C₃-C₁₀ cycloalkyl), —(C R_{d}Rₑ)_{q}C(O)(CR_{d}Rₑ)ₜ(aryl), —(CR_{d}Rₑ)_{q}C(O)(CR_{d}Rₑ)ₜ(4-10 membered heterocycloalkyl), —(CR_{d}Rₑ)_{q}C(O)(CR_{d}Rₑ)ₜ(4-10 membered heteroaryl), —(CR_{d}Rₑ)ₜO(CR_{d}Rₑ)_{q}(C₃-C₁₀ cycloalkyl), —(CR_{d}Rₑ)ₜO(CR_{d}Rₑ)_{q}(aryl), —(CR_{d}Rₑ)ₜO(CR_{d}Rₑ)_{q}(4-10 membered heterocycloalkyl), —(CR_{d}Rₑ)ₜO(CR_{d}Rₑ)_{q}(4-10 membered heteroaryl), —(CR_{d}Rₑ)_{q}SO₂(CR_{d}Rₑ)ₜ(C₃-C₁₀ cycloalkyl), —(CR_{d}Rₑ)_{q}SO₂(CR_{d}Rₑ)ₜ(aryl), and —(CR_{d}Rₑ)_{q}SO₂(CR_{d}Rₑ)ₜ(4-10 membered heterocycloalkyl), —(CR_{d}Rₑ)_{q}SO₂(CR_{d}Rₑ)ₜ(4-10 membered heteroaryl), wherein Rₐ is selected from the group consisting of halo, hydroxyl, —NR_{d}Rₑ C₁-C₆ alkyl, trifluoromethyl, C₁-C₆ alkoxyl, and trifluoromethoxy, R_{b} and R_{c} are independently selected from H, C₁-C₆ alkyl, —(CR_{d}Rₑ)ₜ(C₃-C₁₀ cycloalkyl), —(CR_{d}Rₑ)ₜ(aryl), —(CR_{d}Rₑ)ₜ(4-10 membered heterocycloalkyl), and
—(CR_{d}Rₑ)ₜ(4-10 membered heteroaryl), wherein q and t are each independently an integer from 0 to 5, R_{d} and Rₑ are independently H or C₁-C₆ alkyl, wherein 1 or 2 ring carbon atoms of the heterocyclic and heteroaryl moieties of the foregoing R₁₀ groups are unsubstituted or substituted with an oxo (═O) moiety, and the alkyl, alkenyl, alkynyl, aryl and heterocyclic and heteroaryl moieties of the foregoing R₁₀ groups are unsubstituted or substituted with 1 to 3 substituents independently selected from halo, cyano, nitro, trifluoromethyl, trifluoromethoxy, azido, —OR_{b}, —C(O)R_{b}, —C(O)OR_{b}, —NR_{b}C(O)R_{c}, —C(O)NR_{b}R_{c}, —NR_{b}R_{c}, —NR_{b}OR_{c}, C₁-C₆ alkyl, C₂-C₆ alkenyl, C₂-C₆ alkynyl, —(CR_{d}Rₑ)ₜ(C₃-C₁₀ cycloalkyl), —(CR_{d}Rₑ)ₜ(aryl), —(CR_{d}Rₑ)ₜ(4-10 membered heterocycloalkyl), and
—(CR_{d}Rₑ)ₜ(4-10 membered heteroaryl);
and wherein any of the above-mentioned substituents comprising a CH₃ (methyl), CH₂ (methylene), or CH (methane) group which is not attached to a halogeno, SO or SO₂ group or to a N, O, or S is unsubstituted or substituted with a substituent from the group selected from hydroxyl, halo, C₁-C₄ alkyl, C₁-C₄ alkoxy and —NR_{d}Rₑ wherein R_{d} and Rₑ are as defined above;
or a pharmaceutically acceptable salt of a compound of the Formula (I), or a multimer, prodrug or pharmaceutically active metabolite of a compound of the Formula (I) or pharmaceutically acceptable salt thereof.

5. A compound according to claim 1 having the structure: 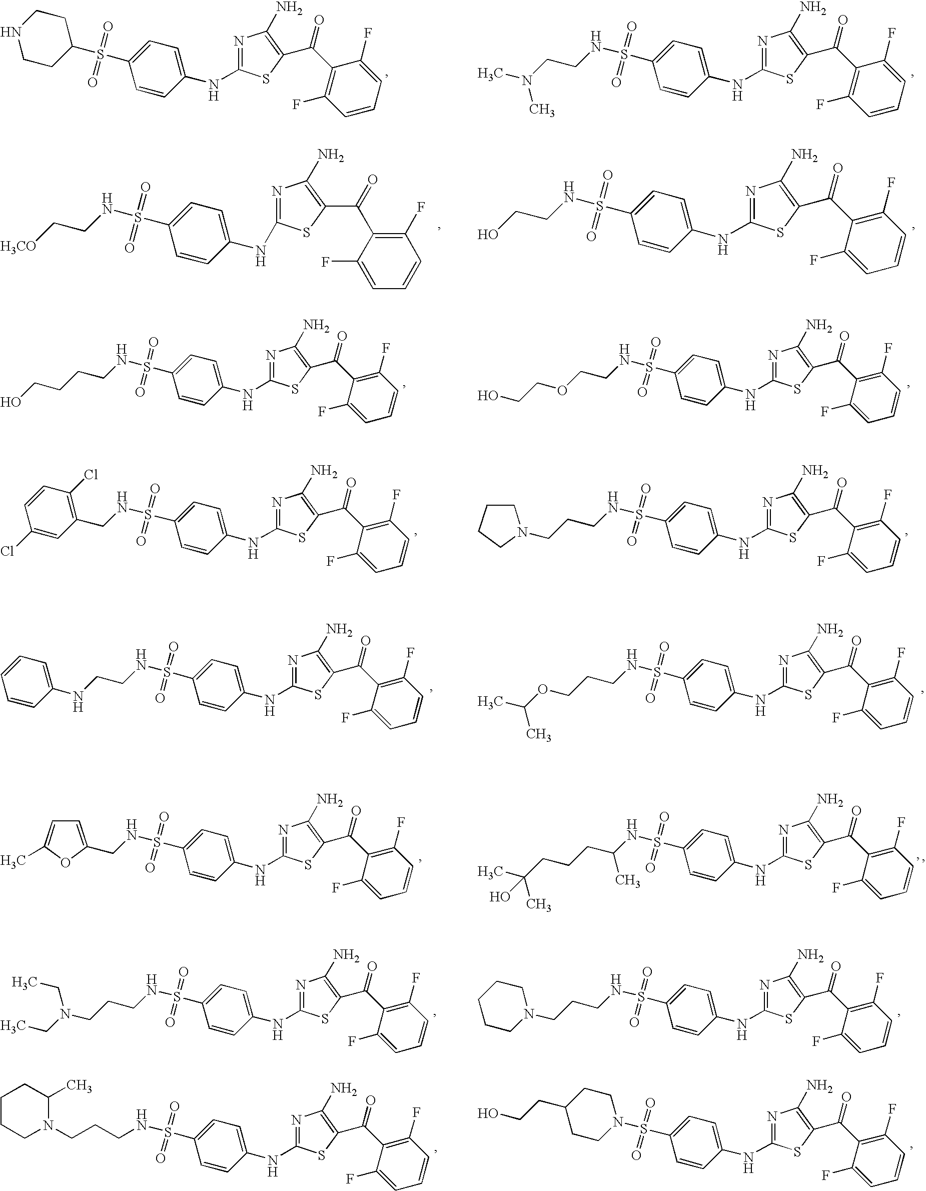 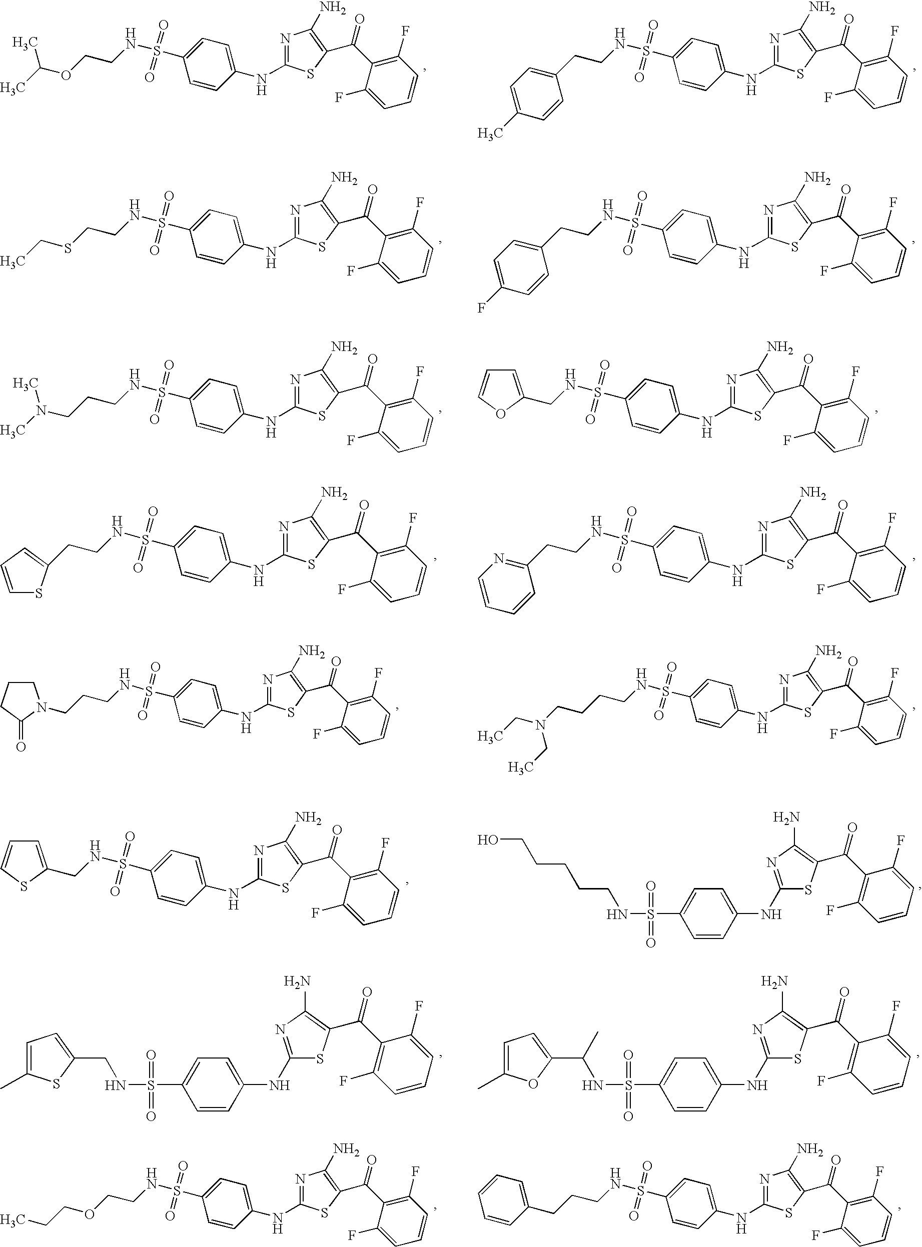 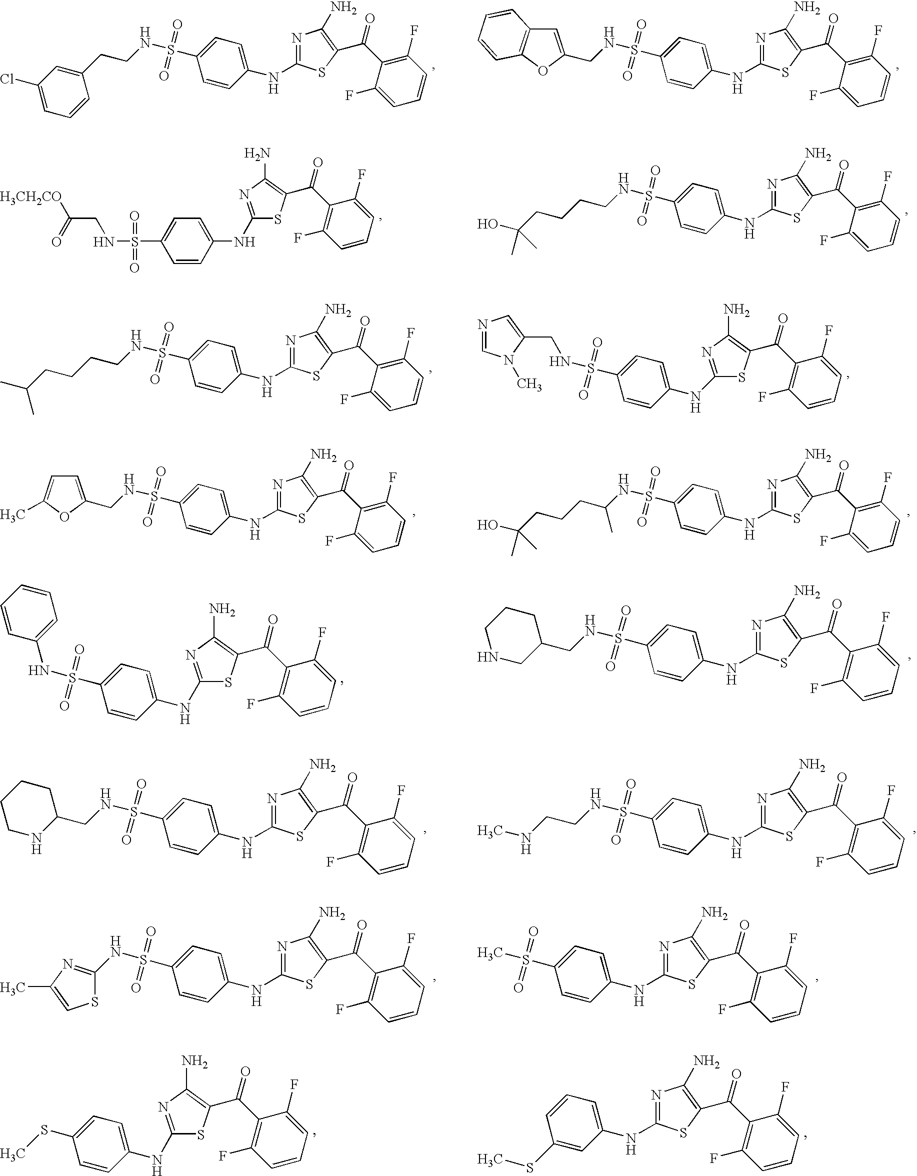 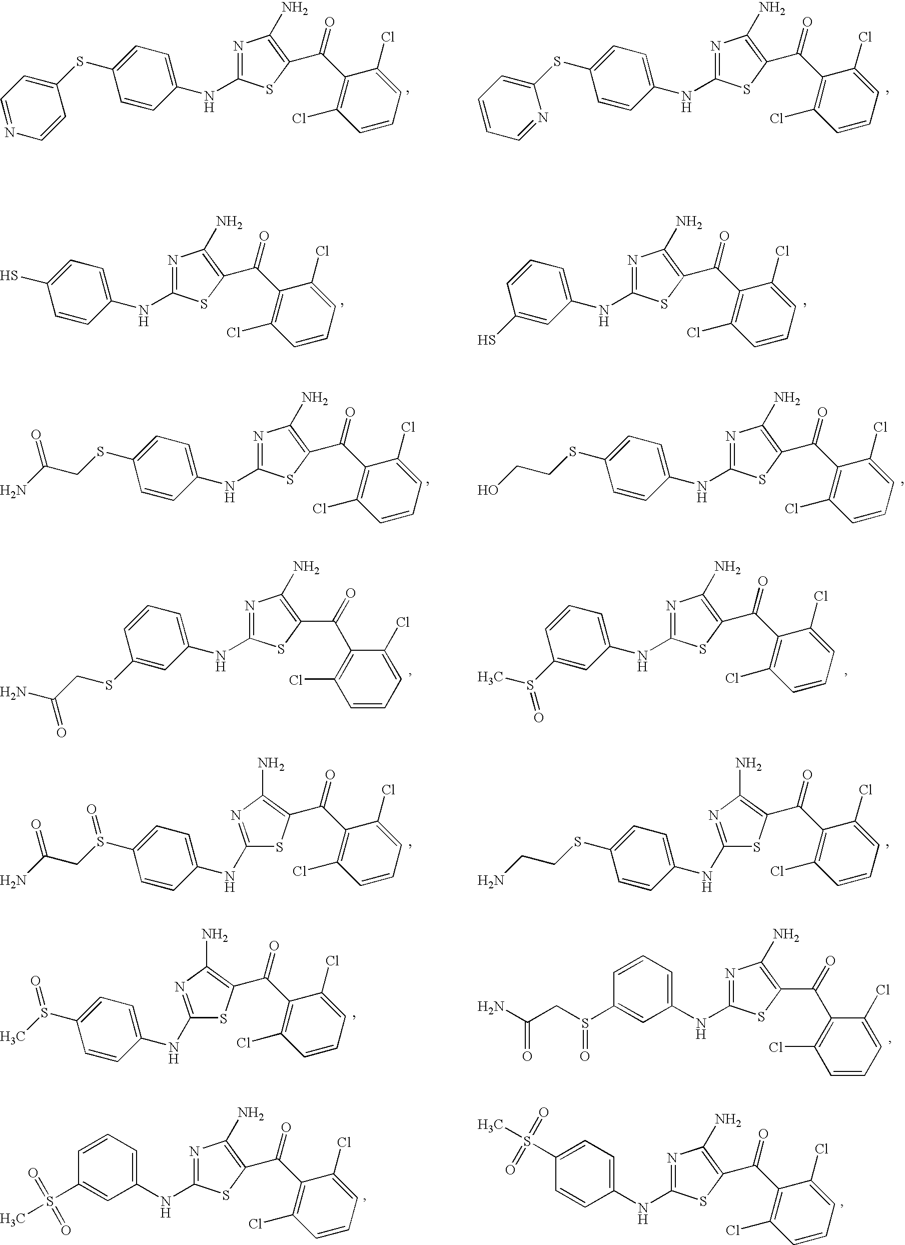 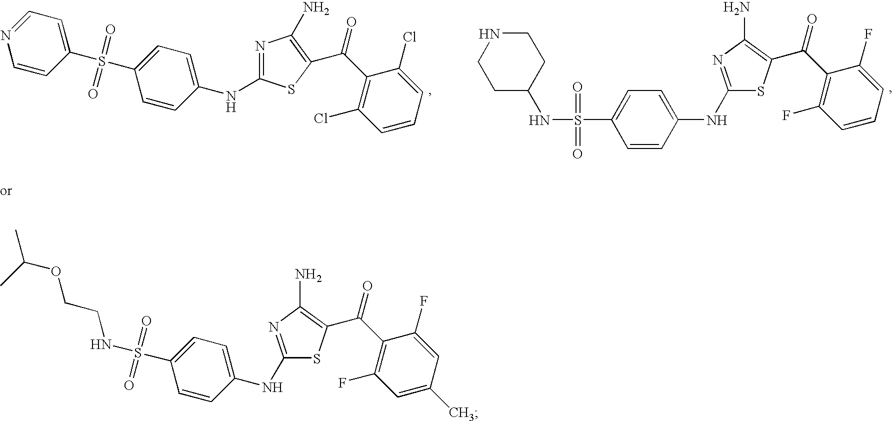
and multimers, pharmaceutically acceptable salts, prodrugs, and active metabolites thereof.

6. A pharmaceutical composition comprising an effective amount of an agent to inhibit cellular proliferation and a pharmaceutically acceptable carrier, said agent being selected from the group consisting of compounds, multimers, pharmaceutically acceptable salts, prodrugs, and active metabolites as defined in any of claims 1, 2, 3, and 4.

7. A method of inhibiting a CDK selected from CDK2, CDK4, CDK6 or CDK complex, comprising administering an effective amount of a compound, multimer, pharmaceutically acceptable salt, prodrug, or active metabolite as defined in any of claims 1, 2, 3, and 4.

8. A method of treating cellular proliferative diseases, comprising administering an effective amount of a compound, multimer, pharmaceutically acceptable salt, prodrug, or active metabolite as defined in any of claims 1, 2, 3 and 4.

9. A method according to claim 8, wherein the disease is cancer, autoimmune disease, viral disease, fungal disease, neurodegenerative disorder or cardiovascular disease.
